(19) United States
(12) Patent Application Publication
(10) Pub. No.: US 2023/0416239 A1
(43) Pub. Date: Dec. 28, 2023

(54) COMPOUNDS AND METHODS FOR TREATING A CYTOKINE-MEDIATED DISEASE

(71) Applicants: CONSERVATOIRE NATIONAL DES ARTS ET MÉTIERS, Paris (FR); PEPTINOV, Paris (FR)

(72) Inventors: Hadley MOUHSINE, Paris (FR); Aïda MASCRET, Paris (FR); Marc PORT, Deuil la Barre (FR); Jean-François ZAGURY, Le Vésinet (FR); Matthieu MONTES, Paris (FR); Maite SYLLA, Paris (FR); Christophe RICCO, Le Perreux sur Marne (FR); Damien CABRERA, Paris (FR); Mohamed BENCHEKROUN, Paris (FR)

(21) Appl. No.: 18/036,860

(22) PCT Filed: Nov. 12, 2021
(86) PCT No.: PCT/EP2021/081601

§ 371 Date: May 12, 2023

(30) Foreign Application Priority Data
Nov. 12, 2020 (EP) 20306371.4

(51) Int. Cl. C07D 413/14; C07D 401/12; C07D 401/14; C07D 471/04; C07D 403/12

(52) U.S. Cl. CPC C07D 413/14; C07D 401/12; C07D 401/14; C07D 471/04; C07D 403/12

(57) ABSTRACT

The present invention relates to a compound of the following formula (I):

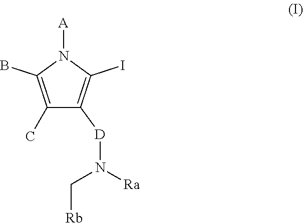

in particular for use use in the diagnostic, prevention or treatment of a cytokine-mediated disease in an individual.

### FIELD OF THE INVENTION

The present invention relates to compounds and pharmaceutical compositions useful in the diagnostic, prevention or treatment of a cytokine-mediated disease in an individual.

### BACKGROUND OF THE INVENTION

Chronic inflammatory diseases are the most significant cause of death in the world. The World Health Organization (WHO) ranks chronic diseases as the greatest threat to human health. The prevalence of diseases associated with chronic inflammation is anticipated to increase persistently for the next 30 years in the United States. In 2000, nearly 125 million Americans were living with chronic conditions and 61 million (21%) had more than one.

Some of the common signs and symptoms that develop during chronic inflammation are body pain, arthralgia, myalgia, chronic fatigue, insomnia, depression, anxiety, mood disorders, gastrointestinal complications like constipation, diarrhea, and acid reflux, weight gain or weight loss and frequent infections.

By way of example of chronic inflammation-mediated diseases it is possible to cite rheumatoid Arthritis (RA), psoriasis or Crohn disease.

Rheumatoid Arthritis affects approximately 17.6 million people worldwide. It is a disabling and painful chronic inflammatory disease which typically affects the small joints in hands and feet and potentially other body parts and organs. As the disease progresses, symptoms may spread to the wrists, knees, ankles, elbows, hips, and shoulders. As a result, RA causes pain, inflammation, and eventually joint damage and malformation.

Tumor Necrosis Factor alpha (TNFa) is a homotrimeric cytokine of the immune system which overproduction has been associated with several chronic inflammatory diseases such as RA, Crohn's disease or psoriasis. The binding of TNFa homotrimer to its receptors TNFRI and TNFRII leads to the activation of transcription factors involved in cell survival and inflammatory responses. TNFa is produced as a type II transmembrane protein (tmTNFa), which can be proteolytically cleaved by TNFa converting enzyme into soluble TNFa (sTNFa).

Recently, molecules targeting TNFa have been developed in order to alleviate inflammatory diseases. By way of example, anti-TNFa antibodies such as infliximab (Lipsky et al. (2000) N. Engl J Med., 30: 1594-602) and golimumab (Frampton (2017) BioDrugs, 31:263-274) work by neutralizing the binding of sTNFa to its receptors and by inducing antibody-dependent cytotoxicity by directly binding to tmTNFa.

However, the use of these marketed biotherapies in inflammatory conditions exhibit several drawbacks due to their limited tissue penetration, their administration route (subcutaneously or intravenous), their high production cost and high prices, their immunogenicity and treatment resistance due to immune reactions, and the increased risk of opportunistic infections.

Besides, small molecule inhibitors of TNFa have also been designed, such as SPD304 (He et al. (2005) Science 310:1022-1025) which prevents binding of TNFa to the TNF Receptor 1 (TNFR1) by promoting dissociation of the TNF trimers. However, SPD304 is potentially toxic.

Accordingly, there is a need for alternative therapeutic strategies which would treat chronic inflammation-mediated diseases and circumvent the above cited obstacles.

### SUMMARY OF THE INVENTION

The present invention arises from the unexpected findings, by the inventors, that newly synthesized compounds had a cytokine-inhibitory activity.

Thus, the present invention relates to a compound of the following formula (I):

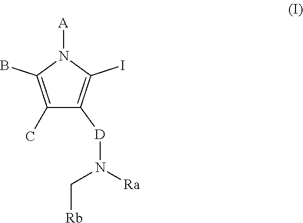

wherein:

### A is:

a hydrogen atom;
a linear or branched C₁₋₁₀ alkyl or heteroalkyl group, C₂₋₁₁ alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of: a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH, —R₁COR₁′, —R₁COOR₁′, —R₁CONHR₁′, —R₁NHCOR₁′ wherein R₁ is selected form the group consisting of a covalent bond, or a C₁₋₅ alkyl, heteroalkyl, alkenyl or heteroalkenyl group, and R₁′ is selected from the group consisting of a hydrogen atom, a C₁₋₅ alkyl, heteroalkyl or haloalkyl group, a C₂₋₆ alkenyl, heteroalkenyl or haloalkenyl group, a C₃₋₁₂ cycloalkyl, heterocycloalkyl, aryl, or heteroaryl group, —NO₂, and —SO₃H; or
a SO₂A′ group wherein A′ is selected from the group consisting of a C₁₋₅ alkyl, heteroalkyl, alkenyl or heteroalkenyl group and a C₃₋₁₂ cycloalkyl, cycloalkenyl, heterocycloalkyl, heterocycloalkenyl, aryl or heteroaryl group which may be substituted by at least one group selected from the group consisting of:
a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH, —R₁COR₁′, —R₁COOR₁′, —R₁CONHR₁′, —R₁NHCOR₁′ wherein R₁ and R₁′ are as defined above;
   B and C, which may be the same or different, independently represent:
a hydrogen atom; or
a linear or branched C₁₋₁₀ alkyl or heteroalkyl group, C₂₋₁₁ alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of: a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH, —R₁COR₁′, —R₁COOR₁′, —R₁CONHR₁′, —R₁NHCOR₁′ wherein R₁ and R₁′ are as defined above; or
   B and C are joined such that when taken together with the carbon atoms to which they are attached they form a C₃₋₁₂ cycloalkyl, cycloalkenyl heterocycloalkyl, heterocycloalkenyl, aryl or heteroaryl ring which may be substituted by at least one group selected from the group consisting of:
   a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, a heteroatom selected form N, O or S, —COOH, —R₁COR₁′, —R₁COOR₁′, —R₁CONHR₁′, —R₁NHCOR₁′ wherein R₁ and R₁′ are as defined above;
   D is —CH₂, —C(O), —NHC(O)—, SO₂, or —NHSO₂;
   Ra is R₂—NR₃R₄ wherein:
R₂ is a linear or branched C₁₋₅ alkyl or heteroalkyl group, or C₂₋₆ alkenyl, or heteroalkenyl group;
R₃ is:
   a hydrogen atom, or
   a linear of branched C₁₋₅ alkyl or heteroalkyl group, a C₂₋₆ alkenyl or heteroalkenyl group, or a C₃₋₁₂ cycloalkyl, cycloalkenyl, heterocycloalkyl, heterocycloalkenyl, cycloalkylalkyl, cycloalkylalkenyl, heterocycloalkylalkyl, heterocycloalkylalkenyl, aryl, heteroaryl, arylalkyl, or heteroarylalkyl group, each of which may be substituted by at least one group selected from the group consisting of a trifluoromethyl, a C₁₋₅ alkyl or alkoxy group, a C₂₋₆ alkenyl group, a halogen atom including F, Cl, Br, and I, a hydroxy group, an amino group, and —COOH;
   R₄ is:
      a hydrogen atom;
      a linear or branched C₁₋₁₀ alkyl or heteroalkyl group, C₂₋₁₁ alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of:
   a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH;
   —SO₂NR₅R₆ or —C(O)NR₅R₆ wherein R₅ and R₆, which may be the same or different, independently represent a hydrogen atom, a C₁₋₅ linear of branched alkyl, alkenyl, heteroalkyl or heteroalkenyl, group, or R₅ and R₆ are joined such that when taken together with the nitrogen atom to which they are attached they form a C₃₋₁₂ cycloalkyl, cycloalkenyl, heterocycloalkyl, heterocycloalkenyl, aryl or heteroaryl ring;
   —SO₂R₇, wherein R₇ is a hydrogen atom, a C₁₋₅ linear of branched alkyl, alkenyl, heteroalkyl or heteroalkenyl group; a C₃₋₁₂ cycloalkyl, cycloalkenyl, heterocycloalkyl, heterocycloalkenyl, aryl or heteroaryl group which may be substituted by a C₁₋₅ alkyl group, a halogen atom including F, Cl, Br, and I, an amino group, or —NO₂; or
   —C(O)R₇ wherein R₇ is as defined above; or
      Ra is a compound of the following formula:

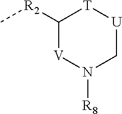

wherein
R₂ is as defined above;
T, U, V which are the same or different, independently represent —(CH₂)ₙ— wherein n is an integer from 1 to 3, or an heteroatom selected from the group consisting of O, N, and S;
R₈ is:
   a hydrogen atom; or
   a linear or branched C₁₋₁₀ alkyl or heteroalkyl group, C₂₋₁₁ alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of: a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH or R₁COR₁′, —R₁COOR₁′, —R₁CONHR₁′, —R₁NHCOR₁′ wherein R₁ and R₁′ are as defined above;

### Rb is

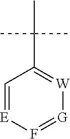

wherein
E, F, G and W which may be the same or different, independently represent —N—, —CH—, —N-oxide, —CNO₂, —CN(R₁″)₄ wherein R₁″ is a C₁₋₄ alkyl group, —CF—, —C¹⁸F or —C¹⁹F; or

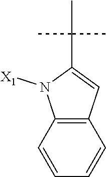

wherein
X₁ is selected from a SO₂A′ group with A′ as defined above or an aryl or heteroaryl group optionally substituted by at least one group selected from the group consisting of a trifluoromethyl and a C₁₋₅ alkyl group;
I is —H, —OH, —SH, —CF₃, a halogen atom including F, Cl, Br, and I, a linear or branched C₁₋₁₀ alkyl, heteroalkyl, or haloalkyl group, or C₂₋₁₁ alkylene, heteroalkylene, or haloalkylene group,
or a pharmaceutically acceptable salt or hydrate thereof.

In one embodiment of the present invention, the compound or pharmaceutically acceptable salt or hydrate thereof as defined above is for use in the diagnostic, prevention or treatment of a cytokine-mediated disease in an individual.

In one embodiment of the invention, the compound of formula (I), or the pharmaceutically acceptable salt or hydrate thereof, for use as defined above, is in combination with at least one additional active substance.

The present invention also relates to a pharmaceutical composition comprising as active substance at least one compound of formula (I) as defined above or a pharmaceutically acceptable salt or hydrate thereof, optionally in association with at least one pharmaceutically acceptable vehicle.

In one embodiment of the present invention, the pharmaceutical composition as defined above is for use in the diagnostic, prevention or treatment of a cytokine-mediated disease in an individual.

In one embodiment of the invention, the above defined pharmaceutical composition further comprises at least one additional active substance.

The present invention also relates to products comprising:
a compound of formula (I) or a pharmaceutical acceptable salt or hydrate thereof, as defined above,
at least one additional active substance.
as a combined preparation for simultaneous, separated or sequential use in the prevention or treatment of a cytokine-mediated disease in an individual.

The present invention also relates to a method for the diagnostic, prevention or treatment of a cytokine-mediated disease in an individual comprising administering to the individual a prophylactically or therapeutically effective amount of a compound of formula (I), or a pharmaceutically acceptable salt or hydrate thereof, as defined above.

In one embodiment of the invention, the method as defined above further comprises the administration of at least one additional active substance.

The present invention also relates to the use of a compound of formula (I), as defined above, for the manufacture of a medicament for the diagnostic, prevention or treatment of a cytokine-mediated disease in an individual.

In one embodiment, the medicament as defined above further comprises at least one additional active substance.

The present invention also relates to the synthesis intermediates for synthesizing the compounds according to the invention, in particular the synthesis intermediates 1 to 32b and 2.1. to 2.23. described in the following Examples.

### DETAILED DESCRIPTION OF THE INVENTION

### Definitions

As intended herein, the term “comprising” has the meaning of “including” or “containing”, which means that when an object “comprises” one or several elements, other elements than those mentioned may also be included in the object. In contrast, when an object is said to “consist of” one or several elements, the object is limited to the listed elements and cannot include other elements than those mentioned.

The expression “linear or branched alkyl” refers to linear or branched chain, saturated, hydrocarbon groups having preferably 1 to 10 carbon atoms. By way of example, the term “C₁₋₅ alkyl” refers to an alkyl group containing 1 to 5 carbon atoms. Example of linear or branched alkyl groups include, but are not limited to, methyl, ethyl, propyl, isopropyl, butyl, isobutyl, t-butyl, pentyl, heptyl, and octyl.

The term “alkenyl” refers to a linear or branched hydrocarbon group containing at least one carbon-carbon double bond and having preferably 1 to 10 carbon atoms. Byway of example, the term “C₁₋₆ alkenyl” refers to an alkenyl group containing 1 to 6 carbon atoms. Examples of alkenyl groups include, but are not limited to, ethenyl, propenyl, butenyl, 2-butenyl, pentenyl, hexenyl, heptenyl, octenyl, and nonenyl.

The term “heteroalkyl” refers to a linear or branched alkyl chain preferably having 2 to 10 carbon atoms in the chain, one or more of which has been replaced by a heteroatom selected from the group consisting of O, N, and S. Examples of heteroalkyl groups include, but are not limited to, methoxyethane, dimethyl ether, diethyl ether, and trimethylamine.

The term “haloalkyl” refers to a linear or branched alkyl chain comprising at least one halogen atom selected from the group consisting of F, Cl, Br, and I. By way of example, the term “C₁₋₅ haloalkyl” refers to a haloalkyl comprising 1 to 5 atoms and at least one of F, Cl, Br, and I. Examples of haloalkyl groups include, but are not limited to, fluoromethyl, chloromethyl, bromomethyl, iodomethyl, difluoromethyl, dichloromethyl, dibromomethyl, diiodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-difluoroethyl, 2,2-dichloroethyl, 2,2-dibromomethyl, 2-2-diiodomethyl, 3-fluoropropyl, 3-chloropropyl, 3-bromopropyl, 2,2,2-trifluoroethyl, and 1,1,2,2,2-pentafluoroethyl.

The term “cycloalkyl” refers to an unsubstituted or substituted cyclic hydrocarbon group having preferably 3 to 12 carbon atoms. It includes monocyclic, fused, and polycyclic rings. Examples of cycloalkyl groups include, but are not limited to, cyclopropyl, cyclobutyl, cyclopentyl, cyclohexyl, cycloheptyl, cyclooctyl, and decalin.

The term “cycloalkenyl” refers to an unsubstituted or substituted monocyclic or polycyclic hydrocarbon ring having preferably 4 to 12 carbon atoms and containing at least one carbon-carbon double bond. Examples of cycloalkenyl groups include, but are not limited to, cyclobutenyl, cyclopentenyl, cyclohexenyl, cycloheptenyl, and cyclobutenyl.

The term “heterocycloalkyl” refers to an unsubstituted or substituted monocyclic or polycyclic ring preferably having 3 to 12 carbon atoms in each ring, one or more of which has been replaced by a heteroatom selected from the group consisting of O, N, and S. Examples of heterocycloalkyl groups include, but are not limited to, oxirane, oxetane, hydrofurane, hydropyrane, thiirane, thiethane, hydrothiophene, hydrothiopyrene, aziridine, azetidine, pyrrolidine, piperidine, imidazole, oxazole, and piperazine.

The term “heterocycloalkenyl” refers to a heterocycloalkyl as defined above containing a least one carbon-carbon double bond.

The term “aryl” refers to a substituted or unsubstituted aromatic monocyclic or polycyclic hydrocarbon group having preferably 6 to 12 carbon atoms. Examples of aryl groups include, but are not limited to, phenyl, biphenyl, and naphthyl.

The term “heteroaryl” refers to an unsubstituted or substituted monocyclic or polycyclic aromatic ring having preferably 3 to 12 carbon atoms in each ring, one or more of which has been replaced by a heteroatom selected from the group consisting of O, N, and S. Examples of heteroaryl groups include, but are not limited to, furan, benzofurane, thiophene, pyrrole, pyrazole, pyridine, thiazole, imidazole, pyrimidine, indole, quinoline, isoquinoline oxazole, isoxazole, pyrazine, triazole, thiadiazole, tetrazole, and pyrazole.

The term “alkoxy” refers to an —O-alkyl group (alkoxyalkyl) or an —O-cycloalkyl group (alkoxycycloalyl), wherein alkyl and cycloalkyl are as defined above. The term “C₁₋₅ alkoxy” refers to an alkoxy group comprising 1 to 5 carbon atoms. Examples of —O-alkyl groups include, but are not limited to, methoxy, ethoxy, n-propyloxy, i-propyloxy, n-butyloxy, i-butyloxy, t-butyloxy. Examples of —O-cycloalkyl groups include, but are not limited to, —O-c-propyl, —O-c-butyl, —O-c-pentyl, and —O-c-hexyl.

The term “alkylamino” includes mono-alkylamino and di-alkylamino. Mono-alkylamino refers to a —NH-alkyl group in which alkyl is as defined above. Dialkylamino refers to a —N(alkyl)₂ group, wherein the alkyl groups may be the same or different and are each as defined above. The alkyl group is preferably a C₁₋₁₀ alkyl group.

The term “arylamino” includes mono-arylamino and di-arylamino. Mono-arylamino refers to a group of formula —NH-aryl, in which aryl is as defined above. Di-arylamino refers to a —N(aryl)₂ group in which each aryl may be the same or different and are as defined above.

The term “cycloalkylalkyl” refers to a linear or branched alkyl group as defined above substituted by a cycloalkyl group as defined above. By way of example, a C₁₋₅ cycloalkylalkyl group refers to a C₁₋₅ alkyl substituted by a cycloalkyl. Examples of cycloalkylalkyl groups include, but are not limited to, methyl, ethyl, propyl, i-propyl, n-butyl, i-butyl, t-butyl or pentyl, isopentyl, neopentyl, hexyl, heptyl, and octyl which is substituted with cyclopropyl, 2-methylcyclopropyl, cyclobutyl, cyclopentyl, and cyclohexyl.

The term “cycloalkylalkenyl” refers to a linear or branched alkenyl group as defined above substituted by a cycloalkyl group as defined above.

The term “heterocycloalkylalkyl” refers to a linear or branched alkyl group as defined above substituted by a heterocycloalkyl group as defined above. A C₁₋₅ heterocycloalkylalkyl refers to a C₁₋₅ alkyl substituted by a heterocycle.

The term “heterocycloalkylalkenyl” refers to a linear or branched alkenyl group as defined above substituted by a heterocycloalkyl group as defined above.

The term “arylalkyl” refers to a linear or branched alkyl group as defined above substituted by an aryl group as defined above. A C₁₋₅ arylalkyl refers to a C₁₋₅ alkyl substituted by an aryl group. Examples of arylalkyl groups include, but are not limited to benzyl.

The term “heteroarylalkyl” refers to a linear or branched alkyl group as defined above substituted by a heteroaryl group as defined above. A C₁₋₅ heteroarylalkyl group refers to a C₁₋₅ alkyl substituted by a heteroaryl.

### Compound of Formula (I)

In one embodiment, the compound of formula (I) or the pharmaceutically acceptable salt or hydrate thereof according to the invention is of the following formula (II):

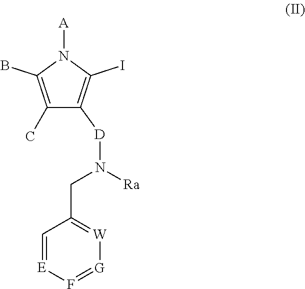

wherein
A, I, B, C, D, Ra, E, F, G and W are as defined above.

Preferably, the compound of formula (II) or the pharmaceutically acceptable salt or hydrate thereof is of the following formula (III):

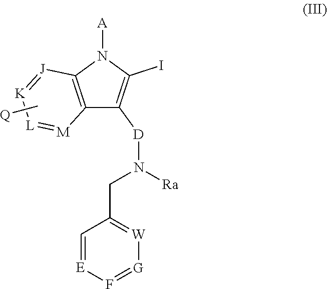

wherein
A, I, D, Ra, E, F, G and W are as defined above; and
J, K, L, M, which are the same or different, independently represent —N—, —O—, —S—, or —CH—;

### Q is:

a hydrogen atom; or
a linear or branched C₁₋₁₀ alkyl or heteroalkyl group, C₂₋₁₁ alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of: a trifluoromethyl, a C₁₋₅ alkyl, alkoxy or alkylamino group, a C₂₋₆ alkenyl, alkenyloxy or alkenyamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH, R₁COR₁′, —R₁COOR₁′, —R₁CONHR₁′, —R₁NHCOR₁′ wherein R₁ and R₁′ are as defined above.

In one embodiment, the compound of formula (I) or the pharmaceutically acceptable salt thereof is of the following formula (IV):

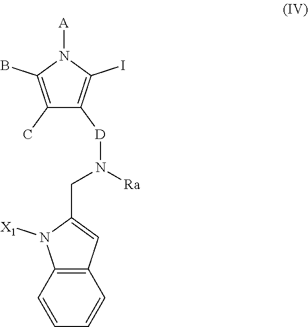

wherein
A, I, B, C, D, Ra and X₁ are as defined above.

Preferably, the compound of formula (IV) or the pharmaceutically acceptable salt thereof as defined above is of the following formula (V):

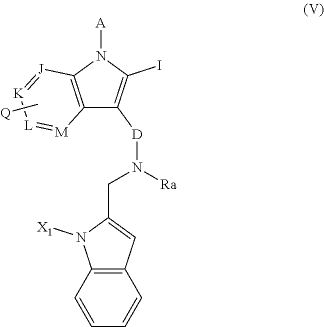

wherein
A, I, J, K, Q, L, M, D, Ra and X₁ are as defined above. Preferably, X₁ is SO₂A′ with A′ selected from an aryl group.

In an embodiment of the compound of formula (I), in particular the compound of formula (II), (III), (IV) or (V), or the pharmaceutically acceptable salt or hydrate thereof, as defined above, Ra is selected from the group consisting of:

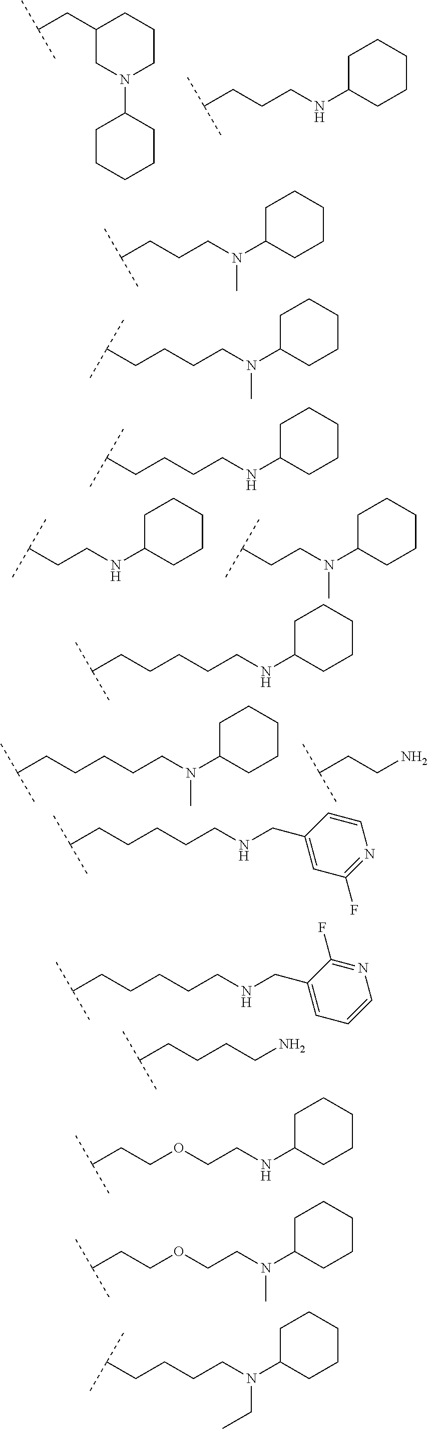

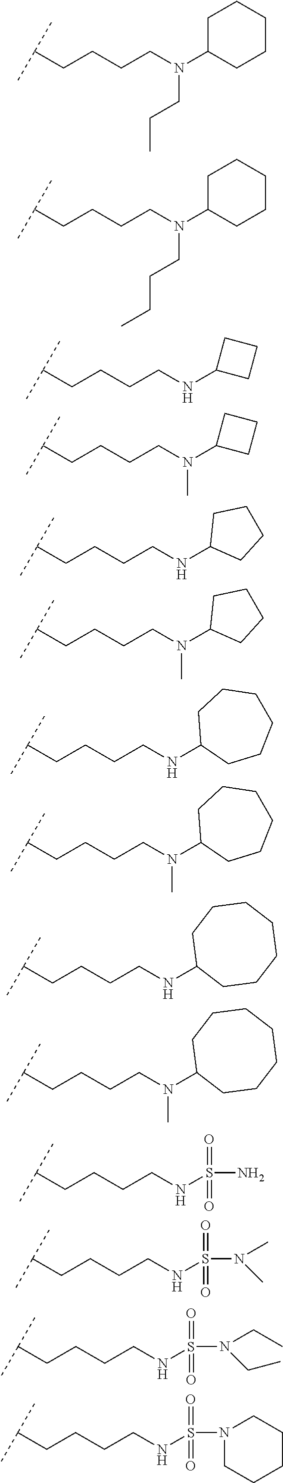

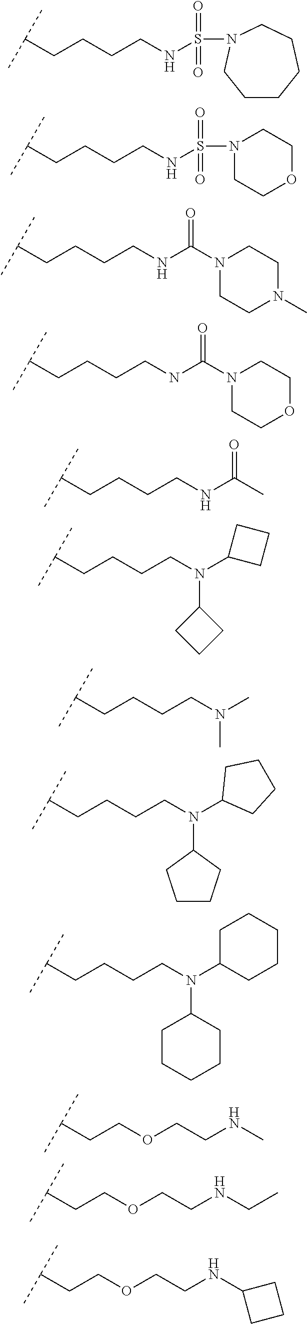

In an embodiment of the compound of formula (I), in particular the compound of formula (II), (III), (IV) or (V), or the pharmaceutically acceptable salt or hydrate thereof, according to the invention, A is selected from:
a linear or branched alkyl, heteroalkyl, or haloalkyl group having 1 to 10 carbon atoms; a cycloalkyl, heterocycloalkyl, aryl or heteroaryl group having 2 to 12 carbon atoms, each of which may be substituted by at least one group selected from the group consisting of a trifluoromethyl, or a C₁₋₅ alkyl group, or
a SO₂A′ group wherein A′ is selected from the group consisting of a cycloalkyl, heterocycloalkyl, aryl or heteroaryl group having 2 to 12 carbon atoms, which may be substituted by at least one group selected from the group consisting of a trifluoromethyl, or a C₁₋₅ alkyl group.

In an embodiment of the compound of formula (I), in particular the compound of formula (II), (III), (IV) or (V), or the pharmaceutically acceptable salt or hydrate thereof, according to the invention, A is selected from:
a linear or branched alkyl, heteroalkyl group having 1 to 5 carbon atoms; a cycloalkyl, heterocycloalkyl, aryl or heteroaryl group having 2 to 6 carbon atoms, each of which may be substituted by at least one group selected from the group consisting of a trifluoromethyl, or a C₁₋₅ alkyl group, or
a SO₂A′ group wherein A′ is selected from the group consisting of a cycloalkyl, heterocycloalkyl, aryl or heteroaryl group having 2 to 6 carbon atoms, which may be substituted by at least one group selected from the group consisting of a trifluoromethyl, or a C₁₋₅ alkyl group.

Preferably, in the compound of formula (I), in particular the compound of formula (II), (III), (IV) or (V), or the pharmaceutically acceptable salt thereof according to the invention, A is selected from:
an aryl group substituted by a trifluoromethyl, or
a SO₂A′ group wherein A′ is an aryl group

Preferably, in the compound of formula (I), in particular the compound of formula (II), (III), (IV) or (V), or the pharmaceutically acceptable salt or hydrate thereof as defined above I is a hydrogen atom.

Preferably, in the compound of formula (I), in particular the compound of formulae (II) to (V), or the pharmaceutically acceptable salt or hydrate thereof as defined above D is CH₂.

More preferably, the compound of formula (I), in particular the compound of formula (II), (III), (IV) or (V), or the pharmaceutically acceptable salt or hydrate thereof according to the invention is represented by the following formulae:

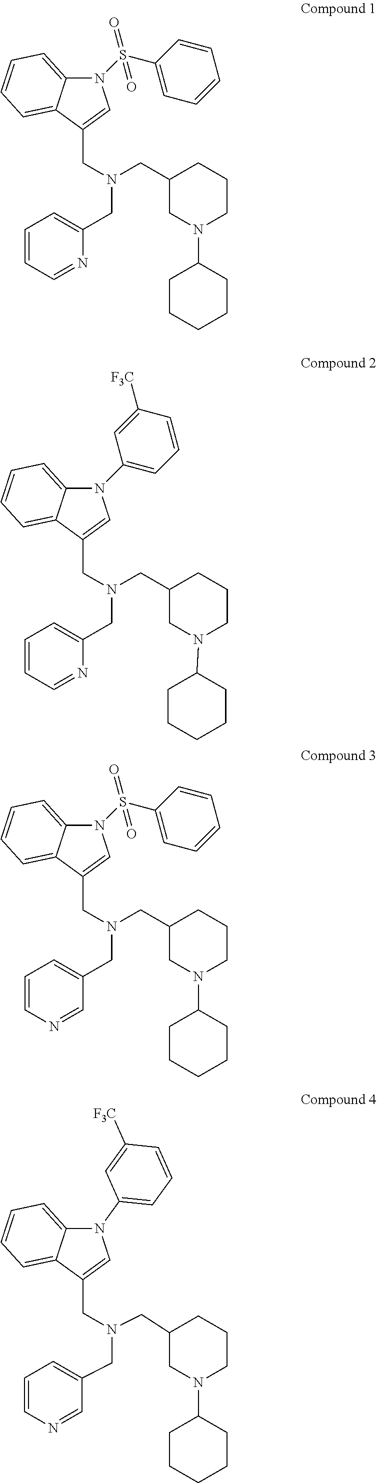 [SMILES: O=S(=O)(c1ccccc1)n1cc(CN(Cc2ccccn2)CC2CCCN(C3CCCCC3)C2)c2ccccc21 ; FC(F)(F)c1cccc(-n2cc(CN(Cc3ccccn3)CC3CCCN(C4CCCCC4)C3)c3ccccc32)c1 ; O=S(=O)(c1ccccc1)n1cc(CN(Cc2cccnc2)CC2CCCN(C3CCCCC3)C2)c2ccccc21 ; FC(F)(F)c1cccc(-n2cc(CN(Cc3cccnc3)CC3CCCN(C4CCCCC4)C3)c3ccccc32)c1]

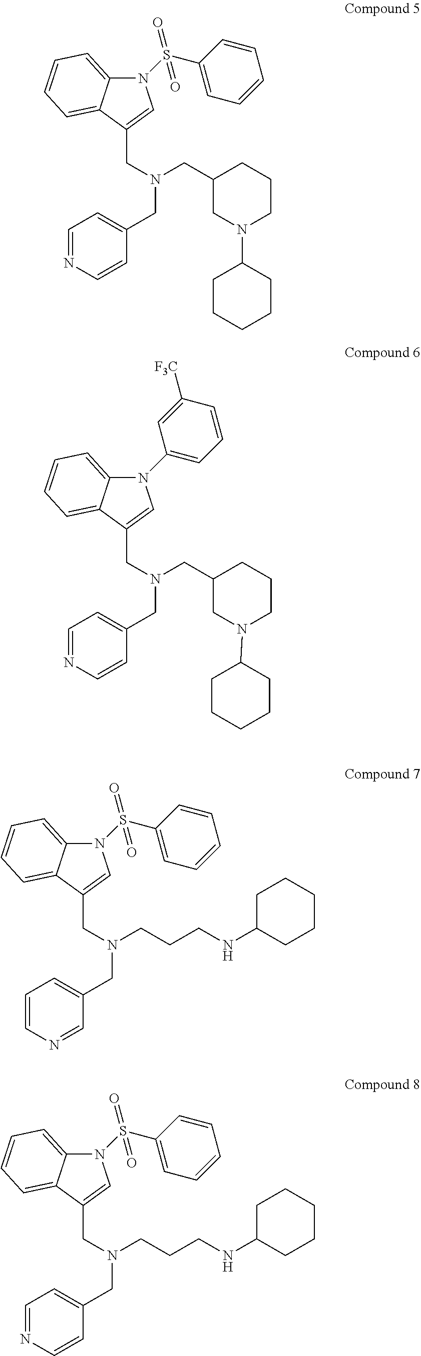 [SMILES: O=S(=O)(c1ccccc1)n1cc(CN(Cc2ccncc2)CC2CCCN(C3CCCCC3)C2)c2ccccc21 ; FC(F)(F)c1cccc(-n2cc(CN(Cc3ccncc3)CC3CCCN(C4CCCCC4)C3)c3ccccc32)c1 ; O=S(=O)(c1ccccc1)n1cc(CN(CCCNC2CCCCC2)Cc2cccnc2)c2ccccc21 ; O=S(=O)(c1ccccc1)n1cc(CN(CCCNC2CCCCC2)Cc2ccncc2)c2ccccc21]

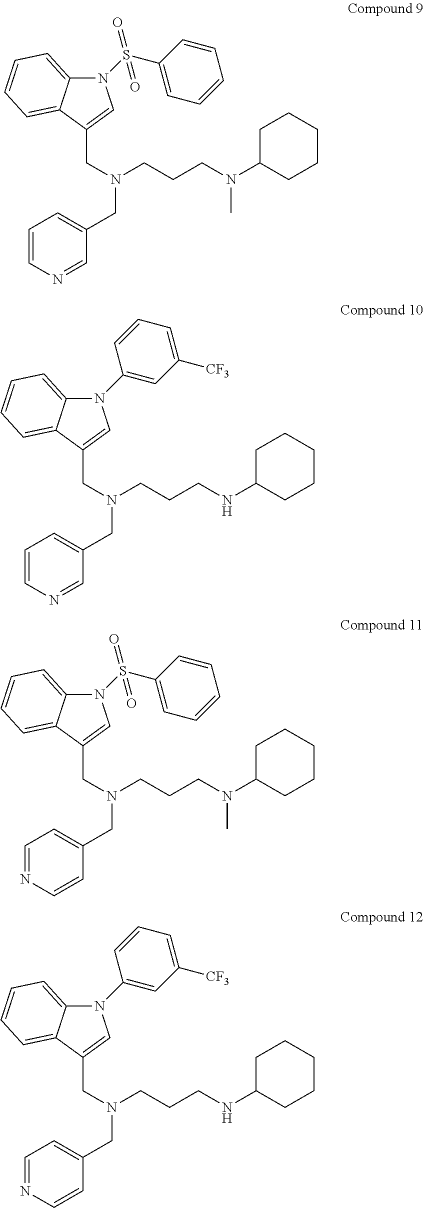 [SMILES: CN(CCCN(Cc1cccnc1)Cc1cn(S(=O)(=O)c2ccccc2)c2ccccc12)C1CCCCC1 ; FC(F)(F)c1cccc(-n2cc(CN(CCCNC3CCCCC3)Cc3cccnc3)c3ccccc32)c1 ; CN(CCCN(Cc1ccncc1)Cc1cn(S(=O)(=O)c2ccccc2)c2ccccc12)C1CCCCC1 ; FC(F)(F)c1cccc(-n2cc(CN(CCCNC3CCCCC3)Cc3ccncc3)c3ccccc32)c1]

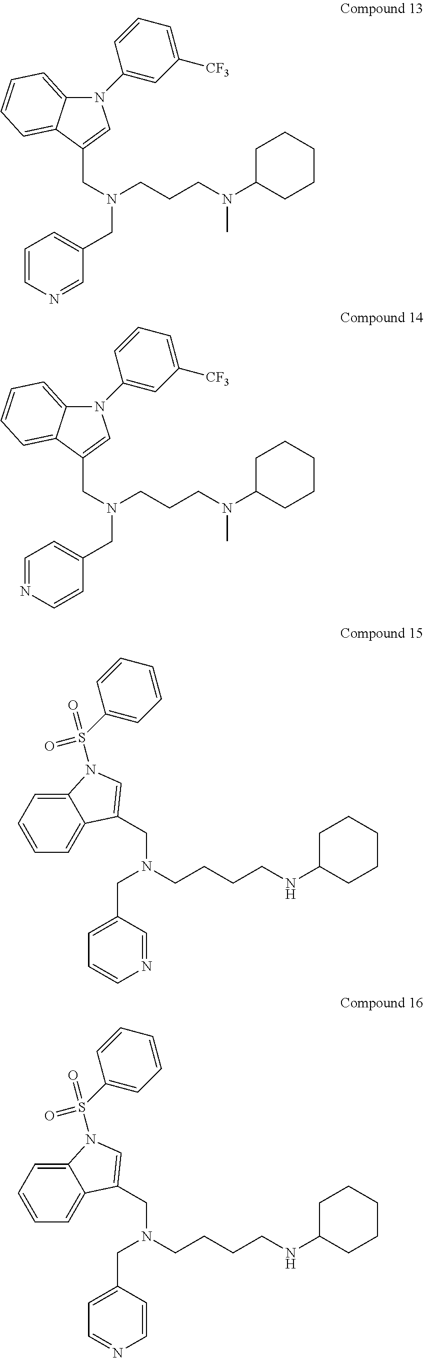

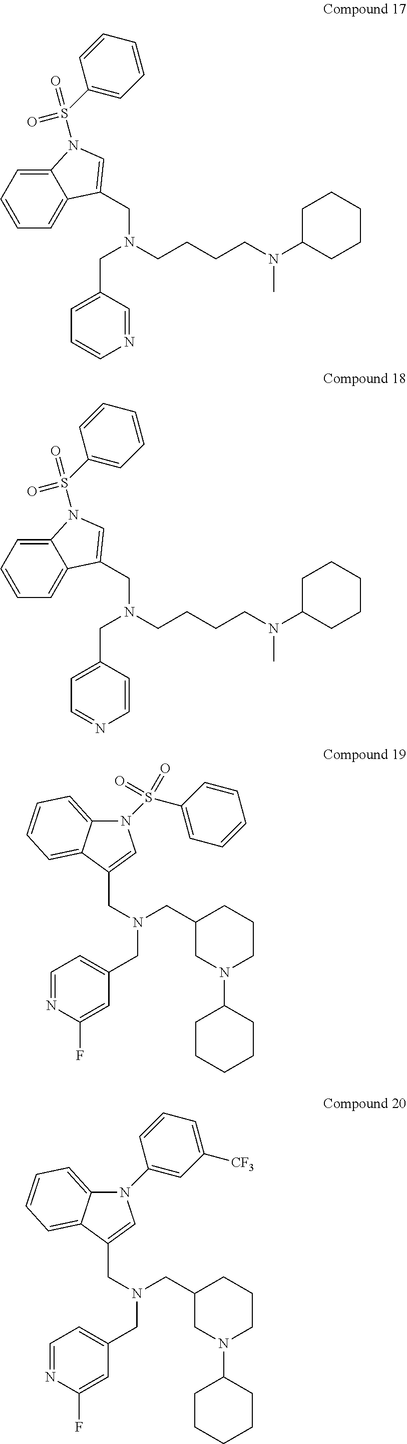

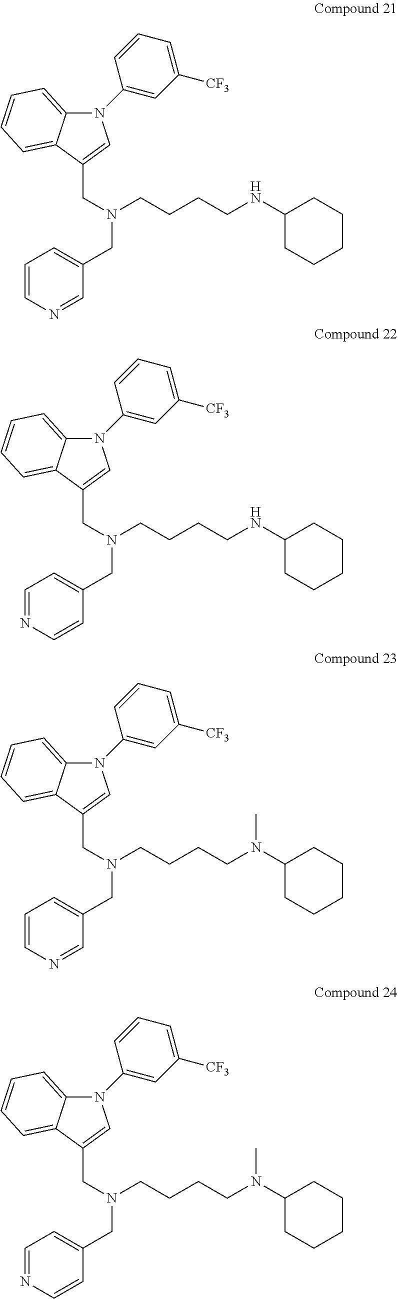 [SMILES: FC(F)(F)c1cccc(-n2cc(CN(CCCCNC3CCCCC3)Cc3cccnc3)c3ccccc32)c1 ; FC(F)(F)c1cccc(-n2cc(CN(CCCCNC3CCCCC3)Cc3ccncc3)c3ccccc32)c1 ; CN(CCCCN(Cc1cccnc1)Cc1cn(-c2cccc(C(F)(F)F)c2)c2ccccc12)C1CCCCC1 ; CN(CCCCN(Cc1ccncc1)Cc1cn(-c2cccc(C(F)(F)F)c2)c2ccccc12)C1CCCCC1]

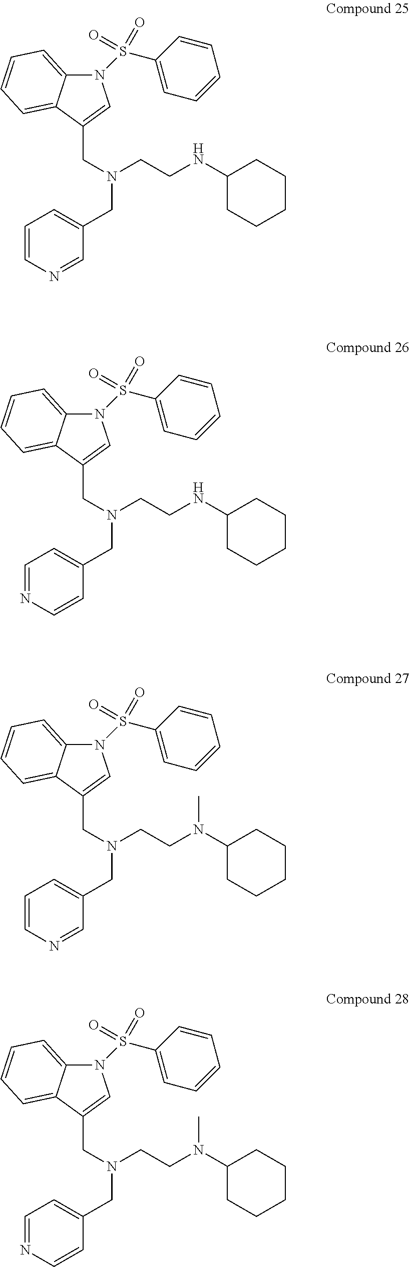 [SMILES: O=S(=O)(c1ccccc1)n1cc(CN(CCNC2CCCCC2)Cc2cccnc2)c2ccccc21 ; O=S(=O)(c1ccccc1)n1cc(CN(CCNC2CCCCC2)Cc2ccncc2)c2ccccc21 ; CN(CCN(Cc1cccnc1)Cc1cn(S(=O)(=O)c2ccccc2)c2ccccc12)C1CCCCC1 ; CN(CCN(Cc1ccncc1)Cc1cn(S(=O)(=O)c2ccccc2)c2ccccc12)C1CCCCC1]

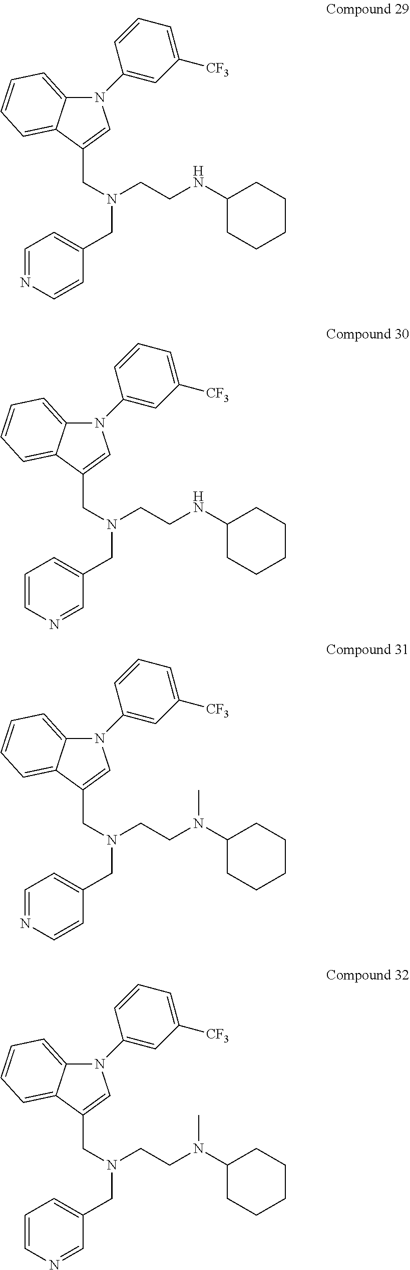 [SMILES: FC(F)(F)c1cccc(-n2cc(CN(CCNC3CCCCC3)Cc3ccncc3)c3ccccc32)c1 ; FC(F)(F)c1cccc(-n2cc(CN(CCNC3CCCCC3)Cc3cccnc3)c3ccccc32)c1 ; CN(CCN(Cc1ccncc1)Cc1cn(-c2cccc(C(F)(F)F)c2)c2ccccc12)C1CCCCC1 ; CN(CCN(Cc1cccnc1)Cc1cn(-c2cccc(C(F)(F)F)c2)c2ccccc12)C1CCCCC1]

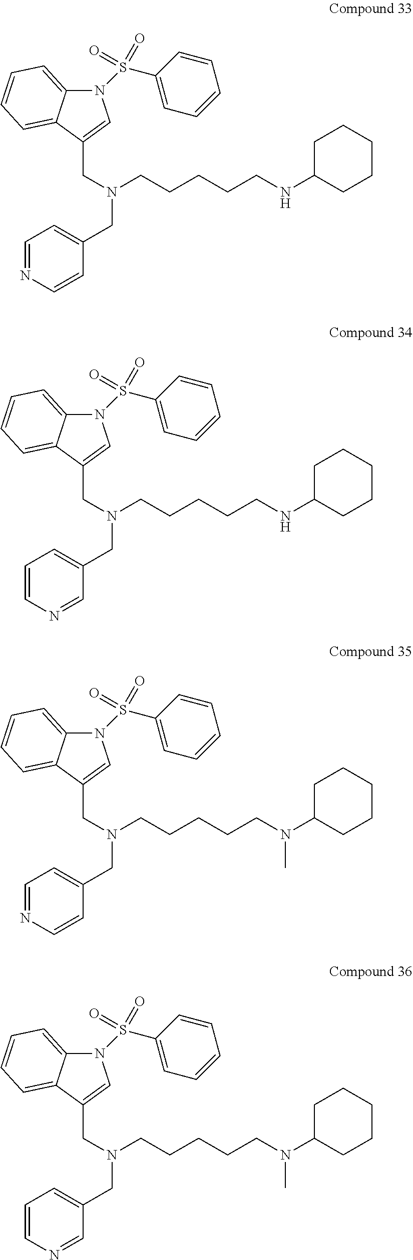 [SMILES: O=S(=O)(c1ccccc1)n1cc(CN(CCCCCNC2CCCCC2)Cc2ccncc2)c2ccccc21 ; O=S(=O)(c1ccccc1)n1cc(CN(CCCCCNC2CCCCC2)Cc2cccnc2)c2ccccc21 ; CN(CCCCCN(Cc1ccncc1)Cc1cn(S(=O)(=O)c2ccccc2)c2ccccc12)C1CCCCC1 ; CN(CCCCCN(Cc1cccnc1)Cc1cn(S(=O)(=O)c2ccccc2)c2ccccc12)C1CCCCC1]

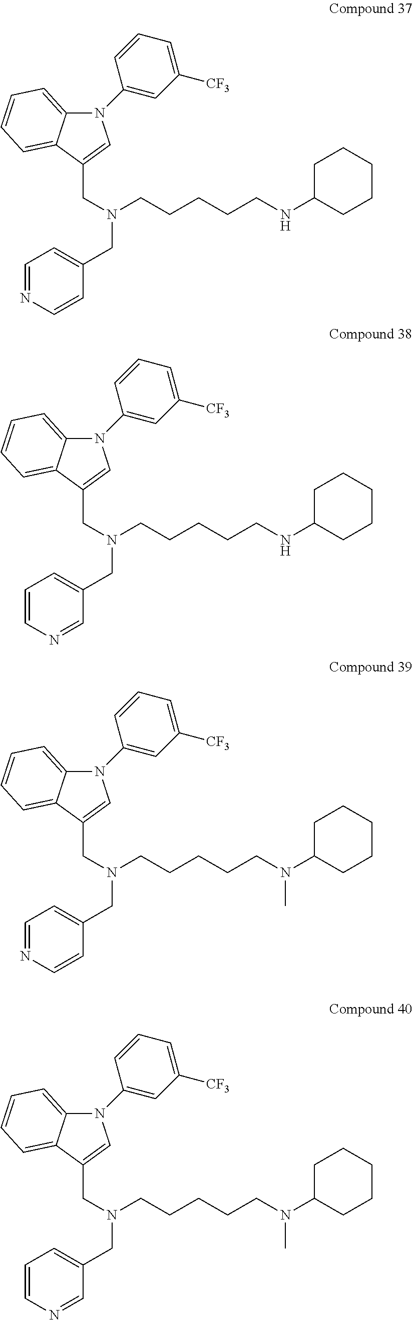 [SMILES: FC(F)(F)c1cccc(-n2cc(CN(CCCCCNC3CCCCC3)Cc3ccncc3)c3ccccc32)c1 ; FC(F)(F)c1cccc(-n2cc(CN(CCCCCNC3CCCCC3)Cc3cccnc3)c3ccccc32)c1 ; CN(CCCCCN(Cc1ccncc1)Cc1cn(-c2cccc(C(F)(F)F)c2)c2ccccc12)C1CCCCC1 ; CN(CCCCCN(Cc1cccnc1)Cc1cn(-c2cccc(C(F)(F)F)c2)c2ccccc12)C1CCCCC1]

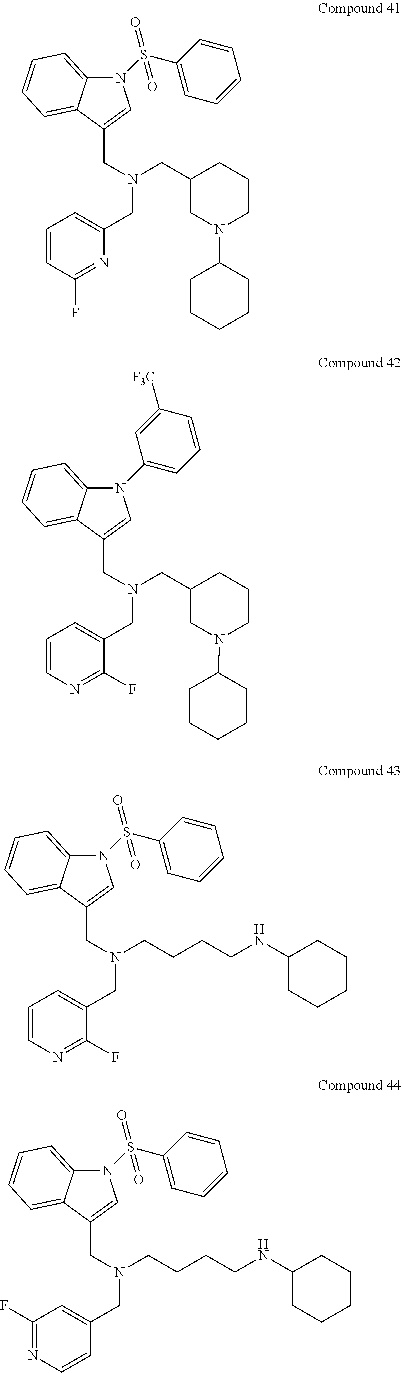 [SMILES: O=S(=O)(c1ccccc1)n1cc(CN(Cc2cccc(F)n2)CC2CCCN(C3CCCCC3)C2)c2ccccc21 ; Fc1ncccc1CN(Cc1cn(-c2cccc(C(F)(F)F)c2)c2ccccc12)CC1CCCN(C2CCCCC2)C1 ; O=S(=O)(c1ccccc1)n1cc(CN(CCCCNC2CCCCC2)Cc2cccnc2F)c2ccccc21 ; O=S(=O)(c1ccccc1)n1cc(CN(CCCCNC2CCCCC2)Cc2ccnc(F)c2)c2ccccc21]

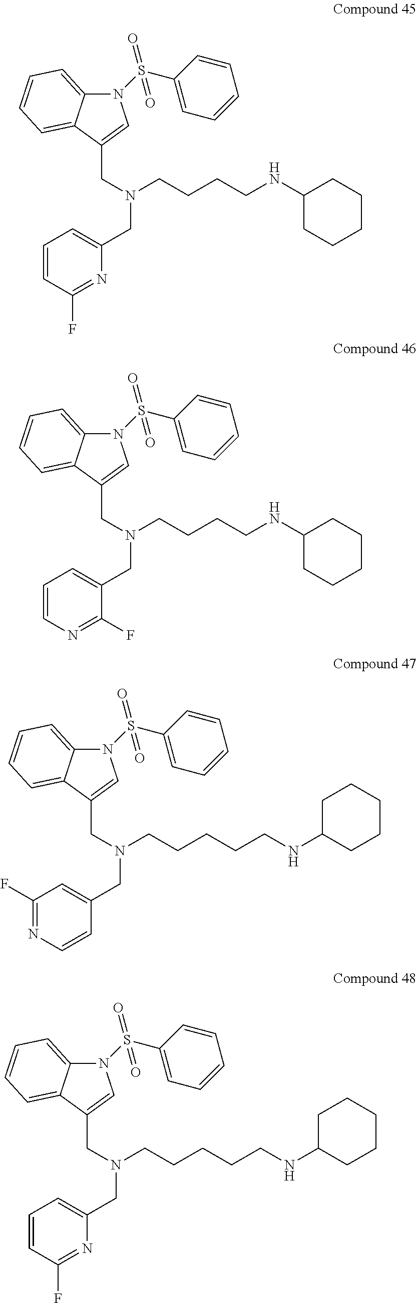 [SMILES: O=S(=O)(c1ccccc1)n1cc(CN(CCCCNC2CCCCC2)Cc2cccc(F)n2)c2ccccc21 ; O=S(=O)(c1ccccc1)n1cc(CN(CCCCNC2CCCCC2)Cc2cccnc2F)c2ccccc21 ; O=S(=O)(c1ccccc1)n1cc(CN(CCCCCNC2CCCCC2)Cc2ccnc(F)c2)c2ccccc21 ; O=S(=O)(c1ccccc1)n1cc(CN(CCCCCNC2CCCCC2)Cc2cccc(F)n2)c2ccccc21]

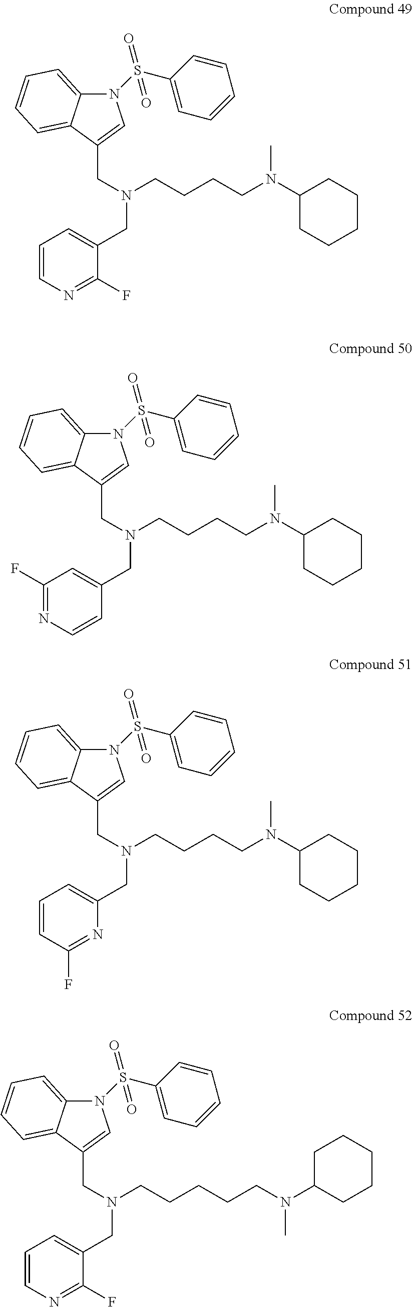 [SMILES: CN(CCCCN(Cc1cccnc1F)Cc1cn(S(=O)(=O)c2ccccc2)c2ccccc12)C1CCCCC1 ; CN(CCCCN(Cc1ccnc(F)c1)Cc1cn(S(=O)(=O)c2ccccc2)c2ccccc12)C1CCCCC1 ; CN(CCCCN(Cc1cccc(F)n1)Cc1cn(S(=O)(=O)c2ccccc2)c2ccccc12)C1CCCCC1 ; CN(CCCCCN(Cc1cccnc1F)Cc1cn(S(=O)(=O)c2ccccc2)c2ccccc12)C1CCCCC1]

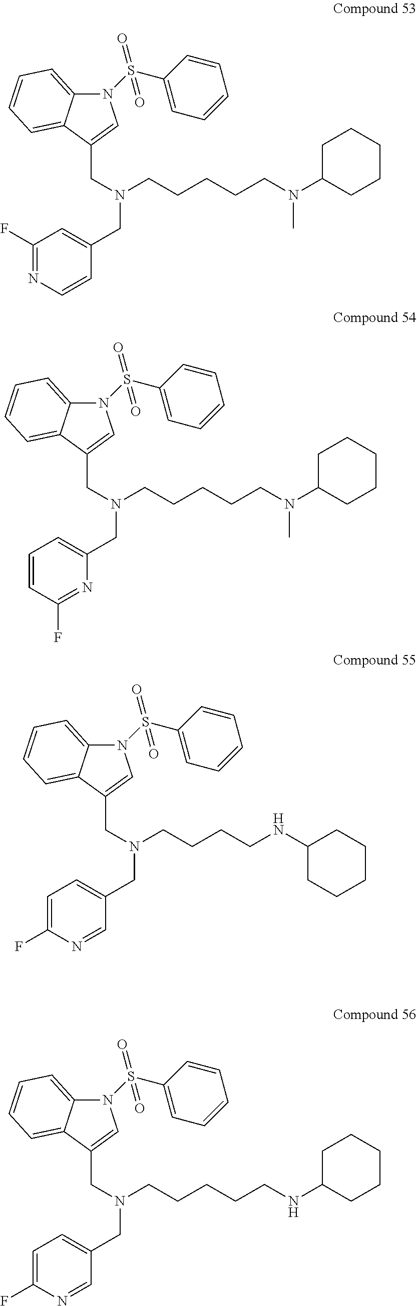 [SMILES: CN(CCCCCN(Cc1ccnc(F)c1)Cc1cn(S(=O)(=O)c2ccccc2)c2ccccc12)C1CCCCC1 ; CN(CCCCCN(Cc1cccc(F)n1)Cc1cn(S(=O)(=O)c2ccccc2)c2ccccc12)C1CCCCC1 ; O=S(=O)(c1ccccc1)n1cc(CN(CCCCNC2CCCCC2)Cc2ccc(F)nc2)c2ccccc21 ; O=S(=O)(c1ccccc1)n1cc(CN(CCCCCNC2CCCCC2)Cc2ccc(F)nc2)c2ccccc21]

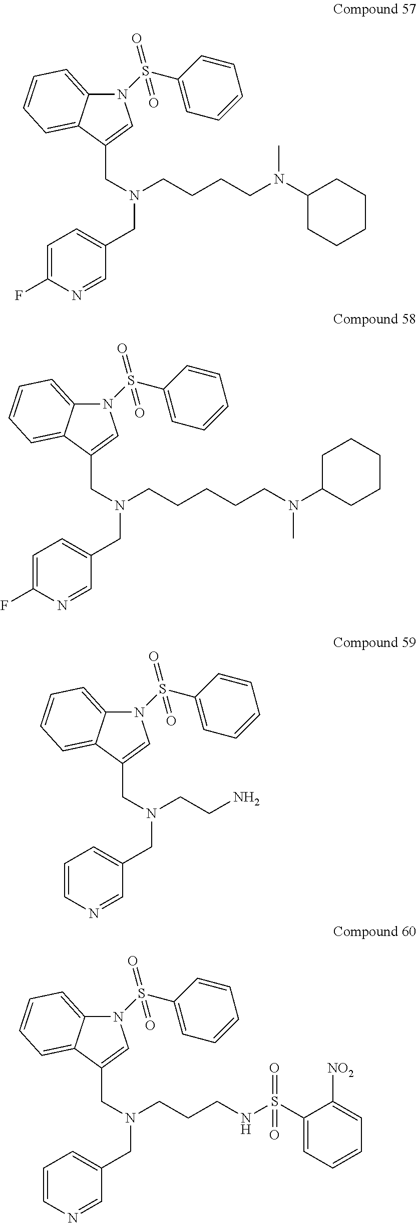 [SMILES: CN(CCCCN(Cc1ccc(F)nc1)Cc1cn(S(=O)(=O)c2ccccc2)c2ccccc12)C1CCCCC1 ; CN(CCCCCN(Cc1ccc(F)nc1)Cc1cn(S(=O)(=O)c2ccccc2)c2ccccc12)C1CCCCC1 ; NCCN(Cc1cccnc1)Cc1cn(S(=O)(=O)c2ccccc2)c2ccccc12 ; O=[N+]([O-])c1ccccc1S(=O)(=O)NCCCN(Cc1cccnc1)Cc1cn(S(=O)(=O)c2ccccc2)c2ccccc12]

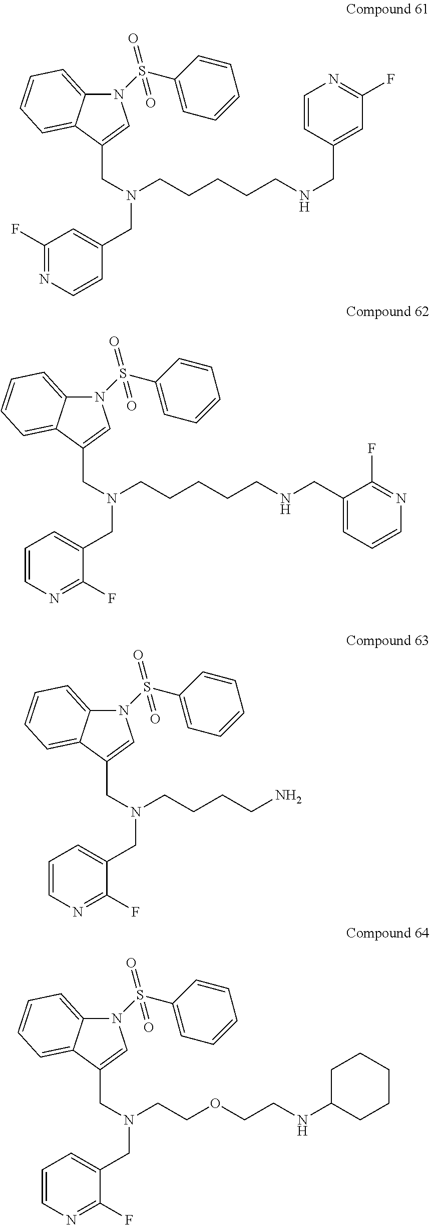 [SMILES: O=S(=O)(c1ccccc1)n1cc(CN(CCCCCNCc2ccnc(F)c2)Cc2ccnc(F)c2)c2ccccc21 ; O=S(=O)(c1ccccc1)n1cc(CN(CCCCCNCc2cccnc2F)Cc2cccnc2F)c2ccccc21 ; NCCCCN(Cc1cccnc1F)Cc1cn(S(=O)(=O)c2ccccc2)c2ccccc12 ; O=S(=O)(c1ccccc1)n1cc(CN(CCOCCNC2CCCCC2)Cc2cccnc2F)c2ccccc21]

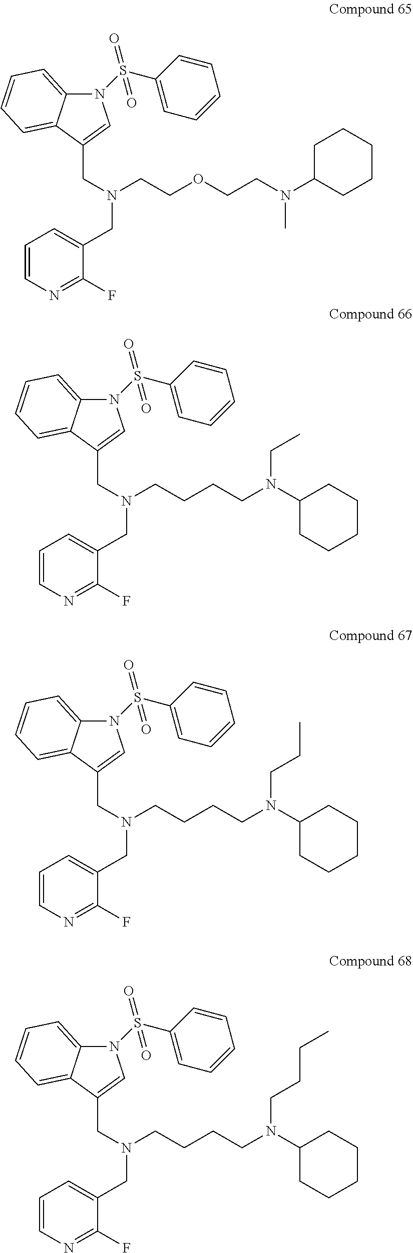 [SMILES: CN(CCOCCN(Cc1cccnc1F)Cc1cn(S(=O)(=O)c2ccccc2)c2ccccc12)C1CCCCC1 ; CCN(CCCCN(Cc1cccnc1F)Cc1cn(S(=O)(=O)c2ccccc2)c2ccccc12)C1CCCCC1 ; CCCN(CCCCN(Cc1cccnc1F)Cc1cn(S(=O)(=O)c2ccccc2)c2ccccc12)C1CCCCC1 ; CCCCN(CCCCN(Cc1cccnc1F)Cc1cn(S(=O)(=O)c2ccccc2)c2ccccc12)C1CCCCC1]

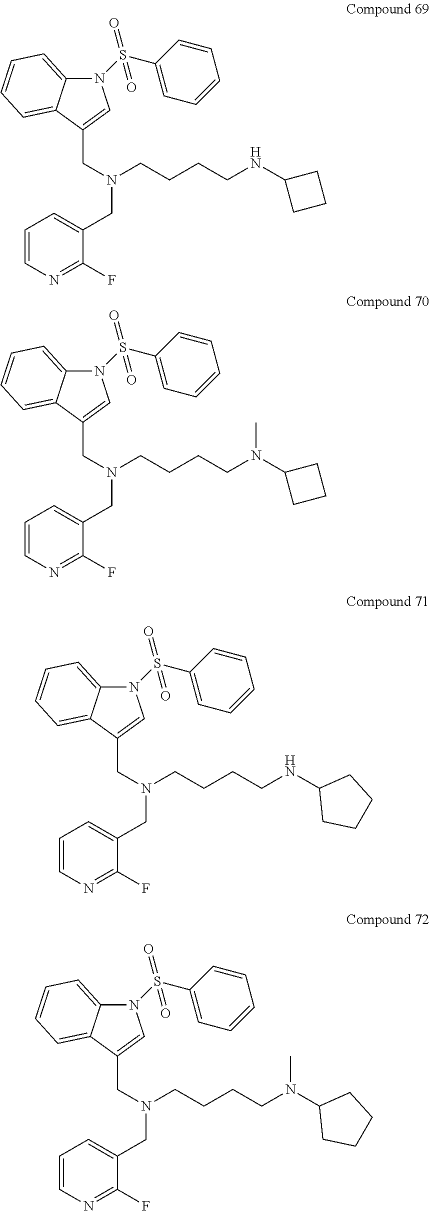

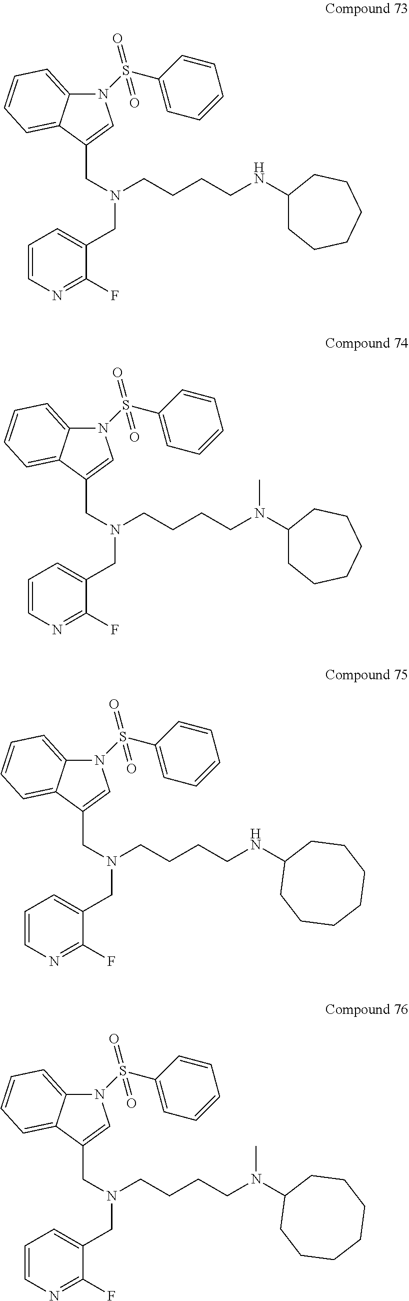 [SMILES: O=S(=O)(c1ccccc1)n1cc(CN(CCCCNC2CCCCCC2)Cc2cccnc2F)c2ccccc21 ; CN(CCCCN(Cc1cccnc1F)Cc1cn(S(=O)(=O)c2ccccc2)c2ccccc12)C1CCCCCC1 ; O=S(=O)(c1ccccc1)n1cc(CN(CCCCNC2CCCCCCC2)Cc2cccnc2F)c2ccccc21 ; CN(CCCCN(Cc1cccnc1F)Cc1cn(S(=O)(=O)c2ccccc2)c2ccccc12)C1CCCCCCC1]

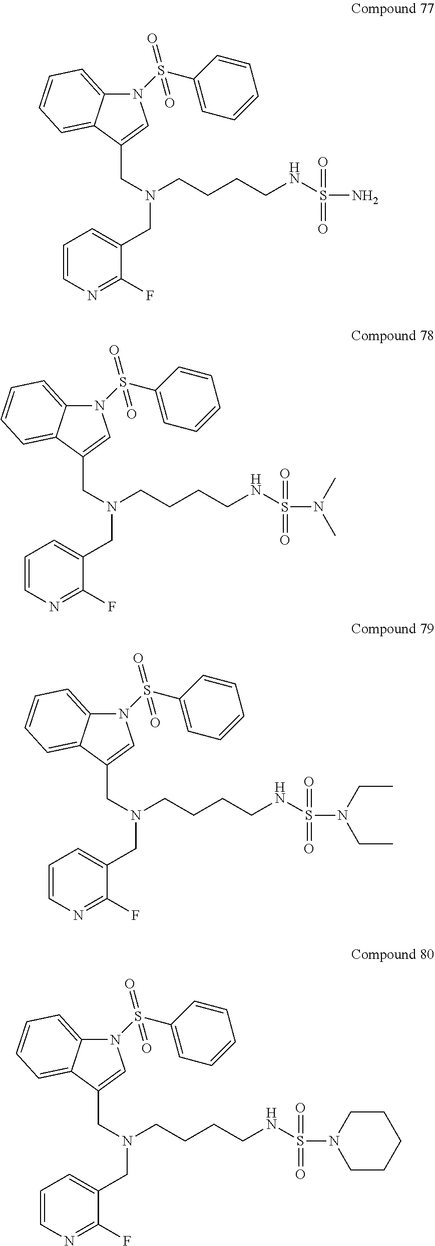 [SMILES: NS(=O)(=O)NCCCCN(Cc1cccnc1F)Cc1cn(S(=O)(=O)c2ccccc2)c2ccccc12 ; CN(C)S(=O)(=O)NCCCCN(Cc1cccnc1F)Cc1cn(S(=O)(=O)c2ccccc2)c2ccccc12 ; CCN(CC)S(=O)(=O)NCCCCN(Cc1cccnc1F)Cc1cn(S(=O)(=O)c2ccccc2)c2ccccc12 ; O=S(=O)(NCCCCN(Cc1cccnc1F)Cc1cn(S(=O)(=O)c2ccccc2)c2ccccc12)N1CCCCC1]

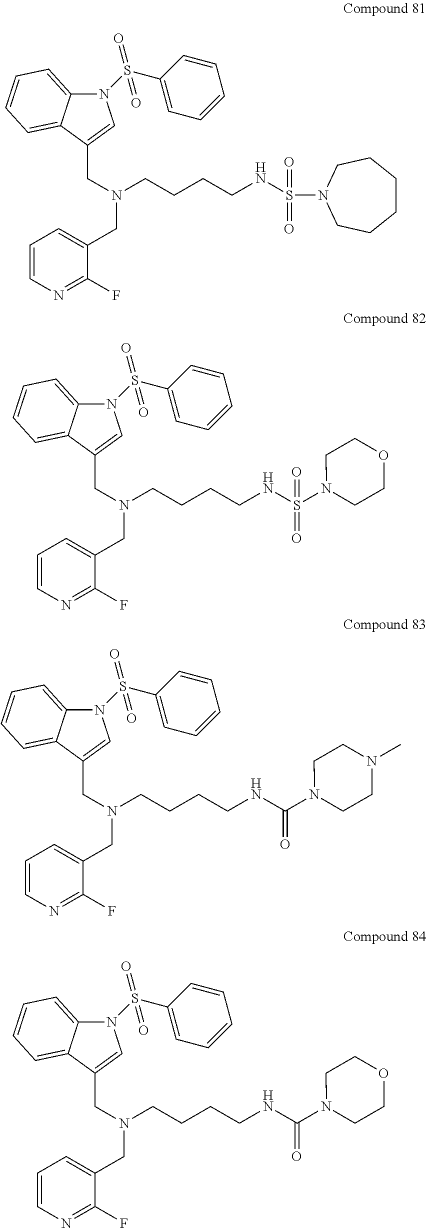 [SMILES: O=S(=O)(NCCCCN(Cc1cccnc1F)Cc1cn(S(=O)(=O)c2ccccc2)c2ccccc12)N1CCCCCC1 ; O=S(=O)(NCCCCN(Cc1cccnc1F)Cc1cn(S(=O)(=O)c2ccccc2)c2ccccc12)N1CCOCC1 ; CN1CCN(C(=O)NCCCCN(Cc2cccnc2F)Cc2cn(S(=O)(=O)c3ccccc3)c3ccccc23)CC1 ; O=C(NCCCCN(Cc1cccnc1F)Cc1cn(S(=O)(=O)c2ccccc2)c2ccccc12)N1CCOCC1]

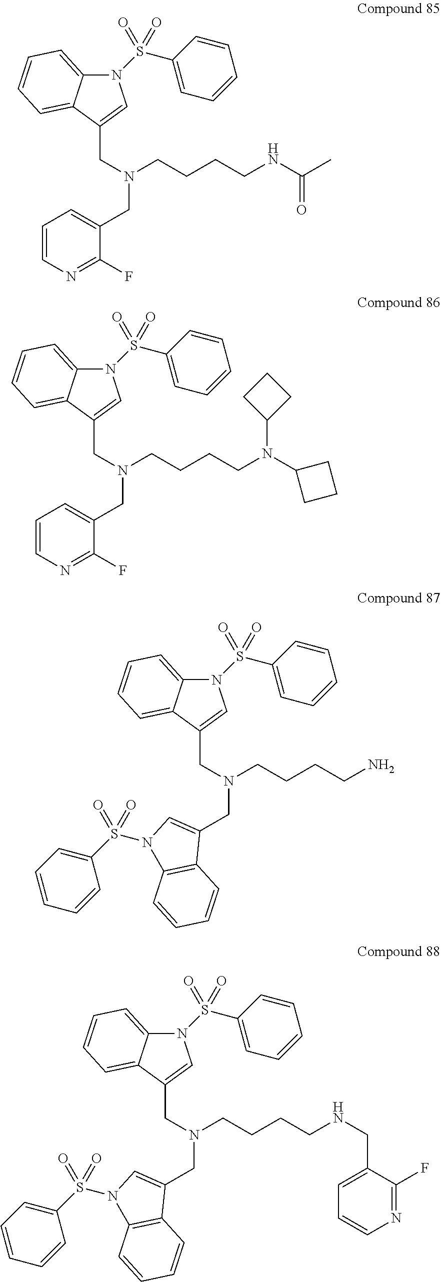

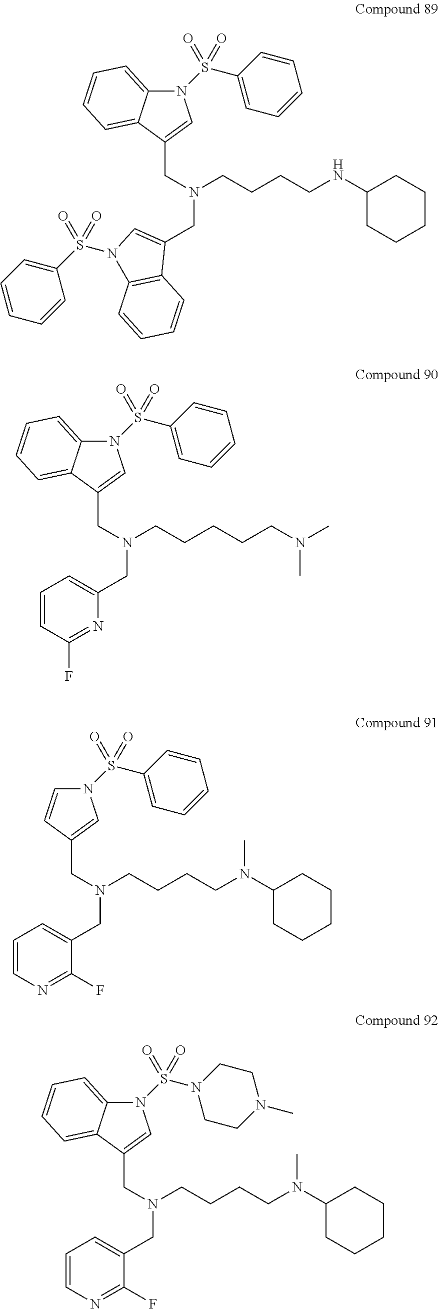 [SMILES: O=S(=O)(c1ccccc1)n1cc(CN(CCCCNC2CCCCC2)Cc2cn(S(=O)(=O)c3ccccc3)c3ccccc23)c2ccccc21 ; CN(C)CCCCCN(Cc1cccc(F)n1)Cc1cn(S(=O)(=O)c2ccccc2)c2ccccc12 ; CN(CCCCN(Cc1ccn(S(=O)(=O)c2ccccc2)c1)Cc1cccnc1F)C1CCCCC1 ; CN1CCN(S(=O)(=O)n2cc(CN(CCCCN(C)C3CCCCC3)Cc3cccnc3F)c3ccccc32)CC1]

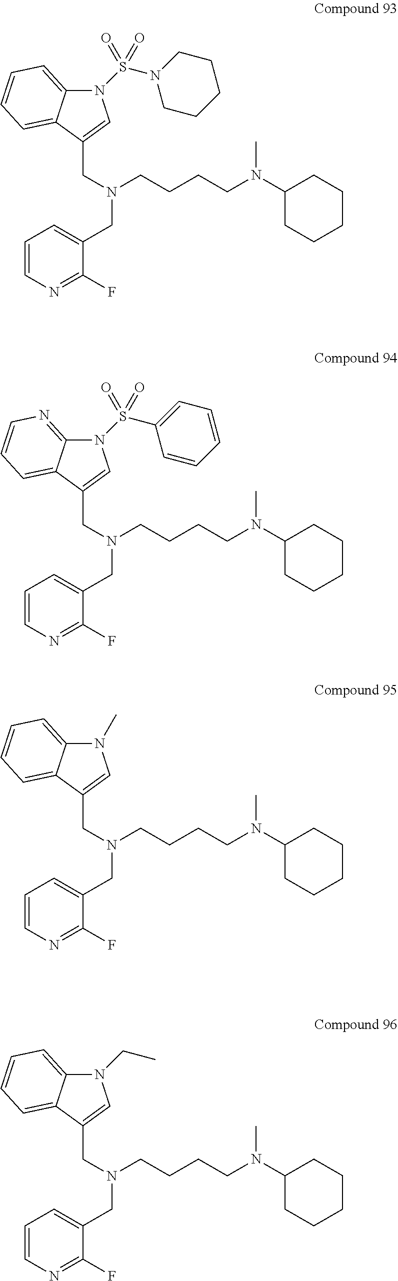 [SMILES: CN(CCCCN(Cc1cccnc1F)Cc1cn(S(=O)(=O)N2CCCCC2)c2ccccc12)C1CCCCC1 ; CN(CCCCN(Cc1cccnc1F)Cc1cn(S(=O)(=O)c2ccccc2)c2ncccc12)C1CCCCC1 ; CN(CCCCN(Cc1cccnc1F)Cc1cn(C)c2ccccc12)C1CCCCC1 ; CCn1cc(CN(CCCCN(C)C2CCCCC2)Cc2cccnc2F)c2ccccc21]

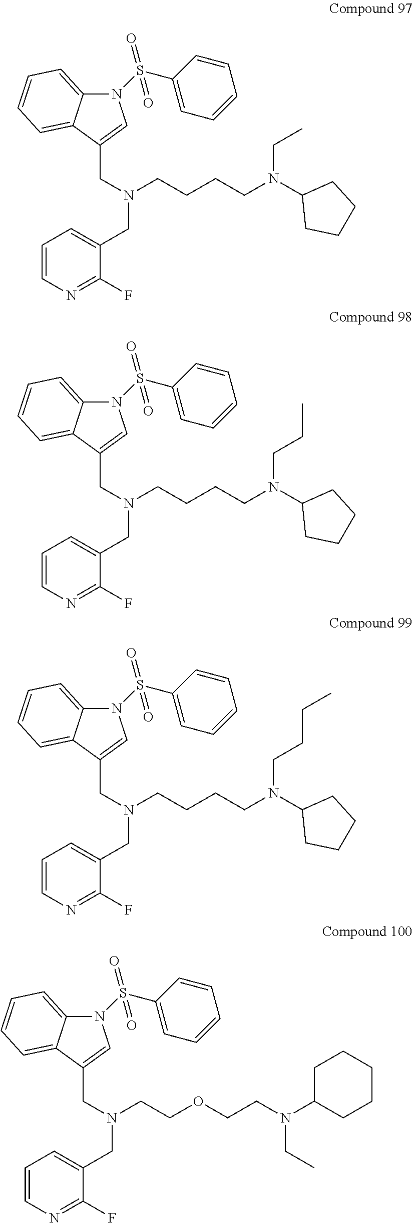 [SMILES: CCN(CCCCN(Cc1cccnc1F)Cc1cn(S(=O)(=O)c2ccccc2)c2ccccc12)C1CCCC1 ; CCCN(CCCCN(Cc1cccnc1F)Cc1cn(S(=O)(=O)c2ccccc2)c2ccccc12)C1CCCC1 ; CCCCN(CCCCN(Cc1cccnc1F)Cc1cn(S(=O)(=O)c2ccccc2)c2ccccc12)C1CCCC1 ; CCN(CCOCCN(Cc1cccnc1F)Cc1cn(S(=O)(=O)c2ccccc2)c2ccccc12)C1CCCCC1]

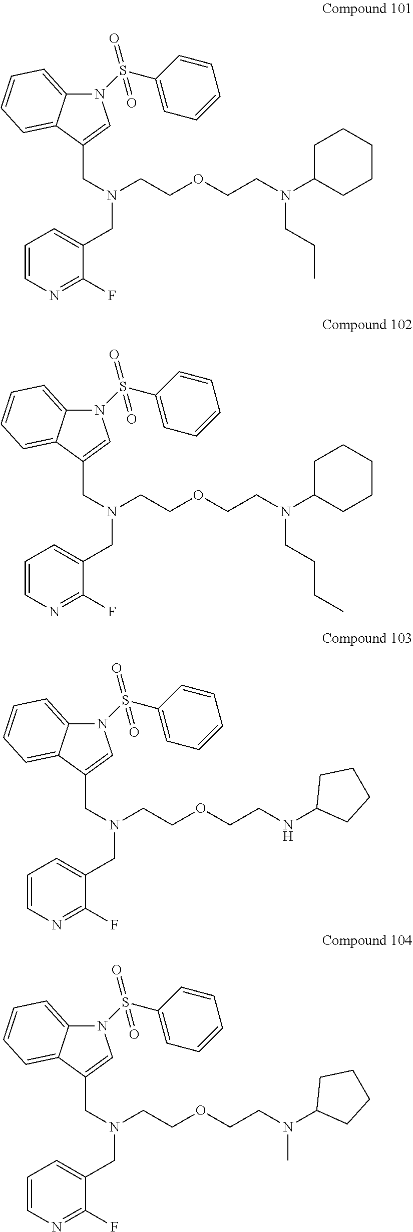

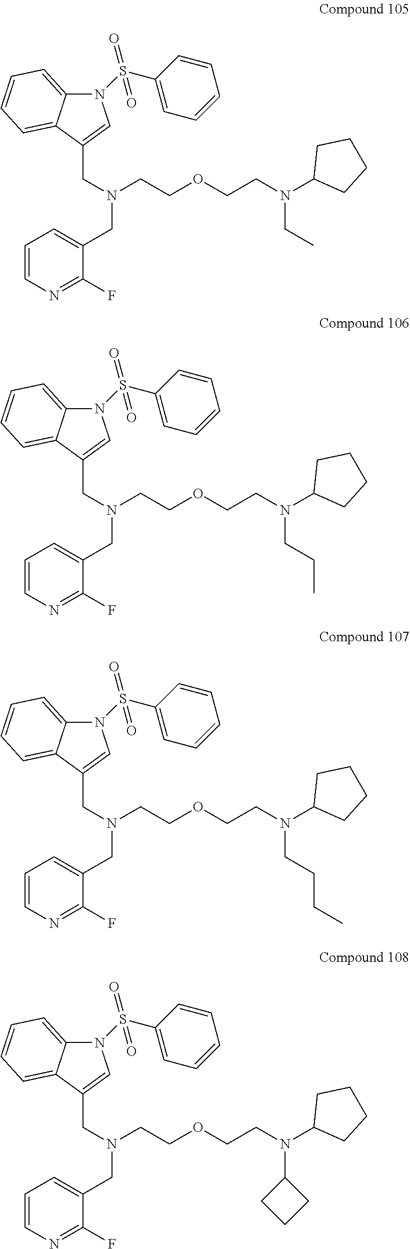 [SMILES: CCN(CCOCCN(Cc1cccnc1F)Cc1cn(S(=O)(=O)c2ccccc2)c2ccccc12)C1CCCC1 ; CCCN(CCOCCN(Cc1cccnc1F)Cc1cn(S(=O)(=O)c2ccccc2)c2ccccc12)C1CCCC1 ; CCCCN(CCOCCN(Cc1cccnc1F)Cc1cn(S(=O)(=O)c2ccccc2)c2ccccc12)C1CCCC1 ; O=S(=O)(c1ccccc1)n1cc(CN(CCOCCN(C2CCCC2)C2CCC2)Cc2cccnc2F)c2ccccc21]

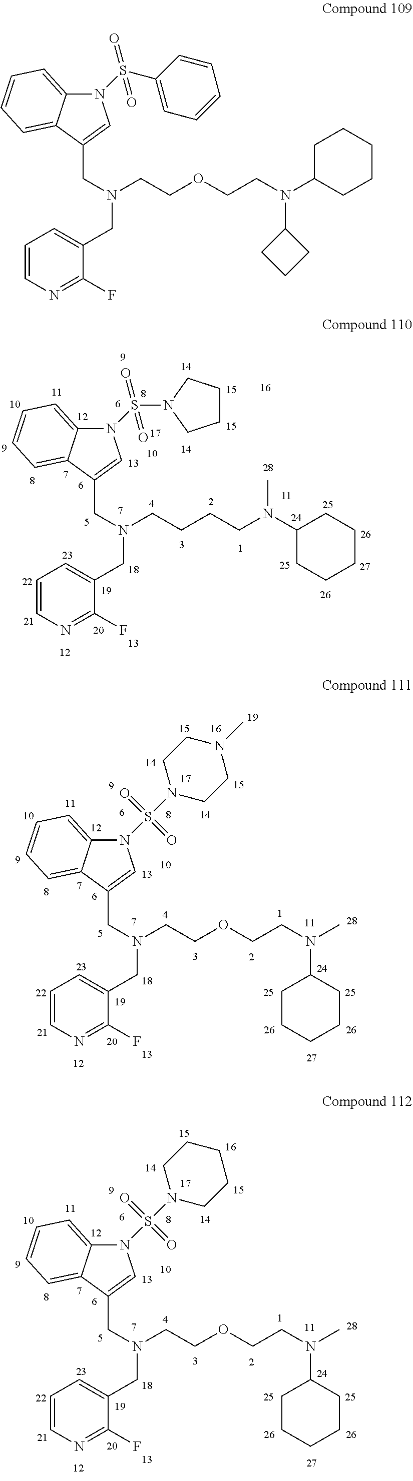 [SMILES: O=S(=O)(c1ccccc1)n1cc(CN(CCOCCN(C2CCCCC2)C2CCC2)Cc2cccnc2F)c2ccccc21 ; CN(CCCCN(Cc1cccnc1F)Cc1cn(S(=O)(=O)N2CCCC2)c2ccccc12)C1CCCCC1 ; CN1CCN(S(=O)(=O)n2cc(CN(CCOCCN(C)C3CCCCC3)Cc3cccnc3F)c3ccccc32)CC1 ; CN(CCOCCN(Cc1cccnc1F)Cc1cn(S(=O)(=O)N2CCCCC2)c2ccccc12)C1CCCCC1]

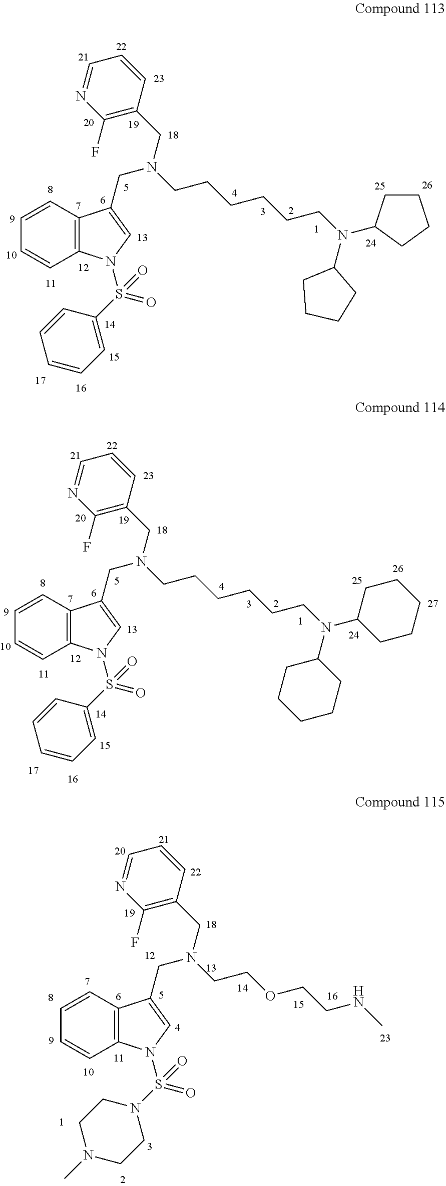 [SMILES: O=S(=O)(c1ccccc1)n1cc(CN(CCCCCCN(C2CCCC2)C2CCCC2)Cc2cccnc2F)c2ccccc21 ; O=S(=O)(c1ccccc1)n1cc(CN(CCCCCCN(C2CCCCC2)C2CCCCC2)Cc2cccnc2F)c2ccccc21 ; CNCCOCCN(Cc1cccnc1F)Cc1cn(S(=O)(=O)N2CCN(C)CC2)c2ccccc12]

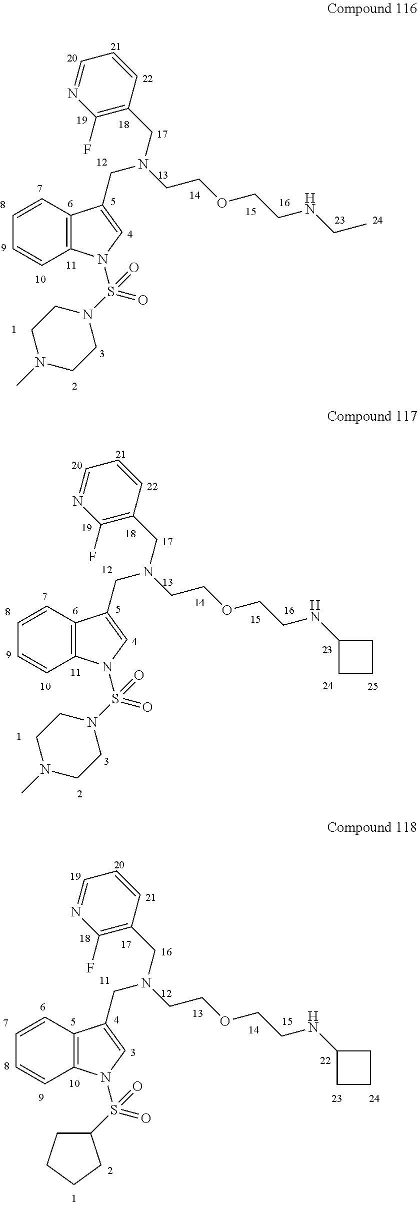 [SMILES: CCNCCOCCN(Cc1cccnc1F)Cc1cn(S(=O)(=O)N2CCN(C)CC2)c2ccccc12 ; CN1CCN(S(=O)(=O)n2cc(CN(CCOCCNC3CCC3)Cc3cccnc3F)c3ccccc32)CC1 ; O=S(=O)(C1CCCC1)n1cc(CN(CCOCCNC2CCC2)Cc2cccnc2F)c2ccccc21]

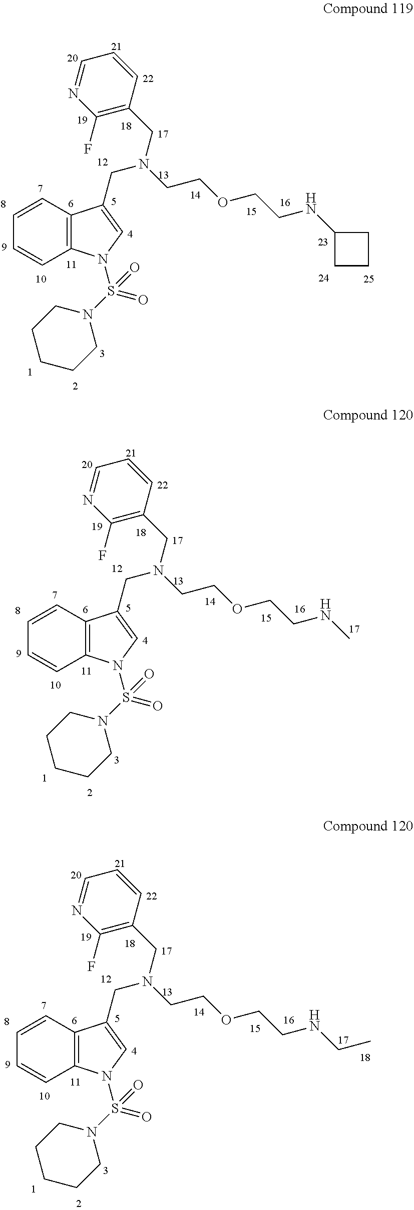 [SMILES: O=S(=O)(N1CCCCC1)n1cc(CN(CCOCCNC2CCC2)Cc2cccnc2F)c2ccccc21 ; CNCCOCCN(Cc1cccnc1F)Cc1cn(S(=O)(=O)N2CCCCC2)c2ccccc12 ; CCNCCOCCN(Cc1cccnc1F)Cc1cn(S(=O)(=O)N2CCCCC2)c2ccccc12]

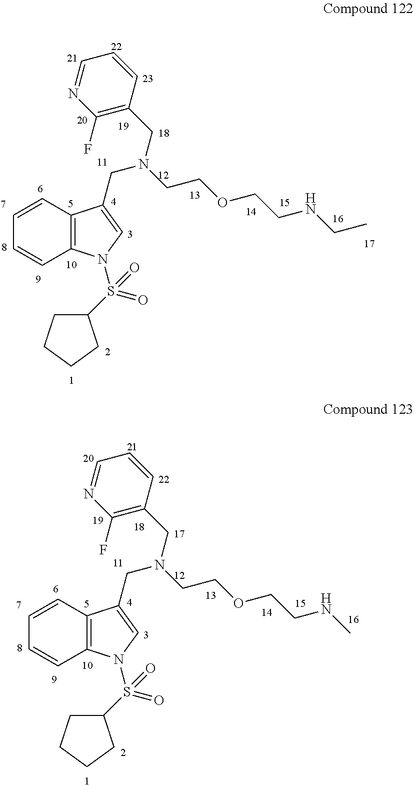 [SMILES: CCNCCOCCN(Cc1cccnc1F)Cc1cn(S(=O)(=O)C2CCCC2)c2ccccc12 ; CNCCOCCN(Cc1cccnc1F)Cc1cn(S(=O)(=O)C2CCCC2)c2ccccc12]

As should be clear to one of skilled into the art, all the stereochemical configurations of the compounds according to the invention are intended to be covered by the formulae shown herein. In particular, as is intended herein when the stereoconfiguration of a bond is not specified, the bond may represents any of an upward bond, a downward bond, a mixture of the two, in particular a 1/1 mixture of the two.

The pharmaceutically acceptable salt or hydrate according to the invention can be of any type. By way of example of suitable pharmaceutically acceptable salts of the compound of formula (I) it is possible to cite hydrochloric salt, sulphuric salt, fumaric salt, maleic salt, succinic salt, acetic salt, benzoic salt, citric salt, tartaric salt or phosphoric salt.

### ¹⁸F-Radiolabeled Compound

In one embodiment, the present invention relates to the preparation of a ¹⁸F-radiolabeled compound of formula (I) and a radiopharmaceutical composition comprising the ¹⁸F-radiolabeled compound of formula (I) according to the invention.

As intended herein, “a ¹⁸F-radiolabeled compound of formula (I)” relates to compound of formula (I) comprising ¹⁸F-labeled moieties. Preferably, the ¹⁸F-radiolabeled compound of formula (I) according to the invention comprise a ¹⁸F-radiolabeled Rb group.

The structures below include examples of compound of formula (I) according to the invention radiolabeled with ¹⁸F:

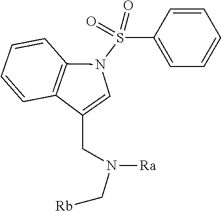

| Ra | Rb |
|---|---|
| 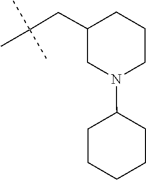 | 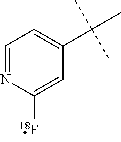 |
| 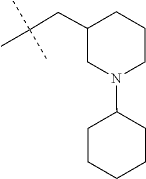 | 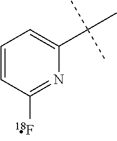 |
| 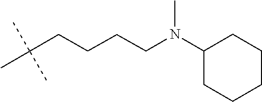 | 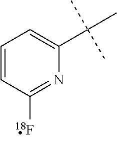 |
| 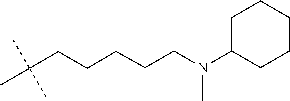 | 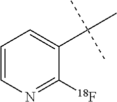 |
| 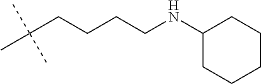 | 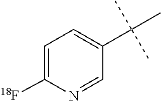 |
| 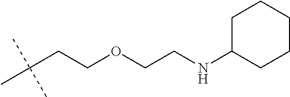 | 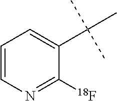 |
| 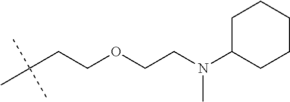 | 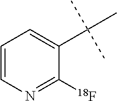 |
| 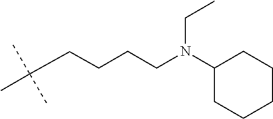 | 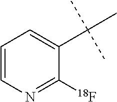 |
| 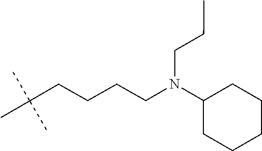 | 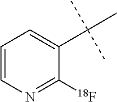 |
| 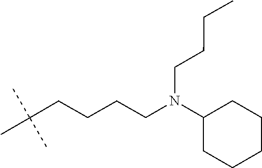 | 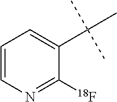 |
| 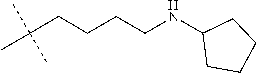 | 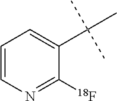 |
| 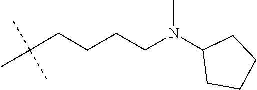 | 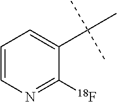 |
| 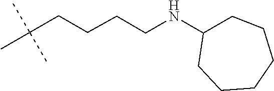 | 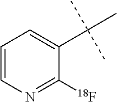 |
| 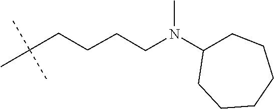 | 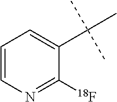 |
| 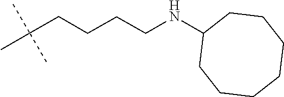 | 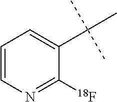 |
| 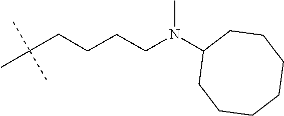 | 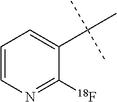 |
| 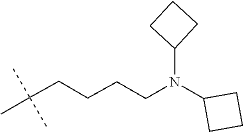 | 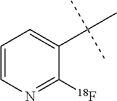 |
| 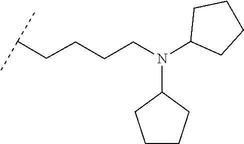 | 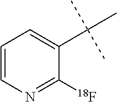 |
| 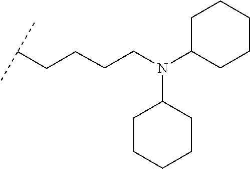 | 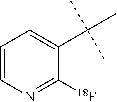 |

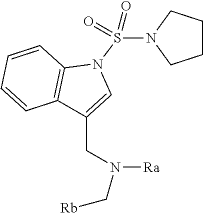

| Ra | Rb |
|---|---|
| 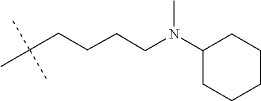 | 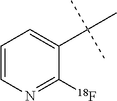 |
| 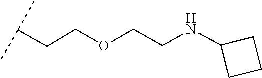 | 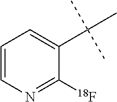 |
| 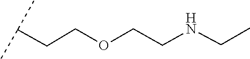 | 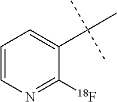 |
| 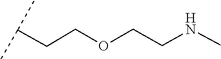 | 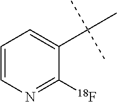 |

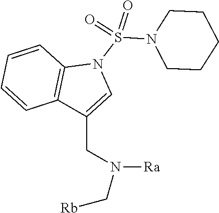

| Ra | Rb |
|---|---|
| 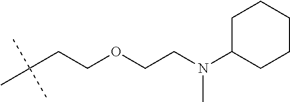 | 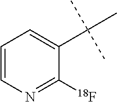 |
| 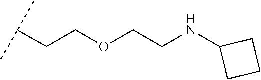 | 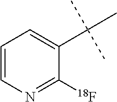 |
| 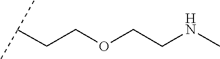 | 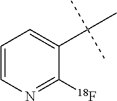 |
| 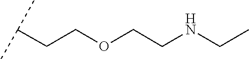 | 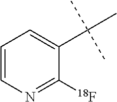 |

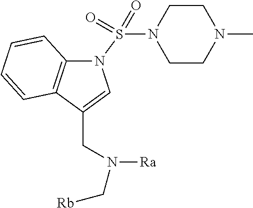

| Ra | Rb |
|---|---|
| 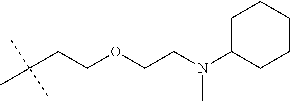 | 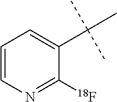 |
| 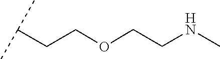 | 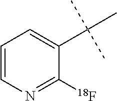 |
| 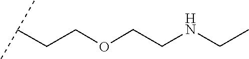 | 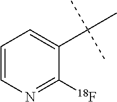 |
| 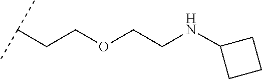 | 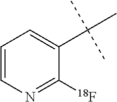 |

The compound of formula (I) according to the invention radiolabeled with ¹⁸F can be obtained by isotopic exchange or by a nucleophilic aromatic substitution on nitro or ammonium 2 pyridine precursors.

Preferably, the compound of formula (I) according to the invention radiolabeled with ¹⁸F and the radiopharmaceutical composition according to the invention are used in are used as molecular imaging radiotracers for Positron Emission Tomograpghy (PET) imaging.

As intended herein “radiotracers” also known as radiopharmaceutical or radionuclide, relates to molecules linked or labeled with radioactive material, such as ¹⁸F, which can be detected under the positron emission tomography scan. These radiotracers are preferably injected into the bloodstream and accumulates in the area of the body under examination. The radiotracers typically take about 30 to 120 minutes to travel through the body and be absorbed by the area under examination.

Preferably, the compound of formula (I) according to the invention radiolabeled with ¹⁸F or the radiopharmaceutical composition according to the invention is used for the detection and diagnosis of diseased tissue in an individual.

Preferably, the compound of formula (I) radiolabeled with ¹⁸F or the radiopharmaceutical composition as defined above is used in at least one of the following situations:
to diagnose a cytokine-mediated disease according to the invention,
to determine the stage of evolution of the disease,
to determine and predict early the therapy response; and
to monitor the therapeutic follow-up of a treatment.

More preferably, the compound of formula (I) according to the invention radiolabeled with ¹⁸F or the radiopharmaceutical composition according to the invention is used for the detection and diagnosis of inflammatory zones, cancer, neurological and neurodegenerative disease, cardiovascular disorder and metabolic disorder.

Even more preferably, the compound of formula (I) radiolabeled with ¹⁸F or the radiopharmaceutical composition according to the invention is used for the detection and diagnosis of rheumatoid arthritis (RA).

In an embodiment of the present invention, the compound of formula (I) radiolabeled with ¹⁸F or the radiopharmaceutical composition as defined above is used in one or more of the following situations:
to confirm rheumatoid arthritis (RA) pathology by detecting tmTNFa and sTNFa to localize sites of inflammation in patients suspected to display RA;
to demonstrate the presence of an ongoing inflammation process where there is a suspicion of inflammation without a systemic inflammatory response;
to determine the staging of the RA disease by diagnosing the status of activity in inflamed organs or tissues;
to determine and predict early the response to therapy. In particular, the evaluation of the non-responsiveness to TNFa-blocking agents can be evaluated based on the detection and localization of tmTNFa and sTNFa by PET imaging;
to carry out the therapeutic follow-up of an anti-TNFa treatment by assessing inflammatory activity state before and during the treatment.

Preferably, the compound of formula (I) radiolabeled with ¹⁸F or the radiopharmaceutical composition according to the invention is in a form suitable for injection by intravenous route.

### Prevention and Treatment

The cytokines according to the invention are preferably selected from the group consisting of TNFa and an interleukin, such as IL-4, IL-5, IL-13 and IL-6.

As intended herein “cytokine-mediated disease” encompasses diseases associated with an abnormal or pathological level of cytokine, preferably TNF-a, IL-4, IL-5, IL-13 and IL-6, which is more preferably abnormally or pathologically elevated.

Preferably, the present invention relates to the prevention or treatment of at least one symptom, disorder or disease caused by, or associated to, an overproduction, an up-regulation, a dysregulation, an excess or a high, elevated or above-normal level of cytokines, in particular TNF-a, IL-4, IL-5, IL-13 and IL-6. The overproduction or excess of cytokine, in particular TNF-a, IL-4, IL-5, IL-13 and IL-6, or high or elevated cytokine level, in particular TNF-a, IL-4, IL-5, IL-13 and IL-6 level, can be acquired, for instance as a consequence of a disorder or inherited, for instance as a genetic disorder.

More preferably, the present invention relates to the prevention or treatment of a cytokine-mediated disease. Preferably, the cytokine-mediated disease according to the invention is selected from the group consisting of an inflammatory or autoimmune disorder, a neurological or neurodegenerative disorder, pain, a nociceptive disorder, a cardiovascular disorder, a metabolic disorder, an ocular disorder, and an oncological disorder.

Inflammatory and autoimmune disorders are well known to the person skilled in the art and preferably include systemic autoimmune disorders, autoimmune endocrine disorders and organ-specific autoimmune disorders, osteo arthritis.

Systemic autoimmune disorders are preferably selected from the group consisting of systemic lupus erythematosus (SLE), psoriasis, psoriatic arthropathy, vasculitis, polymyositis, scleroderma, multiple sclerosis, systemic sclerosis, ankylosing spondylitis, rheumatoid arthritis, non-specific inflammatory arthritis, juvenile inflammatory arthritis, juvenile idiopathic arthritis, anemia of chronic disease (ACD), Still's disease, Beliefs disease and Sjogren's syndrome.

Autoimmune endocrine disorders is preferably thyroiditis.

Organ-specific autoimmune disorders are preferably selected from the group consisting of Addison's disease, haemolytic or pernicious anaemia, acute kidney injury (AKI; including cisplatin-induced AKI), diabetic nephropathy (DN), obstructive uropathy (including cisplatin-induced obstructive uropathy), glomerulonephritis (including Goodpasture's syndrome, immune complex-mediated glomerulonephritis and antineutrophil cytoplasmic antibodies (ANCA)-associated glomerulonephritis), lupus nephritis (LN), minimal change disease, Graves' disease, idiopathic thrombocytopenic purpura, inflammatory bowel disease, including Crohn's disease, ulcerative colitis, indeterminate colitis and pouchitis, pemphigus, atopic dermatitis, autoimmune hepatitis, primary biliary cirrhosis, autoimmune pneumonitis, autoimmune carditis, myasthenia gravis, spontaneous infertility, osteoporosis, osteopenia, erosive bone disease, chondritis, cartilage degeneration and/or destruction, fibrosing disorders, including various forms of hepatic and pulmonary fibrosis, asthma, rhinitis, chronic obstructive pulmonary disease (COPD), respiratory distress syndrome, sepsis, fever, muscular dystrophy, including Duchenne muscular dystrophy, and organ transplant rejection, including kidney allograft rejection.

Neurological and neurodegenerative disorders are well known to the person skilled in the art and preferably include Alzheimer's disease, Parkinson's disease, Huntington's disease, ischemia, stroke, amyotrophic lateral sclerosis, spinal cord injury, head trauma, seizures and epilepsy.

Cardiovascular disorders are well known to the person skilled in the art and preferably include thrombosis, cardiac hypertrophy, hypertension, irregular contractility of the, and sexual disorders.

Metabolic disorders are well known to the person skilled in the art and preferably include diabetes, including insulin-dependent diabetes mellitus and juvenile diabetes, dyslipidemia and metabolic syndrome.

Ocular disorders are well known to the person skilled in the art and preferably include retinopathy, including diabetic retinopathy, proliferative retinopathy, non-proliferative retinopathy and retinopathy of prematurity, macular oedema, including diabetic macular oedema, age-related macular degeneration (ARMD), vascularization, including corneal vascularisation and neovascularisation, retinal vein occlusion, and various forms of uveitis and keratitis.

Oncological disorders, which may be acute or chronic, are well known to the person skilled in the art preferably include proliferative disorders, especially cancer, and cancer-associated complications, including skeletal complications, cachexia and anemia. Cancers according to the invention are preferably selected from the group consisting of haemato-logical malignancy, including leukemia and lymphoma, and non-haemato-logical malignancy, including solid tumor cancer, sarcoma, meningioma, glioblastoma multiforme, neuroblastoma, melanoma, gastric carcinoma and renal cell carcinoma. Varieties of leukemia preferably include lymphoblastic T cell leukemia, chronic myelogenous leukemia (CML), chronic lymphocytic/lymphoid leukemia (CLL), hairy-cell leukemia, acute lymphoblastic leukemia (ALL), acute myelogenous leukemia (AML), myelodysplasia syndrome, chronic neutrophilic leukemia, acute lymphoblastic T cell leukemia, plasmacytoma, immunoblastic large cell leukemia, mantle cell leukemia, multiple myeloma, acute megakaryoblastic leukemia, acute megakaryocytic leukemia, promyelocytic leukemia and erythroleukemia. Varieties of lymphoma preferably include malignant lymphoma, Hodgkin's lymphoma, non-Hodgkin's lymphoma, lymphoblastic T cell lymphoma, Burkitt's lymphoma, follicular lymphoma, MALTI lymphoma and marginal zone lymphoma. Varieties of non-haemato-logical malignancy include cancer of the prostate, lung, breast, rectum, colon, lymph node, bladder, kidney, pancreas, liver, ovary, uterus, cervix, brain, skin, bone, stomach and muscle.

More preferably, the cytokine-mediated disease according to the invention is selected form the group consisting of osteo arthritis, rheumatoid Arthritis (RA), psoriasis, Crohn disease, cancer asthma and a cardiovascular disorder.

### Individual

The individual according to the invention is preferably a mammal, more preferably a human.

Preferably, the individual according to the invention presents with an abnormal or pathological level of cytokine, in particular TNF-a, IL-4, IL-5 and IL-6, which is more preferably abnormally or pathologically elevated.

Preferably, the individual according to the invention presents an overproduction or an excess of cytokine, in particular TNF-a, IL-4, IL-5, IL-13 and IL-6, or a high or elevated cytokine level, in particular TNF-a, IL-4, IL-5, IL-13 and IL-6 level.

In an embodiment of the invention, the individual according to the invention suffers from a cytokine-mediated disease. Preferably, the individual according to the invention suffers from an inflammatory or autoimmune disorder, a neurological or neurodegenerative disorder, pain, a nociceptive disorder, a cardiovascular disorder, a metabolic disorder, an ocular disorder, and an oncological disorder.

More preferably, the individual according to the invention suffers from rheumatoid arthritis.

### Pharmaceutical Composition

As intended herein “pharmaceutically acceptable carrier or excipient” refers to any material suitable with a pharmaceutical composition. Preferably, the pharmaceutically acceptable carrier or excipient according to the invention is suitable for an administration by the oral, parenteral, topical, intravenous, intramuscular, nasal or rectal route.

Preferably, the pharmaceutically acceptable carrier or excipient according to the invention, includes but is not limited to any of the standard carrier or excipient known to one of skilled in the art such as water, cyclodextrine, glycerin, alcohol, oil emulsion, water emulsion, buffered saline solution, preservative, stabilizer and wetting agents.

In an embodiment, the pharmaceutical composition according to the invention comprises at least one additional active substance.

Preferably, the additional compound according to the invention is a compound intended for diagnosing, preventing or treating a cytokine mediated disease, in particular selected from the group consisting of an inflammatory or autoimmune disorder, a neurological or neurodegenerative disorder, pain, a nociceptive disorder, a cardiovascular disorder, a metabolic disorder, an ocular disorder, and an oncological disorder.

Preferably, the additional compound according to the invention relates to any compound intended to alleviate one or more symptoms of or to treat of prevent a cytokine mediated-disease, in particular an inflammatory or autoimmune disorder, a neurological or neurodegenerative disorder, pain, a nociceptive disorder, a cardiovascular disorder, a metabolic disorder, an ocular disorder, and an oncological disorder.

More preferably, the additional compound according to the invention is an anti-inflammatory compound or a disease-modifying antirheumatic drug. Preferably, the anti-inflammatory compound according to the invention is a non-steroidal antiinflmmatory compound, in particular selected from the group consisting of ibuprofen, flubirpofen, indomethacin, naproxen, prednisolone, sulindac, ketoprofen, phenylbutazone, celecoxib, fenbufene, diflusinal, piroxicam and diclofenac, or a steroidal anti-inflammatory compound, in particular selected from the group consisting of prednisolone and prednisone. More preferably, the disease-modifying antirheumatic drug is selected from the group consisting of chloroquine, hydroxychloroquine, methotrexate, tofacitinib, baricitinib and apremilast.

### Administration

As intended herein, “combined” or “in combination” means that the compound of formula (I), in particular the compound of formula (II), as defined above, is administered at the same time than another compound of product, either together, i.e. at the same administration site, or separately, or at different times, provided that the time period during which the compound of formula (I), in particular the compound of formula (II), as defined above exerts its effect on the individual and the time period during which the additional agent or product exerts its pharmacological effects on the individual, at least partially intersect.

Preferably, the compound of formula (I) or the pharmaceutical composition or medicament comprising the compound of formula (I) according to the invention is administered in a prophylactically or therapeutically effective amount for preventing or treating a cytokine-mediated disease.

The administration of the compound of formula (I) or the pharmaceutical composition or medicament comprising the compound of formula (I) according to the invention can proceed by any method known in the art. Preferably, the compound of formula (I) or the pharmaceutical composition or medicament comprising the compound of formula (I) according to the invention is in a form suitable for administration by the oral, parenteral, topical, intravenous, intramuscular, nasal or rectal route. More preferably, the compound of formula (I) or the pharmaceutical composition or medicament comprising the compound of formula (I) according to the invention is administered orally.

Preferably, the compound of formula (I) or the pharmaceutical composition or medicament comprising the compound of formula (I) according to the invention is in the form of powder, sachets, tablets, capsules, suspension, solution, or suppositories.

Preferably, the compound of formula (I) as defined above or the pharmaceutically acceptable salt or hydrate thereof, is to be administered at a unit dose of from 0.05 mg to 5000 mg, preferably from 1 mg to 3000 mg, more preferably from 10 mg to 3000 mg, more preferably from 100 to 1000 mg.

Preferably also, the compound of formula (I) as defined above or the pharmaceutically acceptable salt thereof is to be administered with a daily dosage regimen of from 0.01 mg/kg body weight to 100 mg/kg body weight, more preferably from 0.05 mg/kg body weight to 50 mg/kg body weight.

The present invention will be further described by the following non-limiting FIGURE and Examples.

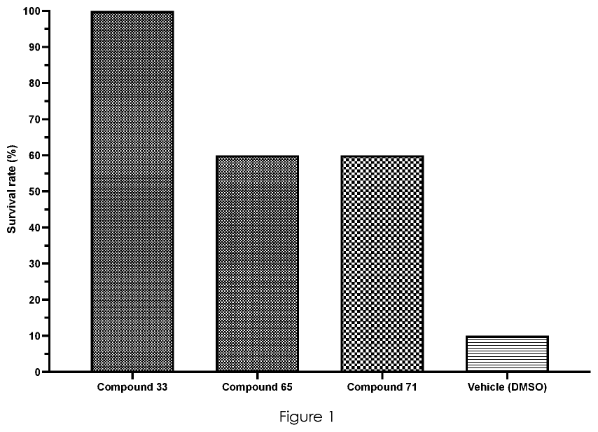

FIG. 1

FIG. 1 represents the survival rate (in %) of mice submitted to hepatic shock induced by LPS/D-Galactosamine and having been administered compound 33, compound 65, compound 71 or DMSO (negative control).

### EXAMPLES

### Example 1: Synthesis Protocol 1

Equivalent, similar or suitable solvents, reagents or reaction conditions may be substituted for those particular solvents, reagents or reaction conditions described without departing from the general scope of the method.

### 1. General Procedure 1: Mono-Protection of Diamine Alkyl

### 1.1. Preparation of tert-butyl 4-aminobutylcarbamate, (Intermediate 1, C₉H₂ON₂O₂, MW=188.27 g/mol)

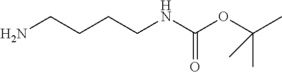

A solution of di-tert-butyl dicarbonate (807.5 mg, 3.7 mmol, 1 equiv) in DCM (16 mL, 0.23 M) was added dropwise, during 30 min, at 0° C. to a solution of butane-1,4-diamine (3.29 g, 37.37 mmol, 10.1 equiv) in DCM (30 mL, 1.25 M). The mixture was stirred from 0° C. to room temperature during 24 h. The mixture was concentrated under reduce pressure. The white solid obtained was re-dissolved in DCM (50 mL) and this solution was washed with saturated solution of NaHCO₃ (2×50 mL). The combined aqueous phases were extracted with DCM (3×100 mL). Some of the product still remaining in the aqueous phase. The aqueous phase was basified with NaOH 2 M (10 mL) and was extracted with diethyl ether (7×100 mL). The combined organic phases were dried over anhydrous MgSO₄ and concentrated to afford 779.6 mg of a white solid. The crude product was purified by flash column chromatography on silica gel, using a mobile phase gradient (DCM/MeOH·NH₃ 3% to 10%) to afford Intermediate 1 (643.7 mg, 92%) an amorphous yellow solid.

TLC: Eluent DCM/MeOH·NH₃ 5% R_{f}=0.2

¹H NMR (400 MHz, DMSO-d₆) δ 6.79 (1H, br, NH), 2.89 (2H, q, J=6.8 Hz, H4), 2.51-2.48 (2H, m, H1), 1.37-1.27 (13H, m, H7, H3 and H2).

¹³C NMR (100 MHz, DMSO-d₆) δ 155.58 (C5), 77.27 (C6), 41.42 (C1), 39.52 (C4), 30.65 (C2 or C3), 28.29 (C7), 27.05 (C2 or C3).

### 1.2. Preparation of N-(5-aminopentyl)pivalamide (Intermediate 2, C₁₀H₂₂N₂O₂, MW=202.29 g/mol)

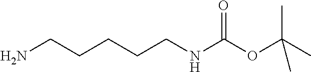

According to general procedure 1. Scale: pentane-1,5-diamine (3.82 g, 37.37 mmol, 5 equiv), di-tert-butyl dicarbonate (1.64 g, 7.5 mmol, 1 equiv), (DCM 60 mL). The crude product (1.51 g of a yellow oil) was purified by flash column chromatography on silica gel, using a mobile phase gradient (DCM/MeOH·NH₃ 3% to 10%) to afford Intermediate 2 (1.21 g, 86%) a colourless oil.

TLC: Eluent DCM/MeOH·NH3 5% Rf=0.17

1H NMR (400 MHz, DMSO-d6) δ 6.76 (1H, br, NH), 2.88 (2H, q, J=6.8 Hz, H5), 2.53-2.43 (2H, m, H1), 1.41-1.26 (13H, m, H8, H4 and H2), 1.23 (2H, m, H3).

13C NMR (100 MHz, DMSO-d6) δ 155.57 (C6), 77.26 (C7), 41.52 (C1), 41.21 (C5), 32.86 (C2), 29.39 (C4), 28.27 (C8), 23.69 (C3).

### 1.3. Preparation of tert-butyl (2-(2-aminoethoxy)ethyl)carbamate (Intermediate 23, C₉H₂ON₂O₃, MW=204.27 g/mol)

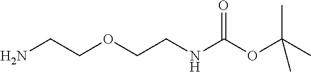

According to general procedure 1. Scale: 2,2′-Oxydiethanamine (508 mg, 4.87 mmol, 3.9 equiv), di-tert-butyl dicarbonate (275 mg, 1.26 mmol, 1 equiv), (DCM 23 mL). The crude product (243 mg of a yellow oil) was purified by flash column chromatography on silica gel, using a mobile phase gradient (DCM/MeOH·NH₃ 4% to 7%) to afford Intermediate 23 (190 mg, 74%) a colourless oil.

TLC: Eluent DCM/MeOH·NH3 5% Rf=0.42

¹H NMR (400 MHz, Chloroform-d) δ 3.48 (4H, m, H2 and H3), 3.30 (2H, m, H4), 2.84 (2H, t, J=5.2 Hz H1), 1.37 (9H, s, 1H7).

¹³C NMR (101 MHz, CDCl3) δ 156.08 (C5), 79.37 (C6), 73.30 (C2), 70.09 (C3), 41.92 (C1), 40.52 (C4), 28.52 (C7).

### 2. Procedure 1: Vilsmeier Haack Reaction

### 2.1. Preparation of 1H-indole-3-carbaldehyde (Intermediate 3, C₉H₇NO, MW=145.16 g/mol)

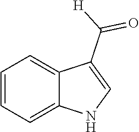 [SMILES: O=Cc1c[nH]c2ccccc12]

Under argon atmosphere, indole (5 g, 42.7 mmol, 1 equiv), was dissolved in anhydrous DMF (10 mL). Under argon atmosphere phosphorus oxychloride (4.4 mL, 47 mmol, 1.1 equiv) was added dropwise to anhydrous DMF (10 mL) the mixture was stirred during 25 min. The indole solution was added to the phosphorus oxychloride mixture at 0° C. The reactional mixture was stirred overnight from 0° C. to RT. After, NaOH 6M (30 mL) was added until to reach pH 9. The reactional mixture was heated to 100° C. during 2 h and then cooled to RT during 3 h. The reaction was quenched with a saturated solution of NaHCO₃(150 mL), the product was extracted with EtOAc (3×150 mL), the combined organic phases were washed with a saturated solution of NaHCO₃(4×100 mL), dried over Na₂SO₄ and concentrated to afford Intermediate 3 (5.1 g, 82%) as a red/orange solid.

¹H NMR (400 MHz, DMSO-d6) δ 12.13 (1H, br, H1), 9.93 (1H, s, H11), 8.29 (1H, s, H2), 8.09 (1H, m, H4), 7.51 (1H, m, H7), 7.24 (2H, m, H6 and H5).

¹³C NMR (100 MHz, DMSO-d6) δ 185.03 (C10), 138.50 (C2), 137.09 (C3), 124.15 (C9), 123.51 (C6), 122.17 (C5), 120.87 (C4), 118.21 (C8), 112.46 (C7).

### 3. General Procedure 1b

### 3.1. Preparation of 1-(phenylsulfonyl)-1H-indole-3-carbaldehyde (Intermediate 4, C₁₅H₁₁NO₃S, MW=285.32 g/mol)

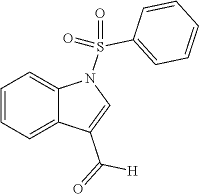 [SMILES: O=Cc1cn(S(=O)(=O)c2ccccc2)c2ccccc12]

Under argon atmosphere, Intermediate 3 (2 g, 13.78 mmol, 1 equiv), was dissolved in dry THF (35 mL). NaH (1.1 g, 27.56 mmol, 2 equiv) was added portion-wise to the Intermediate 3 solution at 0° C. The reactional mixture was stirred at 0° C. during 1 h. After, benzenesulfonyl chloride (2.92 g, 16.54 mmol, 1.2 equiv) was added dropwise to the reaction mixture, at 0° C. The reactional mixture was stirred overnight from 0° C. to RT. The mixture was concentrated under reduce pressure. The white solid obtained was with cold water. The crude product was purified by flash column chromatography on silica gel, using a Cyclohaxane/EtOAc 70/30 as the mobile phase to afford Intermediate 4 (3.325 g, 86%) a pink/red solid.

TLC: Eluent Cyclohexane/EtOAc 70/30 Rf=0.51

¹H NMR (400 MHz, DMSO-d6) δ 10.09 (1H, s, H11), 8.92 (1H, s, H2), 8.12 (3H, m, H7 and H13), 7.98 (1H, d, J=8.3 Hz, H4), 7.76 (1H, m, H15), 7.65 (2H, m, H14), 7.48-7.38 (m, 2H, H5 and H6).

¹³C NMR (100 MHz, DMSO-d6) δ 186.84 (C10), 138.59 (C2), 136.26 (C12), 135.46 (C15), 134.38 (C8), 130.23 (C14), 127.23 (C13), 126.36 (C5), 125.73 (C9), 125.21 (C6), 121.90 (C7), 121.66 (C13), 113.22 (C4).

### 3.2. Preparation of 1-(phenylsulfonyl)-1H-pyrrole-3-carbaldehyde (Intermediate 27, C11H9NO₃S, MW=235.26 g/mol)

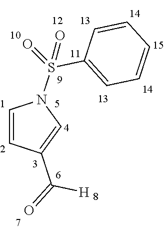 [SMILES: O=Cc1ccn(S(=O)(=O)c2ccccc2)c1]

According to general procedure 1b. Scale: 1H-pyrrole-3-carbaldehyde (212 mg, 2.18 mmol, 1 equiv), NaH 60% w/w in mineral oil (131 mg, 3.28 mmol, 1.5 equiv), benzenesulfonyl chloride (428 μL, 3.28 mmol, 1.5 equiv), DMF (10 mL). The crude product was purified by flash column chromatography on silica gel, using Cyclohexane/EtOAc 70/30 as eluent to afford Intermediate 27 (223 mg, 43%) as a brown sticky wax.

TLC: Eluent Cyclohexane/EtOAc 70/30 Rf=0.52

¹H NMR (400 MHz, Chloroform-d) δ 9.81 (s, 1H, H8), 7.98-7.90 (m, 2H, H13), 7.81-7.77 (m, 1H, H1), 7.74-7.62 (m, 1H, H15), 7.65-7.51 (m, 2H, H14), 7.19 (ddd, J=3.1, 2.2, 0.8 Hz, 1H, H4), 6.71 (dd, J=3.4, 1.6 Hz, 1H, H2).

¹³C NMR (101 MHz, CDCl3) δ 185.30 (C6), 138.00 (C11), 134.93 (C15), 129.95 (C14), 129.76 (C3), 128.22 (C1), 127.36 (C13), 122.56 (C4), 111.16 (C2).

### 3.3. Preparation of 1-(phenylsulfonyl)-1H-pyrrolo[2,3-b]pyridine-3-carbaldehyde (Intermediate 28, C14H10N2O3S MW=286.31 g/mol)

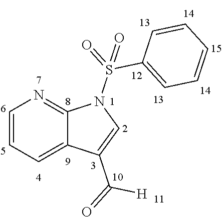 [SMILES: O=Cc1cn(S(=O)(=O)c2ccccc2)c2ncccc12]

According to general procedure 1b. Scale: 1H-pyrrolo[2,3-b]pyridine-3-carbaldehyde (254 mg, 1.74 mmol, 1 equiv), NaH 60% w/w in mineral oil (104 mg, 2.60 mmol, 1.5 equiv), benzenesulfonyl chloride (333 μL, 2.60 mmol, 1.5 equiv), DMF (10 mL). The crude product was purified by flash column chromatography on silica gel, using Cyclohexane/EtOAc 70/30 as eluent to afford Intermediate 28 (122 mg, 24%) as an colourless amorphous solid.

TLC: Eluent Cyclohexane/EtOAc 80/20 Rf=0.40

δ ¹H NMR (400 MHz, Chloroform-d) δ 10.05 (s, 1H, H11), 8.55-8.48 (m, 2H, H4 and H6), 8.40 (s, 1H, H2), 8.31-8.26 (m, 2H, H13), 7.68-7.61 (m, 1H, H15), 7.58-7.51 (m, 2H, H14), 7.32 (dd, J=7.8, 4.9 Hz, 1H, H5).

¹³C NMR (101 MHz, CDCl3) δ 185.29 (C10), 147.52 (C8), 146.87 (C6), 137.33 (C12), 135.83 (C2), 135.03 (C15), 131.51 (C4), 129.44 (C14), 128.73 (C13), 120.88 (C5), 119.61 (C9), 119.11 (C3).

### 3.4. Preparation of 1-(pyrrolidin-1-ylsulfonyl)-1H-indole-3-carbaldehyde (Intermediate 28b, C13H14N2O3S, MW=278.33 g/mol)

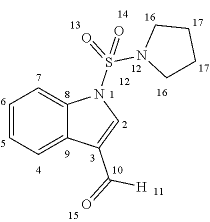 [SMILES: O=Cc1cn(S(=O)(=O)N2CCCC2)c2ccccc12]

According to general procedure 1b. Scale:1H-indole-3-carbaldehyde (253 mg, 1.74 mmol, 1 equiv), NaH 60% w/w in mineral oil (104 mg, 2.6 mmol, 1.5 equiv), pirrolidin-1-sulfonyl chloride (331 μL, 2.6 mmol, 1.5 equiv), DMF (2 mL). The crude product was purified by flash column chromatography on silica gel, using Cyclohexane/EtOAc 70/30 as eluent to afford Intermediate X (205 mg, 42%) as a colourless amorphous solid.

IR (ATR, cm-1): 3127, 2895, 1673, 1540, 1383, 1164, 1122, 1073, 750.

1H NMR (400 MHz, CDCl3): 10.09 (s, 1H, H11), 8.43-8.21 (m, 1H, H4), 8.13 (s, 1H, H2), 8.05-7.82 (m, 1H, H7), 7.52-7.36 (m, 2H, H5 and H6), 3.60-3.36 (m, 4H, H16), 1.92-1.71 (m, 4H, H17).

13C (400 MHz, CDCl3): 185.53 (C10), 137.44 (C2), 136.05 (C9), 126.06 (C5), 125.97 (C8), 124.80 (C6), 122.53 (C4), 120.61 (C3), 113.61 (C7), 48.95 (C16), 25.54 (C17).

### 3.5. Preparation of 1-((4-methylpiperazin-1-yl)sulfonyl)-1H-indole-3-carbaldehyde (MBP77) (Intermediate 28c, C14H17N3O3S, MW=307.37 g/mol)

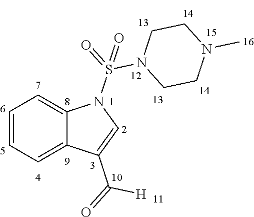 [SMILES: CN1CCN(S(=O)(=O)n2cc(C=O)c3ccccc32)CC1]

According to general procedure 1b. Scale: indole-3-carbaldehyde (244 mg, 1.68 mmol, 1 equiv), NaH 60% w/w in mineral oil (101 mg, 2.50 mmol, 1.5 equiv), 4-methylpiperazine-1-sulfonyl chloride (501 mg, 2.60 mmol, 1.5 equiv), DMF (10 mL). The crude product was purified by flash column chromatography on silica gel, using Cyclohexane/EtOAc 70/30 as eluent to afford Intermediate X (260 mg, 52%) as a brown amorphous solid.

IR (ATR, cm-1): 3134, 2923, 2826, 1663, 1395, 1290, 1184, 1126, 947.

1H NMR (400 MHz, CDCl3): 10.10 (s, 1H, H11), 8.40-8.22 (m, 1H, H4), 8.06 (s, 1H, H2), 7.97-7.82 (m, 1H, H7), 7.48-7.33 (m, 2H, H5 and H6), 3.37 (t, J=5.1 Hz, 4H, H13), 2.49 (t, J=5.1 Hz, 4H, H14), 2.28 (s, 3H, H16).

13C (400 MHz, CDCl3): 185.38 (C10), 136.88 (C2), 135.95 (C9), 126.29 (C5), 125.99 (C8), 125.08 (C6), 122.70 (C4), 121.19 (C3), 113.69 (C7), 53.63 (C14), 46.43 (C13), 45.53 (C16).

### 3.6. Preparation of 1-(piperidin-1-ylsulfonyl)-1H-indole-3-carbaldehyde (Intermediate 28d, C14H16N2O3S, MW=292.35 g/mol)

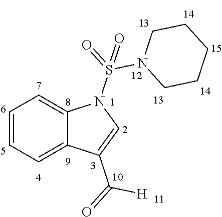 [SMILES: O=Cc1cn(S(=O)(=O)N2CCCCC2)c2ccccc12]

According to general procedure 1b. Scale: 1H-indole-3-carbaldehyde (171 mg, 1.18 mmol, 1 equiv), NaH 60% w/w in mineral oil (236 mg, 3.54 mmol, 3 equiv), piperidine-1-sulfonyl chloride (650 mg, 3.54 mmol, 3 equiv), DMF (2 mL). The crude product was purified by flash column chromatography on silica gel, using Cyclohexane/EtOAc 70/30 as eluent to afford Intermediate X (320 mg, 93%) as a colourless amorphous solid.

IR (ATR, cm-1): 3136, 2924, 2855, 1676, 1389, 1184, 1125, 1055, 938.

1H NMR (400 MHz, CDCl3): δ 10.09 (s, 1H), 8.42-8.17 (m, 1H), 8.08 (s, 1H), 8.02-7.85 (m, 1H), 7.51-7.34 (m, 2H), 3.38-3.21 (m, 4H), 1.67-1.49 (m, 6H).

13C (400 MHz, CDCl3): 185.50 (C10), 137.25 (C2), 135.96 (C9), 126.07 (C5), 125.92 (C8), 124.84 (C6), 122.57 (C4), 120.80 (C3), 113.81 (C7), 47.80 (C13), 25.03 (C14), 23.08 (C15).

### 4. General Procedure 2: First Reductive Amination, Between Primary Amine and Carbaldehyde Indole Sulphonamide

### 4.1. Preparation of tert-butyl 4-((1-(phenylsulfonyl)-1H-indol-3-yl)methylamino)butylcarbamate (Intermediate 5, C₂₄H₃₁N₃O₄S, MW=457.59 g/mol)

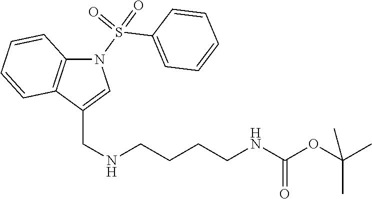 [SMILES: CC(C)(C)OC(=O)NCCCCNCc1cn(S(=O)(=O)c2ccccc2)c2ccccc12]

Under argon atmosphere, Intermediate 1 (400 mg, 2.12 mmol, 1 equiv) was dissolved in anhydrous MeOH (12 mL, 0.18 M), Intermediate 4 (604.9 mg, 2.12 mmol, 1 equiv) was added to the reactional mixture. After 1 h of stirring at RT, NaBH₄ (128.8 mg, 3.39 mmol, 1.6 equiv) was added at 0° C. The mixture was stirred from 0° C. to RT during 4 h, then concentrated under reduce pressure. The obtained mixture was re-dissolved in DCM (50 mL) and washed with a saturated solution of NaHCO₃(2×25 mL). Combined aqueous layers were extracted with DCM (3×50 mL). Combined organic layers were dried over MgSO4 and concentrated under reduce pressure, to afford 906 mg of an amorphous yellow solid. The crude product was purified by flash column chromatography on silica gel, using a mobile phase gradient (DCM/MeOH·NH₃ 1% to 4%) to afford Intermediate 5 (629.9 mg, 65%) as a yellow oil.

TLC: Eluent DCM/MeOH·NH3 3% Rf=0.58.

¹H NMR (400 MHz, Chloroform-d) δ 7.99 (1H, d, J=8.3 Hz, H11), 7.90-7.85 (2H, m, H15), 7.58-7.49 (2H, m, H8 and H17), 7.48 (1H, s, H13), 7.42 (2H, m, H16), 7.35-7.29 (1H, m, H10), 7.24 (1H, m, H9), 3.88 (2H, s, H5), 3.12 (2H, m, H1), 2.66 (2H, t, J=6.6 Hz, H4), 1.52 (4H, m, H2 and H3), 1.44 (9H, s, H20).

¹³C NMR (101 MHz, CDCl3) δ 156.14 (C18), 138.37 (C14), 135.56 (C12), 133.86 (C17), 130.39 (C7), 129.35 (C16), 126.87 (C15), 124.99 (C10), 123.64 (C13), 123.36 (C9), 121.99 (C6), 119.83 (C8), 113.86 (C11), 79.18 (C19), 49.34 (C4), 44.69 (C5), 40.56 (C1), 28.56 (C20), 28.01 (C2), 27.41 (C3) LRMS m/z (ESI+, CV 30) 458.3 [M+H]+.

### 4.2. Preparation of tert-butyl 5-((1-(phenylsulfonyl)-1H-indol-3-yl)methylamino)pentylcarbamate (Intermediate 6, C₂₅H₃₃N₃O₄S, MW=471.61 g/mol)

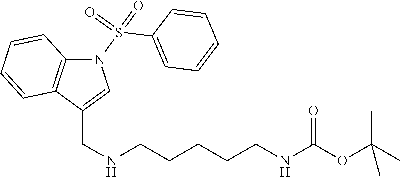 [SMILES: CC(C)(C)OC(=O)NCCCCCNCc1cn(S(=O)(=O)c2ccccc2)c2ccccc12]

According to general procedure 2. Scale: Intermediate 2 (1.21 g, 5.98 mmol, 1 equiv), Intermediate 4 (1.85 g, 6.5 mmol, 1.09 equiv), NaBH₄ (393.4 mg, 10.4 mmol, 1.74 equiv), MeOH (37 mL). The crude product (3.31 g of an orange oil) was purified by flash column chromatography on silica gel, using a mobile phase gradient (DCM/MeOH·NH3 1% to 4%) to afford Intermediate 6 (2.43 g, 86%) as an orange oil.

TLC: Eluent DCM/MeOH·NH3 2% Rf=0.44.

¹H NMR (400 MHz, Chloroform-d) δ 7.99 (1H, dt, J=8.3, 0.8 Hz, H12), 7.87 (2H, dd, J=8.5, 1.2 Hz, H16), 7.58-7.49 (2H, m, H9 and H18), 7.48 (1H, s, H14), 7.42 (2H, m, H17), 7.35-7.29 (1H, m, H11), 7.24-7.21 (1H, m, H10), 3.88 (2H, s, H6), 3.10 (2H, q, J=6.4 Hz, H1), 2.64 (2H, t, J=7.1 Hz, H5), 1.56-1.46 (4H, m, H2 and H4), 1.43 (9H, s, H21), 1.35 (2H, m, H3).

¹³C NMR (100 MHz, CDCl3) δ 156.11 (C19), 138.37 (C15), 135.56 (C13), 133.86 (C18), 130.41 (C8), 129.34 (C17), 126.88 (C16), 124.97 (C11), 123.60 (C14), 123.35 (C10), 122.06 (C7), 119.83 (C9), 113.86 (C12), 79.18 (C20), 49.63 (C5), 44.74 (C6), 40.63 (C1), 30.09 (C4), 29.81 (C2), 28.55 (C21), 24.66 (C3).

LRMS m/z (ESI+, CV 30) 472.2 [M+H]+.

### 4.3. Preparation of tert-butyl (2-(2-(((1-(phenylsulfonyl)-1H-indol-3-yl)methyl)amino)ethoxy)ethyl) carbamate (Intermediate 24, C₂₄H₃₁N₃O₅S, MW=473.59 g/mol)

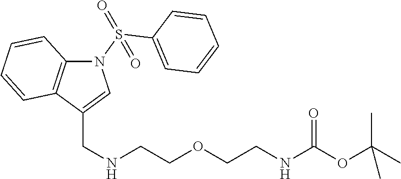 [SMILES: CC(C)(C)OC(=O)NCCOCCNCc1cn(S(=O)(=O)c2ccccc2)c2ccccc12]

According to general procedure 2. Scale: Intermediate 23 (190 mg, 0.93 mmol, 1 equiv), 1-(phenylsulfonyl)-1H-indole-3-carbaldehyde x (265 mg, 0.93 mmol, 1 equiv), NaBH(OAc)₃ (591 mg, 2.78 mmol, 3 equiv), DCM (9 mL). The crude product (493 mg of an orange amorphous solid) was purified by flash column chromatography on silica gel, using a mobile phase gradient (DCM/MeOH·NH₃ 1% to 4%) to afford Intermediate 24 (177 mg, 40%) an orange amorphous solid.

TLC: Eluent DCM/MeOH·NH3 2% Rf=0.53

1H NMR (400 MHz, Chloroform-d) δ 7.99 (1H, m, H11), 7.90-7.85 (2H, m, H15), 7.56 (1H, m, H8), 7.54-7.49 (2H, m, H13 and H17), 7.42 (2H, m, H16), 7.32 (1H, m, H10), 7.24 (1H, m, H9), 4.95 (1H, br, NH), 3.92 (2H, s, H5), 3.56 (2H, t, J=5.1 Hz, H3), 3.48 (2H, t, J=5.2 Hz, H2), 3.30 (2H, m, H1), 2.82 (2H, t, J=5.1 Hz, H4), 1.43 (9H, s, H20).

13C NMR (101 MHz, CDCl3) δ 156.09 (C18), 138.37 (C14), 135.57 (C12), 133.86 (C17), 130.38 (C7), 129.35 (C16), 126.89 (C15), 125.00 (C10), 123.73 (C13), 123.36 (C9), 121.84 (C6), 119.82 (C8), 113.86 (C11), 79.41 (C19), 70.44 (C3), 70.22 (C2), 48.98 (C4), 44.63 (C5), 40.54 (C1), 28.53 (C20).

LRMS m/z (ESI+, CV 30) 474.2 [M+H]+.

### 5. General Procedure 3: Second Reductive Amination, Between Secondary Amine and Fluoro-Pyridine Carbaldehyde

### 5.1. Preparation of tert-butyl 4-(((2-fluoropyridin-3-yl)methyl)((1-(phenylsulfonyl)-1H-indol-3-yl)methyl)amino)butylcarbamate (Intermediate 7, C₃₀H₃₅FN₄O₄S, MW=566.69 g/mol)

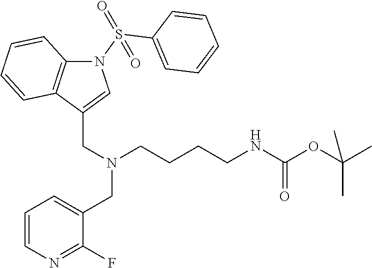 [SMILES: CC(C)(C)OC(=O)NCCCCN(Cc1cccnc1F)Cc1cn(S(=O)(=O)c2ccccc2)c2ccccc12]

Under argon atmosphere, Intermediate 5 (171 mg, 0.374 mmol, 1 equiv) was dissolved in anhydrous DCM (6 mL, 0.06 M), 2-Fluoro-3-pyridinecarboxaldehyde (53 mg, 0.424 mmol, 1.13 equiv) was added to the reactional mixture. After 1 h of stirring at RT, NaBH(OAc)3 (119 mg, 0.562 mmol, 1.5 equiv) was added at 0° C. The mixture was stirred from 0° C. to RT during 4 h. Then, NaBH(OAc)₃ (119 mg, 0.562 mmol, 1.5 equiv) was added to the mixture, under stirring at RT. After 25 h, the mixture was re-dissolved in DCM (25 mL) and washed with a saturated solution of NaHCO₃(1×25 mL). The aqueous layer was extracted DCM (5×50 mL). The combined organic layers were dried over MgSO4 and concentrated under reduce pressure, to afford 242.2 mg of an orange oil. The crude product was purified by flash column chromatography on silica gel, using a solvent gradient (DCM/MeOH·NH3 0% to 5%) to afford Intermediate 7 (157.5 mg, 74%) an amorphous solid.

TLC: Eluent DCM/MeOH·NH3 2% Rf=0.43.

¹H NMR (400 MHz, DMSO-d6) δ 8.08 (1H, m, H21), 7.91-7.82 (4H, m, H11, H15 and H23), 7.72 (1H, s, H13), 7.62 (1H, t, J=7.5 Hz, H17), 7.57-7.49 (3H, m, H8 and H16), 7.33 (1H, t, J=7.3 Hz, H10), 7.26-7.21 (2H, m, H9 and H22), 6.75 (1H, br, NH), 3.68 (2H, s, H5), 3.55 (2H, s, H18), 2.82 (2H, q, J=6.5 Hz, H1), 2.34 (2H, t, J=7.1 Hz, H4), 1.45 (2H, m, H3), 1.35 (9H, s, H26), 1.25 (2H, m, H2).

¹³C NMR (100 MHz, DMSO-d6) δ 160.22 (C20), 155.62 (C24), 145.92 (C21), 141.76 (C23), 136.87 (C14), 134.87 (C12), 134.50 (C17), 130.48 (C7), 129.70 (C16), 126.51 (C15), 125.22 (C13), 124.96 (C10), 123.38 (C9), 121.95 (C22), 120.86 (C19), 120.78 (C6), 120.56 (C8), 113.29 (C11), 77.36 (C25), 52.99 (C4), 50.38 (C18), 48.52 (C5), 39.52 (C1), 28.27 (C26), 27.35 (C2), 23.47 (C3).

LRMS m/z (ESI+, CV 30) 567.3 [M+H]+.

### 5.2. Preparation of tert-butyl 4-(((2-fluoropyridin-4-yl)methyl)((1-(phenylsulfonyl)-1H-indol-3-yl)methyl)amino)butylcarbamate (Intermediate 8, C₃₀H₃₅FN₄O₄S, MW=566.69 g/mol)

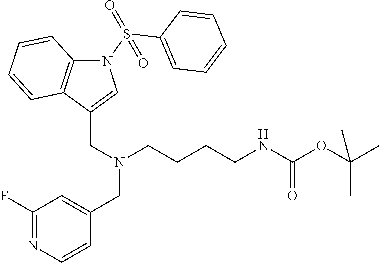 [SMILES: CC(C)(C)OC(=O)NCCCCN(Cc1ccnc(F)c1)Cc1cn(S(=O)(=O)c2ccccc2)c2ccccc12]

According to general procedure 3. Scale: Intermediate 5 (315 mg, 0.688 mmol, 1 equiv), 3-Fluoropyridine-4-carboaxaldehyde (97 mg, 0.775 mmol, 1.13 equiv), NaBH(OAc)3 (438 mg, 2.067 mmol, 3 equiv), DCM (II mL). The crude product (390 mg of an amorphous yellow solid) was purified by flash column chromatography on silica gel, using a solvent gradient (DCM/MeOH·NH₃ 1% to 3%) to afford Intermediate 8 (229.4 mg, 59%) a colourless oil.

TLC: Eluent DCM/MeOH·NH3 2% R_{f}=0.85

¹H NMR (400 MHz, DMSO-d₆) δ 8.08 (1H, d, J=5.1 Hz, H21), 7.92-7.89 (3H, m, H11 and H15), 7.74 (1H, s, H13), 7.65-7.59 (2H, m, H8 and H17), 7.50 (2H, t, J=7.7 Hz, H16), 7.37-7.32 (1H, m, H10), 7.28-7.24 (1H, m, H9), 7.19 (1H, m, H20), 6.97 (1H, s, H23), 6.78 (1H, br, NH), 3.69 (2H, s, H5), 3.56 (2H, s, H18), 2.85 (2H, q, J=6.6 Hz, H1), 2.35 (2H, t, J=7.2 Hz, H4), 1.47 (2H, p, J=8.1, 7.7 Hz, H3), 1.35-1.27 (11H, s, H2 and H26).

¹³C NMR (100 MHz, DMSO-d₆) δ 164.51 (C22), 162.18 (C19), 155.61 (C24), 147.16 (C21), 136.85 (C14), 134.86 (C12), 134.47 (C17), 130.46 (C7), 129.66 (C16), 126.50 (C15), 125.43 (C13), 124.99 (10), 123.42 (C9), 121.58 (C20), 120.60 (C6), 120.55 (C8), 113.33 (C11), 108.26 (23), 77.33 (C25), 56.20 (C18), 53.40 (C4), 48.67 (C5), 39.37 (C1), 28.25 (C26), 27.34 (C2), 23.59 (C3).

### 5.3. Preparation of tert-butyl 4-(((6-fluoropyridin-2-yl)methyl)((1-(phenylsulfonyl)-1H-indol-3-yl)methyl)amino)butylcarbamate (Intermediate 9, C₃₀H₃₅FN₄O₄S, MW=566.69 g/mol)

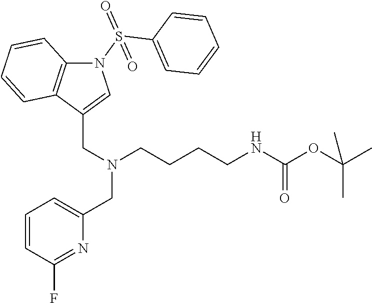 [SMILES: CC(C)(C)OC(=O)NCCCCN(Cc1cccc(F)n1)Cc1cn(S(=O)(=O)c2ccccc2)c2ccccc12]

According to general procedure 3. Scale: Intermediate 5 (269 mg, 0.588 mmol, 1 equiv), 3-Fluoropyridine-2-carboaxaldehyde (82 mg, 0.655 mmol, 1.11 equiv), NaBH(OAC)₃ (561 mg, 2.65 mmol, 4.5 equiv), DCM (10 mL). The crude product (347.2 mg of an amorphous brown solid) was purified by flash column chromatography on silica gel, using a solvent gradient (DCM/MeOH·NH₃ 1% to 4%) to afford Intermediate 9 (217.9 mg, 65%) an amorphous yellow solid.

TLC: Eluent DCM/MeOH·NH₃ 2% R_{f}=0.90

¹H NMR (400 MHz, Chloroform-d) δ 7.98 (1H, d, J=8.2 Hz, H11), 7.82 (2H, m, H15), 7.69 (1H, q, J=8.1 Hz, H22), 7.62 (1H, d, J=7.5 Hz, H8), 7.49-7.46 (2H, m, H13 and H17), 7.38 (2H, m, H16), 7.34-7.28 (1H, m, H10), 7.26-7.20 (2H, m, H9 and H23), 6.76 (1H, dd, J=8.0, 2.6 Hz, H21), 3.72 (2H, s, H5), 3.62 (2H, s, H18), 3.01 (2H, d, J=6.3 Hz, H1), 2.49 (2H, t, J=7.1 Hz, H4), 1.59-1.49 (2H, m, H3), 1.43 (11H, m, H2 and H26).

¹³C NMR (100 MHz, Chloroform-d) δ 164.20 (C20), 161.82 (C19), 156.08 (C24), 141.37 (C22), 138.20 (C14), 135.71 (C12), 133.87 (C17), 130.85 (C7), 129.32 (C16), 126.78 (C15), 125.03 (C10), 124.93 (C13), 123.37 (C9), 120.95 (C6), 120.54 (C8), 120.03 (C23), 113.87 (C11), 107.22 (C21), 79.22 (C25), 59.36 (C18), 54.09 (C4), 49.79 (C5), 40.48 (C1), 28.55 (C26), 27.99 (C2), 24.55 (C3).

### 5.4. Preparation of tert-butyl 4-(((6-fluoropyridin-3-yl)methyl)((1-(phenylsulfonyl)-1H-indol-3-yl)methyl)amino)butylcarbamate (Intermediate 10, C₃₀H₃₅FN₄O₄S, MW=566.69 g/mol)

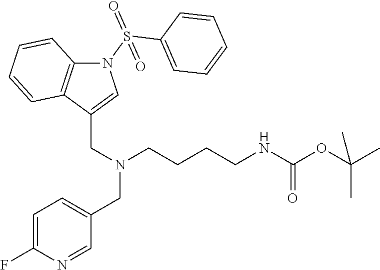 [SMILES: CC(C)(C)OC(=O)NCCCCN(Cc1ccc(F)nc1)Cc1cn(S(=O)(=O)c2ccccc2)c2ccccc12]

According to general procedure 3. Scale: Intermediate 5 (215 mg, 0.470 mmol, 1 equiv), 4-Fluoropyridine-3-carboaxaldehyde (65.8 mg, 0.526 mmol, 1.12 equiv), NaBH(OAC)₃ (298.8 mg, 1.41 mmol, 3 equiv), DCM (7.5 mL). The crude product (297 mg of an amorphous yellow solid) was purified by flash column chromatography on silica gel, using a solvent gradient (DCM/MeOH·NH₃ 0% to 2%) to afford Intermediate 10 (154.1 mg, 58%) an amorphous yellow solid.

TLC: Eluent DCM/MeOH·NH₃ 1% R_{f}=0.84

¹H NMR (400 MHz, Chloroform-d) δ 8.04 (1H, m, H20), 7.99 (1H, d, J=8.3 Hz, H11), 7.84 (2H, m, H15), 7.65 (1H, td, J=8.1, 2.4 Hz, H23), 7.57 (1H, d, J=7.7 Hz, H8), 7.52-7.45 (2H, m, H13 and H17), 7.42-7.36 (2H, m, H16), 7.33 (1H, m, H10), 7.24-7.22 (1H, m, H9), 6.84 (dd, J=8.4, 2.8 Hz, H22), 3.64 (2H, s, H17), 3.47 (2H, s, H18), 2.99 (2H, m, H1), 2.40 (2H, t, J=7.1 Hz, H4), 1.51 (2H, p, J=8.0, 7.5 Hz, H3), 1.43 (9H, s, H26), 1.37 (2H, m, H2).

¹³C NMR (100 MHz, Chloroform-d) δ 164.25 (C21), 161.88 (C19), 156.09 (C24), 147.61 (C20), 141.82 (C23), 138.22 (C14), 135.75 (C12), 133.94 (C17), 130.69 (C7), 129.36 (C16), 126.78 (C15), 125.18 (C10), 124.90 (C13), 123.47 (C9), 120.74 (C6), 120.54 (C8), 113.93 (C11), 109.33 (C22), 79.31 (C25), 54.90 (C18), 53.48 (C4), 49.51 (C5), 40.44 (C1), 28.55 (C26), 28.05 (C2), 24.44 (C3).

LRMS m/z (ESI+, CV 30) 589.2 [M+Na]⁺.

### 5.5. Preparation of tert-butyl 5-(((2-fluoropyridin-3-yl)methyl)((1-(phenylsulfonyl)-1H-indol-3-yl)methyl)amino)pentylcarbamate (Intermediate 11, C₃₁H₃₇FN₄O₄S, MW=580.71 g/mol)

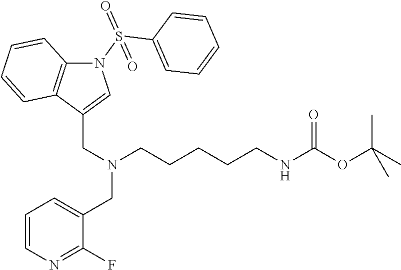 [SMILES: CC(C)(C)OC(=O)NCCCCCN(Cc1cccnc1F)Cc1cn(S(=O)(=O)c2ccccc2)c2ccccc12]

According to general procedure 3. Scale: Intermediate 6 (300 mg, 0.636 mmol, 1 equiv), 2-Fluoropyridine-3-carboaxaldehyde (89 mg, 0.710 mmol, 1.12 equiv), NaBH(OAc)3 (539 mg, 2.543 mmol, 4 equiv), DCM (10 mL). The crude product (421 mg of an amorphous brown solid) was purified by flash column chromatography on silica gel, using a solvent gradient (DCM/MeOH·NH₃ 1% to 4%) to afford Intermediate 11 (227.8 mg, 62%) an amorphous yellow solid.

TLC: Eluent DCM/MeOH·NH₃ 2% R_{f}=0.96

¹H NMR (400 MHz, Chloroform-d) δ 8.06 (1H, m, H22), 7.98 (1H, d, J=8.3 Hz, H12), 7.81 (2H, m, H16), 7.76 (1H, m, H24), 7.56 (1H, d, J=7.8 Hz, H9), 7.51-7.45 (2H, m, H14 and H18), 7.38 (2H, m, H17), 7.34-7.27 (1H, m, H11), 7.25-7.20 (1H, m, H10), 7.11 (1H, m, H23), 3.67 (2H, s, H6), 3.55 (2H, s, H19), 3.01 (2H, q, J=6.4 Hz, H1), 2.42 (2H, m, H5), 1.51 (2H, m, H4), 1.43 (9H, s, H27), 1.36-1.17 (4H, m, H2 and H3).

¹³C NMR (100 MHz, Chloroform-d) δ 160.93 (C21), 156.09 (C25), 146.06 (C22), 141.41 (C24), 138.20 (C15), 135.76 (C13), 133.91 (C18), 130.77 (C8), 129.33 (C17), 126.77 (C16), 125.10 (C11), 124.81 (C14), 123.41 (C10) 121.56 (C23), 121.38 (C20), 121.00 (C7), 120.56 (C9), 113.89 (C12), 79.23 (C26), 53.96 (C5), 50.92 (C19), 49.81 (C6), 40.58 (C1), 29.93 (C2), 28.57 (C27), 26.84 (C4), 24.54 (C3).

LRMS m/z (ESI+, CV 30) 603.2 [M+Na]⁺.

### 5.6. Preparation of tert-butyl 5-(((2-fluoropyridin-4-yl)methyl)((1-(phenylsulfonyl)-1H-indol-3-yl)methyl)amino)pentylcarbamate (Intermediate 12, C₃₁H₃₇FN₄O₄S, MW=580.71 g/mol)

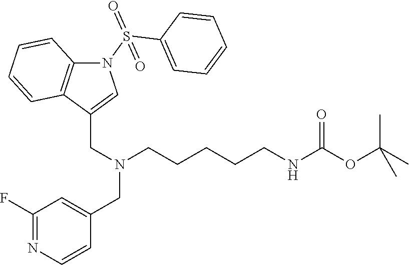 [SMILES: CC(C)(C)OC(=O)NCCCCCN(Cc1ccnc(F)c1)Cc1cn(S(=O)(=O)c2ccccc2)c2ccccc12]

According to general procedure 3. Scale: Intermediate 6 (500 mg, 1.06 mmol, 1 equiv), 3-Fluoropyridine-4-carboaxaldehyde (149 mg, 1.191 mmol, 1.12 equiv), NaBH(OAc)₃ (674 mg, 3.18 mmol, 3 equiv), DCM (17 mL). The crude product (647 mg of a yellow oil) was purified by flash column chromatography on silica gel, using a solvent gradient (DCM/MeOH·NH₃ 1% to 3%) to afford Intermediate 12 (580.2 mg, 94%) an amorphous yellow solid.

TLC: Eluent DCM/MeOH·NH₃

¹H NMR (400 MHz, Chloroform-d) δ 8.08 (1H, d, J=5.1 Hz, H22), 7.99 (1H, d, J=8.3 Hz, H12), 7.83 (2H, m, H16), 7.60 (1H, d, J=7.7 Hz, H9), 7.48 (2H, m, H14 and H18), 7.42-7.30 (3H, m, H11 and H17), 7.29-7.23 (1H, m, H10), 7.07 (1H, m, H21), 6.84 (1H, s, H24), 3.66 (2H, s, H6), 3.51 (2H, s, H19), 3.04 (2H, q, J=6.5 Hz, H1), 2.41 (2H, m, H5), 1.53 (2H, p, J=7.5 Hz, H4), 1.43 (9H, s, H27), 1.35 (2H, m, H2), 1.24 (2H, p, J=7.5, 7.0 Hz, H3).

¹³C NMR (100 MHz, Chloroform-d) δ 163.05 (C23), 156.10 (C25), 155.68 (C20), 147.49 (C21), 138.19 (C15), 135.73 (C13), 133.95 (C18), 130.67 (C8), 129.35 (C17), 126.76 (C16), 125.21 (C11), 124.92 (C14), 123.49 (C10), 121.34 (C21), 120.60 (C7), 120.45 (C9), 113.93 (C12), 109.01 (C24), 79.23 (C26), 57.30 (C19), 54.27 (C5), 49.80 (C6), 40.56 (C1), 29.99 (C2), 28.55 (C27), 26.86 (C4), 24.54 (C3).

LRMS m/z (ESI+, CV 30) 581.2 [M+H]⁺.

### 5.7. Preparation of tert-butyl 5-(((6-fluoropyridin-2-yl)methyl)((1-(phenylsulfonyl)-1H-indol-3-yl)methyl)amino)pentylcarbamate (Intermediate 13, C₃₁H₃₇FN₄O₄S, MW=580.71 g/mol)

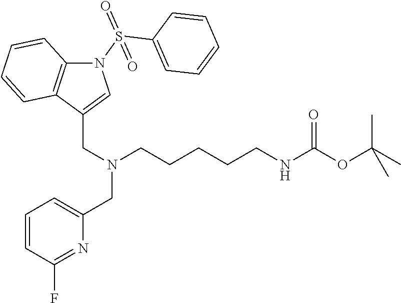 [SMILES: CC(C)(C)OC(=O)NCCCCCN(Cc1cccc(F)n1)Cc1cn(S(=O)(=O)c2ccccc2)c2ccccc12]

According to general procedure 3. Scale: Intermediate 6 (500 mg, 1.06 mmol, 1 equiv), 3-Fluoropyridine-2-carboaxaldehyde (150 mg, 1.199 mmol, 1.13 equiv), NaBH(OAc)3 (674 mg, 3.18 mmol, 3 equiv), DCM (17 mL). The crude product (560.5 mg of an amorphous brown solid) was purified by flash column chromatography on silica gel, using a solvent gradient (DCM/MeOH·NH₃ 1% to 3%) to afford Intermediate 13 (539 mg, 88%) an amorphous yellow solid.

TLC: Eluent DCM/MeOH·NH₃ 2% R_{f}=0.94

¹H NMR (400 MHz, Chloroform-d) δ 7.98 (1H, d, J=8.2 Hz, H12), 7.82 (2H, m, H16), 7.69 (1H, q, J=8.1 Hz, H23), 7.62 (1H, d, J=7.8 Hz, H9), 7.50-7.46 (2H, m, H14 and H18), 7.37 (2H, t, J=7.7 Hz, H17), 7.34-7.29 (1H, m, H11), 7.28-7.21 (2H, m, H10 and H24), 6.75 (1H, dd, J=8.1, 2.6 Hz, H22), 3.71 (2H, s, H6), 3.62 (2H, s, H19), 3.02 (2H, q, J=6.3 Hz, H1), 2.46 (2H, m, H5), 1.52 (2H, m, H4), 1.43 (9H, s, H27), 1.34 (2H, m, H2), 1.23 (2H, m, H3).

¹³C NMR (100 MHz, Chloroform-d) δ 161.80 (C21), 159.62 (C20), 156.08 (C25), 141.36 (C23), 138.20 (C15), 135.72 (13), 133.86 (C18), 130.88 (C8), 129.31 (C17), 126.77 (C16), 125.01 (C11), 124.91 (C14), 123.36 (C10), 121.09 (C7), 120.59 (C9), 120.00 (C24), 113.86 (C12), 107.33 (C22), 79.17 (C26), 59.47 (C19), 54.25 (C5), 49.86 (C6), 40.62 (C1), 29.95 (C2), 28.55 (C27), 26.95 (C4), 24.58 (C3).

### 5.8. Preparation of tert-butyl 5-(((6-methylpyridin-3-yl)methyl)((1-(phenylsulfonyl)-1H-indol-3-yl)methyl)amino)pentylcarbamate (Intermediate 14, C₃₁H₃₇FN₄O₄S, MW=580.71 g/mol)

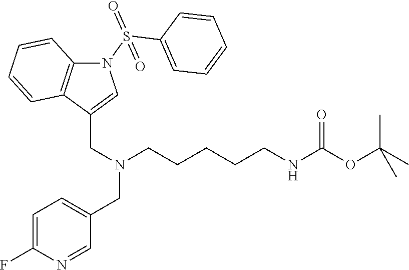 [SMILES: CC(C)(C)OC(=O)NCCCCCN(Cc1ccc(F)nc1)Cc1cn(S(=O)(=O)c2ccccc2)c2ccccc12]

According to general procedure 3. Scale: Intermediate 6 (439 mg, 0.931 mmol, 1 equiv), 4-Fluoropyridine-3-carboaxaldehyde (130 mg, 1.039 mmol, 1.12 equiv), NaBH(OAc)3 (591 mg, 2.789 mmol, 3 equiv), DCM (15 mL). The crude product (563 mg of an amorphous orange solid) was purified by flash column chromatography on silica gel, using a solvent gradient (DCM/MeOH·NH₃ 0% to 3%) to afford Intermediate 14 (370 mg, 69%) a yellow oil.

TLC: Eluent DCM/MeOH·NH₃ 2% R_{f}=0.90

¹H NMR (400 MHz, Chloroform-d) δ 8.05 (1H, m, H21), 7.98 (1H, d, J=8.3 Hz, H12), 7.86-7.81 (2H, m, H16), 7.65 (1H, td, J=8.1, 2.4 Hz, H24), 7.57 (1H, d, J=8.5 Hz, H9), 7.48 (2H, m, H14 and H18), 7.39 (2H, m, H17), 7.35-7.30 (1H, m, H11), 7.25-7.21 (1H, m, H10), 6.84 (1H, dd, J=8.4, 2.8 Hz, H23), 3.63 (2H, s, H6), 3.47 (2H, s, H19), 3.00 (2H, q, J=6.4 Hz, H1), 2.38 (2H, m, H5), 1.51 (2H, m, H4), 1.43 (9H, s, H27), 1.31 (2H, m, H2), 1.20 (2H, m, H3).

¹³C NMR (100 MHz, Chloroform-d) δ 164.24 (C22), 161.87 (C20), 156.09 (C25), 147.59 (C21), 141.83 (C24), 138.23 (C15), 135.76 (C13), 133.93 (C18), 130.73 (C8), 129.35 (C17), 126.78 (C16), 125.16 (C11), 124.89 (C14), 123.45 (C10), 120.87 (C7), 120.60 (C9), 113.91 (C12), 109.31 (C23), 79.24 (C26), 54.90 (C19), 53.57 (C5), 49.58 (C6), 40.57 (C1), 29.93 (C2), 28.56 (C27), 26.75 (C4), 24.52 (C3).

LRMS m/z (ESI+, CV 30) 603.2 [M+Na]⁺.

### 5.9. Preparation of tert-butyl (2-(2-(((2-fluoropyridin-3-yl)methyl) ((1-(phenylsulfonyl)-1H-indol-3-yl)methyl)amino)ethoxy)ethyl)carbamate (Intermediate 25, C₃₀H₃₅FN₄O₅S, Molecular Weight: 582,69)

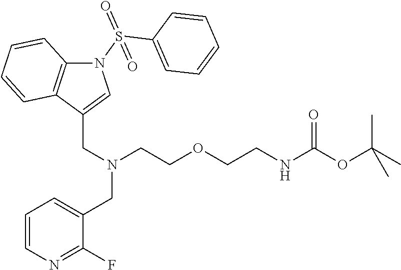 [SMILES: CC(C)(C)OC(=O)NCCOCCN(Cc1cccnc1F)Cc1cn(S(=O)(=O)c2ccccc2)c2ccccc12]

According to general procedure 3. Scale: Intermediate 24 (161 mg, 0.339 mmol, 1 equiv), 2-Fluoropyridine-3-carboaxaldehyde (79 mg, 0.39 mmol, 1.15 equiv), NaBH(OAc)3 (216 mg, 1.02 mmol, 3 equiv), DCM (4 mL). The crude product (247 mg of an orange oil) was purified by flash column chromatography on silica gel, using a solvent gradient (DCM/MeOH·NH₃ 1% to 2%) to afford Intermediate 25 (165 mg, 83%) an amorphous yellow solid.

TLC: Eluent DCM/MeOH·NH3 1% Rf=0.75

1H NMR (400 MHz, CDCl3) δ 8.06 (1H, d, H21), 7.97 (1H, d, J=8.3 Hz, H11), 7.82 (3H, m, H15 and H23), 7.60 (1H, d, J=7.6 Hz, H8), 7.54 (1H, s, H13), 7.48 (1H, tt, J=7.5 Hz, J=1.4 Hz, H17), 7.38 (2H, m, H16), 7.26 (1H, m, H10), 7.23 (1H, m, H9), 7.11 (1H, m, H22), 4.89 (1H, br, NH), 3.78 (2H, s, H5), 3.67 (2H, s, H18), 3.55 (2H, t, J=5.5 Hz, H3), 3.42 (2H, t, J=5.1 Hz, H2), 3.29 (2H, m, H1), 2.71 (2H, t, J=5.5 Hz, H4), 1.42 (9H, s, H26).

13C NMR (101 MHz, CDCl3) δ 160.83 (C20), 156.07 (C24), 146.05 (C21), 141.37 (C23), 138.16 (C14), 135.74 (C12), 133.89 (C17), 130.69 (C7), 129.31 (C16), 126.78 (C15), 125.09 (C10), 124.91 (C13), 123.42 (C9), 121.54 (C22), 121.24 (C19), 120.84 (C6), 120.50 (C8), 113.88 (C11), 79.41 (C25), 70.23 (C2), 69.57 (C3), 53.46 (C4), 51.36 (C18), 50.31 (C5), 40.61 (C1), 28.52 (C26).

19F NMR (376 MHz, Chloroform-d) δ−72.55

LRMS m/z (ESI+, CV 30) 583.3 [M+H]+, 605.3 [M+Na]+.

### 6. General Procedure 4. Deprotection

### 61. Preparation of N′-((2-fluoropyridin-3-yl)methyl)-N′-((1-(phenylsulfonyl)-H-indol-3-yl)methyl)butane-1,4-diamine (Intermediate 15, C₂₅H₂₇FN₄O₂S, MW=466.57 g/mol)

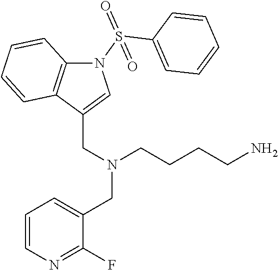 [SMILES: NCCCCN(Cc1cccnc1F)Cc1cn(S(=O)(=O)c2ccccc2)c2ccccc12]

Intermediate 7 (399 mg, 0.704 mmol, 1 equiv) was dissolved in DCM (12 mL, 0.06 M), TFA (1.204 g, 10.56 mmol, 15 equiv) was added. The reactional mixture was stirred at RT during 2 h. After, the reactional mixture was quenched with a solution of NaOH 2M (12 mL). The mixture was washed with saturated solution of NaHCO₃(1×30 mL). The aqueous phases were extracted with DCM (3×40 mL). The combined organic phases were dried over anhydrous MgSO₄ and concentrated to afford the product Intermediate 15 (308 mg, 94%) an orange oil.

TLC: Eluent DCM/MeOH·NH₃ 4% R_{f}=0.22

¹H NMR (400 MHz, Chloroform-d) δ 8.04 (1H, m, H21), 7.97 (1H, d, J=8.3 Hz, H11), 7.82 (2H, m, H15), 7.75 (1H, m, H23), 7.55 (1H, d, J=7.8 Hz, H8), 7.50-7.45 (2H, m, H13 and H17), 7.37 (2H, m, H16), 7.30 (1H, m, H10), 7.22 (1H, m, H9), 7.09 (1H, m, H22), 3.68 (2H, s, H5), 3.55 (2H, s, H18), 2.62 (2H, t, J=7.0 Hz, H1), 2.45 (2H, t, J=7.1 Hz, H4), 1.55 (2H, p, J=8.1, 7.5 Hz, H3), 1.41 (2H, p, J=7.0 Hz, H2).

¹³C NMR (100 MHz, Chloroform-d) δ 160.91 (C20), 146.09 (C21), 141.46 (C23), 138.16 (C14), 135.69 (C12), 133.93 (C17), 130.73 (C7), 129.33 (C16), 126.78 (C15), 125.11 (C10), 124.82 (C13), 123.43 (C19), 121.59 (C22), 121.23 (C19), 120.74 (C6), 120.47 (C8), 113.87 (C11), 53.92 (C4), 51.00 (C18), 49.67 (C5), 41.57 (C1), 30.23 (C2), 24.46 (C3).

LRMS m/z (ESI+, CV 30) 467.2 [M+H]⁺.

### 6.2. Preparation of N1-((2-fluoropyridin-4-yl)methyl)-N1-((1-(phenylsulfonyl)-1H-indol-3-yl)methyl)butane-1,4-diamine (Intermediate 16, C₂₅H₂₇FN₄O₂S, MW=466.57 g/mol)

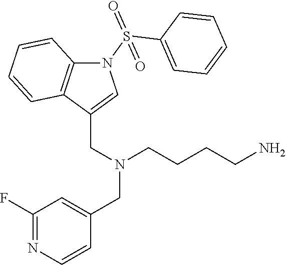 [SMILES: NCCCCN(Cc1ccnc(F)c1)Cc1cn(S(=O)(=O)c2ccccc2)c2ccccc12]

According to general procedure 4. Scale: Intermediate 8 (229 mg, 0.404 mmol, 1 equiv), TFA (716 mg, 6.280 mmol, 15 equiv), DCM (5 mL). To afford Intermediate 16 (158.5 mg, 83%) a brown oil.

TLC: Eluent DCM/MeOH·NH₃ 5% R_{f}=0.51

¹H NMR (400 MHz, DMSO-d₆) δ 8.09 (1H, d, J=5.2 Hz, H21), 7.93-7.87 (3H, m, H11 and H15), 7.75 (1H, s, H13), 7.63 (2H, m, H8 and H17), 7.51 (2H, t, J=7.8 Hz, H16), 7.35 (1H, m, H10), 7.27 (1H, m, H9), 7.20 (1H, m, H20), 6.98 (1H, s, H23), 3.70 (2H, s, H5), 3.57 (2H, s, H18), 2.43 (2H, t, J=6.9 Hz, H1), 2.35 (2H, m, H4), 1.49 (2H, m, H3), 1.24 (2H, p, J=7.1 Hz, H2).

¹³C NMR (100 MHz, DMSO-d₆) δ 156.11 (C22), 147.20 (C21), 136.85 (C14), 134.85 (C12), 134.53 (C17), 131.96 (C19), 130.47 (C7), 129.71 (C16), 126.52 (C15), 125.47 (C13), 125.01 (C10), 123.44 (C9), 121.67 (C20), 121.33 (C6), 120.56 (C8), 113.34 (C11), 108.32 (C23), 56.23 (C18), 53.44 (C4), 48.71 (C5), 40.68 (C1), 29.33 (C2), 23.60 (C3).

### 6.3. Preparation of N1-((6-fluoropyridin-2-yl)methyl)-N1-((1-(phenylsulfonyl)-1H-indol-3-yl)methyl)butane-1,4-diamine (Intermediate 17, C₂₅H₂₇FN₄O₂S, MW=466.57 g/mol)

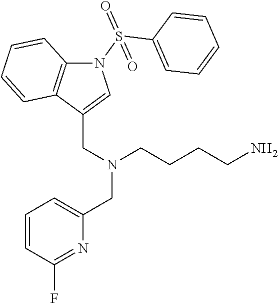 [SMILES: NCCCCN(Cc1cccc(F)n1)Cc1cn(S(=O)(=O)c2ccccc2)c2ccccc12]

According to general procedure 4. Scale: Intermediate 9 (210 mg, 0.371 mmol, 1 equiv), TFA (716 mg, 5.560 mmol, 15 equiv), DCM (5 mL). To afford Intermediate 17 (162.8 mg, 94%) an amorphous yellow solid.

TLC: Eluent DCM/MeOH·NH₃ 5% R_{f}=0.48

¹H NMR (400 MHz, Chloroform-d) δ 7.97 (1H, d, J=8.2 Hz, H11), 7.83 (2H, m, H15), 7.6 (2H, m, H8 and H22), 7.52-7.46 (2H, m, H13 and H17), 7.38 (2H, m, H16), 7.31 (1H, m, H10), 7.27-7.21 (2H, m, H9 and H23), 6.75 (1H, dd, J=8.1, 2.5 Hz, H21), 3.73 (2H, s, H5), 3.63 (2H, s, H18), 2.62 (2H, t, J=7.0 Hz, H1), 2.51 (2H, m, H4), 1.57 (2H, p, J=8.4, 7.6 Hz, H3), 1.41 (2H, dt, J=14.5, 6.9 Hz, H2).

¹³C NMR (100 MHz, Chloroform-d) δ 164.22 (C20), 161.84 (C21), 141.41 (C22), 138.21 (C14), 135.68 (C12), 133.87 (C17), 130.88 (C7), 129.32 (C16), 126.80 (C15), 125.03 (C10), 124.97 (C13), 123.39 (C9), 120.81 (C6), 120.53 (C8), 120.04 (C23), 113.86 (C11), 107.41 (C21), 59.37 (C18), 54.39 (C4), 49.74 (C5), 41.93 (C1), 31.16 (C2), 24.67 (C3).

### 6.4. Preparation of N1-((6-fluoropyridin-3-yl)methyl)-N1-((1-(phenylsulfonyl)-1H-indol-3-yl)methyl)butane-1,4-diamine (Intermediate 18, C₂₅H₂₇FN₄O₂S, MW=466.57 g/mol)

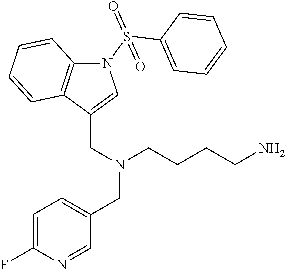 [SMILES: NCCCCN(Cc1ccc(F)nc1)Cc1cn(S(=O)(=O)c2ccccc2)c2ccccc12]

According to general procedure 4. Scale: Intermediate 10 (138 mg, 0.244 mmol, 1 equiv), TFA (317 mg, 3.657 mmol, 15 equiv), DCM (4 mL). To afford Intermediate 18 (87.3 mg, 77%) an amorphous yellow solid.

TLC: Eluent DCM/MeOH·NH₃ 4% R_{f}=0.51

¹H NMR (400 MHz, Chloroform-d) δ 8.05 (1H, m, H20), 7.98 (1H, d, J=8.3 Hz, H11), 7.84 (2H, m, H15), 7.64 (1H, td, J=8.1, 2.4 Hz, H23), 7.58 (1H, d, J=7.8 Hz, H8), 7.51-7.45 (2H, m, H13 and H17), 7.38 (2H, m, H16), 7.32 (1H, m, H10), 7.23 (1H, m, H9), 6.83 (1H, dd, J=8.3, 2.7 Hz, H22), 3.64 (2H, s, H5), 3.47 (2H, s, H18), 2.61 (2H, t, J=7.1 Hz, H1), 2.40 (2H, m, H4), 1.54 (2H, m, H3), 1.39 (2H, m, H2).

¹³C NMR (100 MHz, Chloroform-d) δ 164.21 (C21), 161.84 (C19), 147.58 (C20), 141.86 (C23), 138.21 (C14), 135.72 (C12), 133.93 (C17), 130.71 (C7), 129.36 (C16), 126.80 (C15), 125.16 (C10), 124.89 (C13), 123.47 (C9), 120.75 (C6), 120.57 (C8), 113.89 (C11), 109.31 (C22), 54.88 (C18), 53.59 (C4), 49.51 (C5), 41.68 (C1), 30.47 (C2), 24.42 (C3).

### 6.5. Preparation of N1-((2-fluoropyridin-3-yl)methyl)-N1-((1-(phenylsulfonyl)-1H-indol-3-yl)methyl)pentane-1,5-diamine (Intermediate 19, C₂₆H₂₉FN₄O₂S, MW=480.60 g/mol)

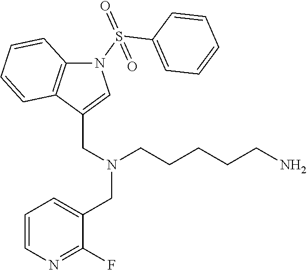 [SMILES: NCCCCCN(Cc1cccnc1F)Cc1cn(S(=O)(=O)c2ccccc2)c2ccccc12]

According to general procedure 4. Scale: Intermediate 11 (285 mg, 0.491 mmol, 1 equiv), TFA (840 mg, 7.367 mmol, 15 equiv), DCM (6 mL). To afford Intermediate 19 (233.7 mg, 99%) an amorphous brown solid.

TLC: Eluent DCM/MeOH·NH₃ 4% R_{f}=0.40

¹H NMR (400 MHz, Chloroform-d) δ 8.05 (1H, m, H22), 7.97 (1H, d, J=8.3 Hz, H12), 7.84-7.79 (2H, m, H16), 7.75 (1H, m, H24), 7.56 (1H, d, J=7.7 Hz, H9), 7.50-7.45 (2H, m, H14 and H18), 7.37 (2H, m, H17), 7.31 (1H, m, H11), 7.22 (1H, m, H10), 7.10 (1H, m, H23), 3.67 (2H, s, H6), 3.55 (2H, s, H19), 2.61 (2H, t, J=7.1 Hz, H1), 2.43 (2H, m, H5), 1.51 (2H, p, J=7.4 Hz, H4), 1.35 (2H, m, H)3, 1.24 (2H, m, H2).

¹³C NMR (100 MHz, Chloroform-d) δ 160.92 (C21), 146.02 (C22), 141.42 (C24), 138.18 (C15), 135.72 (C13), 133.91 (C18), 130.77 (C8), 129.32 (C17), 126.77 (C16), 125.09 (C11), 124.79 (C14), 123.39 (C10), 121.69 (C20), 121.53 (C23), 120.99 (C7), 120.54 (C9), 113.85 (C12) 54.00 (C5), 50.97 (C19), 49.79 (C6), 41.68 (C1), 32.31 (C3), 26.91 (C4), 24.51 (C2).

### 6.6. Preparation of N1-((2-fluoropyridin-4-yl)methyl)-N1-((1-(phenylsulfonyl)-1H-indol-3-yl)methyl) pentane-1,5-diamine (Intermediate 20, C₂₆H₂₉FN₄O₂S, MW=480.60 g/mol)

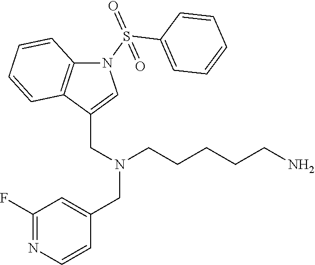 [SMILES: NCCCCCN(Cc1ccnc(F)c1)Cc1cn(S(=O)(=O)c2ccccc2)c2ccccc12]

According to general procedure 4. Scale: Intermediate 12 (580 mg, 0.999 mmol, 1 equiv), TFA (1.709 g, 14.985 mmol, 15 equiv), DCM (6 mL). To afford Intermediate 20 (413.3 mg, 86%) an orange oil.

TLC: Eluent DCM/MeOH·NH₃ 5% R_{f}=0.48

¹H NMR (400 MHz, Chloroform-d) δ 8.07 (1H, d, J=5.1 Hz, H22), 7.98 (1H, m, H12), 7.82 (2H, m, H16), 7.60 (1H, m, H9), 7.47 (2H, m, H14 and H18), 7.40-7.29 (3H, m, H11 and H17), 7.27-7.21 (1H, m, H10), 7.06 (1H, m, H21), 6.84 (1H, s, H24), 3.66 (2H, s, H6), 3.51 (2H, s, H19), 2.61 (2H, t, J=6.8 Hz, H1), 2.42 (2H, m, H5), 1.52 (2H, m, H4), 1.39-1.20 (4H, m, H2 and H3).

¹³C NMR (100 MHz, Chloroform-d) δ 165.38 (C23), 155.75 (C20), 147.42 (C22), 138.13 (C15), 135.68 (C13), 133.91 (C18), 130.66 (C8), 129.31 (C17), 126.73 (C16), 125.16 (C11), 124.88 (C14), 123.44 (C10), 121.32 (C21), 120.62 (C7), 120.43 (C9), 113.88 (C12), 108.98 (C24), 57.30 (C19), 54.37 (C5), 49.77 (C6), 42.08 (C1), 33.38 (C2), 27.09 (C4), 24.61 (C3).

### 6.7. Preparation of N1-((6-fluoropyridin-2-yl)methyl)-N1-((1-(phenylsulfonyl)-1H-indol-3-yl)methyl)pentane-1,5-diamine (Intermediate 21, C₂₆H₂₉FN₄O₂S, MW=480.60 g/mol)

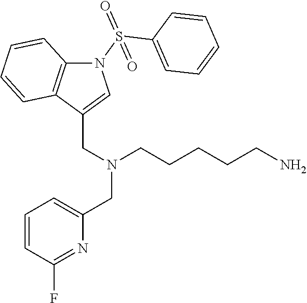 [SMILES: NCCCCCN(Cc1cccc(F)n1)Cc1cn(S(=O)(=O)c2ccccc2)c2ccccc12]

According to general procedure 4. Scale: Intermediate 13 (539 mg, 0.928 mmol, 1 equiv), TFA (1.587 g, 13.929 mmol, 15 equiv), DCM (12 mL). To afford Intermediate 21 (330 mg, 74%) an amorphous yellow solid.

TLC: Eluent DCM/MeOH·NH₃ 5% R_{f}=0.41

¹H NMR (400 MHz, Chloroform-d) δ 7.97 (1H, m, H12), 7.82 (2H, m, H16), 7.69 (1H, q, J=8.1 Hz, H23), 7.63 (1H, m, H9), 7.51-7.45 (2H, m, H14 and H18), 7.37 (2H, m, H17), 7.31 (1H, m, H11), 7.28-7.20 (2H, m, H10 and H24), 6.75 (1H, dd, J=8.1, 2.4 Hz, H22), 3.72 (2H, m, H6), 3.63 (2H, s, H19), 2.61 (2H, t, J=6.9 Hz, H1), 2.48 (2H, m, H5), 1.53 (2H, m, H4), 1.39-1.19 (4H, m, H2 and H3).

¹³C NMR (100 MHz, Chloroform-d) δ 161.82 (C21), 159.69 (C20), 141.34 (C23), 138.22 (C15), 135.71 (C13), 133.84 (C18), 130.91 (C8), 129.30 (C17), 126.79 (C16), 125.00 (C11), 124.90 (C14), 123.34 (C10), 121.09 (C7), 120.58 (C9), 119.99 (C24), 113.85 (C12), 107.33 (C22), 59.51 (C19), 54.41 (C5), 49.84 (C6), 42.16 (C1), 33.44 (C2), 27.12 (C4), 24.64 (C3).

### 6.8. Preparation of N1-((6-methylpyridin-3-yl)methyl)-N1-((1-(phenylsulfonyl)-1H-indol-3-yl)methyl)pentane-1,5-diamine (Intermediate 22, C₂₆H₂₉FN₄O₂S, MW=480.60 g/mol)

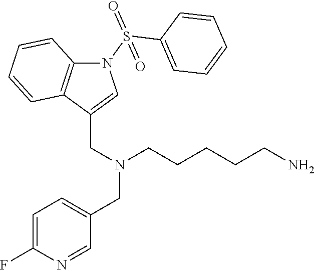 [SMILES: NCCCCCN(Cc1ccc(F)nc1)Cc1cn(S(=O)(=O)c2ccccc2)c2ccccc12]

According to general procedure 4. Scale: Intermediate 14 (312 mg, 0.538 mmol, 1 equiv), TFA (920 mg, 8.069 mmol, 15 equiv), DCM (9 mL). To afford Intermediate 22 (232.6 mg, 90%) an amorphous brown solid.

TLC: Eluent DCM/MeOH·NH₃ 4% R_{f}=0.45

¹H NMR (400 MHz, Chloroform-d) δ 8.05 (1H, m, H21), 7.98 (1H, d, J=8.3 Hz, H12), 7.84 (2H, m, H16), 7.64 (1H, td, J=8.1, 2.4 Hz, H24), 7.57 (1H, d, J=7.8 Hz, H9), 7.51-7.45 (2H, m, H14 and H18), 7.38 (2H, m, H17), 7.32 (1H, m, H11), 7.23 (1H, m, H10), 6.83 (1H, dd, J=8.3, 2.7 Hz, H23), 3.64 (2H, s, H6), 3.47 (2H, s, H19), 2.61 (2H, t, J=7.0 Hz, H1), 2.39 (2H, m, H5), 1.51 (2H, p, J=7.4 Hz, H4), 1.28 (4H, m, H2 and H3).

¹³C NMR (100 MHz, Chloroform-d) δ 161.84 (C22), 147.59 (C21), 141.84 (C24), 138.22 (C15), 135.74 (C13), 133.92 (C18), 132.91 (C20), 130.74 (C8), 129.35 (C17), 126.79 (C16), 125.15 (C11), 124.88 (C14), 123.43 (C10), 120.88 (C7), 120.60 (C9), 113.88 (C12), 109.27 (C23), 54.93 (C19), 53.67 (C5), 49.59 (C6), 41.91 (C1), 32.84 (C2), 26.94 (C4), 24.59 (C3).

### 6.9. Preparation of 2-(2-aminoethoxy)-N-((2-fluoropyridin-3-yl)methyl)-N-((1-(phenylsulfonyl)-1H-indol-3-yl)methyl)ethan-1-amine (Intermediate 26, C₂₅H₂₇FN₄O₃S, MW=482.57 g/mol)

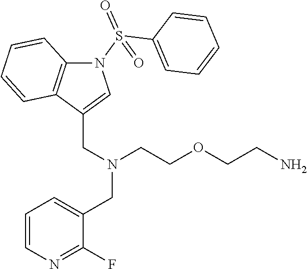 [SMILES: NCCOCCN(Cc1cccnc1F)Cc1cn(S(=O)(=O)c2ccccc2)c2ccccc12]

According to general procedure 4. Scale: Intermediate 25 (151 mg, 0.259 mmol, 1 equiv), TFA (443 mg, 3.885 mmol, 15 equiv), DCM (4 mL). To afford of Intermediate 26 (117 mg, 94%) an amorphous yellow solid.

TLC: Eluent DCM/MeOH·NH3 3% Rf=0.44

1H NMR (400 MHz, CDCl3) δ 8.05 (1H, m, H21), 7.97 (1H, d, J=8.3 Hz, H11), 7.82 (3H, m, H15 and H23), 7.60 (1H, d, J=7.6 Hz, H8), 7.53 (1H, s, H13), 7.48 (1H, tt, J=7.5 Hz, J=1.3 Hz, H17), 7.38 (2H, m, H16), 7.31 (1H, m, H10), 7.22 (1H, m, H9), 7.09 (1H, m, H22), 3.78 (2H, s, H5), 3.67 (2H, s, H18), 3.57 (2H, t, J=5.5 Hz, H3), 3.42 (2H, t, J=5.2 Hz, H2), 2.86 (2H, br, H1), 2.72 (2H, t, J=5.5 Hz, H4).

13C NMR (101 MHz, CDCl3) δ 160.86 (C20), 146.06 (C21), 141.43 (C23), 138.19 (C14), 135.73 (C12), 133.91 (C17), 130.72 (C7), 129.33 (C16), 126.80 (C15), 125.11 (C10), 124.90 (C13), 123.40 (C9), 121.54 (C22), 121.26 (C19), 120.83 (C6), 120.53 (C8), 113.88 (C11), 72.64 (C2), 69.63 (C3), 53.50 (C4), 51.44 (C18), 50.29 (C5), 41.76 (C1).

LRMS m/z (ESI+, CV 30) 483.2 [M+H]⁺.

### 7. General Procedure 5. Third Reductive Amination

### 7.1. Preparation of N1-cyclohexyl-N4-((2-fluoropyridin-3-yl)methyl)-N4-((1-(phenylsulfonyl)-1H-indol-3-yl)methyl)butane-1,4-diamine (Compound 43, C₃₁H₃₇FN₄O₂S, MW=548.71 g/mol)

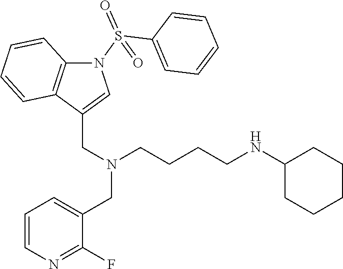 [SMILES: O=S(=O)(c1ccccc1)n1cc(CN(CCCCNC2CCCCC2)Cc2cccnc2F)c2ccccc21]

Under argon atmosphere, Intermediate 15 (80 mg, 0.171 mmol, 1 equiv) was dissolved in anhydrous DCM (2 mL, 0.09 M), cyclohexanone (20 mg, 0.204 mmol, 1.2 equiv) was added to the reactional mixture. After 1 h of stirring at RT, NaBH(OAc)3 (55 mg, 0.260 mmol, 1.5 equiv) was added. The mixture was stirred at RT. After 24 h, the mixture was re-dissolved in DCM (25 mL) and washed with a saturated solution of NaHCO₃(1×20 mL). The aqueous layer was extracted DCM (3×20 mL). The combined organic layers were dried over MgSO₄ and concentrated under reduce pressure, to afford 90.1 mg of an orange oil. The crude product was purified by flash column chromatography on silica gel, using a solvent gradient (DCM/MeOH·NH3 1% to 3%) to afford Compound 43 (68.7 mg, 73%) an amorphous yellow solid.

TLC: Eluent DCM/MeOH·NH₃ 2% R_{f}=0.42

¹H NMR (400 MHz, Chloroform-d) δ 8.05 (1H, m, H21), 7.97 (1H, d, J=8.3 Hz, H11), 7.84-7.74 (3H, m, H15 and H23), 7.58 (1H, d, J=7.6 Hz, H8), 7.48 (2H, m, H13 and H17), 7.38 (2H, m, H16), 7.31 (1H, m, H10), 7.22 (1H, m, H9), 7.10 (1H, m, H22), 3.69 (2H, s, H5), 3.55 (2H, s, H18), 2.51 (2H, m, H1), 2.45 (2H, m, H4), 2.31 (1H, tt, J=14.3, 10.5, H24), 1.81 (2H, m, H25), 1.71 (2H, m, H26), 1.57 (3H, m, H3 and H27), 1.40 (2H, q, J=14.9, H2), 1.29-1.06 (3H, m, H26′ and H27′), 1.06-0.93 (2H, m, H25′).

¹³C NMR (100 MHz, Chloroform-d) δ 160.90 (C20), 146.00 (C21), 141.37 (C23), 138.21 (C14), 135.74 (C12), 133.88 (C17), 130.77 (C7), 129.32 (C16), 126.77 (C15), 125.08 (C10), 124.80 (C13), 123.40 (C9), 121.53 (C22), 121.41 (C19), 120.93 (C6), 120.54 (C8), 113.87 (C11), 57.04 (C24), 54.18 (C4), 50.85 (C18), 49.76 (C5), 46.85 (C1), 33.70 (C25), 28.31 (C2), 26.28 (C3), 25.22 (C26), 25.11 (C27).

LRMS m/z (ESI+, CV 30) 549.2 [M+H]⁺.

### 7.2. Preparation of N1-cyclohexyl-N4-((2-fluoropyridin-4-yl)methyl)-N4-((1-(phenylsulfonyl)-1H-indol-3-yl)methyl)butane-1,4-diamine (Compound 44, C₃₁H₃₇FN₄O₂S, MW=548.71 g/mol)

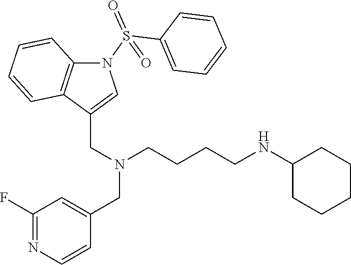 [SMILES: O=S(=O)(c1ccccc1)n1cc(CN(CCCCNC2CCCCC2)Cc2ccnc(F)c2)c2ccccc21]

According to general procedure 5. Scale: Intermediate 6 (144.8 mg, 0.310 mmol, 1 equiv), Cyclohexanone (40 mg, 0.403 mmol, 1.3 equiv), NaBH(OAc)3 (197 mg, 0.930 mmol, 3 equiv), DCM (4 mL). The crude product (205.7 mg of an orange oil) was purified by flash column chromatography on silica gel, using a solvent gradient (DCM/MeOH·NH₃ 0% to 5%) to afford Compound 44 (73.8 mg, 33%) an amorphous yellow solid.

TLC: Eluent DCM/MeOH·NH₃ 5% R_{f}=0.52

¹H NMR (400 MHz, Chloroform-d) δ 8.07 (1H, d, J=5.1 Hz, H21), 7.98 (1H, d, J=8.2 Hz, H11), 7.83 (2H, m, H15), 7.61 (1H, d, J=7.8 Hz, H8), 7.48 (2H, m, H13 and H17), 7.38 (2H, m, H16), 7.33 (1H, m, H10), 7.28-7.23 (1H, m, H9), 7.07 (1H, m, H20), 6.84 (1H, s, H23), 3.66 (2H, s, H5), 3.50 (2H, s, H18), 2.56 (2H, t, J=7.2 Hz, H1), 2.44 (2H, m, H4), 2.39 (1H, m, H24), 1.86 (2H, m, H25), 1.71 (2H, m, H26), 1.65-1.43 (5H, m, H2, H3 and H27), 1.15 (5H, m, H25′, H26′ and H27′).

¹³C NMR (100 MHz, Chloroform-d) δ 163.05 (C22), 155.64 (C19), 147.51 (C21), 138.19 (C14), 135.71 (C12), 133.94 (C17), 130.66 (C7), 129.35 (C16), 126.77 (C15), 125.20 (C10), 124.94 (C13), 123.51 (C9), 121.35 (C20), 120.51 (C6), 120.46 (C8), 113.91 (C11), 109.01 (C23), 57.24 (C18), 57.07 (C24), 54.35 (C4), 49.77 (C5), 46.52 (C1), 33.16 (C25), 27.76 (C2), 26.11 (C27), 25.15 (C3 and C26).

LRMS m/z (ESI+, CV 30) 549 [M+H]⁺.

### 7.3. Preparation of N1-cyclohexyl-N4-((6-fluoropyridin-2-yl)methyl)-N4-((1-(phenylsulfonyl)-1H-indol-3-yl)methyl)butane-1,4-diamine (Compound 45, C₃₁H₃₇FN₄O₂S, MW=548.71 g/mol)

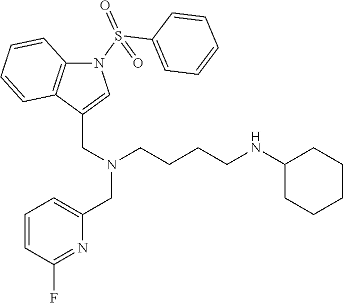 [SMILES: O=S(=O)(c1ccccc1)n1cc(CN(CCCCNC2CCCCC2)Cc2cccc(F)n2)c2ccccc21]

According to general procedure 5. Scale: Intermediate 17 (161 mg, 0.345 mmol, 1 equiv), Cyclohexanone (34 mg, 0.345 mmol, 1 equiv), NaBH₄ (21 mg, 0.552 mmol, 1.6 equiv), MeOH (5 mL). The crude product (207.4 mg of an amorphous yellow solid) was purified by flash column chromatography on silica gel, using a solvent gradient (DCM/MeOH·NH₃ 0% to 5%) to afford Compound 45 (84.4 mg, 45%) an amorphous yellow solid.

TLC: Eluent DCM/MeOH·NH₃ 2% R_{f}=0.34

¹H NMR (400 MHz, Chloroform-d) δ 7.97 (1H, d, J=8.2 Hz, H11), 7.82 (2H, m, H15), 7.70 (1H, q, J=8.1 Hz, H22), 7.64 (1H, d, J=7.8 Hz, H8), 7.52-7.45 (2H, m, H13 and H17), 7.38 (2H, m, H16), 7.31 (1H, m, H10), 7.28-7.21 (2H, m, H9 and H23), 6.76 (1H, dd, J=8.1, 2.5 Hz, H21), 3.75 (2H, s, H5), 3.62 (2H, s, H18), 2.62 (2H, m, H1), 2.53 (2H, m, H4), 2.48 (1H, m, H24), 1.87 (2H, m, H25), 1.72 (2H, m, H26), 1.57 (5H, m, H2, H3 and H27), 1.28-1.13 (5H, m, H25′, H26′ and H27′).

¹³C NMR (100 MHz, Chloroform-d) δ 164.26 (C20), 159.34 (C19), 141.51 (C22), 138.19 (14), 135.63 (C12), 133.88 (C17), 130.93 (C7), 129.33 (C16), 126.79 (C15), 125.10 (C13), 125.02 (C10), 123.44 (C9), 120.63 (C8), 120.40 (C6), 120.22 (C23), 113.81 (C11), 107.50 (C21), 59.02 (C18), 57.06 (C24), 54.27 (C4), 49.52 (C5), 46.16 (C1), 32.48 (C25), 27.30 (C2), 25.89 (C27), 25.17 (C3), 25.07 (C26).

LRMS m/z (ESI+, CV 30) 458.3 [M+H]⁺.

### 7.4. Preparation of N1-cyclohexyl-N4-((6-fluoropyridin-3-yl)methyl)-N4-((1-(phenylsulfonyl)-1H-indol-3-yl)methyl)butane-1,4-diamine (Compound 55, C₃₁H₃₇FN₄O₂S, MW=548.71 g/mol)

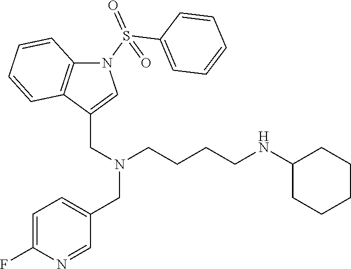 [SMILES: O=S(=O)(c1ccccc1)n1cc(CN(CCCCNC2CCCCC2)Cc2ccc(F)nc2)c2ccccc21]

According to general procedure 5. Scale: Intermediate 18 (77 mg, 0.165 mmol, 1 equiv), Cyclohexanone (16.2 mg, 0.165 mmol, 1 equiv), NaBH₄ (10 mg, 0.264 mmol, 1.6 equiv), MeOH (2.5 mL). The crude product (102 mg of an orange oil) was purified by flash column chromatography on silica gel, using a solvent gradient (DCM/MeOH·NHMeOH·NH₃ 1% to 4%) to afford Compound 55 (46.7 mg, 52%) a colourless oil.

TLC: Eluent DCM/MeOH·NH₃ 2% R_{f}=0.45

¹H NMR (400 MHz, Chloroform-d) δ 8.04 (1H, m, H20), 7.98 (1H, d, J=8.3 Hz, H11), 7.86-7.80 (2H, m, 15H), 7.65 (1H, td, J=8.1, 2.5 Hz, H23), 7.59 (1H, m, H8), 7.51-7.45 (2H, m, H13 and H17), 7.39 (2H, m, H16), 7.32 (1H, m, H10), 7.23 (1H, m, H9), 6.83 (1H, dd, J=8.4, 2.9 Hz, H22), 3.65 (2H, s, H5), 3.47 (2H, s, H18), 2.49 (2H, m, H1), 2.41 (2H, m, H4), 2.30 (1H, tt, J=10.5, 3.7 Hz, H24), 1.80 (2H, m, H25), 1.70 (2H, m, H26), 1.56 (3H, H3 and H27), 1.39 (2H, m, H2), 1.29-1.10 (3H, m, H26′ and H27′), 1.00 (2H, m, H25′).

¹³C NMR (100 MHz, Chloroform-d) δ 164.10 (C21), 159.60 (C19), 147.46 (C20), 141.70 (C23), 138.12 (C14), 135.62 (C12), 133.78 (C17), 130.60 (C7), 129.22 (C16), 126.66 (C15), 125.01 (C10), 124.75 (C13), 123.31 (C9), 120.70 (C6), 120.48 (C8), 113.76 (C11), 109.15 (C22), 56.92 (C24), 54.75 (C18), 53.63 (C4), 49.45 (C5), 46.70 (C1), 33.61 (C25), 28.20 (C2), 26.16 (C27), 25.10 (C26), 24.92 (C3).

LRMS m/z (ESI+, CV 30) 549.2 [M+H]⁺.

### 7.5. Preparation of N1-cyclohexyl-N5-((2-fluoropyridin-3-yl)methyl)-N5-((1-(phenylsulfonyl)-1H-indol-3-yl)methyl)pentane-1,5-diamine (Compound 46, C₃₂H₃₉FN₄O₂S, MW=562.74 g/mol)

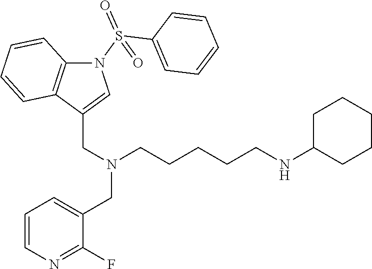 [SMILES: O=S(=O)(c1ccccc1)n1cc(CN(CCCCCNC2CCCCC2)Cc2cccnc2F)c2ccccc21]

According to general procedure 5. Scale: Intermediate 19 (182 mg, 0.379 mmol, 1 equiv), Cyclohexanone (37.2 mg, 0.379 mmol, 1 equiv), NaBH₄ (23 mg, 0.608 mmol, 1.6 equiv), MeOH (5.5 mL). The crude product (136 mg of an orange oil) was purified by flash column chromatography on silica gel, using a solvent gradient (DCM/MeOH·NH₃ 1% to 4%) to afford Compound 46 (77.3 mg, 44%) an amorphous colourless solid.

TLC: Eluent DCM/MeOH·NH₃ 2% R_{f}=0.34

¹H NMR (400 MHz, Chloroform-d) δ 8.05 (1H, m, H22), 7.97 (1H, m, H12), 7.81 (2H, m, H16), 7.76 (1H, m, H24), 7.57 (1H, d, J=7.8 Hz, H9), 7.50-7.45 (2H, m, H14 and H18), 7.37 (2H, m, H17), 7.31 (1H, m, H11), 7.22 (1H, m, H10), 7.10 (1H, m, H23), 3.68 (2H, s, H6), 3.55 (2H, s, H19), 2.52 (2H, m, H1), 2.43 (2H, m, H5), 2.36 (1H, tt, J=10.5, 3.7 Hz, H25), 1.88-1.80 (2H, m, H26), 1.75-1.67 (2H, m, H27), 1.64-1.48 (3H, m, H4 and H28), 1.37 (2H, p, J=14.7, H2), 1.31-1.11 (4H, m, H3, H27′ and H28′), 1.02 (2H, qd, J=12.6, 3.2 Hz, H26′).

¹³C NMR (100 MHz, Chloroform-d) δ 160.90 (C21), 145.97 (C22), 141.29 (C24), 138.21 (C15), 135.75 (C13), 133.87 (C18), 130.79 (C8), 129.31 (C17), 126.76 (C16), 125.06 (C11), 124.78 (C14), 123.37 (C10), 121.76 (C20), 121.48 (C23), 121.04 (C7), 120.54 (C9), 113.87 (C12), 57.04 (C25), 54.25 (C5), 50.89 (C19), 49.78 (C6), 47.02 (C1), 33.77 (C26), 30.47 (C2), 27.15 (C4), 26.32 (C28), 25.24 (C3 and C27).

LRMS m/z (ESI+, CV 30) 563.4 [M+H]⁺.

### 7.6. Preparation of N1-cyclohexyl-N5-((2-fluoropyridin-4-yl)methyl)-N5-((1-(phenylsulfonyl)-1H-indol-3-yl)methyl)pentane-1,5-diamine (Compound 47, C₃₂H₃₉FN₄O₂S, MW=562.74 g/mol)

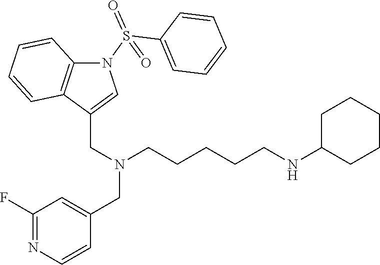 [SMILES: O=S(=O)(c1ccccc1)n1cc(CN(CCCCCNC2CCCCC2)Cc2ccnc(F)c2)c2ccccc21]

According to general procedure 5. Scale: Intermediate 20 (453 mg, 0.943 mmol, 1 equiv), Cyclohexanone (92.5 mg, 0.943 mmol, 1 equiv), NaBH₄ (57 mg, 1.509 mmol, 1.6 equiv), MeOH (5.5 mL). The crude product (597 mg of an orange oil) was purified by flash column chromatography on silica gel, using a solvent gradient (DCM/MeOH·NH₃ 1% to 4%) to afford Compound 47 (277.6 mg, 52%) an amorphous colourless solid.

TLC: Eluent DCM/MeOH·NH₃ 2% R_{f}=0.33

¹H NMR (400 MHz, Chloroform-d) δ 8.07 (1H, d, J=5.1 Hz, H22), 7.98 (1H, d, J=8.3 Hz, H12), 7.83 (2H, m, H16), 7.61 (1H, d, J=7.6 Hz, H9), 7.51-7.46 (2H, m, H14 and H18), 7.41-7.31 (3H, m, H11 and H17), 7.28-7.23 (1H, m, H10), 7.06 (1H, m, H21), 6.84 (1H, s, H24), 3.67 (2H, s, H6), 3.51 (2H, s, H19), 2.56 (2H, m, H1), 2.47-2.34 (3H, m, H5 and H25), 1.86 (2H, m, H26), 1.76-1.67 (2H, m, H27), 1.65-1.49 (3H, m, H4 and H28), 1.41 (2H, p, J=14.7, 7.3 Hz, H2), 1.32-1.11 (5H, m, H3, H27′ and H28′), 1.11-0.99 (2H, m, H26′).

¹³C NMR (100 MHz, Chloroform-d) δ 155.81 (C20), 147.48 (C22), 138.21 (C15), 135.73 (C13), 133.94 (C18), 130.71 (C8), 129.35 (C17), 126.78 (C16), 125.20 (C11), 124.91 (C14), 123.48 (C10), 121.36 (C21), 120.64 (C7), 120.46 (C9), 113.93 (C12), 109.00 (C24), 57.28 (C25), 57.10 (C19), 54.50 (C5), 49.79 (C6), 46.94 (C1), 33.60 (C26), 30.34 (C2), 27.22 (C4), 26.26 (C28), 25.23 (C3 and C27).

LRMS m/z (ESI+, CV 30) 563.4 [M+H]⁺.

### 7.7. Preparation of N1-cyclohexyl-N5-((6-fluoropyridin-2-yl)methyl)-N5-((1-(phenylsulfonyl)-1H-indol-3-yl)methyl)pentane-1,5-diamine (Compound 48, C₃₂H₃₉FN₄O₂S, MW=562.74 g/mol)

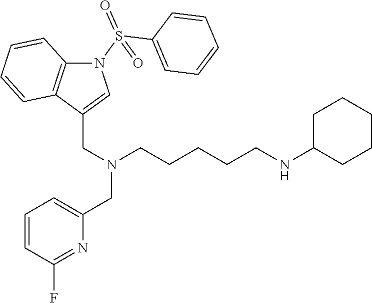 [SMILES: O=S(=O)(c1ccccc1)n1cc(CN(CCCCCNC2CCCCC2)Cc2cccc(F)n2)c2ccccc21]

According to general procedure 5. Scale: Intermediate 21 (140 mg, 0.291 mmol, 1 equiv), Cyclohexanone (34.3 mg, 0.349 mmol, 1.2 equiv), NaBH(OAc)3 (93 mg, 0.437 mmol, 1.5 equiv), DCM (4 mL). The crude product (141 mg of an orange oil) was purified by flash column chromatography on silica gel, using a solvent gradient (DCM/MeOH·NH₃ 2% to 3%) to Compound 48 (125 mg, 76%) an amorphous colourless solid.

TLC: Eluent DCM/MeOH·NH₃ 2% R_{f}=0.45

¹H NMR (400 MHz, Chloroform-d) δ 7.90 (1H, m, H12), 7.76-7.74 (2H, m, H16), 7.62 (1H, J=8.1 Hz, H23), 7.54 (1H, d, J=7.5 Hz, H9), 7.44-7.38 (2H, m, H14 and H18), 7.30 (2H, m, H17), 7.24 (1H, m, H11), 7.19-7.13 (2H, m, H10 and H24), 6.68 (1H, dd, J=8.0, 2.5 Hz, H22), 3.63 (2H, s, H6), 3.53 (2H, s, H19), 2.66-2.53 (3H, m, H1 and H25), 2.40 (2H, m, H5), 1.97 (2H, m, H26), 1.70 (2H, m, H27), 1.64-1.51 (3H, m, H2 and H28), 1.46 (2H, p, J=7.7 Hz, H4), 1.20 (7H, m, H3, H26′, H27′ and H28′).

¹³C NMR (100 MHz, Chloroform-d) δ 161.80 (C21), 159.55 (C20), 141.38 (C23), 138.19 (C15), 135.69 (C13), 133.88 (C18), 130.88 (C8), 129.32 (C17), 126.79 (C16), 124.99 (C11), 124.92 (C14), 123.36 (C10), 120.99 (C7), 120.57 (C9), 119.98 (C24), 113.84 (C12), 107.34 (C22), 59.41 (C19), 57.13 (C25), 54.45 (C5), 49.80 (C6), 45.62 (C1), 31.37 (C25), 28.14 (C2), 27.11 (C4), 25.55 (C28), 25.11 (C3), 24.94 (C27).

LRMS m/z (ESI+, CV 30) 563.3 [M+H]⁺.

### 7.8. Preparation of N1-cyclohexyl-N5-((6-methylpyridin-3-yl)methyl)-N5-((1-(phenylsulfonyl)-1H-indol-3-yl)methyl)pentane-1,5-diamine (Compound 56, C₃₂H₃₉FN₄O₂S, MW=562.74 g/mol)

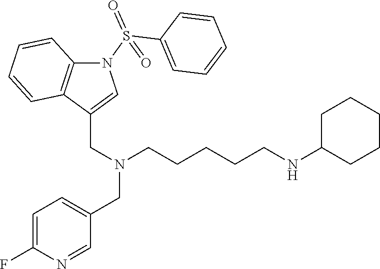 [SMILES: O=S(=O)(c1ccccc1)n1cc(CN(CCCCCNC2CCCCC2)Cc2ccc(F)nc2)c2ccccc21]

According to general procedure 5. Scale: Intermediate 22 (225 mg, 0.468 mmol, 1 equiv), Cyclohexanone (45.9 mg, 0.468 mmol, 1 equiv), NaBH₄ (28.3 mg, 0.748 mmol, 1.6 equiv), MeOH (7 mL). The crude product (312.1 mg of an orange oil) was purified by flash column chromatography on silica gel, using a solvent gradient (DCM/MeOH·NH₃ 1% to 3%) to afford Compound 56 (161 mg, 76%) an amorphous yellow solid.

TLC: Eluent DCM/MeOH·NH₃ 2% R_{f}=0.40

¹H NMR (400 MHz, Chloroform-d) δ 8.04 (1H, H21), 7.98 (1H, d, J=8.3 Hz, H12), 7.82 (2H, m, H16), 7.64 (1H, td, J=8.1, 2.4 Hz, H24), 7.58 (1H, d, J=7.8 Hz, H9), 7.51-7.45 (2H, m, H14 and H18), 7.38 (2H, m, H17), 7.32 (1H, m, H11), 7.23 (1H, td, J=7.6, 1.0 Hz, H10), 6.83 (1H, dd, J=8.4, 2.7 Hz, H23), 3.64 (2H, s, H6), 3.46 (2H, s, H19), 2.52 (2H, m, H1), 2.43-2.31 (3H, m, H5 and H25), 1.85 (2H, m, H26), 1.71 (2H, m, H27), 1.65-1.45 (3H, m, H4 and H28), 1.36 (2H, p, J=14.7, 7H2), 1.31-1.10 (5H, m, H3, H27′ and H28′), 1.03 (2H, qd, J=12.6, 3.2 Hz, H26′).

¹³C NMR (100 MHz, Chloroform-d) δ 147.59 (C21), 141.81 (C24), 138.24 (C15), 135.76 (C13), 133.91 (C18), 132.90 (C20), 130.75 (C8), 129.34 (C17), 126.79 (C16), 125.13 (C11), 124.86 (C14), 123.42 (C10), 120.92 (C7), 120.61 (C9), 113.89 (C12), 109.25 (C23), 57.07 (C25), 54.90 (C19), 53.83 (C5), 49.58 (C6), 47.01 (C1), 33.72 (C26), 30.42 (C2), 27.11 (C4), 26.30 (C28), 25.29 (C3), 25.24 (C27).

LRMS m/z (ESI+, CV 30) 563.4 [M+H]⁺.

### 7.9. Preparation of N-(2-(2-(((2-fluoropyridin-3-yl)methyl) ((1-(phenylsulfonyl)-1H-indol-3-yl)methyl)amino)ethoxy)ethyl)cyclohexanaminee (Compound 64, C₃₁H₃₇FN₄O₃S, MW=564.72 g/mol)

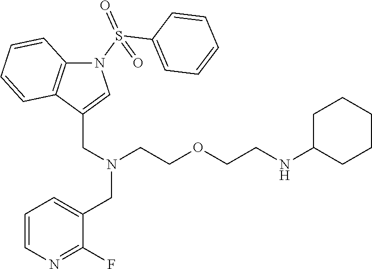 [SMILES: O=S(=O)(c1ccccc1)n1cc(CN(CCOCCNC2CCCCC2)Cc2cccnc2F)c2ccccc21]

According to general procedure 5. Scale: Intermediate 26 (116 mg, 0.24 mmol, 1 equiv), Cyclohexanone (28 mg, 0.288 mmol, 1.2 equiv), NaBH(OAc)3 (76 mg, 0.36 mmol, 1.5 equiv), DCM (2.5 mL). The crude product (137 mg of an orange amorphous solid) was purified by flash column chromatography on silica gel, using DCM/MeOH·NH₃ 2% to afford Compound 64 (110 mg, 81%) an amorphous yellow solid.

TLC: Eluent DCM/MeOH·NH3 2% Rf=0.63

1H NMR (400 MHz, CDCl3) δ 8.05 (1H, m, H21), 7.97 (1H, d, J=8.3 Hz, H11), 7.86-7.79 (3H, m, H15 and H23), 7.62 (1H, d, J=7.7 Hz, H8), 7.51 (1H, s, H13), 7.48 (1H, tt, J=7.5 Hz, J=1.4 Hz H17), 7.37 (2H, m, H16), 7.31 (1H, m, H10), 7.22 (1H, m, H9), 7.09 (1H, m, H22), 3.79 (2H, s, H5), 3.67 (2H, s, H18), 3.58 (2H, t, J=5.6 Hz, H3), 3.50 (2H, t, J=5.3 Hz, H2), 2.78 (2H, t, J=5.3 Hz, H1), 2.72 (2H, t, J=5.6 Hz, H4), 2.38 (tt, J=7.1 Hz, J=3.7 Hz, H24), 1.83 (2H, m, H25 eq), 1.70 (2H, m, H26 eq), 1.60 (1H, m, H27 eq), 1.29-0.97 (5H, m, H25ax, H25ax and H27ax).

13C NMR (101 MHz, CDCl3) δ 160.83 (C20), 145.97 (C21), 141.33 (C23), 138.20 (C14), 135.75 (C12), 133.87 (C17), 130.74 (C7), 129.30 (C16), 126.78 (C15), 125.07 (C10), 124.87 (C13), 123.39 (C9), 121.50 (C22), 121.35 (C19), 120.89 (C6), 120.59 (C8), 113.87 (C11), 70.96 (C2), 69.68 (C3), 56.94 (C24), 53.41 (C4), 51.28 (C18), 50.25 (C5), 46.56 (C1), 33.65 (C25), 26.26 (C27), 25.17 (C26).

19F NMR (376 MHz, Chloroform-d) δ−72.55

LRMS m/z (ESI+, CV 30) 565.3 [M+H]⁺.

### 8. General Procedure 6: Methylation

### 8.1. Preparation of N1-cyclohexyl-N4-((2-fluoropyridin-3-yl)methyl)-N1-methyl-N4-((1-(phenylsulfonyl)-1H-indol-3-yl)methyl)butane-1,4-diamine (Compound 49, C₃₂H₃₉FN₄O₂S, MW=562.74 g/mol)

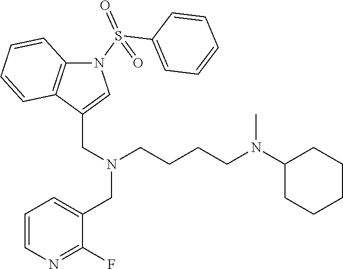 [SMILES: CN(CCCCN(Cc1cccnc1F)Cc1cn(S(=O)(=O)c2ccccc2)c2ccccc12)C1CCCCC1]

Under argon atmosphere, Compound 43 (184 mg, 0.335 mmol, 1 equiv) was dissolved in anhydrous ACN (19 mL, 0.02 M), fromaldehyde (60.3 mg, 2.01 mmol, 6 equiv) was added to the reactional mixture. After 1 h of stirring at RT, NaBH₃CN (84.2 mg, 1.34 mmol, 4 equiv) was added. The mixture was stirred at RT during 24 h. The mixture was concentrated under reduce pressure. The amorphous solid obtained was re-dissolved in DCM (40 mL) and this solution was washed with saturated solution of NaHCO₃(1×40 mL). The combined aqueous phases were extracted with DCM (3×40 mL). The combined organic phases were dried over anhydrous MgSO₄ and concentrated to afford 188 mg of a yellow oil. The crude product was purified by flash column chromatography on silica gel, using DCM/MeOH·NH₃ 2% as the mobile phase, to afford Compound 49 (188 mg, 100%) a yellow oil.

TLC: Eluent DCM/MeOH·NH₃ 3% R_{f}=0.64

¹H NMR (400 MHz, Chloroform-d) δ 8.05 (1H, m, H21), 7.97 (1H, d, J=8.3 Hz, H11), 7.84-7.74 (3H, m, H15 and H23), 7.59 (1H, d, J=7.6 Hz, H8), 7.50-7.45 (2H, m, H13 and H17), 7.37 (2H, m, H16), 7.31 (1H, m, H10), 7.22 (1H, m, H9), 7.09 (1H, m, H22), 3.69 (2H, s, H5), 3.55 (2H, s, H18), 2.46 (2H, m, H4), 2.37-2.25 (3H, m, H1 and H24), 2.18 (3H, s, H28), 1.75 (4H, m, H25 and H26), 1.61 (1H, m, H27), 1.53 (2H, p, J=7.2 Hz, H3), 1.38 (2H, p, J=7.3 Hz, H2), 1.26-1.02 (5H, m, H25′, H26′ and H27′).

¹³C NMR (100 MHz, Chloroform-d) δ 163.27 (C20), 145.96 (C21), 141.36 (C23), 138.21 (C14), 135.75 (C12), 133.89 (C17), 130.79 (C7), 129.32 (C16), 126.77 (C15), 125.07 (C10), 124.80 (C13), 123.39 (C9), 121.78 (C19), 121.52 (C22), 121.02 (C6), 120.58 (C8), 113.87 (C11), 62.81 (C24), 54.29 (C4), 53.43 (C1), 50.86 (C18), 49.79 (C5), 37.89 (C28), 28.62 (C25), 26.48 (C27), 26.16 (C26), 25.73 (C2), 25.18 (C3).

LRMS m/z (ESI+, CV 30) 563.4 [M+H]⁺.

### 8.2. Preparation of N1-cyclohexyl-N4-((2-fluoropyridin-4-yl)methyl)-N1-methyl-N4-((1-(phenylsulfonyl)-1H-indol-3-yl)methyl)butane-1,4-diamine (Compound 50, C₃₂H₃₉FN₄O₂S, MW=562.74 g/mol)

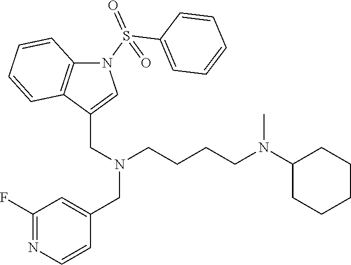 [SMILES: CN(CCCCN(Cc1ccnc(F)c1)Cc1cn(S(=O)(=O)c2ccccc2)c2ccccc12)C1CCCCC1]

According to general procedure 6. Scale: Compound 44 (41.3 mg, 0.075 mmol, 1 equiv), Formaldehyde (13.5 mg, 0.45 mmol, 6 equiv), NaBH₃CN (18.8 mg, 0.3 mmol, 4 equiv), ACN (4 mL). The crude product (49 mg of an amorphous yellow solid) was purified by flash column chromatography on silica gel, using a solvent gradient (DCM/MeOH·NH₃ 1% to 3%) to Compound 50 (29.8 mg, 71%) an amorphous yellow solid.

TLC: Eluent DCM/MeOH·NH₃

¹H NMR (400 MHz, Chloroform-d) δ 8.08 (1H, d, J=5.1 Hz, H21), 7.98 (1H, d, J=8.2 Hz, H11), 7.83 (2H, m, H15), 7.61 (1H, m, H8), 7.48 (2H, m, H13 and H17), 7.38 (2H, m, H16), 7.33 (1H, m, H10), 7.26 (1H, m, H9), 7.08 (1H, m, H20), 6.85 (1H, s, H23), 3.67 (2H, s, H5), 3.52 (2H, s, H18), 2.43 (5H, m, H1, H4 and H24), 2.23 (3H, s, H28), 1.78 (4H, m, H25 and H26), 1.63 (1H, m, H27), 1.53 (2H, q, J=7.5, 7.1 Hz, H3), 1.45 (2H, q, J=8.4, 7.9 Hz, H2), 1.28-1.03 (5H, m, H25′, H26′ and H27′).

¹³C NMR (100 MHz, Chloroform-d) δ 155.66 (C19), 147.52 (C21), 138.14 (C14), 135.69 (C12), 133.96 (C17), 130.66 (C7), 129.36 (C16), 126.76 (C15), 125.20 (C10), 124.94 (C13), 123.51 (C9), 121.43 (C20), 120.57 (C6), 120.49 (C8), 113.89 (C11), 109.05 (C23), 63.15 (C24), 57.34 (C18), 54.27 (C4), 53.32 (C1), 49.84 (C5), 37.55 (C28), 28.27 (C25), 26.23 (C26 and C27), 25.94 (C2), 25.12 (C3).

LRMS m/z (ESI+, CV 30) 563.4 [M+H]⁺.

### 8.3. Preparation of N1-cyclohexyl-N4-((6-fluoropyridin-2-yl)methyl)-N1-methyl-N4-((1-(phenylsulfonyl)-1H-indol-3-yl)methyl)butane-1,4-diamine (Compound 51, C₃₂H₃₉FN₄O₂S, MW=562.74 g/mol)

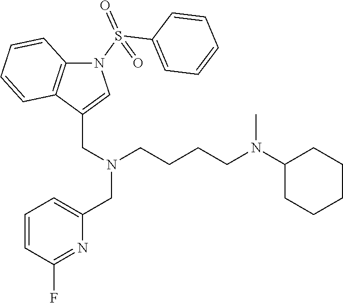 [SMILES: CN(CCCCN(Cc1cccc(F)n1)Cc1cn(S(=O)(=O)c2ccccc2)c2ccccc12)C1CCCCC1]

According to general procedure 6. Scale: Compound 45 (66.4 mg, 0.121 mmol, 1 equiv), Formaldehyde (21.8 mg, 0.726 mmol, 6 equiv), NaBH₃CN (30.4 mg, 0.484 mmol, 4 equiv), ACN (6 mL). The crude product (81 mg of an amorphous brown solid) was purified by flash column chromatography on silica gel, using a solvent gradient (DCM/MeOH·NH₃ 1% to 4%) to afford Compound 51 (49.1 mg, 72%) a yellow oil.

TLC: Eluent DCM/MeOH·NH₃

¹H NMR (400 MHz, Chloroform-d) δ 7.97 (1H, d, J=8.2 Hz, H11), 7.84-7.80 (2H, m, H15), 7.69 (1H, q, J=8.1 Hz, H22), 7.63 (1H, d, J=7.8 Hz, H8), 7.51-7.45 (2H, m, H13 and H17), 7.37 (2H, t, J=7.7 Hz, H16), 7.33-7.26 (2H, m, H10 and H23), 7.23 (1H, m, H9), 6.75 (1H, dd, J=8.0, 2.5 Hz, H21), 3.72 (2H, s, H5), 3.62 (2H, s, H18), 2.51 (2H, m, H4), 2.40-2.29 (3H, m, H1 and H24), 2.20 (3H, s, H28), 1.75 (4H, m, H25 and H26), 1.62 (1H, m, H27), 1.53 (2H, p, J=7.1 Hz, H3), 1.41 (2H, p, J=6.9, 6.3 Hz, H2), 1.26-0.99 (5H, m, H25′, H26′ and H27′).

¹³C NMR (100 MHz, Chloroform-d) δ 164.19 (C20), 159.66 (C19), 141.39 (C22), 138.22 (C14), 135.72 (C12), 133.86 (C17), 130.91 (C7), 129.32 (C16), 126.80 (C15), 125.00 (C20), 124.94 (C13), 123.37 (C9), 121.04 (C6), 120.62 (C8), 120.06 (C23), 113.85 (C11), 107.32 (C21), 62.96 (C24), 59.52 (C18), 54.48 (C4), 53.50 (C1), 49.82 (C5), 37.81 (C28), 28.54 (C25), 26.42 (C27), 26.10 (C26), 25.64 (C2), 25.25 (C3).

LRMS m/z (ESI+, CV 30) 563.3 [M+H]⁺.

### 8.4. Preparation of N1-cyclohexyl-N4-((6-fluoropyridin-3-yl)methyl)-N1-methyl-N4-((1-(phenylsulfonyl)-1H-indol-3-yl)methyl)butane-1,4-diamine (Compound 57, C₃₂H₃₉FN₄O₂S, MW=562.74 g/mol)

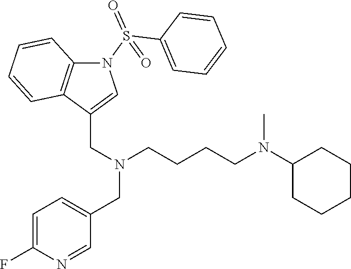 [SMILES: CN(CCCCN(Cc1ccc(F)nc1)Cc1cn(S(=O)(=O)c2ccccc2)c2ccccc12)C1CCCCC1]

According to general procedure 6. Scale: Compound 55 (18.7 mg, 0.034 mmol, 1 equiv), Formaldehyde (6.1 mg, 0.204 mmol, 6 equiv), NaBH₃CN (8.5 mg, 0.136 mmol, 4 equiv), ACN (2 mL). The crude product (16.7 mg of a colourless oil) was purified by flash column chromatography on silica gel, using a solvent gradient (DCM/MeOH·NH₃ 1% to 2%) to afford Compound 57 (13.1 mg, 69%) a yellow oil.

TLC: Eluent DCM/MeOH·NH3 2% Rf=0.48

RMN: 1H NMR (400 MHz, CDCl3) δ 8.05 (m, 1H, H20), 7.97 (d, J=8.4 Hz, 1H, H11), 7.84 (m, 2H, H15), 7.70 (m, 1H, H23), 7.57 (d, J=7.8 Hz, 1H, H8), 7.53-7.45 (m, 2H, H13 and H17), 7.40 (m, 2H, H16), 7.32 (m, 1H, H10), 7.24 (m, 1H, H9), 6.86 (dd, J=8.5, 2.9 Hz, 1H, H22), 3.63 (s, 2H, H5), 3.48 (s, 2H, H18), 2.51-2.37 (m, 5H, H1, H4 and H24), 2.26 (s, 3H, H28), 1.81 (m, 4H, H25 eq and H26 eq), 1.64 (m, 2H, H27 eq), 1.52 (m, 4H, H2 and H3), 1.29-1.14 (m, 6H, H25ax, H26ax and H27ax).

13C NMR (100 MHz, CDCl3) δ 147.62 (d, 3JCF=15.4 Hz, C20), 141.85 (C23), 138.20 (C14), 135.71 (C12), 133.97 (C17), 130.68 (C7), 129.39 (C16), 126.80 (C15), 1215.16 (C10), 124.92 (C13), 123.50 (C9), 120.76 (C6), 120.66 (C8), 113.86 (C13), 112.83 (C22), 63.166 (C24), 55.00 (C18), 53.12 (C1 and C4), 49.57 (C5), 36.92 (C28), 27.80 (C25), 25.78 (C26 and C27), 25.07 (C2 and C3).

LRMS m/z (ESI+, CV 30) 563.3 [M+H]⁺.

### 8.5. Preparation of N1-cyclohexyl-N5-((2-fluoropyridin-3-yl)methyl)-N1-methyl-N5-((1-(phenylsulfonyl)-1H-indol-3-yl)methyl)pentane-1,5-diamine (Compound 52, C₃₃H₄₁FN₄O₂S, MW=576.77 g/mol)

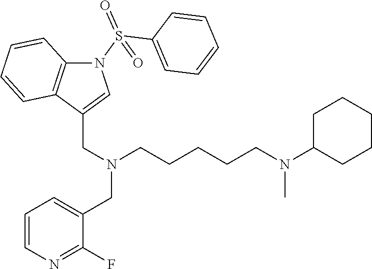 [SMILES: CN(CCCCCN(Cc1cccnc1F)Cc1cn(S(=O)(=O)c2ccccc2)c2ccccc12)C1CCCCC1]

According to general procedure 6. Scale: Compound 46 (50.5 mg, 0.090 mmol, 1 equiv), Formaldehyde (16.2 mg, 0.54 mmol, 6 equiv), NaBH₃CN (22.6 mg, 0.36 mmol, 4 equiv), ACN (4 mL). The crude product (51.4 mg of a colourless oil) was purified by flash column chromatography on silica gel, using a solvent gradient (DCM/MeOH·NH₃ 1% to 2%) to afford Compound 52 (41.1 mg, 79%) a yellow oil.

TLC: Eluent DCM/MeOH·NH₃ 2% R_{f}=0.53

¹H NMR (400 MHz, Chloroform-d) δ 8.05 (1H, m, H22), 7.97 (1H, d, J=8.3 Hz, H12), 7.83-7.73 (3H, m, H16 and H24), 7.59 (1H, d, J=7.8 Hz, H9), 7.47 (2H, m, H14 and H18), 7.37 (2H, m, H17), 7.31 (1H, m, H11), 7.22 (1H, m, H10), 7.09 (1H, m, H23), 3.69 (2H, s, H6), 3.55 (2H, s, H19), 2.44 (2H, m, H5), 2.36-2.28 (3H, m, H1 and H25), 2.19 (3H, s, H29), 1.76 (4H, m, H26 and H27), 1.62 (1H, m, H28), 1.53 (2H, p, J=7.5 Hz, H4), 1.37 (2H, p, J=7.4 Hz, H2), 1.28-1.00 (7H, m, H3, H26′, H27′ and H28′).

¹³C NMR (100 MHz, Chloroform-d) δ 160.88 (C21), 145.92 (C22), 141.31 (C24), 138.21 (C15), 135.76 (C13), 133.87 (C18), 130.81 (C8, 129.30 (C17), 126.76 (C16), 125.05 (C11), 124.77 (C14), 123.38 (C20), 121.81 (C20), 121.50 (C23), 121.09 (C7), 120.55 (C9), 113.88 (C12), 62.79 (C25), 54.41 (C5), 53.67 (C1), 50.87 (C19), 49.76 (C6), 37.95 (C29), 28.68 (C26), 27.99 (C2), 27.20 (C4), 26.52 (C28), 26.18 (C27), 25.49 (C3).

LRMS m/z (ESI+, CV 30) 577.3 [M+H]₊.

### 8.6. Preparation of N1-cyclohexyl-N5-((2-fluoropyridin-4-yl)methyl)-N1-methyl-N5-((1-(phenylsulfonyl)-1H-indol-3-yl)methyl)pentane-1,5-diamine (Compound 53, C₃₃H₄₁FN₄O₂S, MW=576.77 g/mol)

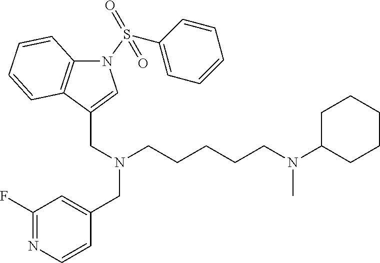 [SMILES: CN(CCCCCN(Cc1ccnc(F)c1)Cc1cn(S(=O)(=O)c2ccccc2)c2ccccc12)C1CCCCC1]

According to general procedure 6. Scale: Compound 47 (154.2 mg, 0.274 mmol, 1 equiv), Formaldehyde (65.76 mg, 1.083 mmol, 8 equiv), NaBH₃CN (68.9 mg, 1.096 mmol, 4 equiv), ACN (13 mL). The crude product (262.7 mg of an amorphous yellow solid) was purified by flash column chromatography on silica gel, using a solvent gradient (DCM/MeOH·NH₃ 1% to 4%) to afford Compound 53 (140.2 mg, 89%) a yellow oil.

TLC: Eluent DCM/MeOH·NH₃

¹H NMR (400 MHz, Chloroform-d) δ 8.06 (1H, d, J=5.1 Hz, H22), 7.98 (1H, d, J=8.2 Hz, H12), 7.82 (2H, m, H16), 7.62 (1H, d, J=7.8 Hz, H9), 7.47 (2H, m, H14 and H18), 7.40-7.30 (3H, m, H11 and H17), 7.28-7.22 (1H, m, H10), 7.05 (1H, m, H21), 6.84 (1H, s, H24), 3.67 (2H, s, H6), 3.50 (2H, s, H19), 2.43 (2H, m, H5), 2.39-2.30 (3H, m, H1 and H25), 2.21 (3H, s, H29), 1.77 (4H, m, H26 and H27), 1.65-1.58 (1H, m, H28), 1.54 (2H, p, J=7.5 Hz, H4), 1.39 (2H, p, J=7.4 Hz, H2), 1.21 (8H, m, H3, H26′, H27′ and H28′).

¹³C NMR (100 MHz, Chloroform-d) δ 163.04 (C23), 155.77 (C20), 147.42 (C22), 138.19 (C15), 135.73 (C13), 133.91 (C18), 130.71 (C8), 129.32 (C17), 126.75 (C16), 125.17 (C11), 124.89 (C14), 123.46 (C10), 121.32 (C21), 120.69 (C7), 120.47 (C9), 113.91 (C12), 108.96 (C24), 62.86 (C25), 57.26 (C19), 54.62 (C5), 53.61 (C1), 49.76 (C6), 37.90 (C29), 28.63 (C26), 27.92 (C2), 27.25 (C4), 26.48 (C28), 26.14 (C27), 25.42 (C3).

LRMS m/z (ESI+, CV 30) 577.3 [M+H]⁺.

### 8.7. Preparation of N1-cyclohexyl-N5-((6-fluoropyridin-2-yl)methyl)-N1-methyl-N5-((1-(phenylsulfonyl)-1H-indol-3-yl)methyl)pentane-1,5-diamine (Compound 54, C₃₃H₄₁FN₄O₂S, MW=576.77 g/mol)

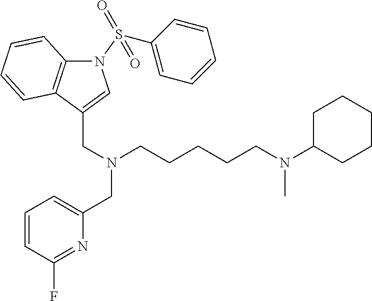 [SMILES: CN(CCCCCN(Cc1cccc(F)n1)Cc1cn(S(=O)(=O)c2ccccc2)c2ccccc12)C1CCCCC1]

According to general procedure 6. Scale: Compound 48 (92.7 mg, 0.165 mmol, 1 equiv), Formaldehyde (44.6 mg, 1.487 mmol, 9 equiv), NaBH₃CN (51.8 mg, 0.825 mmol, equiv), ACN (8 mL). The crude product (202 mg of a yellow oil) was purified by flash column chromatography on silica gel, using a solvent gradient (DCM/MeOH·NH₃ 1% to 3%) to afford Compound 54 (38 mg, 40%) an amorphous yellow solid.

TLC: Eluent DCM/MeOH·NH3 4% Rf=0.60

1H NMR (400 MHz, Chloroform-d) δ 7.97 (1H, d, J=8.2 Hz, H12), 7.81 (2H, m, H16), 7.67 (2H, m, H9 and H23), 7.51-7.44 (2H, m, H14 and H18), 7.37 (2H, m, H17), 7.33-7.27 (2H, m, H11 and H24), 7.23 (1H, m, H10), 6.74 (1H, dd, J=8.0, 2.6 Hz, H22), 3.72 (2H, s, H6), 3.62 (2H, s, H19), 2.48 (2H, m, H5), 2.37-2.27 (3H, m, H1 and H25), 2.20 (3H, s, H29), 1.76 (4H, m, H26 and H27), 1.65-1.57 (1H, m, H28), 1.54 (2H, p, J=7.5 Hz, H4), 1.38 (2H, p, J=7.4 Hz, H2), 1.27-1.01 (7H, m, H3, H26′, H27′ and H28′).

13C NMR (100 MHz, Chloroform-d) δ 13C NMR (100 MHz, CDCl3) δ 162.86 (d, 1JCF=232.8 Hz, C21), 159.77 (d, 3JCF=12.9 Hz, C20), 141.32 (d, 3JCF=6.9 Hz, C23), 138.23 (C15), 135.74 (C13), 133.82 (C18), 130.94 (C8), 129.30 (C17), 126.79 (C16), 124.98 (C11), 124.89 (C14), 123.35 (C10), 121.18 (C7), 120.58 (C9), 119.93 (d, 4JCF=4.6 Hz, C24), 113.87 (C12), 107.25 (d, 2JCF=37.4 Hz, C22), 62.78 (C25), 59.53 (C19), 54.73 (C5), 53.70 (C1), 49.80 (C6), 37.98 (C29), 28.70 (C26), 28.04 (C2), 27.31 (C4), 26.53 (C28), 26.19 (C27), 25.53 (C3).

LRMS m/z (ESI+, CV 30) 577.4 [M+H]⁺.

### 8.8. Preparation of N1-cyclohexyl-N1-methyl-N5-((6-methylpyridin-3-yl)methyl)-N5-((1-(phenylsulfonyl)-1H-indol-3-yl)methyl)pentane-1,5-diamine (Compound 58, C₃₃H₄₁FN₄O₂S, MW=576.77 g/mol)

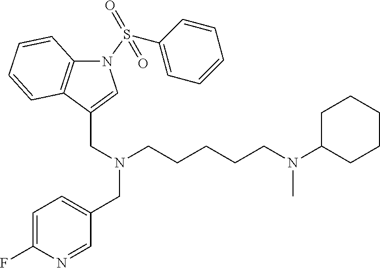 [SMILES: CN(CCCCCN(Cc1ccc(F)nc1)Cc1cn(S(=O)(=O)c2ccccc2)c2ccccc12)C1CCCCC1]

According to general procedure 6. Scale: Compound 56 (63.2 mg, 0.112 mmol, 1 equiv), Formaldehyde (20.2 mg, 0.672 mmol, 6 equiv), NaBH₃CN (28.1 mg, 0.448 mmol, 4 equiv), ACN (6 mL). The crude product (67.3 mg of a colorless oil) was purified by flash column chromatography on silica gel, using a solvent gradient (DCM/MeOH·NH₃ 1% to 3%) to afford Compound 58 (52.7 mg, 81%) a colourless oil.

TLC: Eluent DCM/MeOH·NH₃ 2% R_{f}=0.55

¹H NMR (400 MHz, Chloroform-d) δ 8.04 (1H, m, H21), 7.98 (1H, d, J=8.3 Hz, H12), 7.83 (2H, m, H16), 7.67-7.58 (2H, m, H9 and H24), 7.47 (2H, m, H14 and H18), 7.38 (2H, m, H17), 7.32 (1H, m, H11), 7.23 (1H, m, H10), 6.82 (1H, dd, J=8.4, 2.8 Hz, H23), 3.65 (2H, s, H6), 3.46 (2H, s, H19), 2.40 (2H, m, H5), 2.36-2.27 (3H, m, H1 and H25), 2.19 (3H, s, H29), 1.76 (4H, m, H26 and H27), 1.65 (1H, m, H28), 1.52 (2H, p, J=7.5 Hz, H4), 1.36 (2H, p, J=7.4 Hz, H2), 1.28-1.00 (7H, m, H3, H26′, H27′ and H28′).

¹³C NMR (100 MHz, Chloroform-d) δ 147.57 (C21), 141.79 (C24), 138.24 (C15), 135.77 (C13), 133.90 (C18), 132.95 (C20), 130.77 (C8), 129.34 (C17), 126.78 (C16), 125.12 (C11), 124.85 (C14), 123.43 (C10), 120.97 (C7), 120.63 (C9), 113.89 (C12), 109.24 (C23), 62.82 (C25), 54.89 (C19), 53.97 (C5), 53.67 (C1), 49.57 (C6), 37.93 (C29), 28.67 (C26), 27.98 (C2), 27.16 (C4), 26.52 (C28), 26.18 (C27), 25.47 (C3).

LRMS m/z (ESI+, CV 30) 577.3 [M+H]⁺.

### 8.9. Preparation of N′-cyclohexyl-N′-ethyl-N⁴-((2-fluoropyridin-3-yl)methyl)-N⁴-((1-(phenylsulfonyl)-1H-indol-3-yl)methyl)butane-1,4-diamine (Compound 66, C₃₃H₄₁FN₄O₂S, MW=576.77 g/mol)

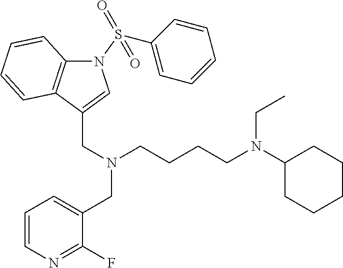 [SMILES: CCN(CCCCN(Cc1cccnc1F)Cc1cn(S(=O)(=O)c2ccccc2)c2ccccc12)C1CCCCC1]

According to general procedure 5. Scale: Intermediate 15 (110 mg, 0.200 mmol, 1 equiv), Acetaldehyde (13.2 mg, 0.300 mmol, 1.5 equiv), NaBH(OAc)3 (67.8 mg, 0.320 mmol, 1.6 equiv), DCM (4 mL). The crude product (117.4 mg of an amorphous yellow solid) was purified by flash column chromatography on silica gel, using DCM/MeOH·NH₃ 2% as the eluent to afford Compound 66 (56.1 mg, 49%) an amorphous colourless solid.

TLC: Eluent DCM/MeOH·NH₃ 2% R_{f}=0.46

¹H NMR (400 MHz, Chloroform-d) δ 8.05 (1H, m, H21), 7.97 (1H, m, H11), 7.84-7.74 (3H, m, H15 and H23), 7.60 (1H, d, J=7.7 Hz, H8), 7.48 (2H, m, H13 and H17), 7.37 (2H, m, H16), 7.31 (1H, m, H10), 7.22 (1H, m, H9), 7.09 (1H, m, H22), 3.69 (2H, s, H5), 3.55 (2H, s, H18), 2.47 (5H, m, H4, H24 and H28), 2.35 (2H, m, H1), 1.72 (4H, m, H25 and H26), 1.60 (1H, m, H27), 1.52 (2H, p, J=7.4 Hz, H3), 1.35 (2H, p, J=7.5 Hz, H2), 1.27-1.01 (5H, m, H25′, H26′ and H27′), 0.97 (3H, t, J=7.1 Hz, H29).

¹³C NMR (100 MHz, Chloroform-d) δ 145.91 (C21), 141.33 (C23), 138.23 (C14), 135.77 (C12), 133.87 (C17), 130.82 (C7), 129.31 (C16), 126.76 (C15), 125.05 (C10), 124.80 (C13), 123.38 (C9), 121.83 (C19), 121.51 (C22), 121.06 (C6), 120.58 (C8), 113.88 (C11), 59.96 (C24), 54.44 (C4), 50.85 (C18), 49.95 (C1), 49.79 (C5), 44.34 (C28), 29.28 (C25), 27.01 (C2), 26.57 (C27), 26.36 (C26), 25.23 (C3), 14.27 (C29).

¹⁹F NMR (376 MHz, Chloroform-d) δ−72.67.

LRMS m/z (ESI+, CV 30) 577.4 [M+H]⁺.

### 8.10. Preparation of N′-cyclohexyl-N⁴-((2-fluoropyridin-3-yl)methyl)-N⁴-((1-(phenylsulfonyl)-1H-indol-3-yl)methyl)-N′-propylbutane-1,4-diamine (Compound 67, C₃₄H₄₃FN₄O₂S, MW=590.79 g/mol)

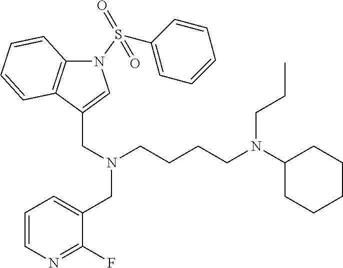 [SMILES: CCCN(CCCCN(Cc1cccnc1F)Cc1cn(S(=O)(=O)c2ccccc2)c2ccccc12)C1CCCCC1]

According to general procedure 5. Scale: Intermediate 15 (80 mg, 0.146 mmol, 1 equiv), Propionaldehyde (10.2 mg, 0.175 mmol, 1.2 equiv), NaBH(OAc)3 (46.4 mg, 0.219 mmol, 1.5 equiv), DCM (3 mL). The crude product (97.6 mg of an orange oil) was purified by flash column chromatography on silica gel, using DCM/MeOH·NH₃ 2% as the eluent to afford Compound 67 (20.8 mg, 24%) an amorphous colourless solid.

TLC: Eluent DCM/MeOH·NH₃ 2% R_{f}=0.50

¹H NMR (400 MHz, Chloroform-d) δ 8.05 (1H, m, H21), 7.97 (1H, d, J=8.3 Hz, H11), 7.84-7.74 (3H, m, H15 and H23), 7.60 (1H, d, J=7.7 Hz, H8), 7.48 (2H, m, H13 and H17), 7.37 (2H, m, H16), 7.31 (1H, m, H10), 7.22 (1H, m, H9), 7.09 (1H, m, H22), 3.69 (2H, s, H5), 3.55 (2H, s, H18), 2.45 (2H, m, H4), 2.33 (5H, m, H1, H24 and H28), 1.72 (4H, m, H25 and H26), 1.59 (1H, m, H27), 1.53 (2H, m, H3), 1.35 (4H, m, H2 and H29), 1.15 (5H, m, H25′, H26′ and H27′), 0.82 (3H, t, J=7.3 Hz, H30).

¹³C NMR (100 MHz, Chloroform-d) δ 156.29 (C20), 145.94 (C21), 141.30 (C23), 138.22 (C14), 135.77 (C12), 133.87 (C17), 130.82 (C7), 129.31 (C16), 126.77 (C15), 125.06 (C10), 124.80 (C13), 123.39 (C9), 121.49 (C22), 121.09 (C6), 120.59 (C8), 113.88 (C11), 60.21 (C24), 54.47 (C4), 52.81 (C28), 50.81 (C18), 50.56 (C1), 49.79 (C5), 29.20 (C25), 27.13 (C2), 26.60 (C27), 26.41 (C26), 25.14 (C3), 22.49 (C29), 12.06 (C30).

¹⁹F NMR (376 MHz, Chloroform-d) δ−72.69.

LRMS m/z (ESI+, CV 30) 591.2 [M+H]⁺

### 8.11. Preparation of N′-butyl-N′-cyclohexyl-N⁴-((2-fluoropyridin-3-yl)methy)-N⁴-((1-(phenylsulfonyl)-1H-indol-3-yl)methyl)butane-1,4-diamine (Compound 68, C₃₅H₄₅FN₄O₂S, MW=604.82 g/mol)

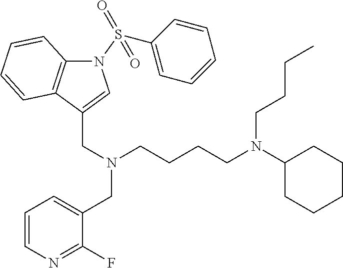 [SMILES: CCCCN(CCCCN(Cc1cccnc1F)Cc1cn(S(=O)(=O)c2ccccc2)c2ccccc12)C1CCCCC1]

According to general procedure 5. Scale: Intermediate 15 (90 mg, 0.164 mmol, 1 equiv), Butionaldehyde (14.2 mg, 0.197 mmol, 1.2 equiv), NaBH(OAc)3 (55.5 mg, 0.262 mmol, 1.6 equiv), DCM (3 mL). The crude product (107.9 mg of an amorphous yellow solid) was purified by flash column chromatography on silica gel, using a solvent gradient (DCM/MeOH·NH₃ 1% to 3%) to afford Compound 68 (76.9 mg, 78%) a colourless solid.

TLC: Eluent DCM/MeOH·NH₃ 2% R_{f}=0.81

¹H NMR (400 MHz, Chloroform-d) δ 8.05 (1H, m, H21), 7.97 (1H, d, J=8.3 Hz, H11), 7.84-7.74 (3H, m, H15 and H23), 7.60 (1H, d, J=7.8 Hz, H8), 7.48 (2H, m, H13 and H17), 7.37 (2H, m, H16), 7.31 (1H, m, H10), 7.22 (1H, m, H9), 7.09 (1H, m, H22), 3.69 (2H, s, H5), 3.55 (2H, s, H18), 2.45 (2H, m, H4), 2.35 (5H, m, H1, H24 and H28), 1.73 (4H, m, H25 and H26), 1.62 (1H, m, H27), 1.53 (2H, m, H3), 1.39-0.99 (11H, m, H2, H25′, H26′, H27′, H29 and H30), 0.88 (3H, t, J=7.2 Hz, H31).

¹³C NMR (100 MHz, Chloroform-d) δ 145.90 (C21), 141.30 (C23), 138.22 (C14), 135.77 (C12), 133.87 (C17), 130.82 (C7), 129.31 (C16), 126.76 (C15), 125.05 (C10), 124.80 (C13), 123.38 (C9), 121.51 (C22), 121.08 (C6), 120.58 (C8), 113.88 (C11), 60.10 (C24), 54.49 (C4), 50.80 (C18), 50.51 (C1), 49.78 (C5), 31.63 (C29), 29.18 (C25), 27.11 (C2) 26.59 (C27), 26.41 (C26), 25.16 (C3), 20.83 (C30), 14.28 (C31).

¹⁹F NMR (376 MHz, Chloroform-d) δ−72.69.

LRMS m/z (ESI+, CV 30) 605.3 [M+H]⁺.

### 8.12. Preparation of N′-cyclobutyl-N⁴-((2-fluoropyridin-3-yl)methyl)-N⁴-((1-(phenylsulfonyl)-1H-indol-3-yl)methyl)butane-1,4-diamine (Compound 69, C₂₉H₃₃FN₄O₂S, MW=520.66 g/mol)

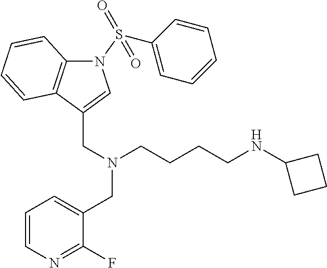 [SMILES: O=S(=O)(c1ccccc1)n1cc(CN(CCCCNC2CCC2)Cc2cccnc2F)c2ccccc21]

According to general procedure 5. Scale: Intermediate 15 (100 mg, 0.214 mmol, 1 equiv), Cyclobutanone (18 mg, 0.257 mmol, 1.2 equiv), NaBH(OAc)3 (68 mg, 0.321 mmol, 1.5 equiv), DCM (3 mL). The crude product (136.3 mg of an amorphous yellow solid) was purified by flash column chromatography on silica gel, using DCM/MeOH·NH₃ 2% as the eluent to afford Compound 69 (10.7 mg, 10%) an amorphous yellow solid.

TLC: Eluent DCM/MeOH·NH₃ 2% R_{f}=0.43

¹H NMR (400 MHz, Chloroform-d) δ 8.06 (1H, m, H21), 7.97 (1H, d, J=8.3 Hz, H11), 7.84-7.74 (3H, m, H15 and H23), 7.58 (1H, d, J=7.8 Hz, H8), 7.48 (2H, m, H13 and H17), 7.38 (2H, m, H16), 7.31 (1H, m, H10), 7.22 (1H, m, H9), 7.10 (1H, m, H22), 3.69 (2H, s, H5), 3.55 (2H, s, H18), 3.15 (1H, m, H24), 2.44 (4H, m, H1 and H4), 2.16 (2H, m, H25), 1.71-1.50 (5H, m, H3, H25′ and H26′), 1.38 (2H, m, H2).

¹³C NMR (100 MHz, Chloroform-d) δ 163.29 (C20), 146.01 (C21), 141.30 (C23), 138.25 (C14), 135.76 (C12), 133.88 (C17), 130.78 (C7), 129.32 (C16), 126.78 (C15), 125.08 (C10), 124.80 (C13), 123.40 (C9), 121.72 (C19), 121.50 (C22), 120.95 (C6), 120.55 (C8), 113.89 (C11), 54.24 (C24), 54.22 (C4), 50.86 (C18), 49.78 (C5), 46.81 (C1), 31.34 (C25), 28.22 (C2), 25.09 (C3), 14.91 (C26).

¹⁹F NMR (376 MHz, Chloroform-d) δ−72.59.

LRMS m/z (ESI+, CV 30) 521.4 [M+H]⁺.

### 8.13. Preparation of N′-cyclopentyl-N⁴-((2-fluoropyridin-3-yl)methyl)-N⁴-((1-(phenylsulfonyl)-1H-indol-3-yl)methyl)butane-1,4-diamine (Compound 71, C₃₀H₃₅FN₄O₂S, MW=534.69 g/mol)

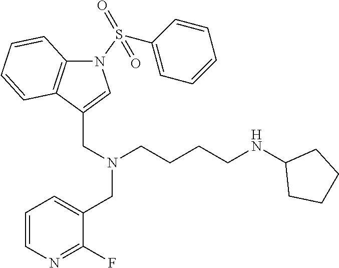 [SMILES: O=S(=O)(c1ccccc1)n1cc(CN(CCCCNC2CCCC2)Cc2cccnc2F)c2ccccc21]

According to general procedure 5. Scale: Intermediate 15 (80 mg, 0.171 mmol, 1 equiv), Cyclopentane (17.2 mg, 0.205 mmol, 1.2 equiv), NaBH(OAc)3 (54.5 mg, 0.257 mmol, 1.5 equiv), DCM (2 mL). The crude product (92 mg of an amorphous yellow solid) was purified by flash column chromatography on silica gel, using a solvent gradient (DCM/MeOH·NH₃ 1% to 3%) to afford Compound 71 (72.1 mg, 79%) an amorphous colourless solid.

TLC: Eluent DCM/MeOH·NH₃ 2% R_{f}=0.40

¹H NMR (400 MHz, Chloroform-d) δ 8.06 (1H, m, H21), 7.97 (1H, d, J=8.3 Hz, H11), 7.84-7.74 (3H, m, H15 and H23), 7.58 (1H, d, J=7.8 Hz, H8), 7.48 (2H, m, H13 and H17), 7.38 (2H, m, H16), 7.31 (1H, m, H10), 7.22 (1H, m, H9), 7.10 (1H, m, H22), 3.69 (2H, s, H5), 3.56 (2H, s, H18), 2.96 (1H, p, J=6.8 Hz, H24), 2.47 (4H, m, H1 and H4), 1.79 (2H, m, H25), 1.71-1.37 (6H, m, H2, H3 and H26), 1.23 (2H, m, H25′).

¹³C NMR (100 MHz, Chloroform-d) δ 146.00 (C21), 141.33 (C23), 138.22 (C14), 135.75 (C12), 133.89 (C17), 130.77 (C7), 129.32 (C16), 126.78 (C15), 125.08 (C10), 124.80 (C13), 123.39 (C9), 121.64 (C22), 120.96 (C6), 120.55 (C8), 113.88 (C11), 60.05 (C24), 54.21 (C4), 50.87 (C18), 49.77 (C5), 48.67 (C1), 33.34 (C25), 28.29 (C2), 25.14 (C3), 24.20 (C26). ¹⁹F NMR (376 MHz, Chloroform-d) δ−72.61.

LRMS m/z (ESI+, CV 30) 535.2 [M+H]⁺.

### 8.14. Preparation of N′-cycloheptyl-N⁴-((2-fluoropyridin-3-yl)methyl)-N⁴-((1-(phenylsulfonyl)-1H-indol-3-yl)methyl)butane-1,4-diamine (Compound 73, C₃₂H₃₉FN₄O₂S, MW=562.74 g/mol)

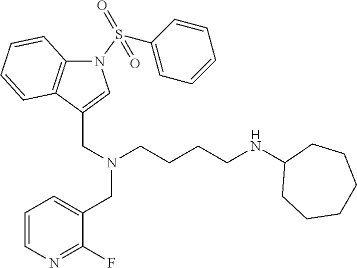 [SMILES: O=S(=O)(c1ccccc1)n1cc(CN(CCCCNC2CCCCCC2)Cc2cccnc2F)c2ccccc21]

According to general procedure 5. Scale: Intermediate 15 (197 mg, 0.422 mmol, 1 equiv), Cycloheptanone (47.3 mg, 0.422 mmol, 1 equiv), NaBH₄ (25.5 mg, 0.675 mmol, 1.6 equiv), MeOH (6 mL). The crude product (249 mg of a brown oil) was purified by flash column chromatography on silica gel, using DCM/MeOH·NH₃ 2% as the eluent to afford Compound 73 (79 mg, 33%) an amorphous colourless solid.

TLC: Eluent DCM/MeOH·NH₃ 3% R_{f}=0.67

¹H NMR (400 MHz, Chloroform-d) δ 8.05 (1H, m, H21), 7.97 (1H, d, J=8.3 Hz, H11), 7.84-7.74 (3H, m, H15 and H23), 7.58 (1H, d, J=7.8 Hz, H8), 7.48 (2H, m, H13 and H17), 7.38 (2H, m, H16), 7.31 (1H, m, H10), 7.22 (1H, m, H9), 7.10 (1H, m, H22), 3.69 (2H, s, H5), 3.55 (2H, s, H18), 2.55-2.41 (5H, m, H1, H4 and H24), 1.81-1.71 (2H, m, H25), 1.67-1.45 (6H, m, H3, H26 and H27), 1.44-1.24 (8H, m, H2, H25′, H26′ and H27′).

¹³C NMR (100 MHz, Chloroform-d) δ 160.89 (C20), 145.98 (C21), 141.34 (C23), 138.20 (C14), 135.74 (C12), 133.88 (C17), 130.77 (C7), 129.31 (C16), 126.77 (C15), 125.07 (C10), 124.79 (C13), 123.39 (C9), 121.72 (C19), 121.50 (C22), 120.96 (C6), 120.54 (C8), 113.87 (C11), 59.38 (C24), 54.21 (C4), 50.86 (C18), 49.76 (C5), 47.53 (C1), 35.09 (C25), 28.39 (C27), 25.14 (C3), 24.60 (C2 and C26).

¹⁹F NMR (376 MHz, Chloroform-d) δ−72.62.

LRMS m/z (ESI+, CV 30) 563.3 [M+H]⁺.

### 8.15. Preparation of N′-cyclooctyl-N⁴-((2-fluoropyridin-3-yl)methyl)-N⁴-((1-(phenylsulfonyl)-1H-indol-3-yl)methyl)butane-1,4-diamine (Compound 75, C₃₃H₄₁FN₄O₂S, MW=576.77 g/mol)

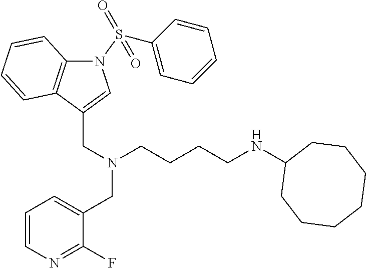 [SMILES: O=S(=O)(c1ccccc1)n1cc(CN(CCCCNC2CCCCCCC2)Cc2cccnc2F)c2ccccc21]

According to general procedure 5. Scale: Intermediate 15 (80 mg, 0.171 mmol, 1 equiv), Cyclooctanone (25.9 mg, 0.205 mmol, 1.2 equiv), NaBH(OAc)3 (54.5 mg, 0.257 mmol, 1.5 equiv), DCM (2 mL). The crude product (82.8 mg of a brown oil) was purified by flash column chromatography on silica gel, using a solvent gradient (DCM/MeOH·NH₃ 1% to 3%) to afford Compound 75 (71.9 mg, 73%) an amorphous colourless solid.

TLC: Eluent DCM/MeOH·NH₃ 3% R_{f}=0.61

¹H NMR (400 MHz, Chloroform-d) δ 8.05 (1H, m, H21), 7.97 (1H, d, J=8.3 Hz, H11), 7.84-7.74 (3H, m, H15 and H23), 7.58 (1H, d, J=7.7 Hz, H8), 7.51-7.45 (2H, m, H13 and H17), 7.38 (2H, m, H16), 7.34-7.28 (1H, m, H10), 7.25-7.19 (1H, m, H9), 7.10 (1H, m, H22), 3.69 (2H, s, H5), 3.55 (2H, s, H18), 2.57 (1H, s, H24), 2.47 (4H, m, H1 and H4), 1.68 (4H, m, H25 and H26), 1.56 (5H, m, H3, H27 and H28), 1.42 (9H, m, H2, H25′, H26′, H27′ and H28′).

¹³C NMR (100 MHz, Chloroform-d) δ 145.87 (C21), 141.20 (C23), 138.09 (C14), 135.62 (C12), 133.76 (C17), 130.65 (C7), 129.19 (C16), 126.65 (C15), 124.95 (C10), 124.68 (C13), 123.27 (C9), 121.58 (C19), 121.41 (C22), 120.81 (C6), 120.41 (C8), 113.75 (C11), 58.04 (C24), 54.10 (C4), 50.73 (C18), 49.63 (C5), 47.27 (C1), 32.70 (C25), 28.12 (C2), 27.25 (C27 or C28), 25.80 (C27 or C28), 25.03 (C3), 24.19 (C26).

¹⁹F NMR (376 MHz, Chloroform-d) δ−72.60.

LRMS m/z (ESI+, CV 30) 577.3 [M+H]⁺.

### 8.16. Preparation of N′-cyclobutyl-N⁴-((2-fluoropyridin-3-yl)methyl)-N′-methyl-N⁴-((1-(phenylsulfonyl)-1H-indol-3-yl)methyl)butane-1,4-diamine (Compound 70, C₃₀H₃₅FN₄O₂S, MW=534.69 g/mol)

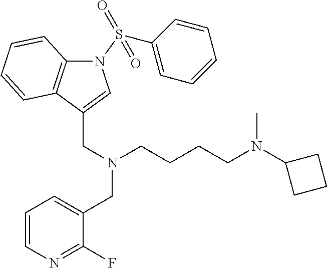 [SMILES: CN(CCCCN(Cc1cccnc1F)Cc1cn(S(=O)(=O)c2ccccc2)c2ccccc12)C1CCC1]

According to general procedure 6. Scale: Compound 69 (35 mg, 0.067 mmol, 1 equiv), Formaldehyde (12.1 mg, 0.402 mmol, 6 equiv), NaBH₃CN (16.8 mg, 0.268 mmol, 4 equiv), ACN (2.5 mL). The crude product (33.3 mg of an amorphous yellow solid) was purified by flash column chromatography on silica gel, using DCM/MeOH·NH₃ 2% as the eluent to afford Compound 70 (35.3 mg, 97%) an amorphous yellow solid.

TLC: Eluent DCM/MeOH·NH3 2% Rf=0.63

Eluent DCM/MeOH·NH3

1H NMR (400 MHz, Chloroform-d) δ 8.06 (m, 1H, H21), 7.97 (m, 1H, H11), 7.82 (m, 2H, H15), 7.77 (m, 1H, H23), 7.56 (m, 1H, H8), 7.48 (m, 2H, H13 and H17), 7.38 (m, 2H, H16), 7.31 (m, 1H, H10), 7.22 (m, 1H, H9), 7.11 (m, 1H, H22), 3.68 (s, 2H, H5), 3.56 (s, 2H, H18), 2.69 (quint, J=7.6 Hz, 1H, H24), 2.46 (t, J=7.1 Hz, 2H, H4), 2.17 (m, 2H, H1), 2.04 (s, 3H, H27), 2.02-1.82 (m, 2H, H25), 1.74-1.56 (m, 4H, H25 and H26), 1.57 (quint, J=8.6 Hz, 2H, H3), 1.41 (m, 2H, H2).

13C NMR (100 MHz, Chloroform-d) δ 162.08 87 (d, 1JCF=234.6 Hz, C20), 146.06 (d, 3JCF=15.6 Hz, C21), 141.51 (d, 3JCF=6.0 Hz, C23), 138.19 (C14), 135.73 (C12), 133.93 (C17), 130.76 (C7), 129.35 (C16), 126.78 (C15), 125.08 (C10), 124.82 (C13), 123.41 (C9), 121.55 (d, 4JCF=4.2 Hz, C22), 121.51 (d, 2JCF=28.3 Hz, C19), 120.92 (C6), 120.56 (C8), 113.87 (C11), 60.60 (C24), 54.07 (C4), 53.90 (C1), 50.88 (C18), 49.82 (C5), 37.64 (C27), 27.74 (C25), 25.25 (C3), 24.35 (C2), 14.06 (C26).

19F NMR (376 MHz, CDCl3) δ−72.54.

LRMS m/z (ESI+, CV 30) 535.4 [M+H]⁺.

### 8.17. Preparation of N′-cyclopentyl-N⁴-((2-fluoropyridin-3-yl)methyl)-N′-methyl-N⁴-((1-(phenylsulfonyl)-1H-indol-3-yl)methyl)butane-1,4-diamine (Compound 72, C₃₁H₃₇FN₄O₂S, MW=548.71 g/mol)

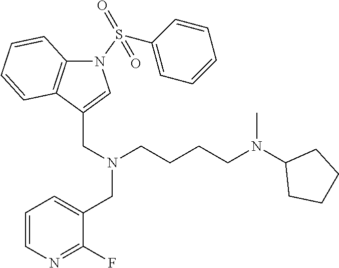 [SMILES: CN(CCCCN(Cc1cccnc1F)Cc1cn(S(=O)(=O)c2ccccc2)c2ccccc12)C1CCCC1]

According to general procedure 6. Scale: Compound 71 (77.3 mg, 0.145 mmol, 1 equiv), Formaldehyde (26.1 mg, 0.870 mmol, 6 equiv), NaBH₃CN (36.4 mg, 0.580 mmol, 4 equiv), ACN (3 mL). The crude product (69.3 mg of an amorphous yellow solid) was purified by flash column chromatography on silica gel, using a solvent gradient (DCM/MeOH·NH₃ 1% to 3%) to afford Compound 72 (68.9 mg, 87%) an amorphous colourless solid.

TLC: Eluent DCM/MeOH·NH₃ 2% R_{f}=0.38

¹H NMR (400 MHz, Chloroform-d) δ 8.05 (1H, m, H21), 7.97 (1H, d, J=8.3 Hz, H11), 7.84-7.73 (3H, m, H15 and H23), 7.59 (1H, d, J=7.6 Hz, H8), 7.48 (2H, m, H13 and H17), 7.37 (2H, m, H16), 7.31 (1H, m, H10), 7.22 (1H, m, H9), 7.09 (1H, m, H22), 3.69 (2H, s, H5), 3.55 (2H, s, H18), 2.56 (1H, m, H24), 2.46 (2H, m, H4), 2.29 (2H, m, H1), 2.15 (3H, s, H27), 1.80-1.70 (2H, m, H25), 1.64 (2H, m, H26), 1.50 (4H, m, H3 and H26′), 1.45-1.23 (4H, m, H2 and H25′).

¹³C NMR (100 MHz, Chloroform-d) δ 160.88 (C20), 146.94 (C21), 141.32 (C23), 138.20 (C14), 135.74 (C12), 133.86 (C17), 130.77 (C7), 129.30 (C16), 126.75 (C15), 125.05 (C10), 124.78 (C13), 123.37 (C9), 121.74 (C19), 121.48 (C22), 120.99 (C6), 120.56 (C8), 113.86 (C11), 66.88 (C24), 56.05 (C1), 54.32 (C4), 50.85 (C18), 49.79 (C5), 40.20 (C27), 30.49 (C25), 25.35 (C3), 24.81 (C2), 24.33 (C26).

¹⁹F NMR (376 MHz, Chloroform-d) δ−72.63.

LRMS m/z (ESI+, CV 30) 549.2 [M+H]⁺.

### 8.18. Preparation of N′-cycloheptyl-N⁴-((2-fluoropyridin-³-yl)methyl)-N′-methyl-N⁴-((1-(phenylsulfonyl)-1H-indol-³-yl)methyl)butane-1,4-diamine (Compound 74, C₃₃H₄₁FN₄O₂S, MW=576.77 g/mol)

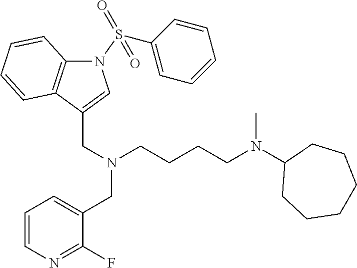 [SMILES: CN(CCCCN(Cc1cccnc1F)Cc1cn(S(=O)(=O)c2ccccc2)c2ccccc12)C1CCCCCC1]

According to general procedure 6. Scale: Compound 73 (40 mg, 0.071 mmol, 1 equiv), Formaldehyde (12.8 mg, 0.426 mmol, 6 equiv), NaBH₃CN (17.8 mg, 0.284 mmol, 4 equiv), ACN (2 mL). The crude product (57.5 mg of an amorphous yellow solid) was purified by flash column chromatography on silica gel, using a solvent gradient (DCM/MeOH·NH₃ 1% to 3%) to afford Compound 74 (36.2 mg, 88%) an amorphous yellow solid.

TLC: Eluent DCM/MeOH·NH₃ 2% R_{f}=0.39

¹H NMR (400 MHz, Chloroform-d) δ 8.05 (1H, m, H21), 7.97 (1H, d, J=8.3 Hz, H11), 7.84-7.74 (3H, m, H15 and H23), 7.59 (1H, d, J=7.6 Hz, H8), 7.48 (2H, m, H13 and H17), 7.37 (2H, m, H16), 7.31 (1H, m, H10), 7.22 (1H, m, H9), 7.09 (1H, m, H22), 3.69 (2H, s, H5), 3.55 (2H, s, H18), 2.52 (1H, m, H24), 2.46 (2H, m, H4), 2.26 (2H, m, H1), 2.15 (3H, s, H28), 1.68 (4H, m, H25 and H26), 1.58-1.43 (6H, m, H3, H27 and H27′), 1.42-1.30 (6H, m, H2, H25′ and H26′).

¹³C NMR (100 MHz, Chloroform-d) δ 160.89 (C20), 145.93 (C21), 141.35 (C23), 138.22 (C14), 135.76 (C12), 133.87 (C17), 130.80 (C7), 129.31 (C16), 126.76 (C15), 125.06 (C10), 124.80 (C13), 123.39 (C9), 121.79 (C19), 121.50 (C22), 121.03 (C6), 120.58 (C8), 113.87 (C11), 64.52 (C24), 54.32 (C4), 53.16 (C1), 50.85 (C18), 49.78 (C5), 37.72 (C28), 29.77 (C25), 28.14 (C27), 26.15 (C2), 25.99 (C26), 25.19 (C3).

¹⁹F NMR (376 MHz, Chloroform-d) δ−72.65.

LRMS m/z (ESI+, CV 30) 577.4 [M+H]⁺.

### 8.19. Preparation of N′-cyclooctyl-N⁴-((2-fluoropyridin-3-yl)methyl)-N′-methyl-N⁴-((1-(phenylsulfonyl)-1H-indol-3-yl)methyl)butane-1,4-diamine (Compound 76, C₃₄H₄₃FN₄O₂S, MW=590.79 g/mol)

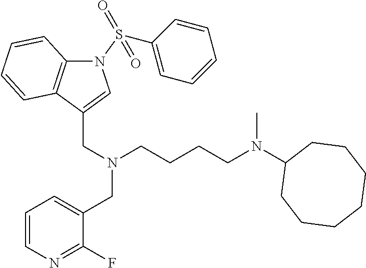 [SMILES: CN(CCCCN(Cc1cccnc1F)Cc1cn(S(=O)(=O)c2ccccc2)c2ccccc12)C1CCCCCCC1]

According to general procedure 6. Scale: Compound 75 (77.3 mg, 0.134 mmol, 1 equiv), Formaldehyde (24.1 mg, 0.804 mmol, 6 equiv), NaBH₃CN (33.7 mg, 0.536 mmol, 4 equiv), ACN (3 mL). The crude product (75.5 mg of an amorphous yellow solid) was purified by flash column chromatography on silica gel, using DCM/MeOH·NH₃ 2% as the eluent to afford Compound 76 (43.7 mg, 55%) an amorphous yellow solid.

TLC: Eluent DCM/MeOH·NH₃ 2% R_{f}=0.45

¹H NMR (400 MHz, Chloroform-d) δ 8.05 (1H, m, H21), 7.97 (1H, d, J=8.3 Hz, H11), 7.84-7.74 (3H, m, H15 and H23), 7.60 (1H, d, J=7.6 Hz, H8), 7.48 (2H, m, H13 and H17), 7.37 (2H, m, H16), 7.31 (1H, m, H10), 7.22 (1H, m, H9), 7.09 (1H, m, H22), 3.69 (2H, s, H5), 3.55 (2H, s, H18), 2.61 (1H, br, H24), 2.46 (2H, m, H4), 2.24 (2H, m, H1), 2.13 (3H, s, H29), 1.76-1.48 (11H, m, H3, H25, H26, H27, H28 and H29), 1.40 (11H, m, H2, H25′, H26′, H27′, H28′ and H29′).

¹³C NMR (100 MHz, Chloroform-d) δ 160.88 (C20), 145.91 (C21), 141.32 (C23), 138.21 (C14), 135.76 (C12), 133.87 (C17), 130.80 (C7), 129.30 (C16), 126.76 (C15), 125.06 (C10), 124.79 (C13), 123.39 (C9), 121.80 (C19), 121.53 (C22), 121.03 (C6), 120.57 (C8), 113.87 (C13), 63.00 (C24), 54.38 (C4), 53.01 (C1), 50.83 (C18), 49.77 (C5), 37.85 (C29), 29.46 (C25), 26.73 (C27 and C28), 26.19 (C2), 25.92 (C26), 25.24 (C3).

¹⁹F NMR (376 MHz, Chloroform-d) δ

LRMS m/z (ESI+, CV 30) 591.3 [M+H]⁺.

### 8.20. Preparation of N-(2-(2-(((2-fluoropyridin-3-yl)methyl)((1-(phenylsulfonyl)-1H-indol-3-yl)methyl)amino)ethoxy)ethyl)-N-methylcyclohexanamine (Compound 65, C₃₂H₃₉FN₄O₃S, MW=578.75 g/mol)

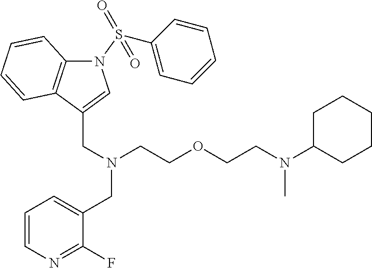 [SMILES: CN(CCOCCN(Cc1cccnc1F)Cc1cn(S(=O)(=O)c2ccccc2)c2ccccc12)C1CCCCC1]

According to general procedure 6. Scale: Compound 64 (51 mg, 0.09 mmol, 1 equiv), Formaldehyde (16 mg, 0.54 mmol, 6 equiv), NaBH₃CN (23 mg, 0.36 mmol, 4 equiv), ACN (2.5 mL). The crude product (52 mg of an amorphous yellow solid) was purified by flash column chromatography on silica gel, using a solvent gradient (DCM/MeOH·NH₃ 1% to 3%) to afford Compound 65 (50 mg, 96%) an amorphous yellow solid.

TLC: Eluent DCM/MeOH·NH3 2% Rf=0.57

1H NMR (400 MHz, CDCl3) δ 8.04 (1H, m, H21), 7.97 (1H, d, J=8.3 Hz, H11), 7.83 (3H, m, H15 and H23), 7.62 (1H, d, J=7.8 Hz, H8), 7.51 (1H, s, H13), 7.47 (1H, tt, J=7.5 Hz, J=1.4 Hz H17), 7.37 (2H, m, H16), 7.31 (1H, m, H10), 7.22 (1H, m, H9), 7.08 (1H, m, H22), 3.79 (2H, s, H5), 3.66 (2H, s, H18), 3.57 (2H, t, J=5.6 Hz, H3), 3.46 (2H, t, J=6.3 Hz, H2), 2.71 (2H, t, J=5.6 Hz, H4), 2.62 (2H, t, J=6.3 Hz, H1), 2.38-2.31 (1H, br, H24), 2.28 (3H, s, H28), 1.77 (4H, m, H25 eq and H26 eq), 1.60 (1H, m, H27 eq), 1.27-1.02 (5H, m, H25ax, H26ax and H27ax).

13C NMR (101 MHz, CDCl3) δ 160.70 (C20), 145.78 (C21), 141.25 (C23), 138.10 (C14), 135.65 (C12), 133.73 (C17), 130.67 (C7), 129.17 (C16), 126.65 (C15), 124.92 (C10), 124.76 (C13), 123.24 (C9), 121.60 (C19), 121.35 (C22), 120.88 (C6), 120.51 (C8), 113.74 (C11), 70.23 (C2), 69.63 (C3), 63.37 (C24), 53.40 (C4), 52.81 (C1), 51.17 (C18), 50.17 (C5), 38.78 (C28), 28.59 (C25), 26.34 (C27), 26.00 (C26).

19F NMR (376 MHz, Chloroform-d) δ−72.58

LRMS m/z (ESI+, CV 30) 579.4 [M+H]⁺.

### 8.21. Preparation of N-ethyl-N-(2-(2-(((2-fluoropyridin-3-yl)methyl)((1-(phenylsulfonyl)-1H-indol-3-yl)methyl)amino)ethoxy)ethyl)cyclohexanamine (Compound 93, C₃₃H₄FN₄O₃S, MW=592.77 g/mol)

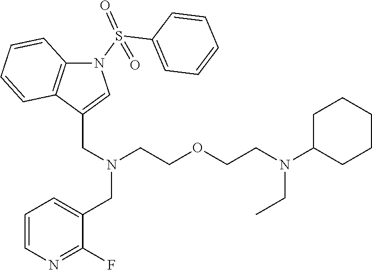 [SMILES: CCN(CCOCCN(Cc1cccnc1F)Cc1cn(S(=O)(=O)c2ccccc2)c2ccccc12)C1CCCCC1]

According to general procedure 5. Scale: Compound 64 (84 mg, 0.149 mmol, 1 equiv), Acetaldehyde (10 mg, 0.224 mmol, 1.5 equiv), NaBH(OAc)3 (50 mg, 0.238 mmol, 1.6 equiv), DCM (3 mL). The crude product (73 mg of an amorphous orange solid) was purified by flash column chromatography on silica gel, using DCM/MeOH·NH₃ 2% as the eluent to afford Compound 93 (53 mg, 60%) an amorphous colourless solid.

TLC: Eluent DCM/MeOH·NH3 2% Rf=0.78

1H NMR (400 MHz, Chloroform-d) δ 8.04 (m, 1H, H21), 7.97 (d, J=8.3 Hz, 1H, H11), 7.87-7.78 (m, 3H, H15 and H23), 7.63 (d, J=7.9 Hz, 1H, H8), 7.51 (s, 1H, H13), 7.50-7.45 (m, 1H, H17), 7.37 (m, 2H, H16), 7.31 (m, 1H, H10), 7.22 (m, 1H, H9), 7.08 (m, 1H, H22), 3.79 (s, 2H, H5), 3.66 (s, 2H, H18), 3.57 (t, J=5.6 Hz, 2H, H3), 3.41 (m, 2H, H2), 2.70 (t, J=5.6 Hz, 2H, H4), 2.67-2.51 (m, 4H, H1 and H28), 2.45 (m, 1H, H24), 1.75 (m, 4H, H25 eq and H26 eq), 1.60 (m, 1H, H27 eq), 1.25-1.08 (m, 5H, H25ax, H26ax and H27ax), 1.00 (t, J=7.1 Hz, 3H, H29).

13C NMR (100 MHz, Chloroform-d) δ 162.00 (d, 1 JCF=238.8 Hz, C20), 145.88 (d, 3JCF=15.3 Hz, C21), 141.36 (d, 3JCF=5.3 Hz, C23), 138.19 (C14), 135.76 (C12), 133.86 (C17), 130.78 (C7), 129.29 (C16), 126.77 (C15), 125.03 (C10), 124.88 (C13), 123.36 (C9), 121.57 (d, 1 JCF=28.2 Hz, C19), 121.50 (d, 4JCF=3.8 Hz, C22), 121.00 (C6), 120.63 (C8), 113.86 (C11), 71.53 (C2), 69.81 (C3), 53.58 (C4), 51.30 (C18), 50.30 (C5), 49.61 (C28), 45.72 (C24), 29.25 (C25), 26.46 (C27), 26.24 (C26), 14.20 (C29).

19F NMR (376 MHz, Chloroform-d) δ—

LRMS m/z (ESI+, CV 30) 593.35 [M+H]+.

### 8.22. Preparation of N-(2-(2-(((2-fluoropyridin-3-yl)methyl) ((1-(phenylsulfonyl)-1H-indol-3-yl)methyl)amino)ethoxy)ethyl)-N-propylcyclohexanamine (Compound 97, C₃₄H₄₃FN₄O₃S, MW=606.80 g/mol)

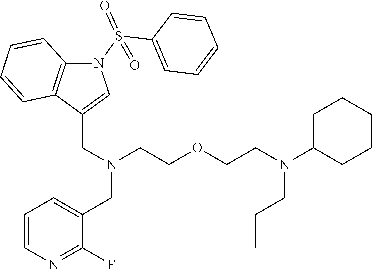 [SMILES: CCCN(CCOCCN(Cc1cccnc1F)Cc1cn(S(=O)(=O)c2ccccc2)c2ccccc12)C1CCCCC1]

According to general procedure 5. Scale: Compound 64 (70 mg, 0.124 mmol, 1 equiv), Propionaldehyde (11 mg, 0.185 mmol, 1.5 equiv), NaBH(OAc)3 (39 mg, 0.185 mmol, 1.5 equiv), DCM (2.5 mL). The crude product (95 mg of an amorphous yellow solid) was purified by flash column chromatography on silica gel, using DCM/MeOH·NH₃ 2% as the eluent to Compound 97 (19 mg, 25%) an amorphous colourless solid.

TLC: Eluent DCM/MeOH·NH3 1% Rf=0.43

1H NMR (400 MHz, Chloroform-d) δ 8.05 (m, 1H, H21), 7.97 (d, J=8.4 Hz, 1H, H11), 7.82 (m, 3H, H15 and H23), 7.62 (m, 1H, H8), 7.54-7.45 (m, 2H, H13 and H17), 7.38 (t, J=7.8 Hz, 2H, H16), 7.31 (m, 1H, H10), 7.22 (m, 1H, H9), 7.10 (m, 1H, H22), 3.79 (s, 2H, H5), 3.66 (s, 2H, H18), 3.57 (t, J=5.6 Hz, 2H, H3), 3.39 (m, 2H, H2), 2.70 (t, J=5.6 Hz, 2H, H4), 2.63 (m, 2H, H1), 2.41 (m, 2H, H24 and H28), 1.75 (m, 4H, H25 eq and H26 eq), 1.58 (m, 1H, H27 eq), 1.39 (m, 2H, H29), 1.17 (m, 6H, H25ax, H26ax and H27ax), 0.83 (t, J=7.4 Hz, 3H, H30).

13C NMR (100 MHz, Chloroform-d) δ 146.06 (C21), 141.39 (d, 3JCF=6.0 Hz, C23), 138.19 (C14), 135.75 (C12), 133.91 (C17), 130.92 (C7), 129.33 (C16), 126.78 (C15), 125.06 (C10), 124.90 (C13), 123.39 (C9), 122.33 (C19), 121.53 (d, 4JCF=4.1 Hz, C22), 120.95 (C6), 120.56 (C8), 113.87 (C11), 70.74 (C2), 69.82 (C3), 61.38 (C28), 53.82 (C24), 53.61 (C4), 51.38 (C18), 50.35 (C1), 50.28 (C5), 29.94 (C25), 25.58 (C26 and C27), 21.84 (C29), 11.80 (C30).

19F NMR (376 MHz, Chloroform-d) δ—−72.58.

LRMS m/z (ESI+, CV 30) 607.31 [M+H]+.

### 8.23. Preparation of N-butyl-N-(2-(2-(((2-fluoropyridin-3-yl)methyl)((1-(phenylsulfonyl)-1H-indol-3-yl)methyl)amino)ethoxy)ethyl)cyclohexanamine (Compound 99, C₃₅H₄₅FN₄O₃S, MW=620.83 g/mol)

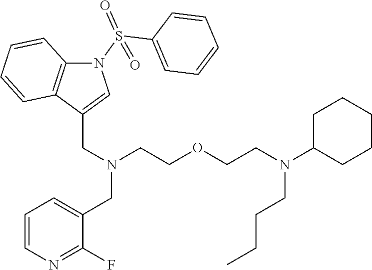 [SMILES: CCCCN(CCOCCN(Cc1cccnc1F)Cc1cn(S(=O)(=O)c2ccccc2)c2ccccc12)C1CCCCC1]

According to general procedure 5. Scale: Compound 64 (70 mg, 0.124 mmol, 1 equiv), Butionaldehyde (11 mg, 0.149 mmol, 1.2 equiv), NaBH(OAc)₃ (42 mg, 0.198 mmol, 1.6 equiv), DCM (2.5 mL). The crude product (88 mg of an amorphous yellow solid) was purified by flash column chromatography on silica gel, using DCM/MeOH·NH₃ 2% as the eluent to afford Compound 99 (66 mg, 86%) an amorphous yellow solid.

TLC: Eluent DCM/MeOH·NH3 2% Rf=0.90

1H NMR (400 MHz, Chloroform-d) δ 8.04 (m, 1H, H21), 7.97 (d, J=8.4 Hz, 1H, H11), 7.86-7.79 (m, 3H, H15 and H23), 7.62 (m, 1H, H8), 7.53-7.45 (m, 2H, H13 and H17), 7.37 (m, 2H, H16), 7.31 (m, 1H, H10), 7.22 (m, 1H, H9), 7.09 (m, 1H, H22), 3.79 (s, 2H, H5), 3.66 (s, 2H, H18), 3.57 (t, J=5.6 Hz, 2H, H3), 3.39 (m, 2H, H2), 2.70 (t, J=5.6 Hz, 2H, H4), 2.63 (m, 2H, H1), 2.45 (m, 3H, H24 and H28), 1.74 (s, 4H, H25 eq and H26 eq), 1.60 (s, 1H, H27 eq), 1.36 (m, 2H, H29), 1.27 (quint, J=7.8 Hz, 2H, H30), 1.19 (m, 5H, H25ax, H26ax and H27ax), 0.87 (t, J=7.3 Hz, 3H, H31).

13C NMR (100 MHz, Chloroform-d) δ 163.21 (C20), 145.86 (d, 3JCF=16.0 Hz, C21), 141.38 (d, 3JCF=5.3 Hz, C23), 138.20 (C14), 135.77 (C12), 133.87 (C17), 130.78 (C7), 129.31 (C16), 126.78 (C15), 125.05 (C10), 124.89 (C13), 123.37 (C9), 121.65 (C19), 121.53 (d, 4JCF=3.8 Hz, C22), 121.00 (C6), 120.63 (C8), 113.87 (C11), 73.04 (C2), 69.85 (C3), 61.39 (C24), 53.61 (C4), 51.95 (C28), 51.41 (C18), 50.33 (C5), 50.22 (C1), 31.50 (C29), 29.23 (C25), 26.31 (C26 and C27), 20.71 (C30), 14.23 (C31).

19F NMR (376 MHz, Chloroform-d) δ—−72.58.

LRMS m/z (ESI+, CV 30) 621.30 [M+H]+.

### 8.24. Preparation of N-(2-(2-(((2-fluoropyridin-3-yl)methyl)((1-(phenylsulfonyl)-1H-indol-3-yl)methyl)amino)ethoxy)ethyl)cyclopentanamine (Compound 103, C₃₀H₃₅FN₄O₃S, MW=550.69 g/mol)

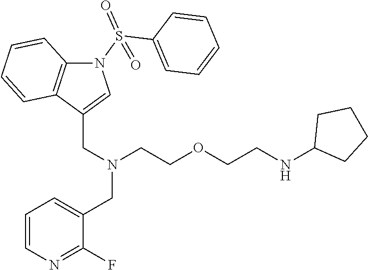 [SMILES: O=S(=O)(c1ccccc1)n1cc(CN(CCOCCNC2CCCC2)Cc2cccnc2F)c2ccccc21]

According to general procedure 5. Scale: Intermediate 26 (526 mg, 1.09 mmol, 1 equiv), Cyclopentanone (110 mg, 1.31 mmol, 1.2 equiv), NaBH(OAc)3 (348 mg, 1.64 mmol, 1.5 equiv), DCM (11 mL). The crude product (599 mg of a brown oil) was purified by flash column chromatography on silica gel, using DCM/MeOH·NH₃ 2% as the eluent to afford Compound 103 (299 mg, 50%) an amorphous yellow solid.

TLC: Eluent DCM/MeOH·NH3 2% Rf=0.37

1H NMR (400 MHz, Chloroform-d) δ 8.05 (m, 1H, H21), 7.97 (d, J=8.4 Hz, 1H, H11), 7.86-7.79 (m, 3H, H15 and H23), 7.64-7.59 (m, 1H, H8), 7.53-7.45 (m, 2H, H13 and H17), 7.37 (m, 2H, H16), 7.31 (m, 1H, H10), 7.22 (m, 1H, H9), 7.09 (m, 1H, H22), 3.79 (s, 2H, H5), 3.67 (s, 2H, H18), 3.58 (t, J=5.6 Hz, 2H, H3), 3.51 (t, J=5.3 Hz, 2H, H2), 3.04 (quint, J=6.9 Hz, 1H, H24), 2.73 (m, 4H, H1 and H4), 1.85-1.75 (m, 2H, H25 eq), 1.70-1.59 (m, 2H, H26 eq), 1.55-1.44 (m, 2H, H26ax), 1.33-1.23 (m, 2H, H25ax).

13C NMR (100 MHz, Chloroform-d) δ 162.01 (d, 1 JCF=238.8 Hz, C20), 145.99 (d, 3JCF=14.5 Hz, C21), 141.35 (d, 3JCF=5.3 Hz, C23), 138.18 (C14), 135.74 (C12), 133.88 (C17), 130.73 (C7), 129.31 (C16), 126.78 (C15), 125.08 (C10), 124.88 (C13), 123.39 (C9), 121.51 (d, 4JCF=3.8 Hz, C22), 121.45 (d, 3JCF=28.2 Hz, C19), 120.87 (C6), 120.58 (C8), 113.87 (C11), 70.76 (C2), 69.64 (C3), 59.94 (C24), 53.38 (C4), 51.24 (C18), 50.22 (C5), 48.28 (C1), 33.18 (C25), 24.21 (C26).

19F NMR (376 MHz, Chloroform-d) δ−72.53.

LRMS m/z (ESI+, CV 30). 551.29 [M+H]+.

### 8.25. Preparation of N-(2-(2-(((2-fluoropyridin-3-yl)methyl)((1-(phenylsulfonyl)-1H-indol-3-yl)methyl)amino)ethoxy)ethyl)-N-methylcyclopentanamine (Compound 105, C₁₃H₃₇FN₄O₃S, MW=564.72 g/mol)

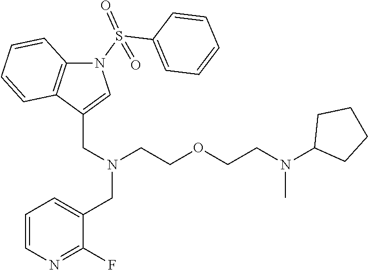 [SMILES: CN(CCOCCN(Cc1cccnc1F)Cc1cn(S(=O)(=O)c2ccccc2)c2ccccc12)C1CCCC1]

According to general procedure 6. Scale: Compound 103 (60 mg, 0.109 mmol, 1 equiv), Formaldehyde (20 mg, 0.654 mmol, 6 equiv), NaBH₃CN (27 mg, 0.436 mmol, 4 equiv), ACN (2 mL). The crude product (63 mg of an amorphous yellow solid) was purified by flash column chromatography on silica gel, using DCM/MeOH·NH₃ 2% as the eluent to afford Compound 105 (58 mg, 94%) an amorphous yellow/white solid.

TLC: Eluent DCM/MeOH·NH3 2% Rf=0.67

1H NMR (400 MHz, Chloroform-d) δ 8.04 (m, 1H, H21), 7.97 (d, J=8.3 Hz, 1H, H11), 7.87-7.79 (m, 3H, H15 and H23), 7.62 (d, J=7.8 Hz, 1H, H8), 7.53-7.45 (m, 2H, H13 and H17), 7.37 (m, 2H, H16), 7.31 (m, 1H, H10), 7.22 (m, 1H, H9), 7.08 (m, 1H, H22), 3.79 (s, 2H, H5), 3.66 (s, 2H, H18), 3.57 (t, J=5.6 Hz, 2H, H3), 3.51 (t, J=6.2 Hz, 2H, H2), 2.72 (m, 3H, H4 and H24), 2.62 (t, J=6.1 Hz, 2H, H1), 2.27 (s, 3H, H27), 1.85-1.76 (m, 2H, H25 eq), 1.70-1.60 (m, 2H, H26 eq), 1.56-1.35 (m, 4H, H25ax and H26ax).

13C NMR (100 MHz, Chloroform-d) δ 162.00 (d, 1 JCF=240.0 Hz, C20), 145.92 (d, 3JCF=15.3 Hz, C21), 141.37 (d, 3JCF=5.3 Hz, C23), 138.19 (C14), 135.75 (C12), 133.87 (C17), 130.76 (C7), 129.30 (C16), 126.77 (C15), 125.05 (C10), 124.87 (C13), 123.37 (C9), 121.54 (d, 3JCF=28.2 Hz, C19), 121.50 (d, 4JCF=3.8 Hz, C22), 120.97 (C6), 120.62 (C8), 113.86 (C11), 69.75 (C3), 69.52 (C2), 67.26 (C24), 55.24 (C1), 53.50 (C4), 51.33 (C18), 50.30 (C5), 41.20 (C27), 30.29 (C25), 24.38 (C26).

19F NMR (376 MHz, Chloroform-d) δ−72.56.

LRMS m/z (ESI+, CV 30) 565.26 [M+H]+.

### 8.26. Preparation of N-ethyl-N-(2-(2-(((2-fluoropyridin-3-yl)methyl)((1-(phenylsulfonyl)-1H-indol-3-yl)methyl)amino)ethoxy)ethyl)cyclopentanamine (Compound 107, C₃₂H₃₉FN₄O₃S, MW=578.75 g/mol)

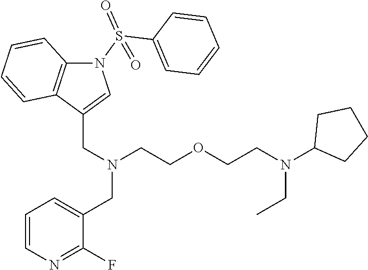 [SMILES: CCN(CCOCCN(Cc1cccnc1F)Cc1cn(S(=O)(=O)c2ccccc2)c2ccccc12)C1CCCC1]

According to general procedure 5. Scale: Compound 103 (90 mg, 0.163 mmol, 1 equiv), Acetaldehyde (11 mg, 0.245 mmol, 1.5 equiv), NaBH(OAc)₃ (55 mg, 0.261 mmol, 1.6 equiv), DCM (3 mL). The crude product (89 mg of an amorphous orange solid) was purified by flash column chromatography on silica gel, using DCM/MeOH·NH₃ 2% as the eluent to afford Compound 107 (47 mg, 50%) an amorphous colourless solid.

TLC: Eluent DCM/MeOH·NH3 2% Rf=0.62

1H NMR (400 MHz, Chloroform-d) δ δ 8.04 (m, 1H, H21), 7.97 (d, J=8.3 Hz, 1H, H11), 7.86-7.79 (m, 3H, H15 and H23), 7.62 (d, J=7.9 Hz, 1H, H8), 7.52-7.45 (m, 2H, H13 and H17), 7.37 (m, 2H, H16), 7.31 (m, 1H, H10), 7.22 (m, 1H, H9), 7.08 (m, 1H, H22), 3.79 (s, 2H, H5), 3.66 (s, 2H, H19), 3.57 (t, J=5.6 Hz, 2H, H3), 3.48 (m, 2H, H2), 3.02 (m, 1H, H24), 2.75-2.61 (m, 6H, H1, H4 and H27), 1.79 (m, 2H, H25 eq), 1.64 (m, 2H, H26 eq), 1.55-1.33 (m, 4H, H25ax and H26ax), 1.01 (t, J=7.2 Hz, 3H, H28).

13C NMR (100 MHz, Chloroform-d) δ 162.00 (d, 1 JCF=238.2 Hz, C20), 145.92 (d, 3JCF=15.3 Hz, C21), 141.35 (d, 1JCF=6.1 Hz, C23), 138.18 (C14), 135.75 (C12), 133.87 (C17), 130.76 (C7), 129.30 (C16), 126.77 (C15), 125.05 (C10), 124.86 (C13), 123.36 (C9), 121.55 (d, 2JCF=28.5 Hz, C19), 121.49 (d, 4JCF=4.6 Hz, C22), 120.97 (C6), 120.61 (C8), 113.85 (C11), 69.85 (C2 and C3), 64.07 (C24), 53.57 (C4), 51.32 (C18), 50.32 (C1 and C5), 46.74 (C27), 30.08 (C25), 24.21 (C26), 11.66 (C28).

19F NMR (376 MHz, Chloroform-d) δ−72.60.—

LRMS m/z (ESI+, CV 30) 579.30 [M+H]+.

### 8.27. Preparation of N-(2-(2-(((2-fluoropyridin-3-yl)methyl)((1-(phenylsulfonyl)-1H-indol-3-yl)methyl)amino)ethoxy)ethyl)-N-propylcyclopentanamine (Compound 108, C₃₃H₄FN₄O₃S, MW=592.77 g/mol)

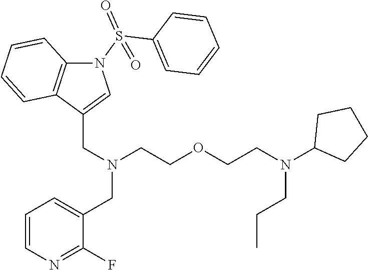 [SMILES: CCCN(CCOCCN(Cc1cccnc1F)Cc1cn(S(=O)(=O)c2ccccc2)c2ccccc12)C1CCCC1]

According to general procedure 5. Scale: Compound 103 (70 mg, 0.127 mmol, 1 equiv), Propionaldehyde (11 mg, 0.190 mmol, 1.5 equiv), NaBH(OAc)3 (40 mg, 0.191 mmol, 1.5 equiv), DCM (2.5 mL). The crude product (87 mg of an amorphous orange solid) was purified by flash column chromatography on silica gel, using DCM/MeOH·NH₃ 2% as the eluent to Compound 108(10 mg, 13%) an amorphous yellow solid.

TLC: Eluent DCM/MeOH·NH3 1% Rf=0.43

1H NMR (400 MHz, CDCl3) δ 8.05 (m, 1H, H21), 7.97 (d, J=8.4 Hz, 1H, H11), 7.82 (m, 3H, H15 and H23), 7.62 (m, 1H, H8), 7.54-7.45 (m, 2H, H13 and H17), 7.38 (t, J=7.8 Hz, 2H, H16), 7.31 (m, 1H, H10), 7.22 (m, 1H, H9), 7.10 (m, 1H, H22), 3.79 (s, 2H, H5), 3.66 (s, 2H, H18), 3.57 (t, J=5.6 Hz, 2H, H3), 3.39 (m, 2H, H2), 2.70 (t, J=5.6 Hz, 2H, H4), 2.63 (m, 2H, H1), 2.41 (m, 2H, H24 and H28), 1.75 (m, 4H, H25 eq and H26 eq), 1.58 (m, 1H, H27 eq), 1.39 (m, 2H, H29), 1.17 (m, 6H, H25ax, H26ax and H27ax), 0.83 (t, J=7.4 Hz, 3H, H30).

13C NMR (100 MHz, CDCl3) δ 146.06 (C21), 141.39 (d, 3JCF=6.0 Hz, C23), 138.19 (C14), 135.75 (C12), 133.91 (C17), 130.92 (C7), 129.33 (C16), 126.78 (C15), 125.06 (C10), 124.90 (C13), 123.39 (C9), 122.33 (C19), 121.53 (d, 4JCF=4.1 Hz, C22), 120.95 (C6), 120.56 (C8), 113.87 (C11), 70.74 (C2), 69.82 (C3), 61.38 (C28), 53.82 (C24), 53.61 (C4), 51.38 (C18), 50.35 (C1), 50.28 (C5), 29.94 (C25), 25.58 (C26 and C27), 21.84 (C29), 11.80 (C30).

19F NMR (376 MHz, CDCl3) δ−72.58.

LRMS m/z (ESI+, CV 30) 607.31 [M+H]+.

### 8.28. Preparation of N-butyl-N-(2-(2-(((2-fluoropyridin-3-yl)methyl)((1-(phenylsulfonyl)-1H-indol-3-yl)methyl)amino)ethoxy)ethyl)cyclopentanamine (Compound 109, C₃₄H₄₃FN₄O₃S, MW=606.80 g/mol)

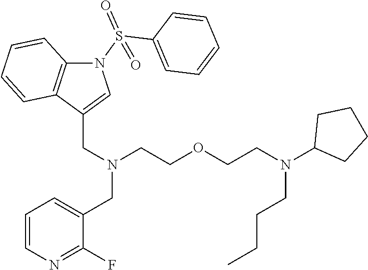 [SMILES: CCCCN(CCOCCN(Cc1cccnc1F)Cc1cn(S(=O)(=O)c2ccccc2)c2ccccc12)C1CCCC1]

According to general procedure 5. Scale: Compound 103 (50 mg, 0.091 mmol, 1 equiv), Butionaldehyde (8 mg, 0.109 mmol, 1.2 equiv), NaBH(OAc)3 (31 mg, 0.146 mmol, 1.5 equiv), DCM (2 mL). The crude product (55 mg of an amorphous yellow solid) was purified by flash column chromatography on silica gel, using DCM/MeOH·NH₃ 2% as the eluent to afford Compound 109 (40 mg, 73%) an amorphous colourless solid.

TLC: Eluent DCM/MeOH·NH3 2% Rf=0.93

1H NMR (400 MHz, Chloroform-d) δ δ 8.05 (m, 1H, H21), 7.97 (d, J=8.4 Hz, 1H, H11), 7.86-7.79 (m, 3H, H15 and H23), 7.62 (m, 1H, H8), 7.52-7.44 (m, 2H, H13 and H17), 7.37 (m, 2H, H16), 7.31 (m, 1H, H10), 7.22 (m, 1H, H9), 7.08 (m, 1H, H22), 3.79 (s, 2H, H5), 3.66 (s, 2H, H18), 3.57 (t, J=5.6 Hz, 2H, H3), 3.47 (m, 2H, H2), 3.03 (m, 1H, H24), 2.71 (m, 4H, H1 and H4), 2.52 (m, 2H, H27), 1.77 (m, 2H, H25 eq), 1.64 (m, 2H, H26 eq), 1.45 (m, 6H, H25ax, H26ax and H28), 1.23 (sex, J=7.3 Hz, 2H, H29), 0.88 (t, J=7.3 Hz, 3H, H30).

13C NMR (100 MHz, Chloroform-d) δ 160.92 (C20), 145.93 (d, 3JCF=14.5 Hz, C21), 141.33 (d, 3JCF=5.1 Hz, d, 1JCF=240.0 Hz, C23), 138.20 (C14), 135.76 (C12), 133.88 (C17), 130.77 (C7), 129.31 (C16), 126.78 (C15), 125.06 (C10), 124.88 (C13), 123.38 (C9), 121.69 (C19), 121.50 (d, 4JCF=4.4 Hz, C22), 120.97 (C6), 120.62 (C8), 113.87 (C11), 69.91 (C3 and C2), 64.52 (C24), 53.59 (C4), 52.82 (C27) 51.35 (C1), 51.14 (C18), 50.35 (C5), 29.77 (C25 and C28), 24.21 (C26), 20.85 (C29), 14.21 (C30).

19F NMR (376 MHz, Chloroform-d) δ−72.60.—

LRMS m/z (ESI+, CV 30) 607.32 [M+H]+.

### 9. General Procedure 7

### 9.1. Reductive Amination Between Primary Amine and Cyclohexanone

### Preparation of tert-butyl (4-(cyclohexylamino)butyl)carbamate (Intermediate 29, C₁₅H₃₀N₂O₂, MW=270.41 g/mol)

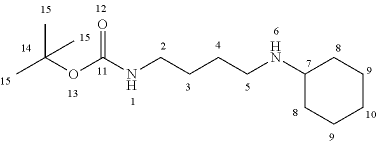 [SMILES: CC(C)(C)OC(=O)NCCCCNC1CCCCC1]

Under argon atmosphere, Intermediate 1 (1.09 g, 5.35 mmol, 1 equiv) was dissolved in anhydrous DCM (7 mL, 0.8 M), cyclohexanone (665 μL, 6.4 mmol, 1.2 equiv) was added to the reactional mixture. After 1 h of stirring at RT, NaBH(OAc)3 (1.3 g, 8.00 mmol, 1.5 equiv) was added. The mixture was stirred at RT during 16 h, then concentrated under reduce pressure. The obtained mixture was washed with a saturated solution of NaHCO₃(2×25 mL). Combined aqueous layers were extracted with DCM (3×50 mL) then dried over MgSO₄ and concentrated under reduce pressure. The resulting crude product was purified by flash column chromatography on silica gel, using DCM/MeOH·NH₃ 1% as eluent to afford Intermediate 29 (795 mg, 55%) as a yellow oil.

TLC: Eluent DCM/MeOH·NH3 1% Rf=0.25.

¹H NMR (400 MHz, Chloroform-d) δ 5.13 (br s, 1H, NH1), 3.15-2.97 (m, 2H, H2), 2.68-2.51 (m, 2H, H5), 2.45-2.27 (m, 1H, H7), 1.93-1.73 (m, 2H, H3), 1.76-1.61 (m, 2H, H4), 1.64-1.52 (m, 1H, H10), 1.53-1.42 (m, 4H, H8 and H9), 1.39 (s, 9H, H15), 1.31-0.93 (m, 5H, H8′, H9′, H10′). NH6 too broad to be seen.

¹³C NMR (101 MHz, CDCl3) δ 156.07 (C11), 78.81 (C14), 56.84 (C7), 46.39 (C5), 40.44 (C2), 33.42 (C3), 28.42 (C15), 27.94 (C4), 27.79 (C8), 26.10 (C10), 25.04 (C9).

LRMS m/z (ESI+, CV 30) 271.33 [M+H]+.

### 9.2. Reductive Amination Between Intermediate 29 and Formaldehyde

### Preparation of tert-butyl (4-(cyclohexyl(methyl)amino)butyl)carbamate (intermediate 30, C₁₆H₃₂N₂O₂, MW=284.44 g/mol)

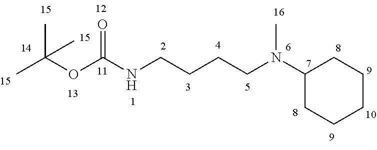 [SMILES: CN(CCCCNC(=O)OC(C)(C)C)C1CCCCC1]

Under argon atmosphere, intermediate 29 (678 mg, 2.51 mmol, 1 equiv) was dissolved in anhydrous MeOH (10 mL, 0.25 M), formaldehyde (452 mg, 15.1 mmol, 6 equiv) was added to the reactional mixture. After 1 h of stirring at RT, NaBH₃CN (631 mg, 10 mmol, 4 equiv) was added. The mixture was stirred at RT during 16 h. After completion, the mixture was concentrated under reduce pressure then re-dissolved in EtOAc (40 mL). The organic layer was washed successively with NaOH 2N (15 mL), water (15 mL) and brine (15 mL). The organic phase was dried over anhydrous MgSO₄ and concentrated under reduced pressure. The resulting crude product was purified by flash column chromatography on silica gel, using DCM/MeOH·NH₃ 1% as eluant, to afford intermediate 30 (547 mg, 77%) as a yellow oil.

TLC: Eluent DCM/MeOH·NH3 1% Rf=0.30.

¹H NMR (400 MHz, Chloroform-d) δ 5.42 (br s, 1H, NH1), 3.08 (q, J=6.2 Hz, 2H, H2), 2.48-2.36 (m, 2H, H5), 2.36-2.27 (m, 1H, H7), 2.20 (s, 3H, H16), 1.87-1.69 (m, 4H, H3 and H4), 1.67-1.54 (m, 1H, H10), 1.56-1.44 (m, 4H, H8 and H9), 1.41 (s, 9H, H15), 1.31-0.94 (m, 5H, H8′, H9′ and H10′).

¹³C NMR (101 MHz, CDCl3) δ 156.26 (C11), 78.82 (C14), 62.89 (C7), 53.51 (C2), 40.68 (C5), 37.25 (C16), 28.57 (C3), 28.46 (C15), 28.12 (C4), 26.46 (C8), 26.13 (C10), 25.49 (C9).

LRMS m/z (ESI+, CV 30) 285.35 [M+H]+.

### 9.3. Deprotection of Intermediate 30

### Preparation of N1-cyclohexyl-N1-methylbutane-1,4-diamine (Intermediate 31, C₁₁H₂₄N₂, MW=184.32 g/mol)

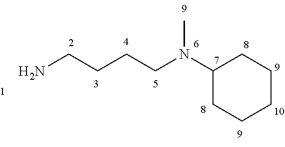

Intermediate 30 (513 mg, 1.80 mmol, 1 equiv) was dissolved in DCM (18 mL, 0.1 M), TFA (1.65 mL, 21.60 mmol, 12 equiv) was added. The reactional mixture was stirred at RT during 2 h. After, the reactional mixture was evaporated and the residue re-dissolved in EtOAc. Under stirring, a solution of NaOH 2M (12 mL) was then added and the phases separated. The organic layer was washed with water (1×15 mL) and brine (1×15 mL) then dried over anhydrous MgSO₄ and concentrated to afford the product Intermediate 31 (244 mg, 74%) as a yellow oil.

TLC: Eluent DCM/MeOH·NH3 1% Rf=0.15.

¹H NMR (400 MHz, Chloroform-d) δ 2.76-2.59 (m, 2H, H2), 2.47-2.37 (m, 2H, H5), 2.36-2.26 (m, 1H, H7), 2.21 (s, 3H, H9), 1.88-1.66 (m, 6H, NH₂, H3 and H4), 1.65-1.52 (m, 1H, H10), 1.52-1.34 (m, 4H, H8 and H9), 1.30-0.95 (m, 5H, H8′, H9′ and H10′).

¹³C NMR (101 MHz, CDCl3) δ 62.64 (C7), 53.62 (C5), 42.13 (C2), 37.79 (C9), 31.82 (C3), 28.61 (C4), 26.48 (C8), 26.14 (C10), 25.49 (C9).

LRMS m/z (ESI+, CV 30) 185.23 [M+H]+.

### 9.4. Reductive Amination Between Intermediate 30 and 2-fluoronicotinaldehyde

### Preparation of N1-cyclohexyl-N4-((2-fluoropyridin-3-yl)methyl)-N1-methylbutane-1,4-diamine (Intermediate 32, C₁₇H₂₈FN₃, MW=293.42 g/mol)

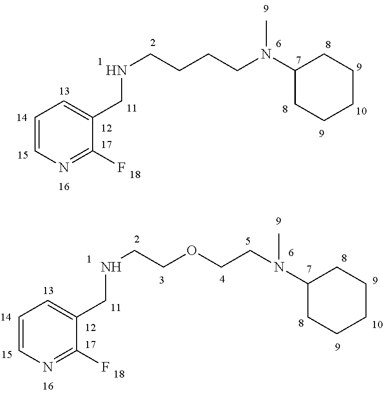 [SMILES: CN(CCCCNCc1cccnc1F)C1CCCCC1 ; CN(CCOCCNCc1cccnc1F)C1CCCCC1]

Under argon atmosphere, Intermediate 31 (220 mg, 1.2 mmol, 1 equiv) was dissolved in anhydrous MeOH (2 mL, 0.5 M), 2-fluoronicotinaldehyde (150 mg, 1.2 mmol, 1 equiv) was added to the reactional mixture. After 1 h of stirring at RT, NaBH(OAC)₃ (295 mg, 1.80 mmol, 1.5 equiv) was added. The mixture was stirred at RT during 16 h. After completion, the mixture was concentrated under reduce pressure then re-dissolved in EtOAc (40 mL). The organic layer was washed successively with NaOH 2N (15 mL), water (15 mL) and brine (15 mL) then dried over anhydrous MgSO₄ and concentrated under reduced pressure. The resulting crude product was purified by flash column chromatography on silica gel, using DCM/MeOH·NH₃ 1% as eluant to afford Intermediate 32 (182 mg, 52%) as a yellow oil.

TLC: Eluent DCM/MeOH·NH3 1% Rf=0.33.

¹H NMR (400 MHz, Chloroform-d) δ 8.09 (dt, J=4.9, 1.5 Hz, 1H, H15), 7.81 (ddd, J=9.5, 7.2, 2.0 Hz, 1H, H13), 7.15 (ddd, J=7.0, 4.9, 1.8 Hz, 1H, H14), 3.82 (s, 2H, H10), 2.63 (t, J=6.5 Hz, 2H, H2), 2.50-2.38 (m, 2H, H5), 2.38-2.30 (m, 1H, H7), 2.23 (s, 3H, H9), 1.86-1.64 (m, 4H, H3 and H4), 1.67-1.55 (m, 1H, H10), 1.56-1.39 (m, 4H, H8 and H9), 1.32-0.94 (m, 5H, H8′, H9′ and H10′). NH too broad to been seen.

¹³C NMR (101 MHz, CDCl3) δ 161.95 (d, ¹JCF=238.6 Hz, C17), 145.99 (d, ³JCF=15.1 Hz, C15), 140.58 (d, ³JCF=6.0 Hz, C13), 122.50 (d, ²JCF=29.7 Hz, C12), 121.59 (d, ⁴JCF=4.4 Hz, C13), 62.74 (C7), 53.59 (C5), 49.42 (C2), 46.94 (C11), 37.84 (C9), 28.61 (C3), 28.16 (C4), 26.48 (C8), 26.14 (C10), 25.79 (C9).

LRMS m/z (ESI+, CV 30) 294.18 [M+H]+.

### 9.5. Reductive Amination with 2-fluoronicotinaldehyde

### Preparation of N-(2-(2-(((2-fluoropyridin-3-yl)methyl)amino)ethoxy)ethyl)-N-methylcyclohexanamine (Intermediate 32b, C₁₇H₂₈FN₃₀, MW=309.43 g/mol)

According the same protocol, and after purification by by flash column chromatography on silica gel, using DCM/MeOH·NH₃ 1% as eluant to afford Intermediate 32b was obtained (231 mg, 48%) as a yellow oil.

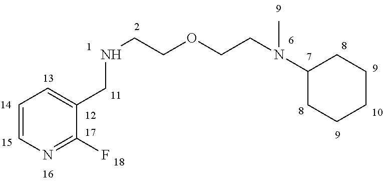 [SMILES: CN(CCOCCNCc1cccnc1F)C1CCCCC1]

TLC: Eluent DCM/MeOHNH3 1% Rf=0.3

¹H NMR (400 MHz, Chloroform-d) δ 8.09 (dt, 1H), 7.84 (ddd, 1H), 7.16 (ddd, 1H), 3.82 (s, 2H), 3.56 (m, 4H), 2.8 (m, 2H), 2.67 (t, 2H), 2.50-2.30 (m, 4H, H5), 1.80-1.10 (m, 9H), NH too broad to been seen.

¹³C NMR (101 MHz, CDCl3) δ 163.14, 160.77, 146.09, 145.95, 140.57, 140.51, 122.53, 122.24, 121.61, 121.58, 70.36, 69.87, 63.48, 52.83, 48.86, 46.75, 38.75, 28.57, 26.40, 26.09

### 9.6. Reductive Amination

### Preparation of N1-cyclohexyl-N4-((2-fluoropyridin-3-yl)methyl)-N1-methyl-N4-((1-(phenylsulfonyl)-1H-pyrrol-3-yl)methyl)butane-1,4-diamine (Compound 91, C28H37FN4O2S, MW=512.68 g/mol)

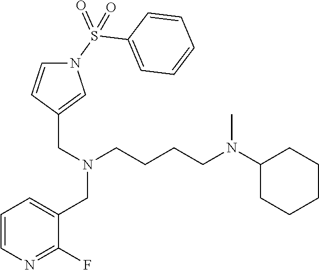 [SMILES: CN(CCCCN(Cc1ccn(S(=O)(=O)c2ccccc2)c1)Cc1cccnc1F)C1CCCCC1]

Under argon atmosphere, Intermediate 32 (82 mg, 0.28 mmol, 1 equiv) was dissolved in anhydrous DCM (1.55 mL, 0.18 M), 1-(phenylsulfonyl)-1H-pyrrole-3-carbaldehyde (66 mg, 0.28 mmol, 1 equiv) was added to the reactional mixture. After 1 h of stirring at RT, NaBH(OAC)₃ (89 mg, 0.42 mmol, 1.5 equiv) was added. The mixture was stirred at RT during 16 h. After completion, more DCM was added (20 mL) and the mixture was washed successively with NaOH 2N (15 mL) and water (2×15 mL). The organic phase was dried over anhydrous MgSO₄ and concentrated under reduced pressure. The resulting crude product was purified by flash column chromatography on silica gel, using DCM/MeOH·NH₃ 1% as eluant to afford Compound 91 (43 mg, 30%) as a gummy solid.

TLC: Eluent DCM/MeOH·NH3 1% Rf=0.45.

¹H NMR (400 MHz, Chloroform-d) δ 8.06 (ddd, J=4.9, 2.0, 1.1 Hz, 1H, H21), 7.92-7.73 (m, 3H, H15 and H23), 7.64-7.51 (m, 1H, H17), 7.51-7.38 (m, 2H, H16), 7.17-7.06 (m, 2H, H12 and H22), 7.03 (t, J=1.9 Hz, 1H, H13), 6.26 (dd, J=3.2, 1.6 Hz, 1H, H7), 3.49 (s, 2H, H5), 3.40 (s, 2H, H18), 2.46-2.28 (m, 5H, H1, H4 and H24), 2.21 (s, 3H, H28), 1.84-1.68 (m, 4H, H2 and H3), 1.67-1.56 (m, 1H, H27), 1.52-1.32 (m, 4H, H25 and H26), 1.32-1.00 (m, 5H, H25′, H26′ and H27′).

¹³C NMR (101 MHz, Chloroform-d) δ 162.03 (d, ¹JCF=238.8 Hz, C20), 145.03 (d, ³JCF=14.5 Hz, C21), 141.17 (d, ³JCF=5.4 Hz, C23), 139.13 (C14), 133.90 (C17), 129.44 (C16), 126.77 (C15), 121.98 (C19), 121.69 (C6), 121.53 (d, 4JCF=4.3 Hz, C22), 121.43 (C12), 119.20 (C13), 115.09 (C7), 62.81 (C24), 53.69 (C4), 53.46 (C1), 50.41 (C18), 50.37 (C5), 37.84 (C28), 28.56 (C3), 26.45 (C2), 26.13 (C25), 25.56 (C27), 25.20 (C26).

¹⁹F (376 MHz, CDCl₃): −72.76 ppm

LRMS m/z (ESI+, CV 30) 513.25 [M+H]+.

### Preparation of N1-cyclohexyl-N4-((2-fluoropyridin-3-yl)methyl)-N1-methyl-N4-((1-(phenylsulfonyl)-1H-pyrrolo[2,3-b]pyridin-3-yl)methyl)butane-1,4-diamine (Compound 94, C₃₁H₃₈FN₅O₂S, MW=563.73 g/mol)

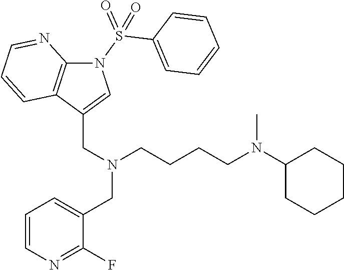 [SMILES: CN(CCCCN(Cc1cccnc1F)Cc1cn(S(=O)(=O)c2ccccc2)c2ncccc12)C1CCCCC1]

As generally described for compound 91: Intermediate 32 (82 mg, 0.28 mmol, 1 equiv), 1-(phenylsulfonyl)-1H-pyrrolo[2,3-b]pyridine-3-carbaldehyde (80 mg, 0.28 mmol, 1 equiv), NaBH(OAc)3 (178 mg, 0.84 mmol, 3 equiv), DCM (1.55 mL). The crude product (205.7 mg of an orange oil) was purified by flash column chromatography on silica gel, using DCM/MeOH·NH₃ 1% as eluent to afford Compound 94 (108 mg, 68%) an a gummy solid.

TLC: Eluent DCM/MeOH·NH3 1% Rf=0.44.

¹H NMR (400 MHz, Chloroform-d) δ 8.40 (dd, J=4.8, 1.6 Hz, 1H, H21), 8.18-8.10 (m, 2H, H15), 8.03 (dt, J=4.8, 1.5 Hz, 1H, H23), 7.91 (dd, J=7.9, 1.7 Hz, 1H, H8), 7.74 (ddd, J=9.5, 7.3, 2.0 Hz, 1H, H17), 7.62 (s, 1H, H13), 7.58-7.50 (m, 1H, H10), 7.49-7.41 (m, 2H, H16), 7.15 (dd, J=7.9, 4.8 Hz, 1H, H9), 7.08 (ddd, J=6.9, 4.8, 1.7 Hz, 1H, H22), 3.67 (s, 2H, H18), 3.58 (s, 2H, H5), 2.54-2.32 (m, 5H, H1, H4 and H24), 2.23 (s, 3H, H28), 1.88-1.68 (m, 4H, H2 and H3), 1.69-1.37 (m, 5H, H25, H26 and H27), 1.35-0.97 (m, 5H, H25′, H26′ and H27′).

¹³C NMR (101 MHz, Chloroform-d) δ 162.12 (d, ¹JCF=238.8 Hz, C20), 147.83 (C14), 146.18 (d, ³JCF=14.6 Hz, C21), 145.29 (C10), 141.55 (d, ³JCF=5.6 Hz, C23), 138.45 (C12), 134.08 (C17), 129.26 (C8), 129.12 (C16), 127.96 (C15), 122.97 (C13), 122.51 (C7), 121.43 (d, ⁴JCF=6.8 Hz, C22), 121.30 (d, ²JCF=39 Hz, C19), 118.90 (C9), 117.32 (C6), 62.93 (C24), 54.05 (C4), 53.28 (C1), 51.28 (C5), 50.18 (C18), 37.49 (C28), 28.23 (C3), 26.23 (C2), 25.94 (C25 and C27), 25.03 (C26).

¹⁹F (376 MHz, CDCl₃) δ−72.27 ppm

LRMS m/z (ESI+, CV 30) 564.27 [M+H]+.

### Preparation of N1-cyclohexyl-N4-((2-fluoropyridin-3-yl)methyl)-N1-methyl-N4-((1-(pyrrolidin-1-ylsulfonyl)-1H-indol-3-yl)methyl)butane-1,4-diamine (Compound 110, C30H42FN5O2S, MW=555.75 g/mol)

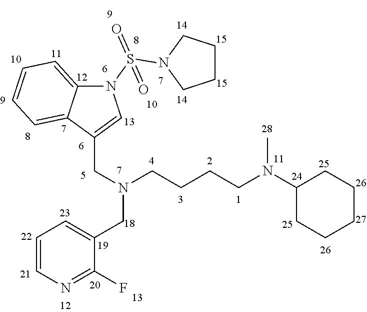 [SMILES: CN(CCCCN(Cc1cccnc1F)Cc1cn(S(=O)(=O)N2CCCC2)c2ccccc12)C1CCCCC1]

As generally described for compound 91, intermediate 32 (82 mg, 0.28 mmol, 1 equiv), 1-(pyrrolidin-1-ylsulfonyl)-1H-indole-3-carbaldehyde (78 mg, 0.28 mmol, 1 equiv), NaBH(OAc)3 (89 mg, 0.42 mmol, 1.5 equiv), DCM (1.55 mL). The crude product was purified by flash column chromatography on silica gel, using a solvent gradient DCM/MeOH·NH3 0% to 10% to afford MBP76 (CM128) (16 mg, 10%) as a colourless gummy solid.

IR (ATR, cm-1): 2924, 2852, 1605, 1578, 1436, 1370, 1169, 1118, 1093, 1016, 973.

1H NMR (400 MHz, CDCl3) δ 8.07 (dt, J=4.8, 1.5 Hz, 1H, H21), 7.93 (d, J=1.0 Hz, 1H, H11), 7.93-7.80 (m, 1H, H23), 7.66-7.57 (m, 1H, H8), 7.40 (s, 1H, H13), 7.33-7.18 (m, 2H, H9 and H10), 7.14 (ddd, J=7.0, 4.8, 1.8 Hz, 1H, H22), 3.71 (s, 2H, H18), 3.62 (s, 2H, H5), 3.36-3.27 (m, 4H, H14), 2.56-2.40 (m, 5H, H1, H4 and H24), 2.25 (s, 3H, H28), 1.87-1.76 (m, 4H, H2 and H3), 1.76-1.70 (m, 4H, H15), 1.68-1.49 (m, 5H, H25, H26 and H27), 1.32-1.01 (m, 5H, H25′, H26′ and H27′).

13C (400 MHz, CDCl3) δ 162.14 (d, 1JCF=238.7 Hz, C20), 146.10 (d, 3JCF=14.7 Hz, C21), 141.72 (d, 3JCF=5.3 Hz, C23), 136.12 (C12), 129.85 (C7), 125.85 (C13), 124.49 (C10), 122.58 (C9), 121.60 (d, 4JCF=4.0 Hz, C22), 121.54 (d, 2JCF=28.9 Hz, C19), 120.35 (C8), 117.66 (C6), 113.77 (C11), 63.10 (C24), 53.77 (C4), 53.11 (C1), 51.20 (C5), 49.90 (C18), 48.66 (C14), 37.03 (C28), 29.83 (C3), 27.87 (C2), 26.04 (C25), 25.77 (C27), 25.47 (C15), 25.07 (C26).

19F (376 MHz, CDCl3) δ−72.59.

LRMS m/z (ESI+, CV 30) [M+H]+(100)

HRMS ESI-TOF [M+H]+m/z calcd. for C30H43FN5O2S 556.3116. Found: 556.3113.

### Preparation of N-(2-(2-(((2-fluoropyridin-3-yl)methyl)((1-((4-methylpiperazin-1-yl)sulfonyl)-1H-indol-3-yl)methyl)amino)ethoxy)ethyl)-N-methylcyclohexanamine (Compound 111, C31H45FN6O3S, MW=600.79 g/mol)

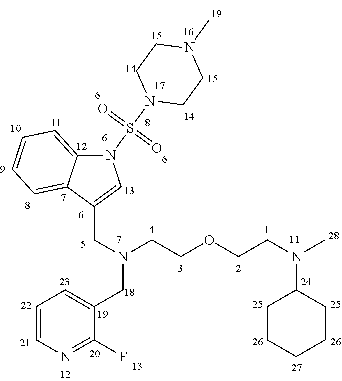 [SMILES: CN1CCN(S(=O)(=O)n2cc(CN(CCOCCN(C)C3CCCCC3)Cc3cccnc3F)c3ccccc32)CC1]

As generally described for compound 91, intermediate 32b (55 mg, 0.18 mmol, 1 equiv), 1-((4-methylpiperazin-1-yl)sulfonyl)-1H-indole-3-carbaldehyde (55 mg, 0.18 mmol, 1 equiv), NaBH(OAc)3 (76 mg, 0.36 mmol, 2 equiv), DCM (1.2 mL). The crude product was purified by flash column chromatography on silica gel, using a solvent gradient DCM/MeOH·NH3 0% to 10% to afford MBP87 (CM129) (84 mg, 78%) as a colourless gummy solid.

IR (ATR, cm-1): 2929, 2855, 2802, 1606, 1436, 1390, 1289, 1245, 1178, 1119, 1067, 950.

1H NMR (400 MHz, CDCl3) δ 8.12-7.99 (m, 1H, H21), 7.96-7.81 (m, 2H, H11 and H23), 7.74-7.62 (m, 1H, H8), 7.38 (s, 1H, H13), 7.35-7.18 (m, 2H, H9 and H10), 7.12 (ddd, J=7.0, 4.8, 1.7 Hz, 1H, H22), 3.82 (s, 2H, H18), 3.72 (s, 2H, H5), 3.60 (t, J=5.6 Hz, 2H, H3), 3.55 (t, J=6.0 Hz, 2H, H4), 3.18 (t, J=4.9 Hz, 4H, H14), 2.74 (t, J=5.6 Hz, 2H, H2), 2.70 (t, J=6.0 Hz, 2H, H1), 2.47 (s, 1H, H24), 2.39-2.29 (m, 7H, H15 and H19), 2.16 (s, 3H, H28), 1.89-1.74 (m, 4H, H25 and H27), 1.67-1.56 (m, 1H, H26), 1.31-1.01 (m, 5H, H25′, H26′ and H27′).

13C (400 MHz, CDCl3) δ 162.03 (d, 1 JCF=238.8 Hz, C20), 145.94 (d, 3JCF=14.7 Hz, C21), 141.47 (d, 3JCF=5.4 Hz, C23), 136.03 (C12), 129.92 (C7), 125.59 (C13), 124.56 (C10), 122.74 (C9), 121.55 (d, 2JCF=28.8 Hz, C19), 121.53 (d, 4JCF=3.9 Hz, C22), 121.40 (C8), 120.44 (C6), 118.22 (C11), 113.94 (C8), 69.78 (C3), 69.66 (C2), 63.75 (C24), 53.89 (C15), 53.54 (C4), 52.91 (C1), 51.45 (C5), 50.34 (C18), 46.69 (C14), 45.74 (C29), 38.56 (C28), 28.34 (C25), 26.24 (C27), 25.95 (C26).

19F (376 MHz, CDCl3) δ−72.53.

LRMS m/z (ESI+, CV 30) 600.88 [M+H]+(100)

HRMS ESI-TOF [M+H]+m/z calcd. for C31H46FN6O3S 601.3331. Found: 601.3325.

### Preparation of N-(2-(2-(((2-fluoropyridin-3-yl)methyl) ((1-(piperidin-1-ylsulfonyl)-1H-indol-3-yl)methyl)amino)ethoxy)ethyl)-N-methylcyclohexanamine (Compound 112, C31H44FN5O3S, MW=585.78 g/mol)

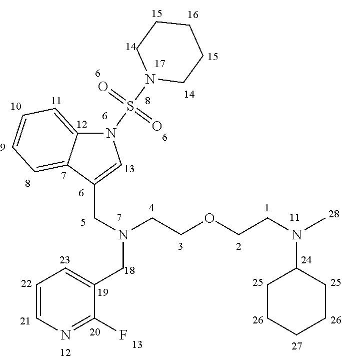 [SMILES: CN(CCOCCN(Cc1cccnc1F)Cc1cn(S(=O)(=O)N2CCCCC2)c2ccccc12)C1CCCCC1]

As generally described for compound 91, intermediate 32b (55 mg, 0.18 mmol, 1 equiv), 1-(piperidin-1-ylsulfonyl)-1H-indole-3-carbaldehyde (53 mg, 0.18 mmol, 1 equiv), NaBH(OAc)3 (76 mg, 0.36 mmol, 2 equiv), DCM (1.2 mL). The crude product was purified by flash column chromatography on silica gel, using a solvent gradient DCM/MeOH·NH3 0% to 10% to afford MBP87 (CM130) (70 mg, 67%) as a colourless gummy solid.

IR (ATR, cm-1): 2928, 2854, 1435, 1387, 1279, 1177, 1118, 1053, 936.

1H NMR (400 MHz, CDCl3) δ 8.13-7.99 (m, 1H, H21), 7.97-7.77 (m, 2H, H11 and H23), 7.76-7.62 (m, 1H, H8), 7.38 (s, 1H, H13), 7.36-7.17 (m, 2H, H9 and H10), 7.10 (ddd, J=7.0, 4.9, 1.8 Hz, 1H, H22), 3.83 (s, 2H, H18), 3.73 (s, 2H, H5), 3.60 (t, J=5.6 Hz, 2H, H3), 3.53 (t, J=6.1 Hz, 2H, H2), 3.13 (t, J=5.5 Hz, 4H, H14), 2.76 (t, J=5.6 Hz, 2H, H4), 2.68 (t, J=6.1 Hz, 2H, H1), 2.44 (s, 1H, H24), 2.33 (s, 3H, H28), 1.89-1.74 (m, 4H, H25 and H27), 1.67-1.57 (m, 1H, H26), 1.51-1.43 (m, 4H, H15), 1.41-1.30 (m, 2H, H16), 1.30-1.00 (m, 5H, H25′, H26′ and H27′).

13C (400 MHz, CDCl3) δ 161.99 (d, 1 JCF=238.7 Hz, C20), 145.88 (d, 3JCF=14.6 Hz, C21), 141.45 (d, 3JCF=5.3 Hz, C23), 135.98 (C12), 129.82 (C7), 125.71 (C13), 124.43 (C10), 122.52 (C9), 121.50 (m, C19 and C22), 120.40 (C8), 117.81 (C6), 113.95 (C11), 69.78 (C2 and C3), 63.70 (C24), 53.63 (C4), 52.91 (C1), 51.45 (C5), 50.39 (C18), 47.65 (C14), 38.61 (C28), 28.40 (C25), 26.28 (C27), 25.98 (C26), 25.09 (C15), 23.22 (C16).

19F (376 MHz, CDCl3) δ−72.60.

LRMS m/z (ESI+, CV 30) 585.78 [M+H]+(100)

HRMS ESI-TOF [M+H]+m/z calcd. for C31H45FN5O3S 586.3222. Found: 586.3218.

### Example 2: Synthesis Protocol 2

### Preparation of INTERMEDIATE 2.1.

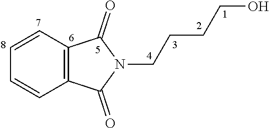 [SMILES: O=C1c2ccccc2C(=O)N1CCCCO]

To a solution of amino 1-aminobutan-4-ol (2.0 g, 22.4 mmol) in toluene (80 mL), phthalic anhydride (3.32 g, 22.4 mmol, 1.0 equiv.) was added quickly. The reaction mixture was heated under reflux in a Dean-Stark apparatus for 3 h. Once water was separated, all volatile matter was then evaporated under vacuum. The crude (5.80 g) was purified by flash chromatography on silica gel, eluting with a gradient mixture of AcOEt/Cyclohexane (30% to 50% of AcOEt) to afford the desired product INTERMEDIATE 2.1. as a white solid (4.57 g, 93% yield).

### Preparation of INTERMEDIATE 2.2.

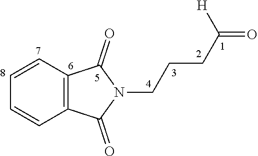 [SMILES: O=CCCCN1C(=O)c2ccccc2C1=O]

A suspension of Celite® (10 g) and INTERMEDIATE 2.1. (1.59 g, 7.25 mmol) in dichloromethane (50 mL) was stirred at room temperature, then pyridinium chlorochromate (2.34 g, 10.88 mmol, 1.5 equiv.) was added portionwise. After stirring at RT for 4 h, the reaction mixture was diluted with diethylether (50 mL) and stirred for 30 min, then filtered through a column of Celite®, washed with diethylether. The solvent was removed under reduced pressure. After purification by flash column chromatography on silica gel eluting with a gradient mixture of AcOEt/Cyclohexane (10% to 30% of AcOEt), a white solid INTERMEDIATE 2.2. (1.28 g, 82%) was obtained.

### Preparation of INTERMEDIATE 2.3.

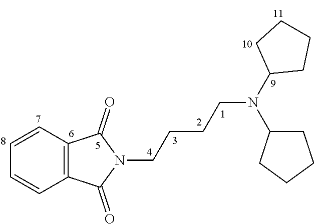 [SMILES: O=C1c2ccccc2C(=O)N1CCCCN(C1CCCC1)C1CCCC1]

To a solution of dicyclopentylamine .HCl (1.73 g, 9.11 mmol, 1.2 eq.) in anhydrous CH₂Cl₂ (100 mL) under argon, diisopropylethylamine (1.6 mL, 9.11 mmol, 1.0 eq.) was added. The reaction mixture was stirred at room temperature during 1 h under argon. INTERMEDIATE 2.2. (1.65 g, 7.6 mmol) was added, the medium was stirred at room temperature for 1 h. Then, the reaction mixture was cooled at 0° C. and NaBH(OAc)3 (2.58 g, 12.15 mmol, 1.6 eq.) was added portionwise. The reaction mixture was stirred at room temperature overnight. A saturated solution of NaHCO₃(100 mL) and CH₂Cl₂ (80 mL) were added, the layers were separated. The aqueous layer was extracted with CH₂Cl₂ (2×50 mL). The combined organic layers were dried over MgSO₄, filtered and concentrated to dryness under vacuo. The crude (3.44 g) was purified by flash chromatography on silica gel, eluting with a gradient mixture of CH₂Cl₂/MeOH NH₃ (7N) (0% to 3% of MeOH NH₃) to afford the desired product INTERMEDIATE 2.3. as a light yellow oil (1.39 g, 52% yield).

### Preparation of INTERMEDIATE 2.4.

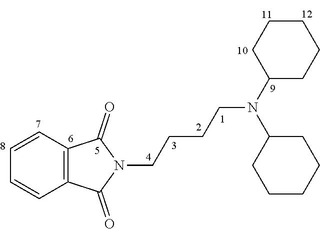 [SMILES: O=C1c2ccccc2C(=O)N1CCCCN(C1CCCCC1)C1CCCCC1]

To a solution of dicyclohexylamine (910 μL, 9.11 mmol, 1.2 eq.) in anhydrous CH₂Cl₂ (100 mL) under argon, INTERMEDIATE 2.2. (1.65 g, 7.6 mmol) was added. The reaction mixture was stirred at room temperature during 1 h under argon. Then, the reaction mixture was cooled at 0° C. and NaBH(OAc)3 (2.58 g, 12.15 mmol, 1.6 eq.) was added portionwise. The reaction mixture was stirred at room temperature overnight. A saturated solution of NaHCO₃(100 mL) and CH₂Cl₂ (80 mL) were added, the layers were separated. The aqueous layer was extracted with CH₂Cl₂ (2×50 mL). The combined organic layers were dried over MgSO₄, filtered and concentrated to dryness under vacuo. The crude (3.44 g) was purified by flash chromatography on silica gel, eluting with a mixture of AcOEt/Cyclohexane (5:5) to afford the desired product INTERMEDIATE 2.4. as a white solid (2.26 g, 78% yield).

### Preparation of INTERMEDIATE 2.5.

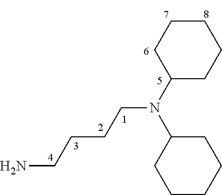 [SMILES: NCCCCN(C1CCCCC1)C1CCCCC1]

The phthalimide INTERMEDIATE 2.4. (1.86 g, 4.86 mmol) was solubilized in a mixture of THF/MeOH (8:2) (40 ml). After addition of hydrazine monohydrate (450 μl, 14.58 mmol, 3.0 equiv) dropwise at 0° C., the mixture was stirred at room temperature overnight. The precipitate was filtered through a Buchner funnel and then washed with THF. The filtrate was evaporated to dryness under reduced pressure.

The crude (2.15 g) was purified by flash chromatography on silica gel, eluting with a gradient mixture of CH₂Cl₂/MeOH NH₃ (7N) (5% to 10% of MeOH NH₃) to afford the desired product INTERMEDIATE 2.5. as a light yellow oil (712 mg, 82% yield) and starting material (546 mg).

### Preparation of INTERMEDIATE 2.6.

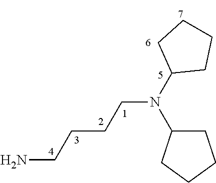 [SMILES: NCCCCN(C1CCCC1)C1CCCC1]

The phthalimide INTERMEDIATE 2.3. (1.39 g, 3.92 mmol) was solubilized in a mixture of THF/MeOH (8:2) (32 ml). After addition of hydrazine monohydrate (370 μl, 11.76 mmol, 3.0 equiv) dropwise at 0° C., the mixture was stirred at room temperature overnight. The precipitate was filtered through a Buchner funnel and then washed with THF. The filtrate was evaporated to dryness under reduced pressure.

The crude (1.54 g) was purified by flash chromatography on silica gel, eluting with a gradient mixture of CH₂Cl₂/MeOH NH₃ (7N) (5% to 10% of MeOH NH₃) to afford the desired product INTERMEDIATE 2.5. as a light yellow oil (609 mg, 82% yield) and starting material (216 mg).

### Preparation of INTERMEDIATE 2.7.

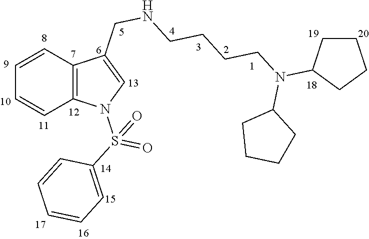 [SMILES: O=S(=O)(c1ccccc1)n1cc(CNCCCCN(C2CCCC2)C2CCCC2)c2ccccc21]

To a solution of INTERMEDIATE 2.6. (374 mg, 1.67 mmol) in anhydrous CH₂Cl₂ (10 mL), aldehyde (476 mg, 1.67 mmol) was added. The reaction mixture was stirred at room temperature during 1 h under argon. Then, NaBH(OAc)3 (565 mg, 2.67 mmol, 1.6 eq.) was added portionwise. The reaction mixture was stirred at room temperature overnight. A saturated solution of NaHCO₃(20 mL) was added, the layers were separated. The aqueous layer was extracted with CH₂Cl₂ (3×10 mL). The combined organic layers were dried over MgSO₄, filtered and concentrated to dryness under vacuo. The crude (938 mg) was purified by flash chromatography on silica gel, eluting with a gradient mixture of CH₂Cl₂/MeOH NH₃ (7N) (0% to 4% of MeOH NH₃) to afford the desired product INTERMEDIATE 2.7. as a light yellow oil (253 mg, 31% yield) and a by-product INTERMEDIATE 2.7.b. from the double reductive amination as a colorless oil (396 mg).

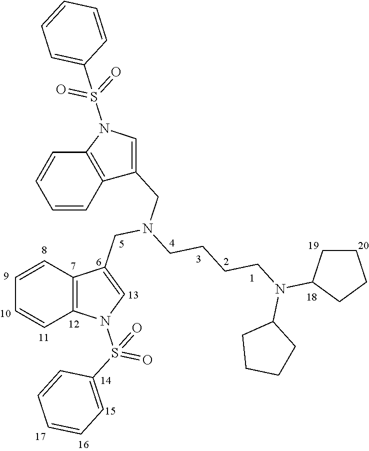 [SMILES: O=S(=O)(c1ccccc1)n1cc(CN(CCCCN(C2CCCC2)C2CCCC2)Cc2cn(S(=O)(=O)c3ccccc3)c3ccccc23)c2ccccc21]

### Preparation of INTERMEDIATE 2.8.

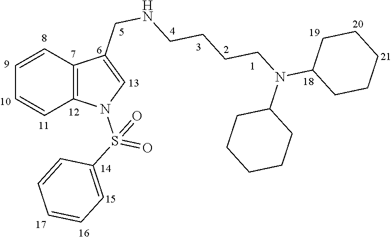 [SMILES: O=S(=O)(c1ccccc1)n1cc(CNCCCCN(C2CCCCC2)C2CCCCC2)c2ccccc21]

To a solution of INTERMEDIATE 2.5. (610 mg, 2.42 mmol) in anhydrous CH₂Cl₂ (15 mL), aldehyde (690 mg, 2.42 mmol) was added. The reaction mixture was stirred at room temperature during 1 h under argon. Then, NaBH(OAc)3 (820 mg, 3.87 mmol, 1.6 eq.) was added portionwise. The reaction mixture was stirred at room temperature overnight. A saturated solution of NaHCO₃(20 mL) was added, the layers were separated. The aqueous layer was extracted with CH₂Cl₂ (3×10 mL). The combined organic layers were dried over MgSO₄, filtered and concentrated to dryness under vacuo. The crude (1.43 g) was purified by flash chromatography on silica gel, eluting with a mixture of CH₂Cl₂/MeOH NH₃ (7N) (98:2) to afford the desired product INTERMEDIATE 2.8. as a light yellow oil (343 mg, 27% yield) and a by-product INTERMEDIATE 2.8.b. from the double reductive amination as a white amorphous solid (722 mg).

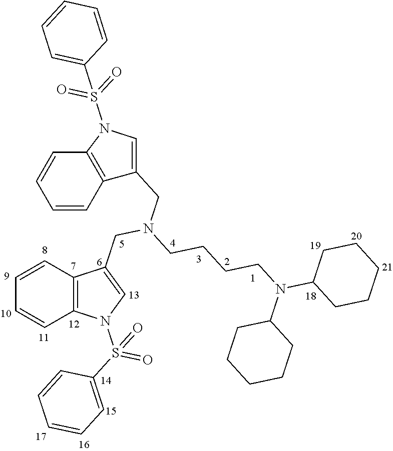 [SMILES: O=S(=O)(c1ccccc1)n1cc(CN(CCCCN(C2CCCCC2)C2CCCCC2)Cc2cn(S(=O)(=O)c3ccccc3)c3ccccc23)c2ccccc21]

### Preparation of COMPOUND 113

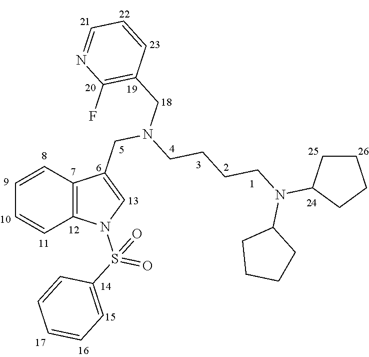 [SMILES: O=S(=O)(c1ccccc1)n1cc(CN(CCCCN(C2CCCC2)C2CCCC2)Cc2cccnc2F)c2ccccc21]

To a solution of INTERMEDIATE 2.7. (221 mg, 0.45 mmol) in anhydrous CH₂Cl₂ (10 mL), aldehyde (63 mg, 0.51 mmol, 1.1 eq.) was added. The reaction mixture was stirred at room temperature during 1 h under argon. Then, NaBH(OAc)3 (285 mg, 1.34 mmol, 3.0 eq.) was added portionwise. The reaction mixture was stirred at room temperature overnight. A saturated solution of NaHCO₃(15 mL) was added, the layers were separated. The aqueous layer was extracted with CH₂Cl₂ (3×10 mL). The combined organic layers were dried over MgSO₄, filtered and concentrated to dryness under vacuo. The crude (305 mg) was purified by flash chromatography on silica gel, eluting with a mixture of CH₂Cl₂/MeOH NH₃ (7N) (98:2) to afford the desired product COMPOUND 113 as a light yellow oil (138 mg, 51% yield).

### Preparation of COMPOUND 114

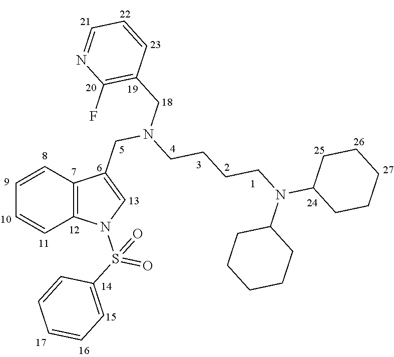 [SMILES: O=S(=O)(c1ccccc1)n1cc(CN(CCCCN(C2CCCCC2)C2CCCCC2)Cc2cccnc2F)c2ccccc21]

To a solution of INTERMEDIATE 2.8. (290 mg, 0.56 mmol) in anhydrous CH₂Cl₂ (10 mL), aldehyde (79 mg, 0.63 mmol, 1.1 eq.) was added. The reaction mixture was stirred at room temperature during 1 h under argon. Then, NaBH(OAc)3 (353 mg, 1.67 mmol, 3.0 eq.) was added portionwise. The reaction mixture was stirred at room temperature overnight. A saturated solution of NaHCO₃(15 mL) was added, the layers were separated. The aqueous layer was extracted with CH₂Cl₂ (3×10 mL). The combined organic layers were dried over MgSO₄, filtered and concentrated to dryness under vacuo. The crude (414 mg) was purified by flash chromatography on silica gel, eluting with a mixture of CH₂Cl₂/MeOH NH₃ (7N) (98:2) to afford the desired product COMPOUND 114 as a light yellow oil (183 mg, 52% yield).

### Preparation of INTERMEDIATE 2.9.

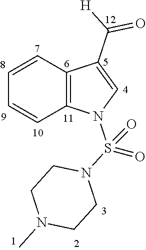 [SMILES: CN1CCN(S(=O)(=O)n2cc(C=O)c3ccccc32)CC1]

To a solution of Indole-3-carboxaldehyde (1.00 g, 6.89 mmol) in anhydrous DMF (10 mL) under argon at 0° C., NaH (60%) (0.55 g, 13.78 mmole, 2.0 eq.) was added portionwise. The reaction mixture was stirred at room temperature for 1 h. Then 4-Methylpiperazine-1-sulfonyl chloride (1.64 mg, 8.27 mmol, 1.2 eq.) in anhydrous DMF (5 mL) was added dropwise. The reaction mixture was stirred at room temperature overnight under argon. The medium was cooled at 0° C. and quenched with ice/water (100 mL), stirred 20 min. The resulting suspension was filtered and then solubilized with CH₂Cl₂ (50 mL). The organic layer was washed with a saturated solution of NaHCO₃(100 mL) and brine (100 mL). The organic layer was dried over MgSO₄, filtered and concentrated to dryness under vacuo. The crude (1.73 g) was purified by flash chromatography on silica gel, eluting with a mixture of AcOEt/Cyclohexane (5:5 to 8:2) to afford the desired product INTERMEDIATE 2.9. as a white solid (1.45 g, 68% yield).

### Preparation of INTERMEDIATE 2.10.

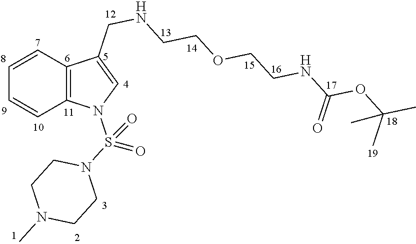 [SMILES: CN1CCN(S(=O)(=O)n2cc(CNCCOCCNC(=O)OC(C)(C)C)c3ccccc32)CC1]

To a solution of INTERMEDIATE 2.9. (1.43 g, 4.65 mmol) in anhydrous CH₂Cl₂ (30 mL), N-Boc-2-(2-amino-ethoxy)-ethylamine (950 mg, 4.65 mmol) was added. The reaction mixture was stirred at room temperature during 1 h under argon. Then, NaBH(OAc)3 (1.58 g, 7.44 mmol, 1.6 eq.) was added portionwise. The reaction mixture was stirred at room temperature overnight. A saturated solution of NaHCO₃(40 mL) was added, the layers were separated. The aqueous layer was extracted with CH₂Cl₂ (2×35 mL). The combined organic layers were dried over MgSO₄, filtered and concentrated to dryness under vacuo. The crude (2.46 g) was purified by flash chromatography on silica gel, eluting with a mixture of CH₂Cl₂/MeOH NH₃ (7N) (0% to 5% MeOH NH₃) to afford the desired product INTERMEDIATE 2.10. as a colorless oil (881 mg, 38% yield) and a by product INTERMEDIATE 2.10.b. (1.11 g) from double reductive amination.

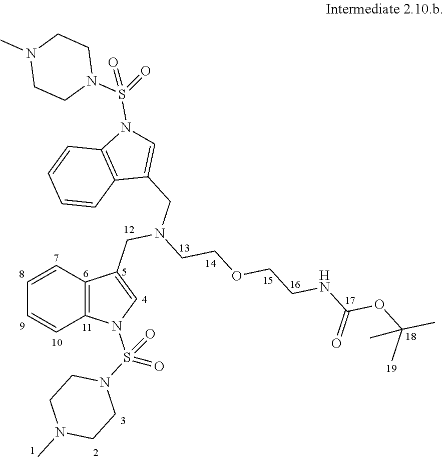 [SMILES: CN1CCN(S(=O)(=O)n2cc(CN(CCOCCNC(=O)OC(C)(C)C)Cc3cn(S(=O)(=O)N4CCN(C)CC4)c4ccccc34)c3ccccc32)CC1]

### Preparation of INTERMEDIATE 2.11.

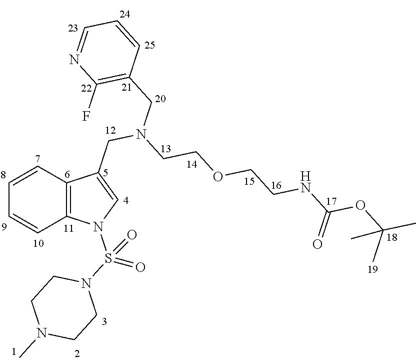 [SMILES: CN1CCN(S(=O)(=O)n2cc(CN(CCOCCNC(=O)OC(C)(C)C)Cc3cccnc3F)c3ccccc32)CC1]

To a solution of INTERMEDIATE 2.10. (1.15 g, 2.32 mmol) in anhydrous CH₂Cl₂ (35 mL), 2-fluoropyridine-3-carboxaldehyde (330 mg, 2.62 mmol, 1.13 eq.) in CH₂Cl₂ (5 mL) was added. The reaction mixture was stirred at room temperature during 1 h under argon. Then, NaBH(OAc)3 (1.48 g, 6.96 mmol, 3.0 eq.) was added portionwise at 0° C. The reaction mixture was stirred at room temperature overnight under argon. A saturated solution of NaHCO₃(50 mL) was added, the layers were separated. The aqueous layer was extracted with CH₂Cl₂ (3×50 mL). The combined organic layers were dried over MgSO₄, filtered and concentrated to dryness under vacuo. The crude (1.50 g) was purified by flash chromatography on silica gel, eluting with a mixture of CH₂Cl₂/MeOH NH₃ (7N) (0% to 2% MeOH NH₃) to afford the desired product INTERMEDIATE 2.11. as a colorless oil (1.20 g, 86% yield).

### Preparation of INTERMEDIATE 2.12.

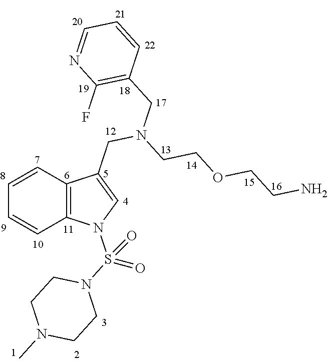 [SMILES: CN1CCN(S(=O)(=O)n2cc(CN(CCOCCN)Cc3cccnc3F)c3ccccc32)CC1]

To a solution of INTERMEDIATE 2.11. (1.20 g, 1.98 mmol) in CH₂Cl₂ (30 mL) at 0° C., trifluoroacetic acid (1.80 mL, 23.81 mmol, 12 eq.) was added dropwise. The reaction mixture was stirred at room temperature overnight. Water (50 mL) was added, the pH was adjusted to 8-9 with a saturated solution of NaHCO₃. The layers were separated and the aqueous layer was extracted twice with CH₂Cl₂ (50 mL). The combined organic layers were dried over MgSO₄, filtered and evaporated to dryness. The residue (1.00 g) was purified by flash chromatography on silica gel, eluting with a mixture of CH₂Cl₂/MeOH NH₃ (7N) (0% to 3% MeOH NH₃) to afford the desired product INTERMEDIATE 2.12. as a light yellow oil (661 mg, 66%).

### Preparation of INTERMEDIATE 2.13.

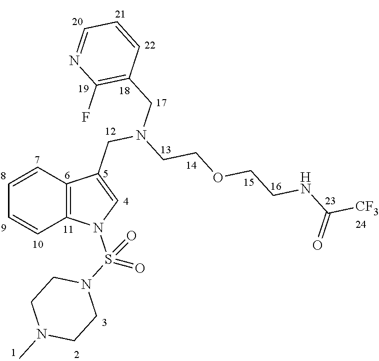 [SMILES: CN1CCN(S(=O)(=O)n2cc(CN(CCOCCNC(=O)C(F)(F)F)Cc3cccnc3F)c3ccccc32)CC1]

To a solution of INTERMEDIATE 2.12. (130 mg, 0.258 mmol) in CH₂Cl₂ (3 mL) at 0° C., trifluoroacetic anhydride (40 μL, 0.283 mmol, 1.1 eq.) was added dropwise. The reaction mixture was stirred at room temperature overnight. CH₂Cl₂ (10 mL) was added, the pH was adjusted to 8-9 with a saturated solution of NaHCO₃(10 mL). The layers were separated and the aqueous layer was extracted three times with CH₂Cl₂ (10 mL). The combined organic layers were dried over MgSO₄, filtered and evaporated to dryness. The residue (150 mg) was purified by flash chromatography on silica gel, eluting with a mixture of CH₂Cl₂/MeOH NH₃ (7N) (0% to 2% MeOH NH₃) to afford the desired product INTERMEDIATE 2.13. as a light yellow oil (136 mg, 88%).

### Preparation of COMPOUND 115

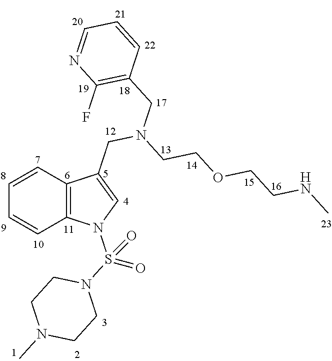 [SMILES: CNCCOCCN(Cc1cccnc1F)Cc1cn(S(=O)(=O)N2CCN(C)CC2)c2ccccc12]

To a solution of INTERMEDIATE 2.13. (136 mg, 0.226 mmol) in anhydrous DMF (4 mL) at room temperature, potassium carbonate (125 mg, 0.906 mmol, 4.0 eq.) was added. The reaction mixture was stirred and heated to 100° C. for 1 h. Then, methyl iodide (16 μL, 0.249 mmol, 1.1 eq.) was added, the medium was stirred and heated at 100° C. overnight, under argon. The reaction mixture was cooled to room temperature, EtOAc (20 mL) and a saturated solution of NaHCO₃(20 mL) were added. The layers were separated and the aqueous layer was extracted twice with EtOAc (20 mL). The combined organic layers were dried over MgSO₄, filtered and evaporated to dryness. The residue (185 mg) was purified by flash chromatography on silica gel, eluting with a mixture of CH₂Cl₂/MeOH NH₃ (7N) (0% to 2% MeOH NH₃) to afford the methylated product as a colorless oil (35 mg, 25%) and an unknown product (48 mg).

To a solution of methylated compound (35 mg, 0.057 mmol) in MeOH (1 mL) at room temperature, lithium hydroxide (1.5 mg, 0.062 mmol, 1.1 eq.) in water (0.5 mL) was added dropwise. The reaction mixture was stirred at room temperature for 3 h. Then, the solvent was removed under vacuum, the residue was taken up with CH₂Cl₂ (10 mL). The layer was washed with a saturated solution of NaHCO₃(10 mL) and the aqueous layer was extracted three times with CH₂Cl₂ (10 mL). The combined organic layers were dried over MgSO₄, filtered and evaporated to dryness to afford COMPOUND 115 as a colorless oil (21 mg, 72%).

### Preparation of COMPOUND 116

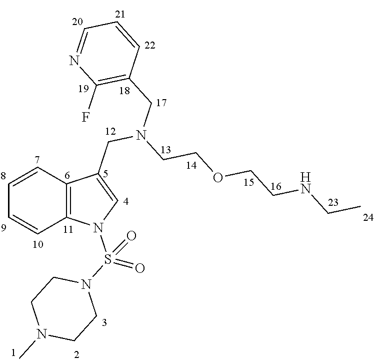 [SMILES: CCNCCOCCN(Cc1cccnc1F)Cc1cn(S(=O)(=O)N2CCN(C)CC2)c2ccccc12]

To a solution of INTERMEDIATE 2.13. (140 mg, 0.233 mmol) in anhydrous DMF (4 mL) at room temperature, potassium carbonate (129 mg, 0.932 mmol, 4.0 eq.) was added. The reaction mixture was stirred and heated to 100° C. for 1 h. Then, ethyl iodide (21 μL, 0.256 mmol, 1.1 eq.) was added, the medium was stirred and heated at 100° C. overnight, under argon. The reaction mixture was cooled to room temperature, EtOAc (20 mL) and a saturated solution of NaHCO₃(20 mL) were added. The layers were separated and the aqueous layer was extracted twice with EtOAc (20 mL). The combined organic layers were dried over MgSO₄, filtered and evaporated to dryness. The residue (147 mg) was purified by flash chromatography on silica gel, eluting with a mixture of CH₂Cl₂/MeOH NH₃ (7N) (0% to 2% MeOH NH₃) to afford the ethylated product as a colorless oil (15 mg, 15%) and starting material (43 mg).

To a solution of ethylated compound (15 mg, 0.024 mmol) in MeOH (1 mL) at room temperature, lithium hydroxide (0.6 mg, 0.026 mmol, 1.1 eq.) in water (0.5 mL) was added dropwise. The reaction mixture was stirred at room temperature for 3 h. Then, the solvent was removed under vacuum, the residue was taken up with CH₂Cl₂ (10 mL). The layer was washed with a saturated solution of NaHCO₃(10 mL) and the aqueous layer was extracted three times with CH₂Cl₂ (10 mL). The combined organic layers were dried over MgSO₄, filtered and evaporated to dryness to afford COMPOUND 116 as a colorless oil (9 mg, 75%).

### Preparation of COMPOUND 117

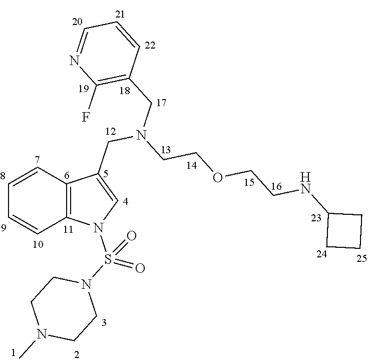 [SMILES: CN1CCN(S(=O)(=O)n2cc(CN(CCOCCNC3CCC3)Cc3cccnc3F)c3ccccc32)CC1]

To a solution of INTERMEDIATE 2.12. (78 mg, 0.154 mmol) in anhydrous MeOH (1 mL), cyclobutanone (11.5 μL, 0.154 mmol) was added. The reaction mixture was stirred at room temperature during 1 h under argon. Then, NaBH₄ (9.5 mg, 0.247 mmol, 3.0 eq.) was added portionwise at 0° C. The reaction mixture was stirred at room temperature overnight under argon. The solvent was removed and the residue was taken up with CH₂Cl₂ (10 mL). A saturated solution of NaHCO₃(10 mL) was added, the layers were separated. The aqueous layer was extracted with CH₂Cl₂ (3×10 mL). The combined organic layers were dried over MgSO₄, filtered and concentrated to dryness under vacuo. The crude (50 mg) was purified by preparative chromatography on silica gel, eluting with a mixture of CH₂Cl₂/MeOH NH₃ (7N) (96:4) to afford the desired product COMPOUND 117 as a colorless oil (11 mg, 18% yield) and starting material (23 mg).

### Preparation of INTERMEDIATE 2.14.

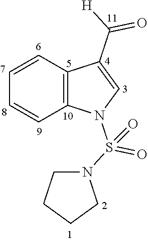 [SMILES: O=Cc1cn(S(=O)(=O)N2CCCC2)c2ccccc12]

To a solution of Indole-3-carboxaldehyde (1.00 g, 6.89 mmol) in anhydrous DMF (10 mL) under argon at 0° C., NaH (60%) (0.55 g, 13.78 mmole, 2.0 eq.) was added portionwise. The reaction mixture was stirred at room temperature for 1 h. Then pyrrolidine-1-sulfonyl chloride (950 μL, 8.27 mmol, 1.2 eq.) in anhydrous DMF (5 mL) was added dropwise. The reaction mixture was stirred at room temperature overnight under argon. The medium was cooled at 0° C. and quenched with ice/water (100 mL), stirred 20 min. The resulting suspension was filtered and then solubilized with CH₂Cl₂ (50 mL). The organic layer was washed with a saturated solution of NaHCO₃(100 mL) and brine (100 mL). The organic layer was dried over MgSO₄, filtered and concentrated to dryness under vacuo. The crude (2.23 g) was purified by flash chromatography on silica gel, eluting with a mixture of AcOEt/Cyclohexane (5:5 to 8:2) to afford the desired product INTERMEDIATE 2.14. as a white solid (1.90 g, 99% yield).

### Preparation of INTERMEDIATE 2.15.

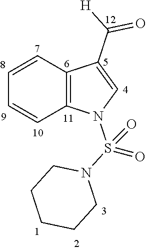 [SMILES: O=Cc1cn(S(=O)(=O)N2CCCCC2)c2ccccc12]

To a solution of Indole-3-carboxaldehyde (1.00 g, 6.89 mmol) in anhydrous DMF (10 mL) under argon at 0° C., NaH (60%) (0.55 g, 13.78 mmole, 2.0 eq.) was added portionwise. The reaction mixture was stirred at room temperature for 1 h. Then Piperidine-1-sulfonyl chloride (1.16 mL, 8.27 mmol, 1.2 eq.) in anhydrous DMF (5 mL) was added dropwise. The reaction mixture was stirred at room temperature overnight under argon. The medium was cooled at 0° C. and quenched with ice/water (100 mL), stirred 20 min. The resulting suspension was filtered and then solubilized with CH₂Cl₂ (50 mL). The organic layer was washed with a saturated solution of NaHCO₃(100 mL) and brine (100 mL). The organic layer was dried over MgSO₄, filtered and concentrated to dryness under vacuo. The crude (2.20 g) was purified by flash chromatography on silica gel, eluting with a mixture of AcOEt/Cyclohexane (5:5 to 8:2) to afford the desired product INTERMEDIATE 2.15. as a white solid (2.00 g, 99% yield).

### Preparation of INTERMEDIATE 2.16.

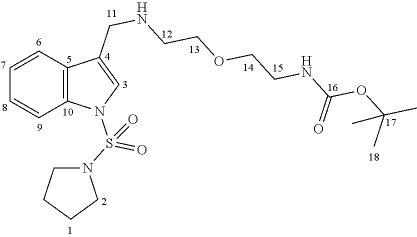 [SMILES: CC(C)(C)OC(=O)NCCOCCNCc1cn(S(=O)(=O)N2CCCC2)c2ccccc12]

To a solution of INTERMEDIATE 2.14. (1.90 g, 6.83 mmol) in anhydrous MeOH (45 mL), N-Boc-2-(2-amino-ethoxy)-ethylamine (1.40 g, 6.83 mmol) was added. The reaction mixture was stirred at room temperature during 1 h under argon. Then, the medium was cooled at 0° C., NaBH₄ (413 mg, 10.92 mmol, 1.6 eq.) was added portionwise. The reaction mixture was stirred at room temperature overnight. MeOH was evaporated under reduced pressure. The crude was taken up with CH₂Cl₂ (100 mL) and a saturated solution of NaHCO₃(50 mL). The layers were separated. The aqueous layer was extracted with CH₂Cl₂ (2×50 mL). The combined organic layers were dried over MgSO₄, filtered and concentrated to dryness under vacuo. The crude (3.65 g) was purified by flash chromatography on silica gel, eluting with a mixture of CH₂Cl₂/MeOH NH₃ (7N) (98:2) to afford the desired product INTERMEDIATE 2.16. as a yellow oil (2.72 g, 86% yield).

### Preparation of INTERMEDIATE 2.17.

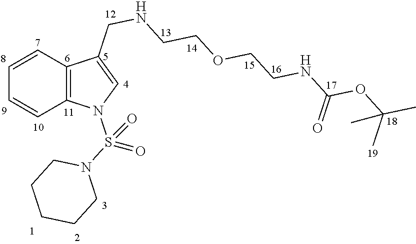 [SMILES: CC(C)(C)OC(=O)NCCOCCNCc1cn(S(=O)(=O)N2CCCCC2)c2ccccc12]

To a solution of INTERMEDIATE 2.15. (2.00 g, 6.84 mmol) in anhydrous MeOH (45 mL), N-Boc-2-(2-amino-ethoxy)-ethylamine (1.40 g, 6.84 mmol) was added. The reaction mixture was stirred at room temperature during 1 h under argon. Then, the medium was cooled at 0° C., NaBH₄ (414 mg, 10.95 mmol, 1.6 eq.) was added portionwise. The reaction mixture was stirred at room temperature overnight. MeOH was evaporated under reduced pressure. The crude was taken up with CH₂Cl₂ (100 mL) and a saturated solution of NaHCO₃(50 mL). The layers were separated. The aqueous layer was extracted with CH₂Cl₂ (2×50 mL). The combined organic layers were dried over MgSO₄, filtered and concentrated to dryness under vacuo. The crude (3.67 g) was purified by flash chromatography on silica gel, eluting with a mixture of CH₂Cl₂/MeOH NH₃ (7N) (98:2) to afford the desired product INTERMEDIATE 2.17. as a yellow oil (2.76 g, 84% yield).

### Preparation of INTERMEDIATE 2.18.

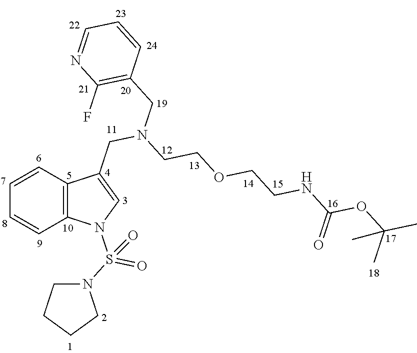 [SMILES: CC(C)(C)OC(=O)NCCOCCN(Cc1cccnc1F)Cc1cn(S(=O)(=O)N2CCCC2)c2ccccc12]

To a solution of INTERMEDIATE 2.16. (2.56 g, 5.49 mmol) in anhydrous CH₂Cl₂ (80 mL), 2-fluoropyridine-3-carboxaldehyde (776 mg, 6.20 mmol, 1.13 eq.) in CH₂Cl₂ (10 mL) was added. The reaction mixture was stirred at room temperature during 1 h under argon. Then, NaBH(OAc)3 (3.49 g, 16.46 mmol, 3.0 eq.) was added portionwise at 0° C. The reaction mixture was stirred at room temperature overnight under argon. A saturated solution of NaHCO₃(100 mL) was added, the layers were separated. The aqueous layer was extracted with CH₂Cl₂ (3×50 mL). The combined organic layers were dried over MgSO₄, filtered and concentrated to dryness under vacuo. The crude (3.44 g) was purified by flash chromatography on silica gel, eluting with a mixture of CH₂Cl₂/MeOH NH₃ (7N) (99:1) to afford the desired product INTERMEDIATE 2.18. as a colorless oil (2.28 g, 72% yield).

### Preparation of INTERMEDIATE 2.19.

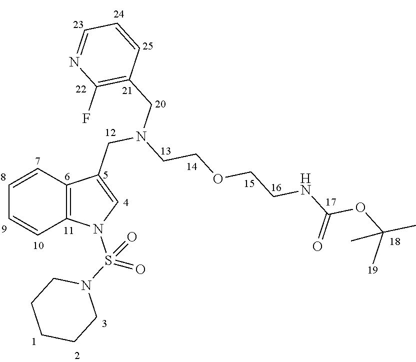 [SMILES: CC(C)(C)OC(=O)NCCOCCN(Cc1cccnc1F)Cc1cn(S(=O)(=O)N2CCCCC2)c2ccccc12]

To a solution of INTERMEDIATE 2.17. (2.57 g, 5.35 mmol) in anhydrous CH₂Cl₂ (80 mL), 2-fluoropyridine-3-carboxaldehyde (756 mg, 6.04 mmol, 1.13 eq.) in CH₂Cl₂ (10 mL) was added. The reaction mixture was stirred at room temperature during 1 h under argon. Then, NaBH(OAc)3 (3.40 g, 16.04 mmol, 3.0 eq.) was added portionwise at 0° C. The reaction mixture was stirred at room temperature overnight under argon. A saturated solution of NaHCO₃(100 mL) was added, the layers were separated. The aqueous layer was extracted with CH₂Cl₂ (3×50 mL). The combined organic layers were dried over MgSO₄, filtered and concentrated to dryness under vacuo. The crude (3.52 g) was purified by flash chromatography on silica gel, eluting with a mixture of CH₂Cl₂/MeOH NH₃ (7N) (99:1) to afford the desired product INTERMEDIATE 2.19. as a colorless oil (2.41 g, 76% yield).

### Preparation of INTERMEDIATE 2.20.

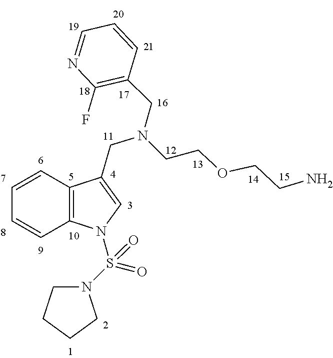 [SMILES: NCCOCCN(Cc1cccnc1F)Cc1cn(S(=O)(=O)N2CCCC2)c2ccccc12]

To a solution of INTERMEDIATE 2.18. (2.28 g, 3.96 mmol) in CH₂Cl₂ (65 mL) at 0° C., trifluoroacetic acid (3.70 mL, 47.53 mmol, 12 eq.) was added dropwise. The reaction mixture was stirred at room temperature overnight. Water (50 mL) was added, the pH was adjusted to 8-9 with a saturated solution of NaHCO₃. The layers were separated and the aqueous layer was extracted three times with CH₂Cl₂ (50 mL). The combined organic layers were dried over MgSO₄, filtered and evaporated to dryness. The desired product INTERMEDIATE 2.20. as a yellow oil (1.90 g, 99%) was pure enough to be engaged in the next step.

### Preparation of INTERMEDIATE 2.21.

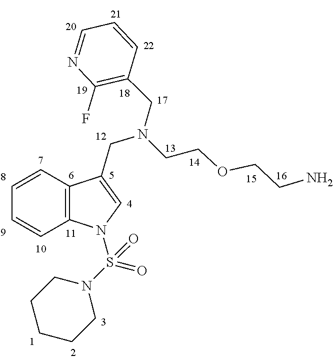 [SMILES: NCCOCCN(Cc1cccnc1F)Cc1cn(S(=O)(=O)N2CCCCC2)c2ccccc12]

To a solution of INTERMEDIATE 2.19. (2.40 g, 4.07 mmol) in CH₂Cl₂ (65 mL) at 0° C., trifluoroacetic acid (3.80 mL, 48.84 mmol, 12 eq.) was added dropwise. The reaction mixture was stirred at room temperature overnight. Water (50 mL) was added, the pH was adjusted to 8-9 with a saturated solution of NaHCO₃. The layers were separated and the aqueous layer was extracted three times with CH₂Cl₂ (50 mL). The combined organic layers were dried over MgSO₄, filtered and evaporated to dryness. The desired product INTERMEDIATE 2.21. as a yellow oil (2.00 g, 99%) was pure enough to be engaged in the next step.

### Preparation of COMPOUND 118

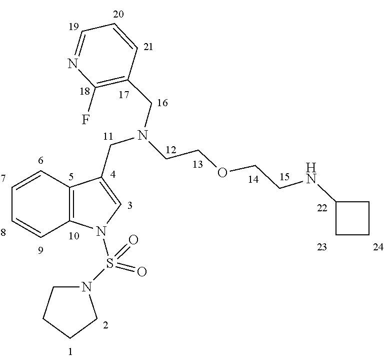 [SMILES: O=S(=O)(N1CCCC1)n1cc(CN(CCOCCNC2CCC2)Cc2cccnc2F)c2ccccc21]

To a solution of INTERMEDIATE 2.20. (200 mg, 0.42 mmol) in anhydrous MeOH (3 mL), cyclobutanone (35 μL, 0.46 mmol, 1.1 eq.) was added. The reaction mixture was stirred at room temperature during 1 h under argon. Then, NaBH₄ (25.5 mg, 0.67 mmol, 1.6 eq.) was added portionwise at 0° C. The reaction mixture was stirred at room temperature overnight under argon. The solvent was removed and the residue was taken up with CH₂Cl₂ (10 mL). A saturated solution of NaHCO₃(10 mL) was added, the layers were separated. The aqueous layer was extracted with CH₂Cl₂ (3×10 mL). The combined organic layers were dried over MgSO₄, filtered and concentrated to dryness under vacuo. The crude (180 mg) was purified by preparative chromatography on silica gel, eluting with a mixture of CH₂Cl₂/MeOH NH₃ (7N) (96:4) to afford the desired product COMPOUND 118 as a colorless oil (30 mg, 20% yield) and starting material (61 mg).

### Preparation of COMPOUND 119

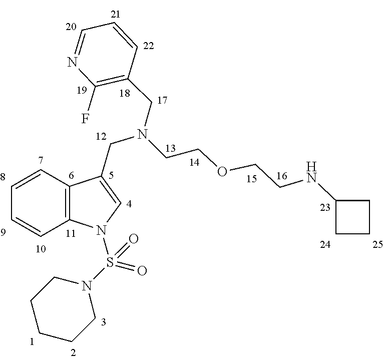 [SMILES: O=S(=O)(N1CCCCC1)n1cc(CN(CCOCCNC2CCC2)Cc2cccnc2F)c2ccccc21]

To a solution of INTERMEDIATE 2.21. (200 mg, 0.41 mmol) in anhydrous MeOH (3 mL), cyclobutanone (34 μL, 0.45 mmol, 1.1 eq.) was added. The reaction mixture was stirred at room temperature during 1 h under argon. Then, NaBH₄ (25 mg, 0.65 mmol, 3.0 eq.) was added portionwise at 0° C. The reaction mixture was stirred at room temperature overnight under argon. The solvent was removed and the residue was taken up with CH₂Cl₂ (10 mL). A saturated solution of NaHCO₃(10 mL) was added, the layers were separated. The aqueous layer was extracted with CH₂Cl₂ (3×10 mL). The combined organic layers were dried over MgSO₄, filtered and concentrated to dryness under vacuo. The crude (215 mg) was purified by preparative chromatography on silica gel, eluting with a mixture of CH₂Cl₂/MeOH NH₃ (7N) (96:4) to afford the desired product COMPOUND 119 as a colorless oil (25 mg, 16% yield) and starting material (60 mg).

### Preparation of INTERMEDIATE 2.22.

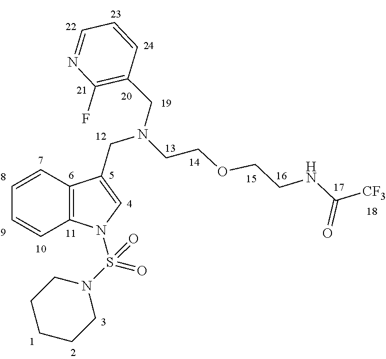 [SMILES: O=C(NCCOCCN(Cc1cccnc1F)Cc1cn(S(=O)(=O)N2CCCCC2)c2ccccc12)C(F)(F)F]

To a solution of INTERMEDIATE 2.21. (500 mg, 1.02 mmol) in CH₂Cl₂ (12 mL) at 0° C., trifluoroacetic anhydride (156 μL, 1.12 mmol, 1.1 eq.) was added dropwise. The reaction mixture was stirred at room temperature overnight. CH₂Cl₂ (10 mL) was added, the pH was adjusted to 8-9 with a saturated solution of NaHCO₃(20 mL). The layers were separated and the aqueous layer was extracted three times with CH₂Cl₂ (20 mL). The combined organic layers were dried over MgSO₄, filtered and evaporated to dryness. The residue (582 mg) was purified by flash chromatography on silica gel, eluting with a mixture of CH₂Cl₂/MeOH NH₃ (7N) (0% to 2% MeOH NH₃) to afford the desired product INTERMEDIATE 2.22. as a light yellow oil (540 mg, 90%).

### Preparation of INTERMEDIATE 2.23.

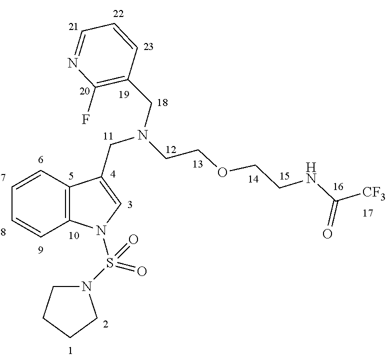 [SMILES: O=C(NCCOCCN(Cc1cccnc1F)Cc1cn(S(=O)(=O)N2CCCC2)c2ccccc12)C(F)(F)F]

To a solution of INTERMEDIATE 2.21. (500 mg, 1.05 mmol) in CH₂Cl₂ (12 mL) at 0° C., trifluoroacetic anhydride (161 μL, 1.16 mmol, 1.1 eq.) was added dropwise. The reaction mixture was stirred at room temperature overnight. CH₂Cl₂ (10 mL) was added, the pH was adjusted to 8-9 with a saturated solution of NaHCO₃(20 mL). The layers were separated and the aqueous layer was extracted three times with CH₂Cl₂ (20 mL). The combined organic layers were dried over MgSO₄, filtered and evaporated to dryness. The residue (480 mg) was purified by flash chromatography on silica gel, eluting with a mixture of CH₂Cl₂/MeOH NH₃ (7N) (0% to 2% MeOH NH₃) to afford the desired product INTERMEDIATE 2.23. as a light yellow oil (472 mg, 78%).

### Preparation of COMPOUND 120

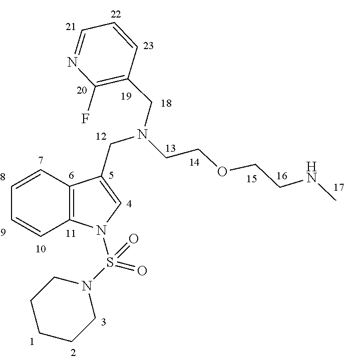 [SMILES: CNCCOCCN(Cc1cccnc1F)Cc1cn(S(=O)(=O)N2CCCCC2)c2ccccc12]

To a solution of INTERMEDIATE 2.22. (260 mg, 0.44 mmol) in anhydrous DMF (8 mL) at room temperature, potassium carbonate (245 mg, 1.78 mmol, 4.0 eq.) was added. The reaction mixture was stirred and heated to 100° C. for 1 h. Then, methyl iodide (30 μL, 0.49 mmol, 1.1 eq.) was added, the medium was stirred and heated at 100° C. overnight, under argon. The reaction mixture was cooled to room temperature, EtOAc (30 mL) and a saturated solution of NaHCO₃(30 mL) were added. The layers were separated and the aqueous layer was extracted twice with EtOAc (30 mL). The combined organic layers were dried over MgSO₄, filtered and evaporated to dryness. The residue (276 mg) was purified by preparative chromatography on silica gel, eluting with a mixture of CH₂Cl₂/MeOH NH₃ (7N) (96:4) to afford the desired methylated compound as a colorless oil (75 mg, 30% yield).

To a solution of methylated compound (75 mg, 0.125 mmol) in MeOH (2 mL) at room temperature, lithium hydroxide (3.3 mg, 0.138 mmol, 1.1 eq.) in water (1 mL) was added dropwise. The reaction mixture was stirred at room temperature overnight. Then, the solvent was removed under vacuum, the residue was taken up with CH₂Cl₂ (10 mL). The layer was washed with a saturated solution of NaHCO₃(10 mL) and the aqueous layer was extracted three times with CH₂Cl₂ (10 mL). The combined organic layers were dried over MgSO₄, filtered and evaporated to dryness to afford COMPOUND 120 as a yellow oil (60 mg, 95%).

### Preparation of COMPOUND 121

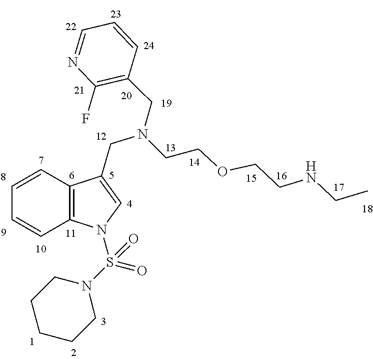 [SMILES: CCNCCOCCN(Cc1cccnc1F)Cc1cn(S(=O)(=O)N2CCCCC2)c2ccccc12]

To a solution of INTERMEDIATE 2.22. (277 mg, 0.47 mmol) in anhydrous DMF (8 mL) at room temperature, potassium carbonate (262 mg, 1.89 mmol, 4.0 eq.) was added. The reaction mixture was stirred and heated to 100° C. for 1 h. Then, ethyl iodide (42 μL, 0.52 mmol, 1.1 eq.) was added, the medium was stirred and heated at 100° C. overnight, under argon. The reaction mixture was cooled to room temperature, EtOAc (30 mL) and a saturated solution of NaHCO₃(30 mL) were added. The layers were separated and the aqueous layer was extracted twice with EtOAc (30 mL). The combined organic layers were dried over MgSO₄, filtered and evaporated to dryness. The residue (177 mg) was purified by preparative chromatography on silica gel, eluting with a mixture of CH₂Cl₂/MeOH NH₃ (7N) (96:4) to afford the desired ethylated compound as a colorless oil (36 mg, 25% yield) and starting material (140 mg).

To a solution of ethylated compound (36 mg, 0.058 mmol) in MeOH (1 mL) at room temperature, lithium hydroxide (1.5 mg, 0.064 mmol, 1.1 eq.) in water (0.5 mL) was added dropwise. The reaction mixture was stirred at room temperature overnight. Then, the solvent was removed under vacuum, the residue was taken up with CH₂Cl₂ (10 mL). The layer was washed with a saturated solution of NaHCO₃(10 mL) and the aqueous layer was extracted three times with CH₂Cl₂ (10 mL). The combined organic layers were dried over MgSO₄, filtered and evaporated to dryness to afford COMPOUND 121 as a yellow oil (28 mg, 93%).

### Preparation of COMPOUND 122

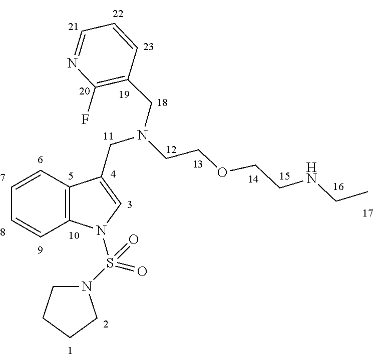 [SMILES: CCNCCOCCN(Cc1cccnc1F)Cc1cn(S(=O)(=O)N2CCCC2)c2ccccc12]

To a solution of INTERMEDIATE 2.23. (225 mg, 0.39 mmol) in anhydrous DMF (8 mL) at room temperature, potassium carbonate (218 mg, 1.57 mmol, 4.0 eq.) was added. The reaction mixture was stirred and heated to 100° C. for 1 h. Then, ethyl iodide (35 μL, 0.43 mmol, 1.1 eq.) was added, the medium was stirred and heated at 100° C. overnight, under argon. The reaction mixture was cooled to room temperature, EtOAc (30 mL) and a saturated solution of NaHCO₃(30 mL) were added. The layers were separated and the aqueous layer was extracted twice with EtOAc (30 mL). The combined organic layers were dried over MgSO₄, filtered and evaporated to dryness. The residue (210 mg) was purified by preparative chromatography on silica gel, eluting with a mixture of CH₂Cl₂/MeOH NH₃ (7N) (96:4) to afford the desired ethylated compound as a colorless oil (57 mg, 52% yield) and starting material (120 mg).

To a solution of ethylated compound (57 mg, 0.095 mmol) in MeOH (2 mL) at room temperature, lithium hydroxide (2.5 mg, 0.105 mmol, 1.1 eq.) in water (1 mL) was added dropwise. The reaction mixture was stirred at room temperature overnight. Then, the solvent was removed under vacuum, the residue was taken up with CH₂Cl₂ (10 mL). The layer was washed with a saturated solution of NaHCO₃(10 mL) and the aqueous layer was extracted three times with CH₂Cl₂ (10 mL). The combined organic layers were dried over MgSO₄, filtered and evaporated to dryness to afford COMPOUND 122 as a yellow oil (43 mg, 89%).

### Preparation of COMPOUND 123

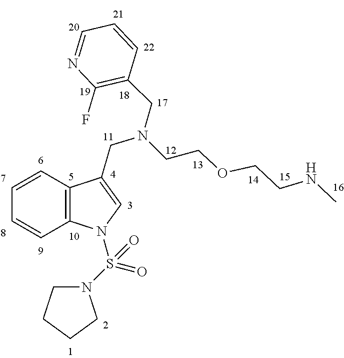 [SMILES: CNCCOCCN(Cc1cccnc1F)Cc1cn(S(=O)(=O)N2CCCC2)c2ccccc12]

To a solution of INTERMEDIATE 2.23. (226 mg, 0.395 mmol) in anhydrous DMF (8 mL) at room temperature, potassium carbonate (218 mg, 1.58 mmol, 4.0 eq.) was added. The reaction mixture was stirred and heated to 100° C. for 1 h. Then, methyl iodide (27 μL, 0.435 mmol, 1.1 eq.) was added, the medium was stirred and heated at 100° C. overnight, under argon. The reaction mixture was cooled to room temperature, EtOAc (30 mL) and a saturated solution of NaHCO₃(30 mL) were added. The layers were separated and the aqueous layer was extracted twice with EtOAc (30 mL). The combined organic layers were dried over MgSO₄, filtered and evaporated to dryness. The residue (276 mg) was purified by preparative chromatography on silica gel, eluting with a mixture of CH₂Cl₂/MeOH NH₃ (7N) (96:4) to afford the desired methylated compound as a colorless oil (45 mg, 35% yield) and starting material (100 mg).

To a solution of methylated compound (45 mg, 0.077 mmol) in MeOH (2 mL) at room temperature, lithium hydroxide (2.0 mg, 0.084 mmol, 1.1 eq.) in water (1 mL) was added dropwise. The reaction mixture was stirred at room temperature overnight. Then, the solvent was removed under vacuum, the residue was taken up with CH₂Cl₂ (10 mL). The layer was washed with a saturated solution of NaHCO₃(10 mL) and the aqueous layer was extracted three times with CH₂Cl₂ (10 mL). The combined organic layers were dried over MgSO₄, filtered and evaporated to dryness to afford COMPOUND 123 as a yellow oil (25 mg, 67%).

### Example 3: Biological In Vitro Protocol

1. Immunoassay: Binding Tests

Microtiter plates (96 wells) are covered with 100 μl of receptor per well, and incubated overnight at 4° C. The following day, after 2 washes in PBS/Tween20, the plates are saturated with 150 μl of PBS/BSA 2% per well, for 2 h at 37° C. Synthetic compounds are diluted in series (final concentrations: 100 to 0.78 μM) then a fixed amount of cytokine in PBS/BSA 1% is added to the compounds, in a final volume of 120 μl per well. The compound SPD304 is diluted in series (final concentrations: 100 to 3.125 μM), then a fixed amount of cytokine in PBS/BSA 1% is added to the SPD304 in a final volume of 120 μl per well. The cytokine is diluted in series (six concentrations) in a final volume of 120 μl per well. The cytokine is used in the absence of small molecule as well as the range of SPD304 as positive controls and the absence of cytokine and synthetic molecule as negative control. The different mixtures are incubated for 2 h at 37° C. 100 μl of each mixture are then transferred to the microtiter plate, respecting the plate plan previously defined and incubated overnight at 4° C. After three washes, 100 μl of biotinylated anti-cytokine secondary antibody are added to each well, then incubated for 2 h at 37° C. After three washes, 100 μl of Avidin-HRP (1/500th dilution) are added and incubated for 30 minutes at 37° C. After three washes, the plates are revealed by the addition of 100 μl of TMB per well and incubated for 10 minutes at room temperature before stopping the reaction by the addition of 50 μl per well of 1 M H2SO₄. The plates are then read with a spectrophotometer at 450 nm, measuring the optical density for each well.

This binding test is carried out with different cytokines: TNFa, IL-4, IL-6 and IL-13. For IL-6, the incubation of molecules with the cytokine also requires the presence of IL-6Ra at a final concentration of 200 ng/ml.

The final concentrations used for each of the cytokines are presented below:

TABLE 1
Different concentrations used in receptor, cytokine and biotinylated
antibody for TNFα, IL-4, IL-6 and IL-13 binding tests.

| | | | | Biotinylated |
|---|---|---|---|---|
| | Receptor | Cytokine | Range | antibody |
| | (ng/ml) | (ng/ml) | (ng/ml) | (ng/ml) |
| TNFα | 100 | 10 | 20 to 0.625 | 300 |
| IL-4 | 100 | 400 | 800 to 25 | 300 |
| IL-13 | 100 | 400 | 800 to 25 | 300 |
| IL-6* | 1000 (gp130) | 2 | 8 to 0.25 | 200 |

2. TNFa/TNFRI Interaction Displacement Test

Microtiter plates (96 wells) are covered with 100 μl per well of human TNFRI, at a concentration of 100 ng/ml, then incubated overnight at 4° C. The following day, after two washes in PBS/Tween20, the plates are saturated with 150 μl of PBS/BSA 2% for 2 h at 37° C.

TNFa is diluted in series (final concentrations: 80 ng/ml to 0.078125 ng/ml, reason for 2), then the compounds (final concentration: 50 μM) or SPD304 (final concentration: 20 μM) TNFa dilutions, in a final volume of 120 μI per well then incubated for 2 h at 37° C. The range of TNFa in the absence of synthetic molecule is used as well as the range of TNFa with 20 μM of SPD304 as positive controls and the absence of TNFa and of small synthetic molecule as negative control. 100 μl of each mixture are then transferred to the microtiter plate, respecting the established plate plan and incubated overnight at 4° C.

After three washes, 100 μl of biotinylated anti-cytokine secondary antibody are added to each well and incubated for 2 h at 37° C. After three washes, 100 Avl Avidin-HRP (dilution 1/500th) are added and incubated for 30 minutes at 37° C. After three washes, the plates are revealed by the addition of 100 μl of TMB and incubated for 10 minutes at room temperature before stopping the reaction by the addition of 50 μl of 1 M H2SO₄. The plates are then read with a spectrophotometer at 450 nm, measuring the optical density (OD) for each well.

3. Neutralization of the Biological Activity of TNFa on L929 Cells

In plates treated for cell culture (flat bottom), 40,000 L929 cells are deposited per well, in 100 μl of DMEM containing fetal calf serum (10%), L-Glutamine (2 mM) and a mixture Penicillin/Streptomycin (100 U/ml, 100 μg/ml). The plates are incubated overnight at 37° C., 5% CO₂. Small synthetic molecules are diluted in series (final concentration: from 100 to 0.78125 μM, reason for 2). 4 μg/ml of actinomycin and 300 μg/ml of TNFa are added to the small molecules in a final volume of 150 μl and incubated for 2 hours at 37° C., 5% CO2. 100 μl are then transferred to the cells in accordance with the plate plans and incubated for 24 hours at 37° C., 5% CO₂. The next day, the cell supernatant is removed by inversion of the plates, then 100 μl of MTT at 0.5 mg/ml are added to each well and incubated for 2 hours at 37° C., 5% CO₂. MTT is a reagent that is reduced by the mitochondrial succinate dehydrogenase of living cells, this reduction confers a purple coloration on the cells having metabolized it. MTT is removed by plate inversion and 200 μl of DMSO per well are added. After homogenization, the plates are read using a spectrophotometer at 570 nm, measuring the optical density for each well.

The percentage of survival is calculated according to the following equation 1:

$$\% survival = ( \frac{\begin{matrix}OD cells in the presence of the compound alone - \\ OD without cell\end{matrix}}{\begin{matrix}OD cells alone - \\ DO without cell\end{matrix}} )$$

The percentage of neutralization is calculated according to the following equation 2:

$$\% Neutra = ( \frac{\begin{matrix}OD cells in the presence of the compound and TNF α - \\ OD cells in the presence of TNF α alone\end{matrix}}{\begin{matrix}OD cells alone - \\ OD cells in the presence of TNF α alone\end{matrix}} ) \times 100$$

4. Neutralization of the Biological Activity of Different Cytokines on HEK-Blue™ Cells

The small synthetic molecules are diluted in series (final concentrations: 100 to 0.78125 μM, reason for 2). The cytokine of interest** is added to the small molecules and incubated for 2 hours at 37° C., 5% CO₂. After incubation, 50,000 HEK-Blue™** cells are added to 40 μl of DMEM containing fetal calf serum (10%), L-Glutamine (2 mM), a mixture of Penicillin/Streptomycin (100 U/ml, 100 μg/ml) and Normocin™ (100 μg/ml) for a final volume of 120 μl.

The plates are then incubated for 24 hours at 37° C., 5% C02. The next day, 20 μl of each supernatant are taken and diluted in 180 μl of QUANTI-Blue™ in order to reveal the potential neutralization of the biological activity of the cytokine by small molecules. On the other hand, 45 μl of XTT is added to each well in order to reveal the possible toxicity of the compound. XTT is a reagent which is reduced by the mitochondrial succinate dehydrogenase of living cells, this reduction confers a yellow/orange coloration on the cells having metabolized it. The different mixtures are incubated for 2 hours (QUANTI-Blue™) or 3 hours (XTT) at 37° C., 5% CO₂. The optical density for each plate is then read using a spectrophotometer at 620 nm (QUANTI-Blue plates) or 450 nm (XTT plates).

** This cell test was carried out with different lines of HEK-Blue cells: HEK-Blue™ TNFa cells, HEK-Blue™ IL-4/IL-13 cells and HEK-Blue™ IL-6 cells. Depending on the line, the corresponding cytokine was used.

The final concentrations used for each of the cytokines are presented below:

TABLE 2
Different concentrations of TNFa, IL-4, IL-6 or IL-13
used for the HEK-Blue ™ cell tests.

| | TNFα | IL-4 | IL-13 | IL-6 |
|---|---|---|---|---|
| | (pg/ml) | (pg/ml) | (pg/ml) | (pg/ml) |
| Concentration | 400 | 400 | 400 | 1000 |
| used | | | | |

The percentage of survival is calculated according to the following equation 3:

$$\% survical = ( \frac{\begin{matrix}OD cells in the presence of the compound alone - \\ OD without cell\end{matrix}}{OD cells alone - DO without cell} ) \times 100$$

The percentage of neutralization is calculated according to the following equation 4:

$$\% Neutra = [ 100 - ( \frac{\begin{matrix}OD cells in the presence of the compound of the cytokine - \\ OD cells alone\end{matrix}}{\begin{matrix}OD cells in the presence of the cytokine alone - \\ OD cells alone\end{matrix}} ) ]$$

5. Results

The results are shown in the following table:

| | | | Displacement | BINDING IL4 | | BINDING IL13 | | |
|---|---|---|---|---|---|---|---|---|
| | | | EC50 TNFa in | % | | % | | BINDING |
| | BINDING | | presence of | Neutra | | Neutra | | IL6 |
| Com- | TNF IC50 | Displace- | compound | at 100 | IC₅₀ | at 100 | IC₅₀ | % Neutra |
| pound | (μM) | ment (%) | (ng/ml) | μM | (μM) | μM | (μM) | at 100 μM |
| 1 | 11.9 | 178 | SPD304 = 18.9 | 97.5 | 4.1 | 94.6 | 23.5 | 5.5 |
| | | | Compound = | | | | | |
| | | | 28.0 | | | | | |
| | | | Difference = | | | | | |
| | | | 9.1 | | | | | |
| 2 | 19.9 | 183 | SPD304 = 18.9 | 97.8 | 3.3 | 97.1 | 1.4 | 2 |
| | | | Compound = | | | | | |
| | | | 33.8 | | | | | |
| | | | Difference = | | | | | |
| | | | 14.9 | | | | | |
| 3 | 4.6 | 196 | SPD304 = 11 | 96.4 | 6.6 | 96.9 | | 5 |
| | | | Compound = | | | | | |
| | | | 18.4 | | | | | |
| | | | Difference = | | | | | |
| | | | 7.4 | | | | | |
| 4 | 21.3 | 221 | SPD304 = 11 | 98.3 | 4 | 92.2 | 9.9 | 5.2 |
| | | | Compound = | | | | | |
| | | | 20 | | | | | |
| | | | Difference = 9 | | | | | |
| 5 | 11.1 | 194 | SPD304 = 16.1 | 96.9 | 7.8 | 84.9 | >50 | 2.1 |
| | | | Compound = | | | | | |
| | | | 24.4 | | | | | |
| | | | Difference = | | | | | |
| | | | 8.3 | | | | | |
| 6 | 16.6 | 153 | SPD304 = 16.1 | 95.7 | 3.6 | 98.6 | 3.1 | 8.7 |
| | | | Compound = | | | | | |
| | | | 19.5 | | | | | |
| | | | Difference = | | | | | |
| | | | 3.4 | | | | | |
| 7 | 9.4 | 146 | SPD304 = 19.7 | 97.3 | 8.5 | 86.2 | >50 | 3.3 |
| | | | Compound = | | | | | |
| | | | 27.5 | | | | | |
| | | | Difference = | | | | | |
| | | | 7.8 | | | | | |
| 8 | 12.2 | 168 | SPD304 = 19.7 | 98.1 | 5.3 | 81.7 | >50 | 3.3 |
| | | | Compound = | | | | | |
| | | | 30.6 | | | | | |
| | | | Difference = | | | | | |
| | | | 10.9 | | | | | |
| 9 | 7.3 | 175 | SPD304 = 12 | 94.4 | 8 | 59.7 | >50 | 21 |
| | | | Compound = | | | | | |
| | | | 16.3 | | | | | |
| | | | Difference = | | | | | |
| | | | 4.3 | | | | | |
| 10 | 23 | 121 | SPD304 = 12 | 96.4 | 5.5 | 91.1 | >50 | 10.7 |
| | | | Compound = | | | | | |
| | | | 13 | | | | | |
| 11 | 4.4 | 215 | SPD304 = 16.3 | 97.6 | 6.2 | 90.2 | >50 | −3 |
| | | | Compound = | | | | | |
| | | | 38.4 | | | | | |
| 12 | 6.8 | 172 | SPD304 = 16.3 | 96.3 | 12.3 | 58.2 | >50 | 1.6 |
| | | | Compound = | | | | | |
| | | | 30.5 | | | | | |
| | | | Difference = | | | | | |
| | | | 14.2 | | | | | |
| 13 | 4.3 | 186 | SPD304 = 11.2 | 97.9 | 5 | 85.2 | >50 | 8.9 |
| | | | Compound = | | | | | |
| | | | 17.6 | | | | | |
| | | | Difference = | | | | | |
| | | | 6.4 | | | | | |
| 14 | 11.8 | 149 | SPD304 = 11.2 | 98.4 | 5.3 | 91.2 | >50 | 2.8 |
| | | | Compound = | | | | | |
| | | | 13.7 | | | | | |
| 15 | 8.1 | 167 | SPD304 = 18.1 | 99 | 6.3 | 94.8 | >50 | 11.5 |
| | | | Compound = | | | | | |
| | | | 23.2 | | | | | |
| | | | Difference = | | | | | |
| | | | 5.1 | | | | | |
| 16 | 5.4 | 165 | SPD304 = 18.1 | 99.5 | 4.8 | 88.2 | >50 | 15.7 |
| | | | Compound = | | | | | |
| | | | 22.8 | | | | | |
| | | | Difference = | | | | | |
| | | | 4.7 | | | | | |
| 17 | 1.1 | 133 | SPD304 = 17.2 | 97.9 | 5 | 87.7 | >50 | 20.7 |
| | | | Compound = | | | | | |
| | | | 20.8 | | | | | |
| 18 | 2.2 | 145 | SPD304 = 17.2 | 98.4 | 5.3 | 94.2 | 80.9 | 16.3 |
| | | | Compound = | | | | | |
| | | | 23.2 | | | | | |
| | | | Difference = 6 | | | | | |
| 19 | 10.9 | 169 | SPD304 = 18.9 | 99 | 6.3 | 98.1 | >50 | 12.6 |
| | | | Compound = | | | | | |
| | | | 28.8 | | | | | |
| | | | Difference = | | | | | |
| | | | 9.9 | | | | | |
| 20 | 19.7 | 88 | SPD304 = 18.9 | 99.5 | 4.8 | 97.5 | 6.2 | 18.5 |
| | | | Compound = | | | | | |
| | | | 14.9 | | | | | |
| 21 | 14.1 | 141 | SPD304 = 14.3 | 98 | 2.1 | 94.6 | 19.5 | 3.7 |
| | | | Compound = | | | | | |
| | | | 19.2 | | | | | |
| | | | Difference = | | | | | |
| | | | 4.9 | | | | | |
| 22 | 16.9 | 120 | SPD304 = 14.3 | 98.4 | 4.1 | 95.2 | >50 | -1.3 |
| | | | Compound = | | | | | |
| | | | 15.1 | | | | | |
| 23 | 9.3 | 140 | SPD304 = 14.1 | 98.1 | 4.9 | 85.8 | >50 | 5.6 |
| | | | Compound = | | | | | |
| | | | 19.6 | | | | | |
| | | | Difference = | | | | | |
| | | | 5.5 | | | | | |
| 24 | 13.5 | 158 | SPD304 = 14.1 | 98.8 | 3.1 | 85.3 | >50 | 3.6 |
| | | | Compound = | | | | | |
| | | | 20.4 | | | | | |
| | | | Difference = | | | | | |
| | | | 6.3 | | | | | |
| 25 | 22.6 | 99 | SPD304 = 19.9 | 94.6 | 7.8 | 61.2 | >50 | −15.3 |
| | | | Compound = | | | | | |
| | | | 18.6 | | | | | |
| 26 | 14.1 | 107 | SPD304 = 19.9 | 95.5 | 8.4 | 66 | >50 | −17.2 |
| | | | Compound = | | | | | |
| | | | 20.5 | | | | | |
| 27 | 13.8 | 121 | SPD304 = 20.2 | 96.3 | 7.8 | 53.9 | >50 | −12 |
| | | | Compound = | | | | | |
| | | | 18.2 | | | | | |
| 28 | 14.5 | 125 | SPD304 = 20.2 | 96.6 | 8.1 | 38.3 | >50 | 12.4 |
| | | | Compound = | | | | | |
| | | | 19.6 | | | | | |
| 29 | 18.3 | 103 | SPD304 = 16.9 | 96.4 | 6.2 | 86.8 | >50 | 17.3 |
| | | | Compound = | | | | | |
| | | | 15.5 | | | | | |
| 30 | 19.9 | 106 | SPD304 = 16.9 | 97.3 | 4 | 93.6 | >50 | 16.8 |
| | | | Compound = | | | | | |
| | | | 15.4 | | | | | |
| 31 | 13.4 | 120 | SPD304 = 16.3 | 96 | 5.7 | 81 | >50 | 1.7 |
| | | | Compound = | | | | | |
| | | | 15.2 | | | | | |
| 32 | 15.9 | 125 | SPD304 = 16.3 | 97.7 | 3.9 | 76.3 | >50 | 15.5 |
| | | | Compound = | | | | | |
| | | | 16 | | | | | |
| 33 | 4.6 | 273 | SPD304 = 8.8 | 96 | 4.1 | 95.4 | 10.5 | −11.8 |
| | | | Compound = | | | | | |
| | | | 20.4 | | | | | |
| | | | Difference = | | | | | |
| | | | 11.6 | | | | | |
| 34 | 7.2 | 256 | SPD304 = 8.8 | 95.4 | 5.9 | 97.9 | >50 | −4.8 |
| | | | Compound = | | | | | |
| | | | 18.7 | | | | | |
| | | | Difference = | | | | | |
| | | | 9.9 | | | | | |
| 35 | 4.6 | 181 | SPD304 = 13.7 | 96.1 | 5.6 | 97.1 | >50 | −21.5 |
| | | | Compound = | | | | | |
| | | | 18.4 | | | | | |
| 36 | 3.9 | 165 | SPD304 = 13.7 | 97.4 | 3.4 | 95 | 19.8 | −1.8 |
| | | | Compound = | | | | | |
| | | | 16.9 | | | | | |
| 37 | 19.1 | 123 | SPD304 = 15.3 | 96.3 | 4 | 96.4 | 7.4 | 20.4 |
| | | | Compound = | | | | | |
| | | | 16.4 | | | | | |
| 38 | >50 | 139 | SPD304 = 15.3 | 95.7 | 3.9 | 98.9 | 1.5 | 24.9 |
| | | | Compound = | | | | | |
| | | | 17.8 | | | | | |
| 39 | 14.7 | 159 | SPD304 = 16.2 | 96 | 4.7 | 94.8 | >50 | 23.7 |
| | | | Compound = | | | | | |
| | | | 20.3 | | | | | |
| | | | Difference = | | | | | |
| | | | 4.2 | | | | | |
| 40 | 19.9 | 157 | SPD304 = 16.2 | 96.4 | 2.7 | 94.2 | 11.2 | 16.4 |
| | | | Compound = | | | | | |
| | | | 20.3 | | | | | |
| | | | Difference = | | | | | |
| | | | 4.2 | | | | | |
| 41 | 7 | 152 | SPD304 = 15.0 | 94.7 | 4.8 | 87.6 | 9.2 | 0 |
| | | | Compound = | | | | | |
| | | | 19.7 | | | | | |
| | | | Difference = | | | | | |
| | | | 4.7 | | | | | |
| 42 | 15.6 | 177 | SPD304 = 15.0 | 94.4 | 6.4 | 86.3 | 21.3 | 0 |
| | | | Compound = | | | | | |
| | | | 23.3 | | | | | |
| | | | Difference = | | | | | |
| | | | 8.3 | | | | | |
| 43 | 8.8 | 87 | | 93.2 | 6.7 | 85.1 | >50 | 0 |
| 44 | 8.6 | 109 | | | | | | |
| 45 | 10.4 | 114 | | 90.5 | 7.6 | 86.5 | >50 | 0 |
| 46 | 10.8 | 124 | | | | | | |
| 47 | 8.9 | 99 | | 93.3 | 5.5 | 93.1 | 17.8 | 0 |
| 48 | 13.7 | 91 | | 91.2 | 7.0 | 89.9 | 17.8 | 1 |
| 49 | 8.2 | 127 | | 89.5 | 5.6 | 88.8 | 23.9 | 0 |
| 50 | 7.2 | 138 | | 90.6 | 6.3 | 90.3 | 26.2 | 0 |
| 51 | 7.9 | 153 | | 87.2 | 6.7 | 89.4 | 25.9 | 0 |
| 52 | 9.1 | 145 | | 91.7 | 5.0 | 88.1 | 15.8 | 0 |
| 53 | 8.4 | 115 | | 92.7 | 5.2 | 91.6 | 15.2 | 0 |
| 54 | 11.7 | 85 | | 88.4 | 3.0 | 92.5 | 47.4 | 0 |
| 55 | 7.8 | 172 | | | | | | |
| 56 | 13.2 | 124 | | 91.9 | 2.6 | 93.4 | 10.2 | 0 |
| 57 | 9.4 | 113 | | | | | | |
| 58 | 6.7 | 126 | | 92.5 | 2.1 | 89.1 | 10.4 | 1 |
| 59 | 35.8 | 78 | | 89.4 | 43.9 | 59.3 | >50 | 0 |
| 60 | >50 | 2 | | 36.4 | 23.4 | 0 | / | 0 |
| 61 | 29.8 | 64 | | | | | | |
| 62 | 26.8 | 105 | | 81.5 | 7.8 | 67.9 | 38.5 | 0 |
| 63 | 26.5 | 88 | | 85.9 | 16.0 | 55.4 | >50 | 0 |
| 64 | 8.9 | 197 | | 90.7 | 5.1 | 83.9 | 28.2 | 0 |
| 65 | 5.9 | 224 | | 86.5 | 5.5 | 79.3 | >50 | 0 |
| 66 | 6.4 | 215 | | 89.1 | 4.7 | 85.1 | 36.0 | 3 |
| 67 | 6.7 | 219 | | | | | | |
| 68 | 10 | 219 | | 86.0 | 4.2 | 81.6 | 17.3 | 0 |
| 69 | 12.1 | 121 | | 93.6 | 5.6 | 79.7 | >50 | 0 |
| 70 | 11.5 | 126 | | 92.4 | 13.5 | 69.6 | >100 | 1 |
| 71 | 9.8 | 199 | | 92.4 | 6.0 | 89.7 | >50 | 2 |
| 72 | 9.7 | 208 | | 89.8 | 8.1 | 92.1 | >50 | 3 |
| 73 | 8.4 | 161 | | 91.0 | 6.0 | 87.2 | 14.9 | 0 |
| 74 | 6.6 | 160 | | 91.9 | 5.7 | 87.3 | >50 | 0 |
| 75 | 11.2 | 147 | | 92.0 | 8.5 | 85.8 | >50 | 0 |
| 76 | 7.9 | 154 | | 92.8 | 4.6 | 90.0 | 8.3 | 1 |
| 77 | >50 | 35 | | 13.7 | >100 | 0 | / | 0 |
| 78 | >50 | 11 | | 33.5 | >50 | 0 | / | 0 |
| 79 | >50 | 3 | | 31.0 | >100 | 0 | / | 1 |
| 80 | >50 | 0 | | 29.1 | >100 | 0 | / | 0 |
| 81 | >50 | 0 | | 39.3 | >50 | 0 | / | 1 |
| 82 | >50 | 18 | | 27.9 | >100 | 0 | / | 5 |
| 83 | 21 | 93 | | 84.6 | >50 | 1 | / | 0 |
| 84 | 21.2 | 111 | | 0 | / | 0 | / | 3 |
| 85 | 44.3 | 45 | | 36.6 | >100 | 0 | / | 3 |
| 86 | 5 | 188 | | 92.6 | 4.9 | 87.9 | >50 | 6 |
| 87 | 18.8 | 28 | | 95.5 | 4.4 | 94.2 | 8.2 | 6 |
| 88 | >50 | 5 | | 69.0 | 5.3 | 8 | / | 9 |
| 89 | 15 | 63 | | 95.6 | 2.0 | 94.7 | 2.6 | 0 |
| 90 | 15.5 | 137 | | 94.0 | 5.2 | 69.7 | >50 | 0 |
| 91 | 27.5 | 87 | | 87.5 | 16.4 | 35.8 | >50 | 0 |
| 92 | 8.4 | 117 | | 92.3 | 7.7 | 72.0 | >50 | 2 |
| 93 | 11.2 | 160 | | 89.8 | 2.8 | 91.3 | 7.8 | 0 |
| 94 | 19 | 101 | | 95.8 | 34.5 | 75.2 | >50 | 0 |
| 95 | >50 | 16 | | 75.3 | >50 | 0 | / | 0 |
| 96 | >50 | 26 | | 74.5 | 62.3 | 0 | / | 1 |
| 97 | 10.5 | 153 | | 94.6 | 6.6 | 73.9 | >50 | 0 |
| 98 | 9.9 | 127 | | 93.4 | 6.2 | 90.2 | >50 | 0 |
| 99 | 8.2 | 173 | | 87.4 | 4.4 | 91.8 | >50 | 0 |
| 100 | 8 | 128 | | 87.7 | 8.6 | 83.0 | >50 | 0 |
| 101 | 17.4 | 152 | | 81.9 | 7.9 | 56.3 | 35.5 | 0 |
| 102 | 7.2 | 138 | | 83.2 | 6.3 | 39.2 | >25 | 0 |
| 103 | 10.2 | 159 | | 91.1 | 10.5 | 89.1 | 38.9 | 0 |
| 104 | 9.2 | 123 | | 88.2 | 13.2 | 79.8 | >50 | 0 |
| 105 | 7.5 | 162 | | 91.2 | 11.5 | 74.3 | >50 | 0 |
| 106 | 11.5 | 132 | | 84.1 | 14.6 | 32.1 | >50 | 0 |
| 107 | 7.2 | 169 | | 74.9 | 5.1 | 66.1 | 25.9 | 0 |
| 108 | 9.6 | 165 | | 76.7 | 9.1 | 58.8 | 40.4 | 0 |
| 109 | 11.5 | 207 | | 86.3 | 9.6 | 7.8 | / | 1 |
| 110 | 17 | 83 | | | | | | |
| 111 | 6 | 224 | | | | | | |
| 112 | 13 | 245 | | | | | | |

### Example 4: Biological In Vivo Protocol: LPS/D-Galactosamine Induced Lethal Shock

7 week-old Balb/C mice (n=10 per group) were force-fed with 100 μl of a solution of DMSO containing 5 mg of compounds 33, 65 or 71 eight hours before receiving an intraperitoneal injection of 200 μl of PBS containing 0.2 μg of LPS and 20 mg of D-Galactosamine. A negative control group (vehicle) was force-fed with 100 μl of a solution of DMSO alone eight hours before to be injected with 200 μl of LPS/D-Galactosamine solution. Mice survival was monitored for 48 hours after the injection of LPS/D-Galactosamine. The results show that treatment with compound 33 protects against hepatic shock induced by LPS/D-Galactosamine with a maximum efficacy (100% survival). Compounds 65 and 71 exhibit significant protection with with 60% survival 48 hours after hepatic shock induction (FIG. 1).

1. A compound of the following formula (I): 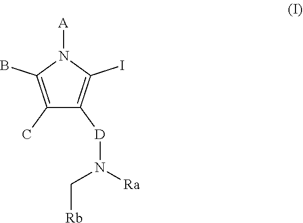
wherein:
A is:
a hydrogen atom;
a linear or branched C₁₋₁₀ alkyl or heteroalkyl group, C₂₋₁₁ alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of:
a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH, —R₁COR₁′, —R₁COOR₁′, —R₁CONHR₁′, —R₁NHCOR₁′ wherein R₁ is selected form the group consisting of a covalent bond, or a C₁₋₅ alkyl, heteroalkyl, alkenyl or heteroalkenyl group, and R₁′ is selected from the group consisting of a hydrogen atom, a C₁₋₅ alkyl, heteroalkyl or haloalkyl group, a C₂₋₆ alkenyl, heteroalkenyl or haloalkenyl group, a C₃₋₁₂ cycloalkyl, heterocycloalkyl, aryl, or heteroaryl group, —NO₂, and —SO₃H; or
a SO₂A′ group wherein A′ is selected from the group consisting of a C₁₋₅ alkyl, heteroalkyl, alkenyl or heteroalkenyl group and a C₃₋₁₂ cycloalkyl, cycloalkenyl, heterocycloalkyl, heterocycloalkenyl, aryl or heteroaryl group which may be substituted by at least one group selected from the group consisting of:
a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH, —R₁COR₁′, —R₁COOR₁′, —R₁CONHR₁′, —R₁NHCOR₁′ wherein R₁ and R₁′ are as defined above;
B and C, which may be the same or different, independently represent:
a hydrogen atom; or
a linear or branched C₁₋₁₀ alkyl or heteroalkyl group, C₂₋₁₁ alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of:
a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH, —R₁COR₁′, —R₁COOR₁′, —R₁CONHR₁′, —R₁NHCOR₁′ wherein R₁ and R₁′ are as defined above; or
B and C are joined such that when taken together with the carbon atoms to which they are attached they form a C₃₋₁₂ cycloalkyl, cycloalkenyl heterocycloalkyl, heterocycloalkenyl, aryl or heteroaryl ring which may be substituted by at least one group selected from the group consisting of:
a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, a heteroatom selected form N, O or S, —COOH, —R₁COR₁′, —R₁COOR₁′, —R₁CONHR₁′, —R₁NHCOR₁′ wherein R₁ and R₁′ are as defined above;
D is —CH₂, —C(O), —NHC(O)—, SO₂, or —NHSO₂;
Ra is R₂—NR₃R₄ wherein:
R₂ is a linear or branched C₁₋₅ alkyl or heteroalkyl group, or C₂₋₆ alkenyl, or heteroalkenyl group;
R₃ is:
a hydrogen atom, or
a linear of branched C₁₋₅ alkyl or heteroalkyl group, a C₂₋₆ alkenyl or heteroalkenyl group, or a C₃₋₁₂ cycloalkyl, cycloalkenyl, heterocycloalkyl, heterocycloalkenyl, cycloalkylalkyl, cycloalkylalkenyl, heterocycloalkylalkyl, heterocycloalkylalkenyl, aryl, heteroaryl, arylalkyl, or heteroarylalkyl group, each of which may be substituted by at least one group selected from the group consisting of a trifluoromethyl, a C₁₋₅ alkyl or alkoxy group, a C₂₋₆ alkenyl group, a halogen atom including F, Cl, Br, and I, a hydroxy group, an amino group, and —COOH;
R₄ is:
a hydrogen atom;
a linear or branched C₁₋₁₀ alkyl or heteroalkyl group, C₂11 alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of:
a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH;
—SO₂NR₅R₆ or —C(O)NR₅R₆ wherein R₅ and R₆, which may be the same or different, independently represent a hydrogen atom, a C₁₋₅ linear of branched alkyl, alkenyl, heteroalkyl or heteroalkenyl, group, or R₅ and R₆ are joined such that when taken together with the nitrogen atom to which they are attached they form a C₃₋₁₂ cycloalkyl, cycloalkenyl, heterocycloalkyl, heterocycloalkenyl, aryl or heteroaryl ring;
—SO₂R₇, wherein R₇ is a hydrogen atom, a C₁₋₅ linear of branched alkyl, alkenyl, heteroalkyl or heteroalkenyl group; a C₃₋₁₂ cycloalkyl, cycloalkenyl, heterocycloalkyl, heterocycloalkenyl, aryl or heteroaryl group which may be substituted by a C₁₋₅ alkyl group, a halogen atom including F, Cl, Br, and I, an amino group, or —NO₂; or
—C(O)R₇ wherein R₇ is as defined above; or
Ra is a compound of the following formula: 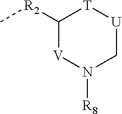
wherein
R₂ is as defined above;
T, U, V which are the same or different, independently represent —(CH₂)ₙ— wherein n is an integer from 1 to 3, or an heteroatom selected from the group consisting of 0, N, and S;
R₈ is:
a hydrogen atom; or
a linear or branched C₁₋₁₀ alkyl or heteroalkyl group, C₂₋₁₁ alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of:
a trifluoromethyl, a C₁₋₅ alkyl, alkoxy or alkylamino group, a C₂₋₆ alkenyl, alkenyloxy or alkenylamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH or R₁COR₁′, —R₁COOR₁′, —R₁CONHR₁′, —R₁NHCOR₁′ wherein R₁ and R₁′ are as defined above;
Rb is 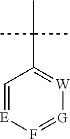
wherein
E, F, G and W which may be the same or different, independently represent —N—, —CH—, —N-oxide, —CNO₂, —CN(R₁″)₄ wherein R₁″ is a C₁₋₄ alkyl group, —CF—, —C¹⁸F or —C¹⁹F; or 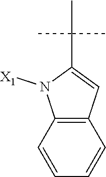
wherein
X1 is selected from a SO₂A′ group with A′ as defined above or an aryl or heteroaryl group optionally substituted by at least one group selected from the group consisting of a trifluoromethyl and a C₁₋₅ alkyl group;
I is —H, —OH, —SH, —CF₃, a halogen atom including F, Cl, Br, and I, a linear or branched C₁₋₁₀ alkyl, heteroalkyl, or haloalkyl group, or C₂₋₁₁ alkylene, heteroalkylene, or haloalkylene group, or a pharmaceutically acceptable salt or hydrate thereof.

2. The compound or pharmaceutically acceptable salt or hydrate thereof according to claim 1, of the following formula (III): 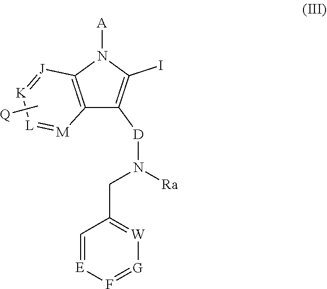
wherein
A, I, D, Ra, E, F, G and W are as defined above;
J, K, L, M, which are the same or different, independently represent —N—, —O—, —S—, or —CH—;
Q is:
a hydrogen atom; or
a linear or branched C₁₋₁₀ alkyl or heteroalkyl group, C₂₋₁₁ alkenyl, or heteroalkenyl, or a C₃₋₁₂ cycloalkyl, cycloalkenyl, heterocycloalkyl, heterocycloalkenyl, aryl or heteroaryl group, each of which may be substituted by at least one group selected from the group consisting of:
a trifluoromethyl, a C₁₋₅ alkyl, alkoxy or alkylamino group, a C₂₋₆ alkenyl, alkenyloxy or alkenyamino group, a C₃₋₁₂ cycloalkyl or heterocycloalkyl group, a C₃₋₁₂ aryl, arylamino or heteroaryl group, a halogen atom including F, Cl, Br, and I, a hydroxy group, an amino group, —COOH, R₁COR₁′, —R₁COOR₁′, —R₁CONHR₁′, —R₁NHCOR₁′ wherein R₁ and R₁′ are as defined above.

3. The compound or pharmaceutically acceptable salt or hydrate thereof according to claim 1, wherein Ra is selected from the group consisting of:

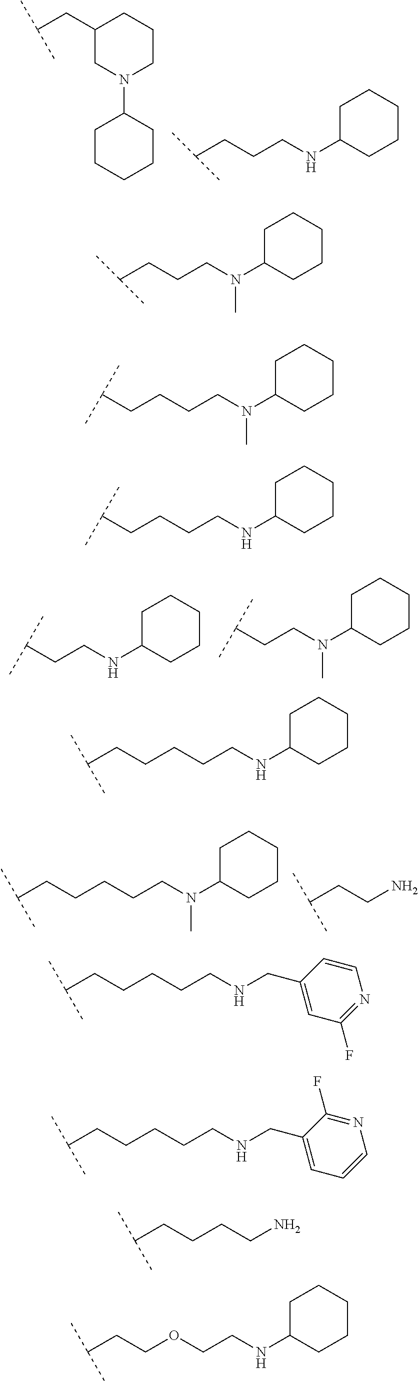

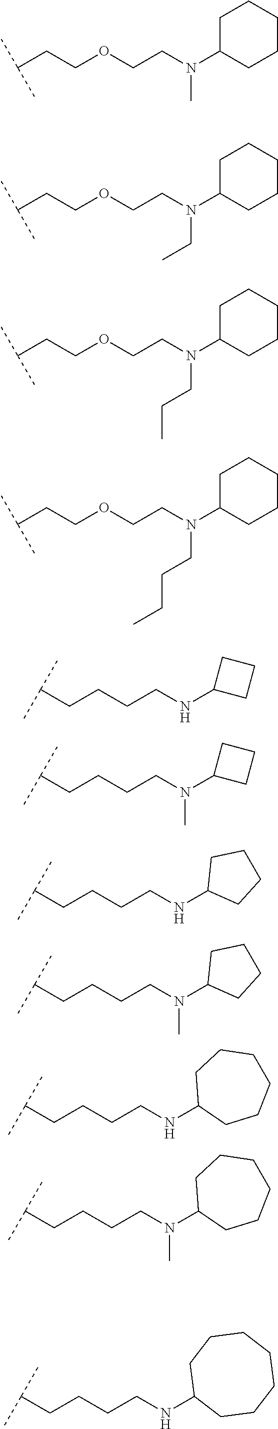

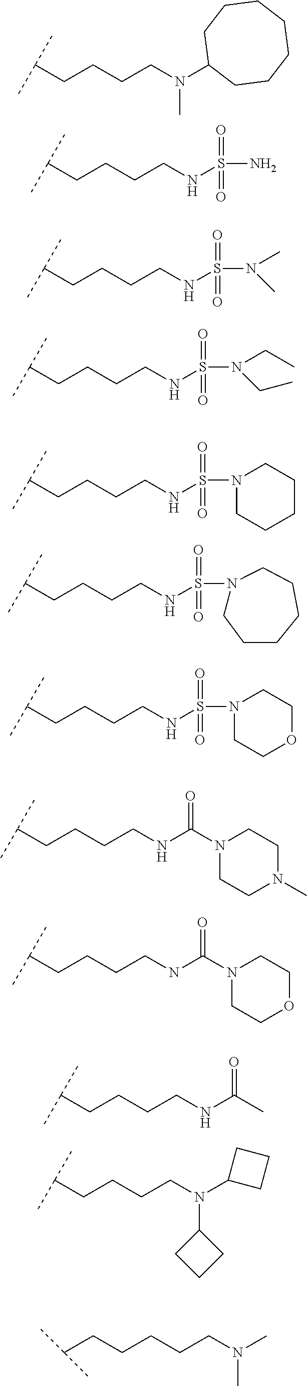

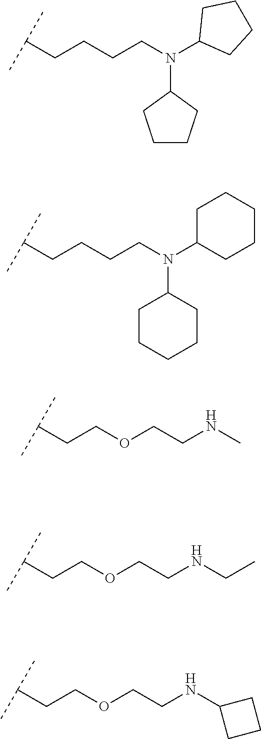

4. The compound or pharmaceutically acceptable salt or hydrate thereof according to claim 1, wherein A is:
a linear or branched alkyl, heteroalkyl, or haloalkyl group having 1 to 10 carbon atoms; a cycloalkyl, heterocycloalkyl, aryl or heteroaryl group having 2 to 12 carbon atoms, each of which may be substituted by at least one group selected from the group consisting of a trifluoromethyl, or a C₁₋₅ alkyl group,
a SO₂A′ group wherein A′ is selected from the group consisting of a cycloalkyl, heterocycloalkyl, aryl or heteroaryl group having 2 to 12 carbon atoms, which may be substituted by at least one group selected from the group consisting of a trifluoromethyl, or a C₁₋₅ alkyl group,

5. The compound or pharmaceutically acceptable salt or hydrate thereof according to claim 1, wherein I is a hydrogen atom and/or D is CH₂.

6. The compound or pharmaceutically acceptable salt or hydrate thereof according to claim 1, wherein at least one of E, F, G and W represents —C¹⁸F or —C¹⁹F.

7. The compound or pharmaceutically acceptable salt or hydrate thereof according to claim 1, selected from the group consisting of:

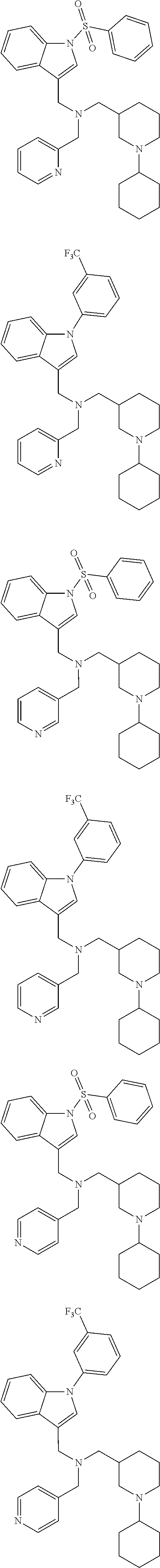

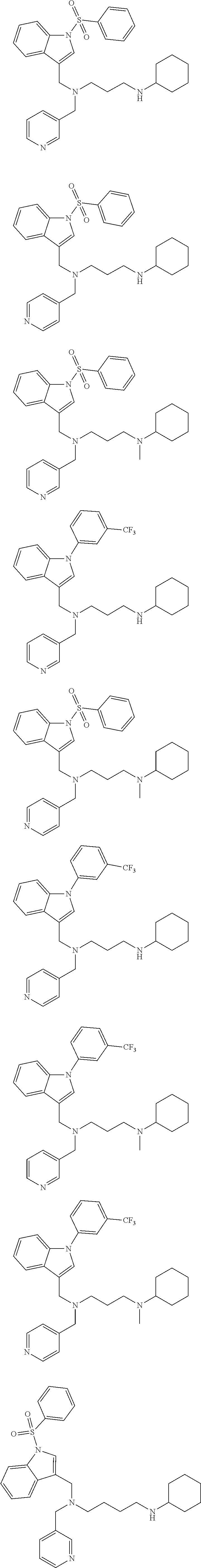

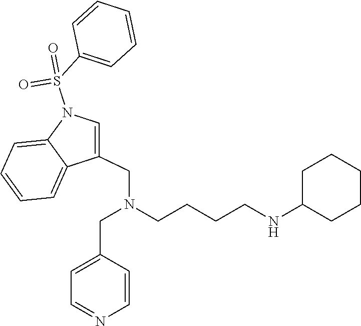 [SMILES: O=S(=O)(c1ccccc1)n1cc(CN(CCCCNC2CCCCC2)Cc2ccncc2)c2ccccc21]

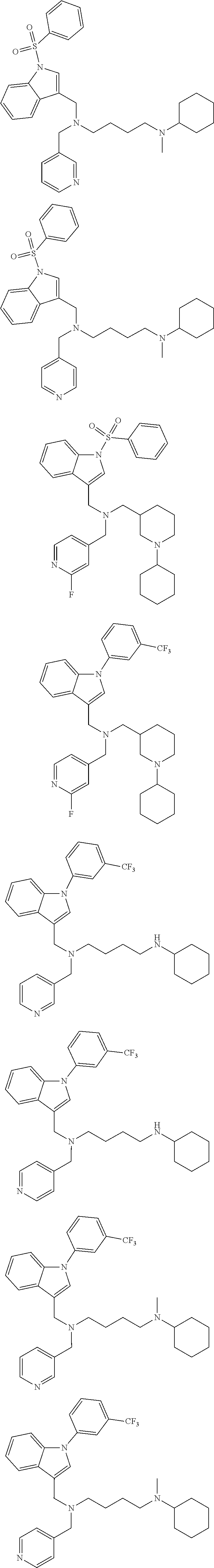

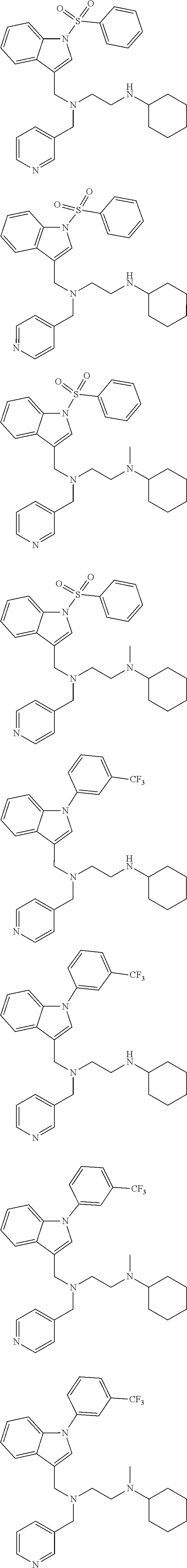

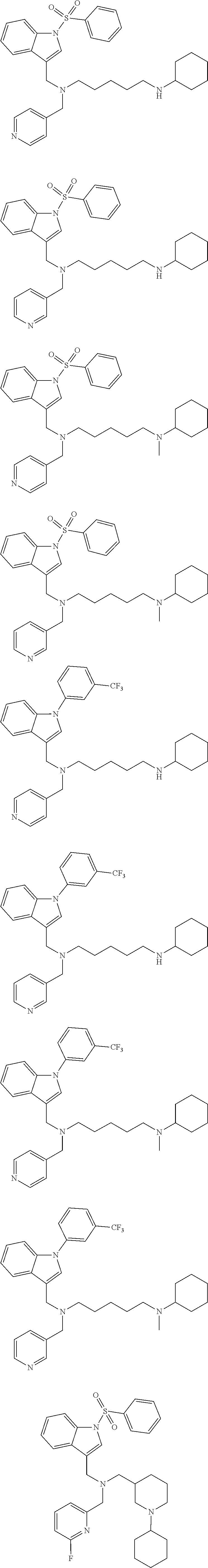

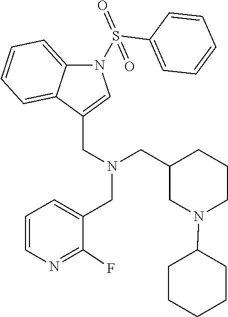 [SMILES: O=S(=O)(c1ccccc1)n1cc(CN(Cc2cccnc2F)CC2CCCN(C3CCCCC3)C2)c2ccccc21]

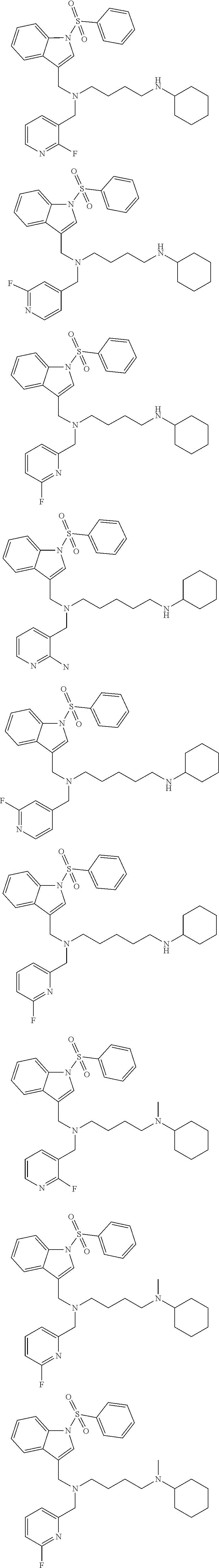

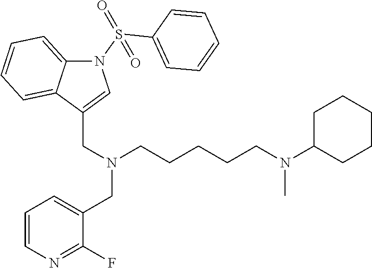 [SMILES: CN(CCCCCN(Cc1cccnc1F)Cc1cn(S(=O)(=O)c2ccccc2)c2ccccc12)C1CCCCC1]

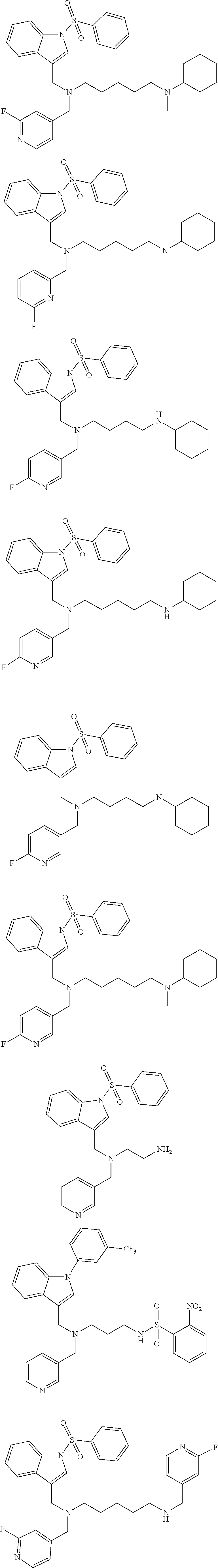

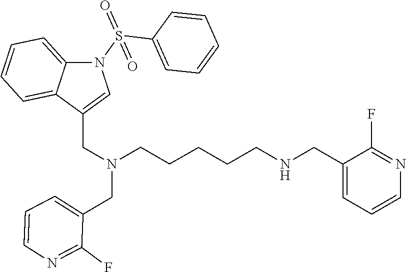 [SMILES: O=S(=O)(c1ccccc1)n1cc(CN(CCCCCNCc2cccnc2F)Cc2cccnc2F)c2ccccc21]

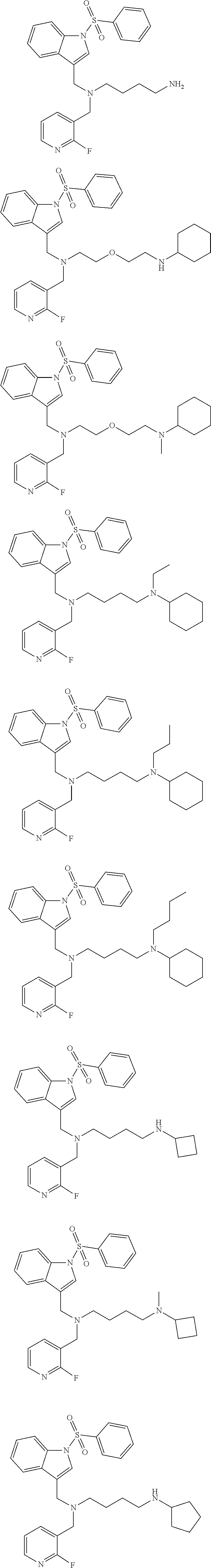

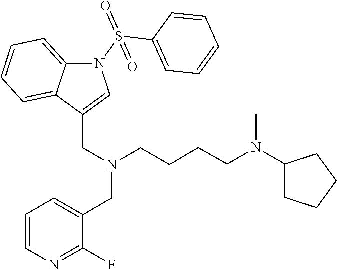 [SMILES: CN(CCCCN(Cc1cccnc1F)Cc1cn(S(=O)(=O)c2ccccc2)c2ccccc12)C1CCCC1]

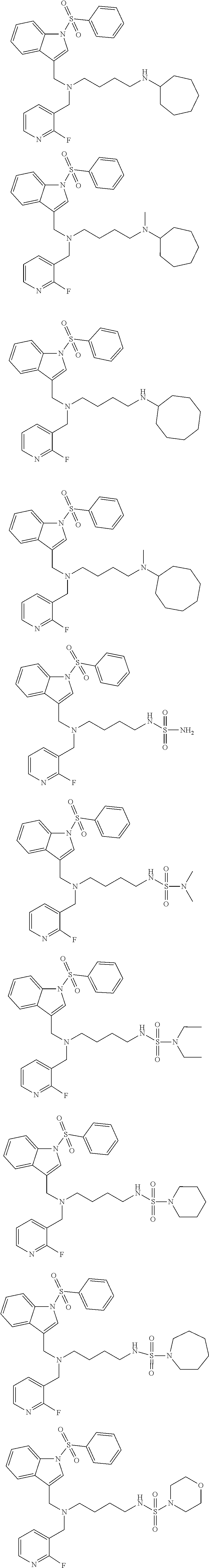

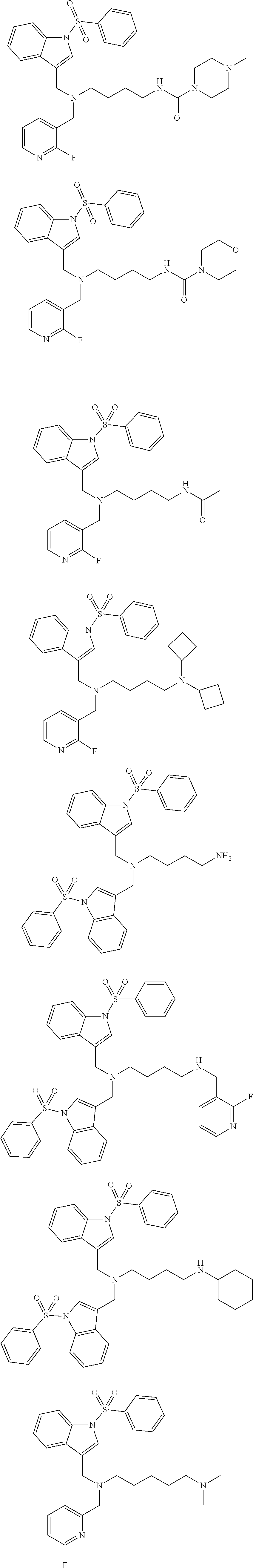

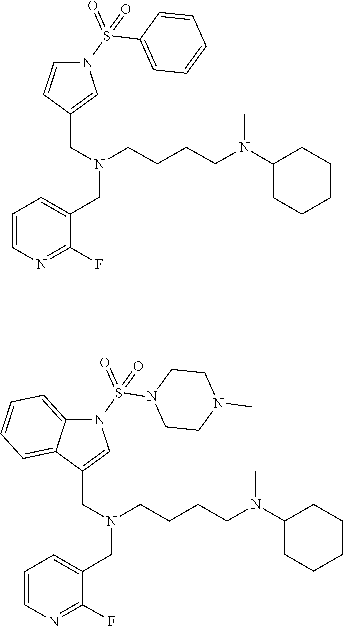 [SMILES: CN(CCCCN(Cc1ccn(S(=O)(=O)c2ccccc2)c1)Cc1cccnc1F)C1CCCCC1 ; CN1CCN(S(=O)(=O)n2cc(CN(CCCCN(C)C3CCCCC3)Cc3cccnc3F)c3ccccc32)CC1]

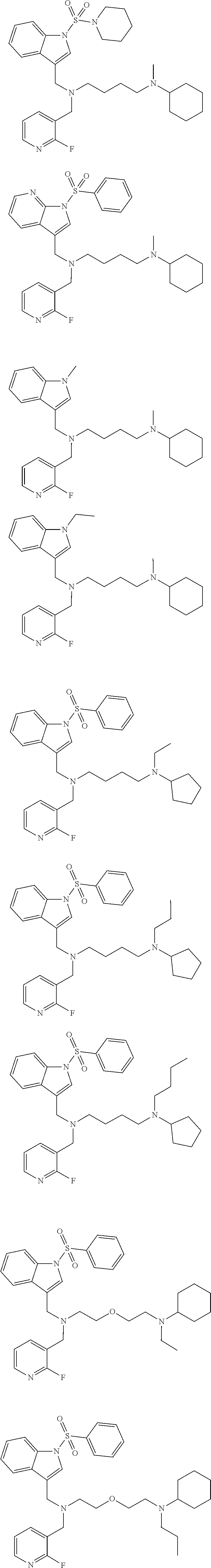

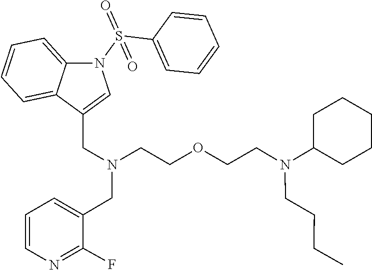 [SMILES: CCCCN(CCOCCN(Cc1cccnc1F)Cc1cn(S(=O)(=O)c2ccccc2)c2ccccc12)C1CCCCC1]

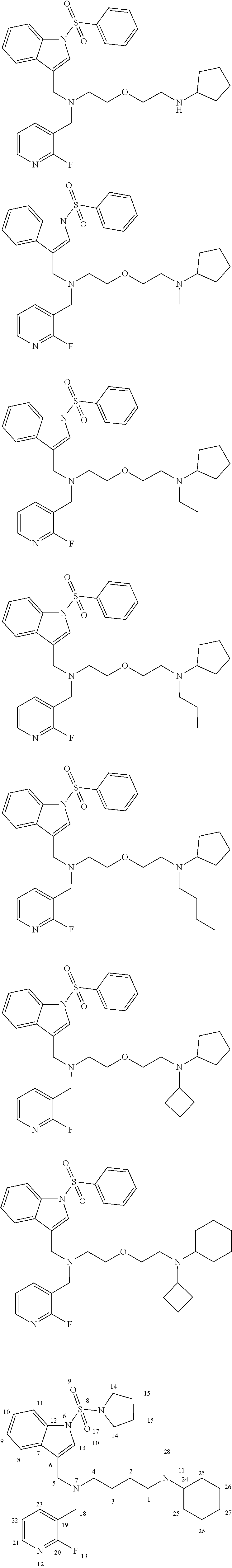

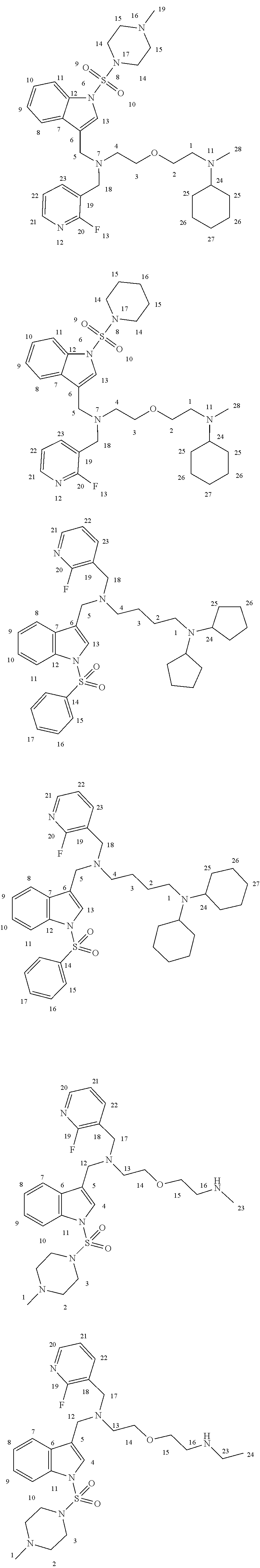 [SMILES: CN1CCN(S(=O)(=O)n2cc(CN(CCOCCN(C)C3CCCCC3)Cc3cccnc3F)c3ccccc32)CC1 ; CN(CCOCCN(Cc1cccnc1F)Cc1cn(S(=O)(=O)N2CCCCC2)c2ccccc12)C1CCCCC1 ; O=S(=O)(c1ccccc1)n1cc(CN(CCCCN(C2CCCC2)C2CCCC2)Cc2cccnc2F)c2ccccc21 ; O=S(=O)(c1ccccc1)n1cc(CN(CCCCN(C2CCCCC2)C2CCCCC2)Cc2cccnc2F)c2ccccc21 ; CNCCOCCN(Cc1cccnc1F)Cc1cn(S(=O)(=O)N2CCN(C)CC2)c2ccccc12 ; CCNCCOCCN(Cc1cccnc1F)Cc1cn(S(=O)(=O)N2CCN(C)CC2)c2ccccc12]

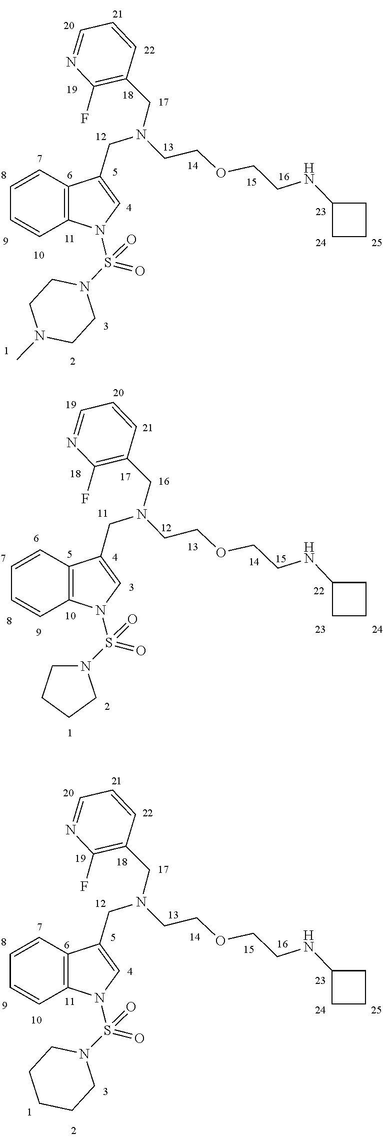 [SMILES: CN1CCN(S(=O)(=O)n2cc(CN(CCOCCNC3CCC3)Cc3cccnc3F)c3ccccc32)CC1 ; O=S(=O)(N1CCCC1)n1cc(CN(CCOCCNC2CCC2)Cc2cccnc2F)c2ccccc21 ; O=S(=O)(N1CCCCC1)n1cc(CN(CCOCCNC2CCC2)Cc2cccnc2F)c2ccccc21]

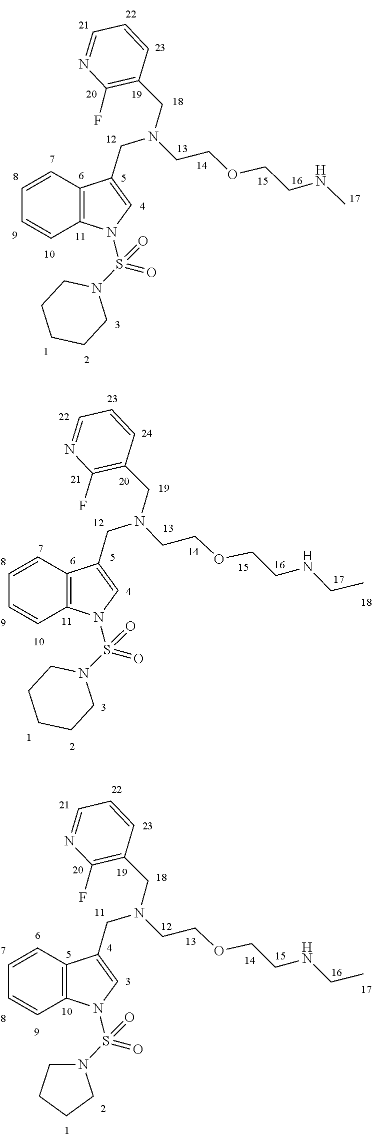 [SMILES: CNCCOCCN(Cc1cccnc1F)Cc1cn(S(=O)(=O)N2CCCCC2)c2ccccc12 ; CCNCCOCCN(Cc1cccnc1F)Cc1cn(S(=O)(=O)N2CCCCC2)c2ccccc12 ; CCNCCOCCN(Cc1cccnc1F)Cc1cn(S(=O)(=O)N2CCCC2)c2ccccc12]

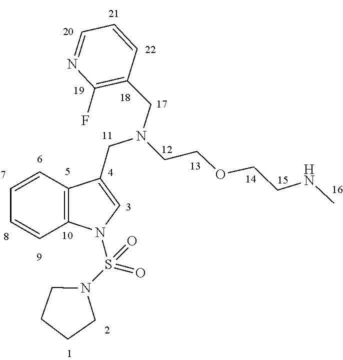 [SMILES: CNCCOCCN(Cc1cccnc1F)Cc1cn(S(=O)(=O)N2CCCC2)c2ccccc12]

8. A method for diagnosing, preventing, or treating a cytokine-mediated disease in an individual, comprising administering the compound or pharmaceutically acceptable salt or hydrate thereof according to claim 1 to the individual.

9. The method according to claim 8, wherein the cytokine is selected from the group consisting of IL-4, IL-5, IL-6, and TNFα.

10. The method according to claim 8, wherein the cytokine-mediated disease is selected from the group consisting of an inflammatory or autoimmune disorder, a neurological or neurodegenerative disorder, pain, a nociceptive disorder, a cardiovascular disorder, a metabolic disorder, an ocular disorder, and an oncological disorder.

11. The method according to claim 8, wherein the cytokine-mediated disease is Rheumatoid Arthritis (RA).

12. A pharmaceutical composition comprising as active substance at least one compound of formula (I) as defined in claim 1, or a pharmaceutically acceptable salt or hydrate thereof, optionally in association with at least one pharmaceutically acceptable vehicle.

13. A method for diagnosing, preventing or treating a cytokine-mediated disease in an individual, comprising administering the pharmaceutical composition of claim 12 to the individual.

14. The method according to claim 13, wherein the cytokine is selected from the group consisting of IL-4, IL-5, IL-6, and TNFα.

15. The method according to claim 13, wherein the cytokine-mediated disease is selected from the group consisting of an inflammatory or autoimmune disorder, a neurological or neurodegenerative disorder, pain, a nociceptive disorder, a cardiovascular disorder, a metabolic disorder, an ocular disorder, and an oncological disorder.

16. The pharmaceutical composition according to claim 12, further comprising at least one additional active substance.
